(19) United States
(12) Patent Application Publication
(10) Pub. No.: US 2015/0178462 A1
(43) Pub. Date: Jun. 25, 2015

(54) Methods and Compositions for Determining Smoking Status

(71) Applicants: Steven Rosenberg, Oakland, CA (US); Michael Reid Elashoff, Redwood City, CA (US); Phillip Beineke, Mountain View, CA (US); James A. Wingrove, Sunnyvale, CA (US)

(72) Inventors: Steven Rosenberg, Oakland, CA (US); Michael Reid Elashoff, Redwood City, CA (US); Phillip Beineke, Mountain View, CA (US); James A. Wingrove, Sunnyvale, CA (US)

(73) Assignee: CardioDx, INC., CA (US)

(21) Appl. No.: 14/241,156

(22) PCT Filed: Aug. 24, 2012
(86) PCT No.: PCT/US12/52303

§ 371 Date: Jul. 31, 2014

Related U.S. Application Data
(60) Provisional application No. 61/528,616, filed on Aug. 29, 2011.

(51) Int. Cl. G06F 19/00; C12Q 1/68

(52) U.S. Cl. CPC G06F 19/3431; C12Q 1/6883; C12Q 2600/158; C12Q 2600/16; C12Q 2600/118

(57) ABSTRACT

Peripheral blood markers are provided whose expression levels correlate with smoking status. Predictive models developed using highly informative markers are disclosed, along with systems, kits, and methods for using the markers to provide a biochemical surrogate for a subject's smoking status. In some embodiments, the smoking-related disease is chronic obstructive pulmonary disease, chronic bronchitis, emphysema, lung cancer, and/or asthma.

### CROSS REFERENCE TO RELATED APPLICATIONS

This application claims the benefit of U.S. Provisional Application No. 61/528,616, filed Aug. 29, 2011, the entire disclosure of which is hereby incorporated by reference in its entirety for all purposes.

### BACKGROUND

1. Field

The invention relates to predictive models for determining smoking status based on marker expression measurements, to their methods of use, and to computer systems and software for their implementation.

2. Description of the Related Art

Smoking is the leading cause of preventable death in the world, resulting in over 5 million deaths per year worldwide, with ˜500,000 of those deaths occurring in the United States (1,2). Smoking has been shown to be detrimental to human health, increasing the risk of multiple diseases, including many forms of cancers (lung, pancreatic) and cardiovascular/pulmonary disease (atherosclerosis, chronic obstructive pulmonary disease) (3,4,5). Cigarette smoke contains over 4,000 compounds, many of which have been shown to be carcinogenic or toxic; these compounds are able to enter the circulatory system by way of the pulmonary alveoli, and are distributed to different organs of the body, resulting in damage (6). During this process, circulatory cells of the immune system are exposed to these compounds, which may result in the changes in gene expression which can be assessed using established technologies.

Unmet Clinical and Scientific Need

Cotinine is a metabolite of nicotine and appears in the blood and urine of cigarette smokers. Biochemical measurements of cotinine in blood or urine therefore provide a marker of smoking status, but specialized assays are required. A general assay using readily available and general molecular biology tools such as quantitative RNA measures or nucleic acid sequencing reactions provides an independent way to determine smoking status and can be carried out as part of a parallel or multiplexed series of nucleic-acid based measures obtained from a patient sample.

### SUMMARY

Described herein is a computer-implemented method for scoring a sample obtained from a subject, wherein said score indicates said subject's smoking status, comprising: obtaining a dataset associated with said sample, wherein said dataset comprises quantitative expression data for one or more of marker 1, marker 2, marker 3, marker 4, and/or marker 5, wherein marker 1 is CLDND1 or IL7R, wherein marker 2 is LRRN3 or CCR7, wherein marker 3 is MUC1 or FOXP3, wherein marker 4 is GOPC or MCM3, and wherein marker 5 is LEF1 or CCR7; and determining, by a computer processor, a score from said dataset using an interpretation function, wherein said score is indicative of said subject's smoking status.

In some embodiments, said dataset comprises quantitative expression data for marker 1, marker 2, marker 3, marker 4 and marker 5, wherein marker 1 is CLDND1, wherein marker 2 is LRRN3, wherein marker 3 is MUC1, wherein marker 4 is GOPC, and wherein marker 5 is LEF1. In some embodiments, said dataset comprises quantitative expression data for two or more of marker 1, marker 2, marker 3, marker 4, and marker 5. In some embodiments, said dataset comprises quantitative expression data for three or more of marker 1, marker 2, marker 3, marker 4, and marker 5. In some embodiments, said dataset comprises quantitative expression data for four or more of marker 1, marker 2, marker 3, marker 4, and marker 5. In some embodiments, said dataset comprises quantitative expression data for marker 1, marker 2, marker 3, marker 4, and marker 5.

In some embodiments, the method further comprises, determining, by a computer processor, the subject's risk for developing a smoking-related disease, based on said score. In some embodiments, said smoking-related disease is chronic obstructive pulmonary disease, chronic bronchitis, emphysema, lung cancer, and/or asthma.

In some embodiments, the dataset comprises quantitative expression data for at least one, two, three, four, five, six, seven, eight, nine, ten, eleven, twelve, thirteen, fourteen, fifteen, sixteen, seventeen, eighteen, nineteen, twenty or more additional markers selected from Table 1.

In some embodiments, the dataset further comprises a clinical factor used to calculate the score. In some embodiments, the clinical factor is selected from the group consisting of: gender and hypertension. In some embodiments, the clinical factor is gender.

In some embodiments, said interpretation function is based on a predictive model. In some embodiments, said predictive model is selected from the group consisting of a partial least squares model, a logistic regression model, a linear regression model, a linear discriminant analysis model, a ridge regression model, and a tree-based recursive partitioning model. In some embodiments, said interpretation function is log(pr(Smoker)/(1−Pr(Smoker))=15.78306+0.3876*SEX−3.3368*CLDND1−3.4034*LRRN3−1.4847*MUC1+5.9209*GOPC+2.27166*LEF1, wherein SEX=1 if male, and 0 if female and Pr is probability. In some embodiments, said interpretation function is an interpretation function selected from the group of interpretation functions consisting of those set out in Table 7.

In some embodiments, obtaining said dataset associated with said sample comprises obtaining said sample and processing said sample to experimentally determine said dataset. In some embodiments, obtaining said dataset associated with said sample comprises receiving said dataset directly or indirectly from a third party that has processed said sample to experimentally determine said dataset.

In some embodiments, the dataset is obtained stored on a storage memory. In some embodiments, said quantitative expression data are from hybridization data. In some embodiments, said quantitative expression data are from polymerase chain reaction data. In some embodiments, said quantitative expression data are from sequence data.

Also described herein is a computer-implemented method for scoring a sample obtained from a subject, comprising: obtaining a dataset associated with the sample, wherein the dataset comprises a clinical factor used to calculate a score and quantitative expression level values for at least one marker selected from the group consisting of CLDND1, IL7R, LRRN3, CCR7, MUC1, FOXP3, GOPC, MCM3, LEF1, and CCR7; and determining, by a computer processor, the score from the dataset using an interpretation function, wherein the score is indicative of said subject's smoking status. In some embodiments, said dataset comprises quantitative expression data for CLDND1, LRRN3, MUC1, GOPC, and LEF1. In some embodiments, said dataset comprises quantitative expression data for two or more markers. In some embodiments, said dataset comprises quantitative expression data for three or more markers. In some embodiments, said dataset comprises quantitative expression data for four or more markers. In some embodiments, said dataset comprises quantitative expression data for five or more markers.

Also described herein is a system for scoring a sample obtained from a subject, wherein said score indicates said subject's smoking status, comprising: a storage memory for storing a dataset associated with said sample, wherein said dataset comprises quantitative expression data for one or more of marker 1, marker 2, marker 3, marker 4, and/or marker 5, wherein marker 1 is CLDND1 or IL7R, wherein marker 2 is LRRN3 or CCR7, wherein marker 3 is MUC1 or FOXP3, wherein marker 4 is GOPC or MCM3, and wherein marker 5 is LEF1 or CCR7; and a processor communicatively coupled to the storage memory for determining a score from said dataset using an interpretation function, wherein said score is indicative of said subject's smoking status.

Also described herein is a computer-readable storage medium storing computer-executable program code, the program code comprising: program code for storing a dataset associated with said sample, wherein said dataset comprises quantitative expression data for one or more of marker 1, marker 2, marker 3, marker 4, and/or marker 5, wherein marker 1 is CLDND1 or IL7R, wherein marker 2 is LRRN3 or CCR7, wherein marker 3 is MUC1 or FOXP3, wherein marker 4 is GOPC or MCM3, and wherein marker 5 is LEF1 or CCR7; and program code for determining a score from said dataset using an interpretation function, wherein said score is indicative of said subject's smoking status.

Also described herein is a method for scoring a sample obtained from a subject, wherein said score indicates said subject's smoking status, comprising: obtaining a sample from the subject, wherein the sample comprises a plurality of analytes; contacting the sample with a reagent; generating a plurality of complexes between the reagent and the plurality of analytes; detecting the plurality of complexes to obtain a dataset associated with said sample, wherein said dataset comprises quantitative expression data for one or more of marker 1, marker 2, marker 3, marker 4, and/or marker 5, wherein marker 1 is CLDND1 or IL7R, wherein marker 2 is LRRN3 or CCR7, wherein marker 3 is MUC1 or FOXP3, wherein marker 4 is GOPC or MCM3, and wherein marker 5 is LEF1 or CCR7; and determining a score from the dataset using an interpretation function, wherein said score is indicative of said subject's smoking status.

Also described herein is a kit for scoring a sample obtained from a subject, wherein said score indicates said subject's smoking status, comprising: a set of reagents comprising a plurality of reagents for determining from a sample obtained from the subject quantitative expression data for one or more of marker 1, marker 2, marker 3, marker 4, and/or marker 5, wherein marker 1 is CLDND1 or IL7R, wherein marker 2 is LRRN3 or CCR7, wherein marker 3 is MUC1 or FOXP3, wherein marker 4 is GOPC or MCM3, and wherein marker 5 is LEF1 or CCR7; and instructions for using the plurality of reagents to determine quantitative expression data in a dataset from the sample, wherein the instructions comprise instructions for determining, by a computer processor, a score from said dataset using an interpretation function, wherein said score is indicative of said subject's smoking status.

In an embodiment, the invention provides a method for determining the smoking status of a subject through use of a dataset that includes quantitative expression data for one or more markers listed in Table 1, by analyzing the dataset to determine an expression level of the marker, wherein the expression level of the marker positively or negatively correlates with the smoking status of the subject, thereby determine the subject's smoking status. In an embodiment, the subject's smoking status can be used to assess risk of developing a smoking-related disease such as chronic obstructive pulmonary disease, chronic bronchitis, emphysema, lung cancer, or asthma. In an embodiment, the analyzing step is carried out by comparing the expression level of the marker to a threshold value. In other embodiments, the dataset includes quantitative expression data for at least two, three, four, five, six, seven, eight, nine, ten, eleven, twelve, thirteen, fourteen, fifteen, sixteen, seventeen, eighteen, nineteen, twenty or more markers selected from Table 1. The marker may be positively or negatively correlated with smoking status, and the expression level of the marker may be increased or decreased in a smoker as compared to a non-smoker. In other aspects, the methods of the invention are implemented on one or more computers. In some embodiments, the dataset is obtained by assaying a sample to experimentally determine expression values. In other embodiments, the dataset is obtained directly or indirectly from a third party that has processed the sample to experimentally determine the data. The data in the dataset may reflect measurements made using a nucleotide-based assay such as a qRT-PCR assay, a hybridization assay, or a sequencing reaction assay. In some embodiments, the methods of the invention are implemented using a computer processor. The invention also encompasses systems for determining smoking status of a subject. The system includes a storage memory for storing the dataset, and a processor communicatively coupled to the storage memory for analyzing the dataset to determine the expression level of the marker. In other embodiments, the invention includes a computer-readable storage medium storing computer-executable program code for storing a dataset associated with a sample obtained from the subject, which dataset includes quantitative expression data for a marker selected from Table 1 and program code for analyzing the dataset to determine an expression level of the marker, where the expression level positively or negatively correlates with the subject's smoking status. In yet other embodiments, the system of the intention includes a storage memory for storing a dataset that includes a threshold value for a marker selected from Table 1. The threshold can be associated with expression data obtained from a non-smoking subject or a non-smoking population of subjects. Still other embodiments of the invention include a kit for use in determining a subject's smoking status, that includes a set of reagents for determining from a sample obtained from the subject quantitative expression data for a marker selected from Table 1 and instructions for using the reagents to determine quantitative expression data from the sample and analyzing the dataset to determine an expression level of the marker, which positively or negatively correlates with the subject's smoking status. The instructions may further include threshold values for use in the analysis and/or an interpretation function for generating a score indicative of smoking status. The kit may include reagents for more than one marker selected from Table 1, for example, at least two, three, four, five, six, seven, eight, nine, ten, eleven, twelve, thirteen, fourteen, fifteen, sixteen, seventeen, eighteen, nineteen, twenty or more markers. Embodiments of the invention also incorporate predictive models and associated interpretation functions that operate on the quantitative expression data to generate a score that is indicative of a subject's smoking status. The predictive model can be a partial least squares model, a logistic regression model, a linear regression model, a linear discriminant analysis model, a ridge regression model, and a tree-based recursive partitioning model. In certain embodiments, the markers comprise CDND1, LRRN3, MUC1, GOPC, or LEF1, or markers selected from Table 1 whose expression is correlated with CDND1, LRRN3, MUC1, GOPC, and LEF1. In some embodiments, the interpretation function is log(pr(Smoker)/(1−Pr(Smoker))=15.78306+0.3876*SEX−3.3368*CLDND1−3.4034*LRRN3−1.4847*MUC1+5.9209*GOPC+2.27166*LEF1, wherein SEX=1 if male, and 0 if female and Pr is probability. In other embodiments, the interpretation function is an interpretation function set out in Table 7.

### BRIEF DESCRIPTION OF THE DRAWING

These and other features, aspects, and advantages of the present invention will become better understood with regard to the following description, and accompanying drawing, where:

FIG. 1—is a plot showing relationship between classification by application of predictive model of samples into smoker and non-smoker categories and mean cotinine levels for the same samples.

### DETAILED DESCRIPTION

In general, terms used in the claims and the specification are intended to be construed as having the plain meaning understood by a person of ordinary skill in the art. Certain terms are defined below to provide additional clarity. In case of conflict between the plain meaning and the provided definitions, the provided definitions are to be used.

The term “Ct” refers to cycle threshold and is defined as the PCR cycle number where the fluorescent value is above a set threshold. Therefore, a low Ct value corresponds to a high level of expression, and a high Ct value corresponds to a low level of expression.

The term “Cp” refers to the crossing point and is defined as the intersection of the best fit of the log-linear portion of a standard's amplification curve in a real time PCR instrument such as, e.g., a LightCycler, and the noise band (set according to background fluorescence measurements).

The term “FDR” means to false discovery rate. FDR can be estimated by analyzing randomly-permuted datasets and tabulating the average number of genes at a given p-value threshold.

The terms “GL” “GM” and “GU” respectively refer to 1st percentile, median, and 99th percentile of Cp for that gene in the Algorithm Development data set.

The terms “marker” or “markers” encompass, without limitation, lipids, lipoproteins, proteins, cytokines, chemokines, growth factors, peptides, nucleic acids, genes, and oligonucleotides, together with their related complexes, metabolites, mutations, variants, polymorphisms, modifications, fragments, subunits, degradation products, elements, and other analytes or sample-derived measures. A marker can also include mutated proteins, mutated nucleic acids, variations in copy numbers, and/or transcript variants, in circumstances in which such mutations, variations in copy number and/or transcript variants are useful for generating a predictive model, or are useful in predictive models developed using related markers (e.g., non-mutated versions of the proteins or nucleic acids, alternative transcripts, etc.).

The terms “highly correlated gene expression” or “highly correlated marker expression” refer to gene or marker expression values that have a sufficient degree of correlation to allow their interchangeable use in a predictive model of coronary artery disease. For example, if gene x having expression value X is used to construct a predictive model, highly correlated gene y having expression value Y can be substituted into the predictive model in a straightforward way readily apparent to those having ordinary skill in the art and the benefit of the instant disclosure. Assuming an approximately linear relationship between the expression values of genes x and y such that Y=a+bX, then X can be substituted into the predictive model with (Y−a)/b. For non-linear correlations, similar mathematical transformations can be used that effectively convert the expression value of gene y into the corresponding expression value for gene x. The terms “highly correlated marker” or “highly correlated substitute marker” refer to markers that can be substituted into and/or added to a predictive model based on, e.g., the above criteria. A highly correlated marker can be used in at least two ways: (1) by substitution of the highly correlated marker(s) for the original marker(s) and generation of a new model for predicting smoking status; or (2) by substitution of the highly correlated marker(s) for the original marker(s) in the existing model for predicting smoking status.

The term “mammal” encompasses both humans and non-humans and includes but is not limited to humans, non-human primates, canines, felines, murines, bovines, equines, and porcines.

The term “metagene” refers to a set of genes whose expression values are combined to generate a single value that can be used as a component in a predictive model. (Brunet, J. P., et al. Proc. Natl. Acad. Sciences 2004; 101(12):4164-9)

The term “sample” can include a single cell or multiple cells or fragments of cells or an aliquot of body fluid, taken from a subject, by means including venipuncture, excretion, ejaculation, massage, biopsy, needle aspirate, lavage sample, scraping, surgical incision, or intervention or other means known in the art.

The term “subject” encompasses a cell, tissue, or organism, human or non-human, whether in vivo, ex vivo, or in vitro, male or female.

The term “obtaining a dataset associated with a sample” encompasses obtaining a set of data determined from at least one sample. Obtaining a dataset encompasses obtaining a sample, and processing the sample to experimentally determine the data. The phrase also encompasses receiving a set of data, e.g., from a third party that has processed the sample to experimentally determine the dataset. Additionally, the phrase encompasses mining data from at least one database or at least one publication or a combination of databases and publications. A dataset can be obtained by one of skill in the art via a variety of known ways including stored on a storage memory.

It must be noted that, as used in the specification and the appended claims, the singular forms “a,” “an,” and “the” include plural referents unless the context clearly dictates otherwise.

Methods

Markers and Clinical Factors

The quantity of one or more markers of the invention can be indicated as a value. A value can be one or more numerical values resulting from evaluation of a sample under a condition. The values can be obtained, for example, by experimentally obtaining measures from a sample by an assay performed in a laboratory, or alternatively, obtaining a dataset from a service provider such as a laboratory, or from a database or a server on which the dataset has been stored, e.g., on a storage memory.

In an embodiment, the quantity of one or more markers can be one or more numerical values associated with expression levels of the genes set out in Table 1 resulting from evaluation of a sample under a condition. The column labels of Table 1 indicate the following: “Probe Name” refers to the names of probes found on Agilent Human Whole Genome Arrays (Agilent Technologies, Santa Clara, Calif.); “Gene Name” refers to the names of human genes in accordance with guidelines provided by the Human Genome Organization (HUGO) Gene Nomenclature Committee (HGNC). Further information about each human gene, such as accession number(s) and aliases, can be found by entering the gene name into the search page on the HGNC genenames.org website. For example, entering the term “LRRN3” into the Simple Search field of the HGNC website on Aug. 10, 2011 returns the approved gene name of LRRN3 (leucine rich repeat neuronal 3), the sequence accession IDs of LRRN3 (GenBank AB060967; RefSeq: NM 001099658), and the previous symbols or synonyms for LRRN3 (FIGLER5, F1111129, NLRR3). Further human gene names are provided in the Examples section below. A person of ordinary skill in the art recognizes that the Gene Name information provided in Table 1 unambiguously identifies genes used as biomarkers in the present invention, and is able to use the Gene Name information of Table 1 to obtain protein and nucleic acid sequence information about the named gene without exercising undue experimentation. Such information readily enables the skilled artisan to obtain quantitative expression level data for these markers using any one of a number of methods described in this specification. “Smoking Log Odds” refers to a standard statistical measure of the association of a biomarker with smoking status. A positive value in Table 1 indicates that the marker is positively associated with smoking status, while a negative value indicates that the marker is negatively associated with smoking status (i.e., that the marker is associated with negative (“non-smoking”) smoking status. Thus, if expression goes down with increased smoking, the marker has a negative value, and if expression goes up with increased smoking, the marker has positive value in Table 1. “Smoking p” refers to the statistical significance of a marker's association (positive or negative) with smoking status.

In an embodiment, a marker's associated value can be included in a dataset associated with a sample obtained from a subject. A dataset can include the marker expression value of two or more, three or more, four or more, five or more, six or more, seven or more, eight or more, nine or more, ten or more, eleven or more, twelve or more, thirteen or more, fourteen or more, fifteen or more, sixteen or more, seventeen or more, eighteen or more, nineteen or more, twenty or more, twenty-one or more, twenty-two or more, twenty-three or more, twenty-four or more, twenty-five or more, twenty-six or more, twenty-seven or more, twenty-eight or more, twenty-nine or more, or thirty or more marker(s) set out in Table 1. A dataset can include a subset or a complete set of the markers set out in Table 1 with other markers now known or later determined to be positively or negatively associated with smoking status. For example, a dataset can include the expression values for SASH1, P2RY6, MUC1, LRRN3, MGAT3, and CLDND1. In another embodiment, a dataset can include the expression values for CLDND1, LRRN3, MUC1, GOPC, and LEF1. Other combinations are described in more detail in the Examples section below. A dataset may also combine expression values for markers with a clinical factor, e.g., gender. A dataset may also combine expression values for markers with an indicator of a subject's sex (i.e., an indication of whether the subject is male or female). A dataset may also combine expression values for markers with an indicator of a subject's hypertension status.

In another embodiment, the invention includes obtaining a sample associated with a subject, where the sample includes one or more markers. The sample can be obtained by the subject or by a third party, e.g., a medical professional. Examples of medical professionals include physicians, emergency medical technicians, nurses, first responders, psychologists, medical physics personnel, nurse practitioners, surgeons, dentists, and any other obvious medical professional as would be known to one skilled in the art. A sample can include peripheral blood cells, isolated leukocytes, or RNA extracted from peripheral blood cells or isolated leukocytes. The sample can be obtained from any bodily fluid, for example, amniotic fluid, aqueous humor, bile, lymph, breast milk, interstitial fluid, blood, blood plasma, cerumen (earwax), Cowper's fluid (pre-ejaculatory fluid), chyle, chyme, female ejaculate, menses, mucus, saliva, urine, vomit, tears, vaginal lubrication, sweat, serum, semen, sebum, pus, pleural fluid, cerebrospinal fluid, synovial fluid, intracellular fluid, and vitreous humour. In an example, the sample is obtained by a blood draw, where the medical professional draws blood from a subject, such as by a syringe. The bodily fluid can then be tested to determine the value of one or more markers using an assay. The value of the one or more markers can then be evaluated by the same party that performed the assay using the methods of the invention or sent to a third party for evaluation using the methods of the invention.

Smoking status is well known to correlate with certain smoking-related disease risks. These include chronic obstructive pulmonary disease (COPD), chronic bronchitis, emphysema, lung cancer, asthma (11, 12). Thus, the methods of the invention can be used to provide an independent risk factor to assess an individual's risk of developing one or more smoking-related disease. The result from the methods of the invention can be fed into any one of a number of diagnostic processes that use smoking status to assess a smoking-related disease risk. These results can be used in lieu of or in addition to an individual's self-reported smoking status, for example, in providing patient history data to a physician, to an insurance carrier or to any other entity that was interested in assessing an individual's risk of developing one or more smoking-related diseases.

Interpretation Functions

In an embodiment, an interpretation function can be a function produced by a predictive model. An interpretation function can also be produced by a plurality of predictive models. In an embodiment, an interpretation function can take the form of: log (Pr(Smoker)/(1−Pr(Smoker))=15.7306+0.3876*SEX−3.3368*CLDND1−3.4034*LRRN3−1.4847*MUC1+5.9209*GOPC+2.7166*LEF1 where SEX=1 if male, 0 if female; Pr=probability. Other interpretation functions are set out in Table 7.

In an embodiment, a predictive model can include a partial least squares model, a logistic regression model, a linear regression model, a linear discriminant analysis model, a ridge regression model, and a tree-based recursive partitioning model. In an embodiment, a predictive model can also include Support Vector Machines, quadratic discriminant analysis, or a LASSO regression model. See Elements of Statistical Learning, Springer 2003, Hastie, Tibshirani, Friedman; which is herein incorporated by reference in its entirety for all purposes. Predictive model performance can be characterized by an area under the curve (AUC). In an embodiment, predictive model performance is characterized by an AUC ranging from 0.68 to 0.70. In an embodiment, predictive model performance is characterized by an AUC ranging from 0.70 to 0.79. In an embodiment, predictive model performance is characterized by an AUC ranging from 0.80 to 0.89. In an embodiment, predictive model performance is characterized by an AUC ranging from 0.90 to 0.99. Interpretation functions can be developed using combinations of informative markers as shown in the Examples below, or using a single gene whose expression is highly correlated with smoking status. In certain embodiments, methods for classifying based on a single gene are developed using logistic regression or linear discriminant analysis (LDA).

Assays

Examples of assays for one or more markers include DNA assays, microarrays, sequencing-based assays in which the number of sequenced molecules is counted and the count used to determine expression level. The sequenced molecules can be cDNAs corresponding to mRNA transcripts. Other assays include polymerase chain reaction (PCR), RT-qPCR, sequencing assays, Southern blots, Northern blots, antibody-binding assays, enzyme-linked immunosorbent assays (ELISAs), flow cytometry, protein assays, Western blots, nephelometry, turbidimetry, chromatography, mass spectrometry, immunoassays, including, by way of example, but not limitation, RIA, immunofluorescence, immunochemiluminescence, immunoelectrochemiluminescence, or competitive immunoassays, immunoprecipitation, and the assays described in the Examples section below. The information from the assay can be quantitative and sent to a computer system of the invention. The information can also be qualitative, such as observing patterns or fluorescence, which can be translated into a quantitative measure by a user or automatically by a reader or computer system. In an embodiment, the subject can also provide information other than assay information to a computer system, such as a clinical factor (e.g., gender).

In addition to the use of RT-qPCR to assess expression levels, other modalities such as microarrays or RNA sequencing can be used. For example, to crosswalk a predictive model based on RT-qPCR data to microarray data, the array data is first subjected to standard normalization. A regression line is then fit to predict the PCR value for each of the model genes from its array value. The fitted values of each regression are then inserted into the smoking model as predictors. To crosswalk a predictive model to RNA sequencing, targeted re-sequencing of the model genes is accomplished using a next-generation sequencing platform. Raw sequence reads are aligned to the respective targeted genes, and raw expression levels assessed by calculating depth of coverage. Raw values are normalized by the total number of raw sequences per sample and length of target gene. A regression line is then fit to predict the PCR value for each of the model genes from its normalized sequence value. The fitted values of each regression are inserted into the smoking model as predictors.

Informative Marker Groups

In addition to the specific, exemplary markers identified in this application by name, accession number, or sequence, included within the scope of the invention are all operable predictive models of smoking status and methods for their use to score and optionally classify samples using expression values of variant sequences having at least 90% or at least 95% or at least 97% or greater identity to the exemplified sequences or that encode proteins having sequences with at least 90% or at least 95% or at least 97% or greater identity to those encoded by the exemplified genes or sequences. The percentage of sequence identity may be determined using algorithms well known to those of ordinary skill in the art, including, e.g., BLASTn, and BLASTp, as described in Stephen F. Altschul et al., J. Mol. Biol. 215:403-410 (1990) and available at the National Center for Biotechnology Information website maintained by the National Institutes of Health. As described below, in accordance with an embodiment of the present invention, are all operable predictive models and methods for their use in scoring and optionally classifying samples that use a marker expression measurement that is now known or later discovered to be highly correlated with the expression of an exemplary marker expression value in addition to or in lieu of that exemplary marker expression value. For the purposes of the present invention, such highly correlated genes are contemplated either to be within the literal scope of the claimed inventions or alternatively encompassed as equivalents to the exemplary markers. Identification of markers having expression values that are highly correlated to those of the exemplary markers, and their use as a component of a predictive model is well within the level of ordinary skill in the art. The Examples section below provides numerous examples of methods for identifying highly correlated markers and substituting them for algorithm markers in predictive models of smoking status and methods for their use to score and optionally classify samples.

Computer Implementation

In one embodiment, a computer comprises at least one processor coupled to a chipset. Also coupled to the chipset are a memory, a storage device, a keyboard, a graphics adapter, a pointing device, and a network adapter. A display is coupled to the graphics adapter. In one embodiment, the functionality of the chipset is provided by a memory controller hub and an I/O controller hub. In another embodiment, the memory is coupled directly to the processor instead of the chipset.

The storage device is any device capable of holding data, like a hard drive, compact disk read-only memory (CD-ROM), DVD, or a solid-state memory device. The memory holds instructions and data used by the processor. The pointing device may be a mouse, track ball, or other type of pointing device, and is used in combination with the keyboard to input data into the computer system. The graphics adapter displays images and other information on the display. The network adapter couples the computer system to a local or wide area network.

As is known in the art, a computer can have different and/or other components than those described previously. In addition, the computer can lack certain components. Moreover, the storage device can be local and/or remote from the computer (such as embodied within a storage area network (SAN)).

As is known in the art, the computer is adapted to execute computer program modules for providing functionality described herein. As used herein, the term “module” refers to computer program logic utilized to provide the specified functionality. Thus, a module can be implemented in hardware, firmware, and/or software. In one embodiment, program modules are stored on the storage device, loaded into the memory, and executed by the processor.

The term percent “identity,” in the context of two or more nucleic acid or polypeptide sequences, refer to two or more sequences or subsequences that have a specified percentage of nucleotides or amino acid residues that are the same, when compared and aligned for maximum correspondence, as measured using one of the sequence comparison algorithms described below (e.g., BLASTP and BLASTN or other algorithms available to persons of skill) or by visual inspection. Depending on the application, the percent “identity” can exist over a region of the sequence being compared, e.g., over a functional domain, or, alternatively, exist over the full length of the two sequences to be compared.

For sequence comparison, typically one sequence acts as a reference sequence to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally Ausubel et al., infra).

One example of an algorithm that is suitable for determining percent sequence identity and sequence similarity is the BLAST algorithm, which is described in Altschul et al., J. Mol. Biol. 215:403-410 (1990). Software for performing BLAST analyses is publicly available through the National Center for Biotechnology Information.

Embodiments of the entities described herein can include other and/or different modules than the ones described here. In addition, the functionality attributed to the modules can be performed by other or different modules in other embodiments. Moreover, this description occasionally omits the term “module” for purposes of clarity and convenience.

Kits

The invention provides kits for determining quantitative expression data for one or more markers selected from Table 1 and instructions for using the data to determine a subject's smoking status. Optionally the kid may include packaging. The kit can include reagents for carrying out a nucleotide-based assay such as a qRT-PCR assay, a hybridization assay, or a sequencing assay for determining the expression levels of the one or markers selected from Table 1. The kit can include reagents for carrying out any of the other types of assays described in this specification. The reagents can be probes and primers such as those set out in Table 4, or other similar reagents. The reagents can be probes such as the probes identified in Table 1 or Table 2. The instructions can include an interpretation function that is used to operate on the quantitative expression data. The interpretation function can be generated from a predictive model. The instructions can include thresholds that can be determined from a smoking subject or a smoking population of subjects, or from a non-smoking subject or a non-smoking population of subjects. The instructions can include methods for comparing the quantitative expression data to a threshold for determining smoking status.

### EXAMPLES

Below are examples of specific embodiments for carrying out the present invention. The examples are offered for illustrative purposes only, and are not intended to limit the scope of the present invention in any way. Efforts have been made to ensure accuracy with respect to numbers used (e.g., amounts, temperatures, etc.), but some experimental error and deviation should, of course, be allowed for.

The practice of the present invention will employ, unless otherwise indicated, conventional methods of protein chemistry, biochemistry, recombinant DNA techniques and pharmacology, within the skill of the art. Such techniques are explained fully in the literature. See, e.g., T. E. Creighton, Proteins: Structures and Molecular Properties (W.H. Freeman and Company, 1993); A. L. Lehninger, Biochemistry (Worth Publishers, Inc., current addition); Sambrook, et al., Molecular Cloning: A Laboratory Manual (2nd Edition, 1989); Methods In Enzymology (S. Colowick and N. Kaplan eds., Academic Press, Inc.); Remington's Pharmaceutical Sciences, 18th Edition (Easton, Pa.: Mack Publishing Company, 1990); Carey and Sundberg Advanced Organic Chemistry 3^{rd} Ed. (Plenum Press) Vols A and B(1992).

Materials and Methods

Statistical Methods

All statistical methods were performed using the R software package. The statistical methods used are described and referenced in greater detail below.

Gene Selection

Genes for RT-PCR were selected based on significance, fold-change, pathway analysis, and literature support. Hierarchical clustering based on gene: gene correlations ensured that RT-PCR genes represented multiple clusters. Normalization genes were selected based on low variance, moderate to high expression, and no significant association with case: control status, sex, age, or cell counts.

PCR Statistical Analysis

Clinical/demographic factors were assessed for smoking status association using univariate and multivariate logistic regression. Gene expression association with smoking status and other clinical/demographic factors was assessed by robust logistic regression (unadjusted and sex/age adjusted).⁷

Whole Genome Microarray Analysis

Phase I—PREDICT DISCOVERY.

We performed whole genome microarray analysis on RNA isolated from 210 catheter lab patients enrolled in a prospective clinical trial (PREDICT) designed to identify gene expression signatures that correlate with coronary artery disease.

Blood was collected at the time of catheterization in PAXgen tubes. RNA was isolated by automated method, using the Agencourt RNAdvance system, and quantified using Ribogreen (Invitrogen (now Life Technologies), Carlsbad, Calif.). RNA was labeled with Cy3 using methods recommended by the manufacturer (Agilent, Santa Clara, Calif.) and hybridized to whole genome arrays (Agilent Human Whole Genome Arrays).

Array Normalization

Agilent processed signal values for array normalization were scaled to a trimmed mean of 100 and then log 2 transformed. Standard array QC metrics (percent present, pairwise correlation, and signal intensity) were used for quality assessment, resulting in 12 of 210 PREDICT samples being excluded.

Array Analysis

For the PREDICT array, given the paired design, conditional logistic regression was used. False discovery rates were used to account for multiple comparisons. GOEAST was used to determine over-representation of Gene Ontology (GO) terms.

Array feature data was extracted using Agilent Feature Extraction software and normalized using quantile normalization.

Algorithm Calculation and Transformation

Data Preprocessing and QC Steps
1) The median of triplicate wells for each algorithm gene/sample was computed
   a. If one well had no call, the median of the two remaining wells was used
   b. If two or three wells had no call, the algorithm gene received no call for that sample
2) The median of the algorithm gene SD's was computed. If this value was greater than 0.15, the sample failed for High Replicate SD.
3) For each algorithm gene i, a floor Cp value was created by replacing values less than GLᵢ with GLᵢ This value represents the 1^{st} percentile of Cp for that gene in the Algorithm Development set.
4) For each algorithm gene i, a ceiling Cp value was created by replacing values greater than GUᵢ with GUᵢ. This value represents the 99^{th} percentile of Cp for that gene in the Algorithm Development set.
5) For each algorithm gene i, the absolute value of the difference between its Cp value and GMᵢ was computed, where GMᵢ represents the median Cp for that gene in the Algorithm Development set. This value was summed across the algorithm genes. If the sum was greater than 27.17, the sample failed for Expression Profile Out of Range. 27.17 represents the largest value of this metric within the Algorithm Development set.

In certain cases, an algorithm score was not be calculated for a subject. Reasons for this included low PAXgene® tube blood volume, lab QC failure, etc. The frequency of occurrence of these failures was tabulated, though these subjects were not included in the analysis set.

To identify genes whose expression levels correlated with smoking, a robust linear model was used (8), with smoking status used as the dependent variable and age, sex, and gene expression as the independent variables. Table 1 contains the 4988 probes (representing 4214 genes) which showed significant correlation with smoking status (p<0.05). 1933 probes were down-regulated (indicated by negative smoking Log Odds) in smokers and 3055 were up-regulated (indicated by positive smoking Log Odds) (Table 1).

TABLE 1

| | | | Smoking | |
|---|---|---|---|---|
| Probe Name | Accession Number | Gene Name | Log Odds | Smoking p |
| A_24_P123385 | NM_005909 | MAP1B | −91.68 | 0.008102221 |
| A_23_P105524 | NM_033123 | PLCZ1 | −80.37 | 0.019425846 |
| A_24_P205045 | NM_015576 | ERC2 | −80.04 | 0.032759299 |
| A_24_P316335 | AB070620 | AB070620 | −69.95 | 0.033598626 |
| A_24_P935437 | AK123433 | MBP | −67.56 | 0.037028443 |
| A_23_P363149 | AF034209 | AF034209 | −66.1 | 0.046389678 |
| A_32_P100452 | THC2654273 | THC2654273 | −65.73 | 0.038337597 |
| A_32_P40946 | THC2696194 | THC2696194 | −65.55 | 0.03319935 |
| A_32_P228683 | AW293859 | AW293859 | −65.12 | 0.039119592 |
| A_24_P65121 | NM_001040655 | TTC23 | −63.93 | 0.040492796 |
| A_23_P115261 | NM_000029 | AGT | −58.5 | 0.049559402 |
| A_24_P915300 | NM_032184 | SYDE2 | −56.34 | 0.009652812 |
| A_24_P402090 | BC031604 | C17orf71 | −52.6 | 0.043160989 |
| A_24_P410900 | AF130117 | LOC100129198 | −39.29 | 0.045410565 |
| A_24_P915566 | A_24_P915566 | A_24_P915566 | −38.68 | 0.029926316 |
| A_24_P179225 | NM_030583 | MATN2 | −37.37 | 0.019161645 |
| A_24_P323148 | NM_182573 | LYPD5 | −29.57 | 0.044004222 |
| A_24_P222441 | BC034424 | HEXA | −22.7 | 0.028023605 |
| A_23_P202448 | NM_199168 | CXCL12 | −18.06 | 0.034785065 |
| A_23_P127107 | NM_152311 | CLRN3 | −15.69 | 0.049678973 |
| A_32_P441530 | NM_207335 | KLHDC6 | −15.21 | 0.044856344 |
| A_23_P113111 | NM_000044 | AR | −14.08 | 0.003701352 |
| A_23_P113111 | NM_000044 | AR | −13.43 | 0.001825987 |
| A_23_P113111 | NM_000044 | AR | −12.93 | 0.001410916 |
| A_24_P599340 | NM_015550 | OSBPL3 | −12.29 | 0.027850022 |
| A_23_P113111 | NM_000044 | AR | −12.25 | 0.00299763 |
| A_23_P202245 | NM_020975 | RET | −11.24 | 0.048959904 |
| A_32_P98739 | NM_001080444 | IGSF5 | −10.85 | 0.04847366 |
| A_23_P113111 | NM_000044 | AR | −10.7 | 0.000840556 |
| A_32_P5057 | AF070541 | LOC284244 | −9.923 | 0.022174238 |
| A_24_P484791 | AK094649 | LOC283089 | −9.63 | 0.025602335 |
| A_23_P113111 | NM_000044 | AR | −9.247 | 0.0058246 |
| A_23_P202206 | NM_183239 | GSTO2 | −9.177 | 0.008495699 |
| A_24_P112110 | NM_001518 | GTF2I | −8.852 | 0.043632467 |
| A_23_P167237 | NM_031911 | C1QTNF7 | −8.672 | 0.043389878 |
| A_32_P226186 | NM_020910 | KIAA1549 | −8.274 | 0.023535877 |
| A_23_P113111 | NM_000044 | AR | −8.223 | 0.001886501 |
| A_23_P215828 | NM_022820 | CYP3A43 | −8.161 | 0.013403194 |
| A_24_P69084 | NM_139056 | ADAMTS16 | −8.026 | 0.049403182 |
| A_32_P174805 | AI492422 | AI492422 | −7.762 | 0.038678344 |
| A_24_P134863 | NM_198510 | ITIH5L | −7.641 | 0.016079114 |
| A_23_P113111 | NM_000044 | AR | −7.64 | 0.007411684 |
| A_23_P113111 | NM_000044 | AR | −7.365 | 0.019611991 |
| A_23_P23572 | NM_003557 | PIP5K1A | −7.333 | 0.011014245 |
| A_24_P43810 | NM_207006 | FAM83A | −7.3 | 0.03031582 |
| A_32_P133106 | BM978707 | BM978707 | −6.984 | 0.011484109 |
| A_23_P302672 | NM_145244 | DDIT4L | −6.921 | 0.020803232 |
| A_23_P41344 | NM_001432 | EREG | −6.914 | 0.011241005 |
| A_24_P929957 | THC2500744 | THC2500744 | −6.796 | 0.033695121 |
| A_32_P214020 | AK124256 | AK124256 | −6.662 | 0.015981967 |
| A_24_P936444 | AF323119 | NFE2L2 | −6.612 | 0.002377299 |
| A_24_P158298 | AK025116 | AK025116 | −6.341 | 0.021968079 |
| A_23_P48264 | NM_181783 | TMTC3 | −6.339 | 0.029564695 |
| A_23_P113111 | NM_000044 | AR | −6.314 | 0.010932518 |
| A_23_P167030 | NM_000316 | PTHR1 | −6.061 | 0.023651686 |
| A_24_P42624 | NM_003728 | UNC5C | −5.901 | 0.02161711 |
| A_24_P935804 | AK095688 | CCDC66 | −5.812 | 0.003267218 |
| A_23_P373521 | NM_021973 | HAND2 | −5.706 | 0.036892189 |
| A_23_P20101 | NM_016944 | TAS2R4 | −5.651 | 0.023956476 |
| A_24_P917219 | THC2550160 | THC2550160 | −5.459 | 0.015473638 |
| A_32_P46603 | NM_144992 | VWA3B | −5.448 | 0.00532198 |
| A_24_P187938 | BC002741 | BC002741 | −5.414 | 0.031283971 |
| A_24_P76757 | BC019583 | BC019583 | −5.394 | 0.04322091 |
| A_23_P62387 | NM_018159 | NUDT11 | −5.317 | 0.027884786 |
| A_23_P216679 | NM_033331 | CDC14B | −5.196 | 0.015430582 |
| A_23_P386514 | NM_005506 | SCARB2 | −5.136 | 0.032999596 |
| A_23_P98923 | NM_006390 | IPO8 | −5.113 | 0.043681076 |
| A_32_P35692 | THC2692879 | THC2692879 | −5.042 | 0.049934183 |
| A_23_P35755 | NM_000372 | TYR | −4.998 | 0.034143127 |
| A_32_P179138 | BC039414 | BC039414 | −4.992 | 0.006605846 |
| A_23_P156890 | NM_003206 | TCF21 | −4.986 | 0.02861737 |
| A_23_P347610 | NM_012206 | HAVCR1 | −4.952 | 0.013516988 |
| A_23_P160214 | NM_001080494 | C1orf34 | −4.947 | 0.020710762 |
| A_23_P108427 | THC2604598 | THC2604598 | −4.939 | 0.017777599 |
| A_23_P43898 | NM_173567 | ABHD7 | −4.93 | 0.022614957 |
| A_32_P48842 | CR595668 | LOC644538 | −4.929 | 0.002827534 |
| A_32_P49854 | THC2697511 | THC2697511 | −4.867 | 0.000506624 |
| A_24_P919063 | A_24_P919063 | A_24_P919063 | −4.839 | 0.002347836 |
| A_23_P400716 | NM_001010846 | SHE | −4.821 | 0.018678339 |
| A_23_P1320 | NM_021245 | MYOZ1 | −4.803 | 0.045079048 |
| A_23_P97553 | D42044 | KIAA0090 | −4.775 | 0.024753099 |
| A_23_P349343 | NM_139163 | ALS2CR12 | −4.683 | 0.015659374 |
| A_24_P340771 | XR_016510 | XR_016510 | −4.656 | 0.000639159 |
| A_23_P161962 | AK001998 | AK001998 | −4.646 | 0.001191654 |
| A_23_P137035 | NM_003662 | PIR | −4.634 | 0.007459819 |
| A_23_P73787 | NM_153183 | NUDT10 | −4.617 | 0.025141863 |
| A_24_P225970 | NM_001012409 | SGOL1 | −4.612 | 0.005165034 |
| A_23_P94030 | NM_002291 | LAMB1 | −4.547 | 0.029939799 |
| A_24_P419039 | NM_020766 | PCDH19 | −4.527 | 0.049497023 |
| A_23_P303181 | NM_020126 | SPHK2 | −4.459 | 0.000133044 |
| A_23_P305137 | ENST00000378250 | ENST00000378250 | −4.414 | 0.042897412 |
| A_23_P13137 | AY358815 | UNQ2550 | −4.367 | 0.001625242 |
| A_23_P347541 | NM_133445 | GRIN3A | −4.351 | 0.046351603 |
| A_23_P145846 | NM_000245 | MET | −4.332 | 0.007412903 |
| A_23_P104845 | NM_000741 | CHRM4 | −4.309 | 0.023531908 |
| A_32_P32195 | THC2651958 | THC2651958 | −4.303 | 0.013010357 |
| A_23_P79145 | NM_144694 | ZNF570 | −4.28 | 0.03474037 |
| A_32_P198731 | NR_015355 | LOC54492 | −4.253 | 0.022857478 |
| A_23_P367043 | NR_003595 | CDC14C | −4.238 | 0.005361109 |
| A_23_P205637 | NM_015163 | TRIM9 | −4.237 | 0.030914052 |
| A_24_P32473 | NM_024930 | ELOVL7 | −4.23 | 0.005179115 |
| A_23_P211039 | NM_006988 | ADAMTS1 | −4.224 | 0.038644969 |
| A_32_P96845 | NM_002830 | PTPN4 | −4.212 | 0.002216122 |
| A_23_P169257 | NM_001244 | TNFSF8 | −4.207 | 0.021548268 |
| A_32_P27763 | U92025 | U92025 | −4.199 | 0.009274437 |
| A_23_P167298 | NM_032149 | C4orf17 | −4.135 | 0.049356536 |
| A_23_P365412 | NM_181644 | MFSD4 | −4.082 | 0.016343909 |
| A_32_P111996 | ENST00000400767 | ENST00000400767 | −4.066 | 0.012109227 |
| A_23_P62953 | NM_002585 | PBX1 | −4.056 | 0.006008663 |
| A_23_P203729 | NM_002869 | RAB6A | −4.046 | 0.000497846 |
| A_23_P425766 | NM_052904 | KLHL32 | −4.045 | 0.038176362 |
| A_23_P121676 | NM_025212 | CXXC4 | −4.043 | 0.037218694 |
| A_24_P278156 | NM_018112 | TMEM38B | −3.993 | 0.024099284 |
| A_32_P118657 | AK022044 | AK022044 | −3.953 | 0.001881772 |
| A_23_P37785 | BC094790 | KCTD19 | −3.952 | 0.010605949 |
| A_24_P20623 | A_24_P20623 | A_24_P20623 | −3.931 | 0.016602676 |
| A_23_P207414 | NM_025267 | AARSD1 | −3.93 | 0.010992547 |
| A_23_P130056 | NM_020426 | LYZL6 | −3.882 | 0.024030665 |
| A_23_P2283 | NM_001006667 | TAC3 | −3.866 | 0.000606623 |
| A_23_P127608 | NM_019894 | TMPRSS4 | −3.866 | 0.009293401 |
| A_23_P87421 | BC063022 | BC063022 | −3.855 | 0.016304248 |
| A_23_P107432 | AK093253 | FLJ35934 | −3.851 | 0.001035311 |
| A_23_P411542 | AK095052 | LOC284033 | −3.848 | 0.03844623 |
| A_32_P112655 | AK058000 | AK058000 | −3.841 | 0.016930643 |
| A_32_P203559 | AK127362 | C11orf39 | −3.756 | 0.029859029 |
| A_32_P80597 | ENST00000394607 | ELOVL6 | −3.74 | 0.012984707 |
| A_24_P331882 | NM_020722 | KIAA1211 | −3.734 | 0.014137072 |
| A_23_P206877 | NM_018427 | RRN3 | −3.709 | 0.017630243 |
| A_24_P693986 | NM_001013642 | LOC388610 | −3.7 | 0.010512794 |
| A_23_P92999 | NM_016279 | CDH9 | −3.677 | 0.01425077 |
| A_23_P408167 | NM_001004051 | GPRASP2 | −3.666 | 0.019838144 |
| A_24_P889473 | AF085920 | AF085920 | −3.639 | 0.037747601 |
| A_24_P59596 | NM_014109 | ATAD2 | −3.634 | 0.007742036 |
| A_23_P50990 | NM_024322 | CENPO | −3.632 | 0.038111376 |
| A_24_P3761 | NM_170740 | ALDH5A1 | −3.549 | 0.000384684 |
| A_23_P214079 | NM_003122 | SPINK1 | −3.538 | 0.019906115 |
| A_23_P25746 | BC007251 | C14orf128 | −3.535 | 0.029343235 |
| A_24_P407742 | A_24_P407742 | A_24_P407742 | −3.508 | 0.037220442 |
| A_32_P93442 | AF086125 | AF086125 | −3.493 | 0.006312164 |
| A_23_P137116 | NM_015365 | AMMECR1 | −3.491 | 0.013142809 |
| A_32_P12232 | NM_017637 | BNC2 | −3.479 | 0.017821872 |
| A_23_P206612 | NM_020718 | USP31 | −3.475 | 0.005631503 |
| A_24_P295633 | NM_024841 | FLJ14213 | −3.461 | 0.007547246 |
| A_23_P46369 | NM_002870 | RAB13 | −3.45 | 0.017666247 |
| A_24_P945059 | NM_025107 | MYCT1 | −3.44 | 0.00780361 |
| A_24_P548811 | AK021458 | AK021458 | −3.432 | 0.001478136 |
| A_23_P304304 | NM_004042 | ARSF | −3.432 | 0.017115028 |
| A_23_P123544 | NM_017444 | CHRAC1 | −3.43 | 0.004701373 |
| A_23_P166964 | ENST00000397991 | TFDP2 | −3.426 | 0.003392159 |
| A_23_P364504 | ENST00000357303 | FAM132B | −3.373 | 0.021727567 |
| A_24_P16702 | NM_152793 | C7orf41 | −3.371 | 0.015140514 |
| A_23_P84219 | NM_139248 | LIPH | −3.361 | 0.022004127 |
| A_23_P28530 | NM_017694 | FLJ20160 | −3.357 | 0.008677221 |
| A_24_P175783 | NM_015313 | ARHGEF12 | −3.357 | 0.027998924 |
| A_32_P305888 | NM_024577 | SH3TC2 | −3.339 | 0.004053733 |
| A_23_P72050 | NM_153831 | PTK2 | −3.33 | 0.014143153 |
| A_23_P145824 | NM_017650 | PPP1R9A | −3.326 | 0.006644355 |
| A_24_P496297 | XR_018860 | XR_018860 | −3.318 | 0.033440996 |
| A_23_P346327 | AK055241 | FLJ30679 | −3.311 | 0.039901865 |
| A_24_P23546 | NM_198956 | SP8 | −3.303 | 0.027760623 |
| A_23_P321892 | NM_000412 | HRG | −3.3 | 0.010058418 |
| A_32_P171225 | THC2672305 | THC2672305 | −3.281 | 0.028489451 |
| A_23_P120863 | NM_004861 | GAL3ST1 | −3.265 | 0.013345032 |
| A_24_P886589 | BF115114 | BF115114 | −3.25 | 0.031797835 |
| A_23_P319512 | NM_003597 | KLF11 | −3.231 | 0.010264651 |
| A_23_P117580 | NM_001249 | ENTPD5 | −3.222 | 0.005731109 |
| A_24_P751492 | NM_001012446 | C9orf128 | −3.213 | 0.044996986 |
| A_23_P42128 | NM_031460 | KCNK17 | −3.207 | 0.035786486 |
| A_24_P23190 | NM_173510 | CCDC117 | −3.207 | 0.005910885 |
| A_32_P725218 | NM_181725 | METTL2A | −3.206 | 0.02811637 |
| A_23_P380208 | NM_024621 | VEPH1 | −3.206 | 0.021864562 |
| A_23_P98477 | NM_025004 | CCDC15 | −3.181 | 0.004277288 |
| A_23_P118042 | NM_018296 | LRRC36 | −3.173 | 0.00764061 |
| A_23_P138655 | NM_057157 | CYP26A1 | −3.147 | 0.007007579 |
| A_23_P74162 | NM_018134 | IQCC | −3.138 | 0.013206096 |
| A_23_P166400 | NM_001007279 | RASL10A | −3.102 | 0.006679534 |
| A_23_P350491 | NM_198794 | MAP4K5 | −3.098 | 0.00548186 |
| A_23_P31903 | NM_183057 | VPS28 | −3.089 | 0.005004101 |
| A_24_P13376 | NM_024551 | ADIPOR2 | −3.084 | 0.020832783 |
| A_23_P214578 | NM_006778 | TRIM10 | −3.083 | 0.008670706 |
| A_32_P218707 | A_32_P218707 | A_32_P218707 | −3.082 | 0.022664024 |
| A_23_P165959 | NM_016004 | IFT52 | −3.068 | 0.027126528 |
| A_23_P314805 | NM_152487 | TMEM56 | −3.044 | 0.019148479 |
| A_23_P104493 | NM_001015880 | PAPSS2 | −3.034 | 0.020436386 |
| A_32_P179083 | NR_015363 | MGC21881 | −3.027 | 0.027747285 |
| A_23_P69218 | NM_018447 | TMEM111 | −3.026 | 4.66E−05 |
| A_23_P105691 | NM_018654 | GPRC5D | −3.019 | 0.046463972 |
| A_23_P130376 | NM_022068 | FAM38B | −3.019 | 0.01464945 |
| A_23_P167328 | NM_001775 | CD38 | −2.997 | 0.003288355 |
| A_32_P218543 | THC2719525 | THC2719525 | −2.997 | 0.026539259 |
| A_24_P176484 | NM_015144 | ZCCHC14 | −2.994 | 0.047108031 |
| A_24_P347065 | AJ420378 | CPT1A | −2.993 | 0.018006562 |
| A_32_P64894 | A_32_P64894 | A_32_P64894 | −2.985 | 0.028119282 |
| A_23_P256172 | NM_199417 | C3orf60 | −2.984 | 0.000430327 |
| A_32_P117908 | A_32_P117908 | A_32_P117908 | −2.981 | 0.000944928 |
| A_32_P720220 | NM_182558 | C12orf36 | −2.959 | 0.022729953 |
| A_24_P185394 | NM_019884 | GSK3A | −2.94 | 0.001003966 |
| A_32_P10643 | A_32_P10643 | A_32_P10643 | −2.933 | 0.012813838 |
| A_23_P71946 | NM_017688 | BSPRY | −2.932 | 0.014345911 |
| A_23_P152995 | NM_001045 | SLC6A4 | −2.931 | 0.000171179 |
| A_32_P78783 | NM_182531 | ZNF778 | −2.912 | 0.048433307 |
| A_24_P71781 | AK095050 | TMEM108 | −2.911 | 0.03425584 |
| A_32_P73991 | THC2667995 | THC2667995 | −2.895 | 0.007117595 |
| A_23_P377197 | NM_012219 | MRAS | −2.89 | 0.009392009 |
| A_23_P72050 | NM_153831 | PTK2 | −2.873 | 0.022814829 |
| A_24_P185487 | NM_014910 | ZNF507 | −2.853 | 0.035066965 |
| A_24_P911179 | NM_018136 | ASPM | −2.85 | 0.01304793 |
| A_24_P634602 | BE542385 | BE542385 | −2.849 | 0.002399183 |
| A_23_P12241 | NM_018298 | MCOLN3 | −2.834 | 0.034881431 |
| A_32_P88310 | BM932296 | BM932296 | −2.833 | 0.001799513 |
| A_23_P56578 | NM_053276 | VIT | −2.814 | 0.017040294 |
| A_24_P345209 | NM_001004023 | DYRK3 | −2.795 | 0.020068529 |
| A_23_P8812 | W60781 | W60781 | −2.793 | 0.004399452 |
| A_23_P218743 | NM_020132 | AGPAT3 | −2.763 | 0.022887971 |
| A_32_P228570 | A_32_P228570 | A_32_P228570 | −2.761 | 0.049674718 |
| A_23_P72050 | NM_153831 | PTK2 | −2.76 | 0.019106259 |
| A_23_P10591 | NM_001004431 | METRNL | −2.755 | 0.001318866 |
| A_23_P1331 | NM_005203 | COL13A1 | −2.748 | 0.00196752 |
| A_23_P1331 | NM_005203 | COL13A1 | −2.744 | 0.006166992 |
| A_23_P370666 | NM_080661 | GLYATL1 | −2.726 | 0.015866973 |
| A_23_P82420 | NM_004603 | STX1A | −2.722 | 0.000874493 |
| A_24_P467073 | AK131023 | AK131023 | −2.719 | 0.006756573 |
| A_24_P32574 | NM_152643 | KNDC1 | −2.718 | 0.018421895 |
| A_23_P108404 | NM_001037131 | CENTG2 | −2.714 | 0.028591903 |
| A_23_P72050 | NM_153831 | PTK2 | −2.713 | 0.017015291 |
| A_24_P370471 | NM_031482 | ATG10 | −2.703 | 0.027554654 |
| A_32_P5150 | THC2682672 | THC2682672 | −2.702 | 0.004098814 |
| A_24_P862207 | BC057844 | BC057844 | −2.701 | 0.00809594 |
| A_23_P93912 | NM_019004 | ANKIB1 | −2.698 | 0.042693841 |
| A_23_P28811 | NM_014484 | MOCS3 | −2.694 | 0.032362567 |
| A_32_P4337 | THC2694873 | THC2694873 | −2.692 | 0.041097811 |
| A_23_P202945 | NM_014155 | ZBTB44 | −2.691 | 0.003072906 |
| A_23_P206301 | THC2551912 | THC2551912 | −2.686 | 0.007529704 |
| A_24_P743802 | NM_133374 | ZNF618 | −2.681 | 0.024261594 |
| A_23_P159119 | NM_016507 | CRKRS | −2.679 | 0.04343117 |
| A_23_P130285 | NM_018289 | VPS53 | −2.674 | 0.03024787 |
| A_23_P99906 | NM_199330 | HOMER2 | −2.671 | 0.002446742 |
| A_23_P206212 | NM_003246 | THBS1 | −2.654 | 0.013035007 |
| A_24_P602052 | CA420688 | CA420688 | −2.647 | 0.012147787 |
| A_24_P243278 | NM_004394 | DAP | −2.645 | 0.028994768 |
| A_23_P72050 | NM_153831 | PTK2 | −2.634 | 0.044417054 |
| A_32_P62863 | NM_014575 | SCHIP1 | −2.634 | 0.011541684 |
| A_32_P32338 | CA441889 | CA441889 | −2.62 | 0.019679193 |
| A_23_P71053 | NM_016447 | MPP6 | −2.617 | 0.02741578 |
| A_24_P209109 | NM_030571 | NDFIP1 | −2.617 | 0.006058484 |
| A_23_P134274 | NM_005837 | POP7 | −2.616 | 0.00067525 |
| A_23_P1331 | NM_005203 | COL13A1 | −2.614 | 0.007821696 |
| A_23_P149270 | A_23_P149270 | A_23_P149270 | −2.612 | 0.032552864 |
| A_23_P1331 | NM_005203 | COL13A1 | −2.608 | 0.003126645 |
| A_23_P417363 | AK057267 | CLIP4 | −2.608 | 0.041418146 |
| A_23_P163475 | NM_207380 | C15orf52 | −2.607 | 0.00450898 |
| A_24_P943000 | D31765 | POP1 | −2.602 | 0.04024216 |
| A_23_P152995 | NM_001045 | SLC6A4 | −2.6 | 0.000371848 |
| A_23_P39647 | NM_005070 | SLC4A3 | −2.589 | 0.037824226 |
| A_23_P42265 | NM_019101 | APOM | −2.588 | 0.012647631 |
| A_23_P428326 | CR936670 | TBC1D16 | −2.586 | 0.04230613 |
| A_23_P107750 | NM_030760 | S1PR5 | −2.586 | 0.004909104 |
| A_23_P107612 | NM_004163 | RAB27B | −2.582 | 0.000602003 |
| A_23_P152995 | NM_001045 | SLC6A4 | −2.581 | 0.000637719 |
| A_23_P155425 | THC2665061 | THC2665061 | −2.576 | 0.001179055 |
| A_24_P143440 | NM_014183 | DYNLRB1 | −2.57 | 0.008105945 |
| A_23_P59718 | NM_003130 | SRI | −2.551 | 0.001530166 |
| A_24_P239140 | NM_000819 | GART | −2.533 | 0.033372298 |
| A_24_P899637 | AA203154 | AA203154 | −2.531 | 0.008484042 |
| A_32_P26721 | A_32_P26721 | A_32_P26721 | −2.518 | 0.006767081 |
| A_23_P152995 | NM_001045 | SLC6A4 | −2.513 | 0.000414869 |
| A_23_P218225 | NM_014298 | QPRT | −2.512 | 0.000748108 |
| A_23_P38696 | NM_004948 | DSC1 | −2.506 | 0.032831721 |
| A_23_P15647 | NM_016231 | NLK | −2.505 | 0.001391513 |
| A_23_P42358 | NM_020482 | FHL5 | −2.472 | 0.008071373 |
| A_23_P214144 | NM_000493 | COL10A1 | −2.472 | 0.004516563 |
| A_23_P213638 | NM_024594 | PANK3 | −2.472 | 0.012988047 |
| A_23_P111995 | NM_002318 | LOXL2 | −2.467 | 0.005297728 |
| A_23_P112673 | NM_017975 | ZWILCH | −2.467 | 0.017477697 |
| A_24_P385336 | NM_152696 | HIPK1 | −2.464 | 0.036913264 |
| A_23_P217049 | NM_014286 | FREQ | −2.463 | 0.007527782 |
| A_23_P1331 | NM_005203 | COL13A1 | −2.458 | 0.004420619 |
| A_23_P80032 | NM_005225 | E2F1 | −2.458 | 0.011056709 |
| A_32_P324533 | NM_053282 | SH2D1B | −2.457 | 0.002623455 |
| A_23_P23834 | NM_001017403 | LGR6 | −2.454 | 0.000319282 |
| A_24_P331150 | NM_173483 | CYP4F22 | −2.453 | 0.014099296 |
| A_23_P502224 | NM_007326 | CYB5R3 | −2.453 | 0.000713164 |
| A_23_P96262 | A_23_P96262 | A_23_P96262 | −2.448 | 0.036857396 |
| A_23_P152995 | NM_001045 | SLC6A4 | −2.435 | 0.0006735 |
| A_24_P162319 | NM_001037540 | SCML1 | −2.433 | 0.039311491 |
| A_24_P628384 | NM_001001655 | ALKBH2 | −2.426 | 0.018438737 |
| A_32_P57854 | BC105283 | LOC100128731 | −2.424 | 0.003795672 |
| A_23_P70534 | NM_001003693 | C6orf21 | −2.414 | 0.002425683 |
| A_32_P134167 | BG031574 | CUL5 | −2.411 | 0.001576784 |
| A_32_P210223 | THC2762463 | THC2762463 | −2.407 | 0.008062836 |
| A_23_P151907 | NM_002570 | PCSK6 | −2.406 | 0.005678608 |
| A_23_P45456 | NM_001289 | CLIC2 | −2.406 | 0.021468937 |
| A_23_P1331 | NM_005203 | COL13A1 | −2.404 | 0.013587837 |
| A_23_P22096 | NM_153831 | PTK2 | −2.404 | 0.028845358 |
| A_24_P915652 | AA635126 | AA635126 | −2.399 | 0.03402048 |
| A_23_P111995 | NM_002318 | LOXL2 | −2.394 | 0.004451732 |
| A_32_P6646 | AK090694 | AK090694 | −2.392 | 0.020756236 |
| A_24_P725895 | BE218249 | BE218249 | −2.392 | 0.019049249 |
| A_23_P72050 | NM_153831 | PTK2 | −2.389 | 0.033004836 |
| A_24_P375819 | NR002774 | HTR7P | −2.378 | 0.037415071 |
| A_23_P69226 | NM_018447 | TMEM111 | −2.375 | 0.000369492 |
| A_23_P416536 | THC2539425 | THC2539425 | −2.372 | 0.023908744 |
| A_24_P183150 | NM_002090 | CXCL3 | −2.371 | 0.043914444 |
| A_24_P722473 | THC2702563 | THC2702563 | −2.37 | 0.046040776 |
| A_24_P933538 | A_24_P933538 | A_24_P933538 | −2.363 | 0.018933983 |
| A_23_P356330 | NM_015704 | FAM152B | −2.362 | 0.004564928 |
| A_23_P138717 | NM_001005339 | RGS10 | −2.357 | 0.000225254 |
| A_32_P91180 | THC2679544 | THC2679544 | −2.356 | 0.00688079 |
| A_23_P88559 | NM_000236 | LIPC | −2.355 | 0.000942728 |
| A_23_P324523 | NM_153208 | IQCK | −2.35 | 0.043634599 |
| A_24_P940166 | NM_001015880 | PAPSS2 | −2.339 | 0.01446574 |
| A_24_P109652 | NM_024776 | SGK269 | −2.339 | 0.028111355 |
| A_24_P483083 | NM_007111 | TFDP1 | −2.339 | 0.015480641 |
| A_23_P202823 | NM_005231 | CTTN | −2.338 | 0.003487921 |
| A_23_P152995 | NM_001045 | SLC6A4 | −2.333 | 0.001796829 |
| A_24_P370484 | A_24_P370484 | A_24_P370484 | −2.332 | 0.025903584 |
| A_23_P88351 | NM_181598 | SPG3A | −2.323 | 0.03851526 |
| A_23_P211680 | NM_015166 | MLC1 | −2.311 | 2.16E−05 |
| A_32_P114348 | NM_006913 | RNF5 | −2.311 | 0.001351848 |
| A_23_P405216 | NM_173656 | ZNF619 | −2.307 | 0.03641289 |
| A_24_P122874 | NM_015049 | TRAK2 | −2.305 | 8.53E−05 |
| A_23_P209246 | NM_005270 | GLI2 | −2.303 | 0.035991973 |
| A_24_P487399 | AW014022 | AW014022 | −2.298 | 0.008413861 |
| A_24_P347566 | NM_015059 | TLN2 | −2.297 | 0.030190695 |
| A_23_P214739 | NM_012160 | FBXL4 | −2.296 | 0.002660093 |
| A_32_P234700 | BC019667 | BC019667 | −2.293 | 0.025715278 |
| A_23_P136317 | NM_018254 | RCOR3 | −2.286 | 0.004269842 |
| A_23_P211550 | NM_014248 | RBX1 | −2.279 | 0.000781717 |
| A_32_P175667 | BI832578 | BI832578 | −2.271 | 0.037409603 |
| A_23_P164081 | NM_005964 | MYH10 | −2.259 | 0.013486679 |
| A_32_P35947 | XR_037106 | LOC728763 | −2.259 | 0.003198188 |
| A_24_P134235 | NM_003685 | KHSRP | −2.251 | 0.027766741 |
| A_23_P16866 | NM_007127 | VIL1 | −2.247 | 0.000161166 |
| A_32_P13417 | AK091942 | AK091942 | −2.244 | 0.036731786 |
| A_24_P918808 | NM_004152 | OAZ1 | −2.24 | 0.017980201 |
| A_23_P12514 | NM_175744 | RHOC | −2.239 | 0.000177365 |
| A_32_P203219 | NM_198794 | MAP4K5 | −2.234 | 0.002924426 |
| A_24_P166012 | NM_020640 | DCUN1D1 | −2.233 | 0.000387631 |
| A_23_P406135 | NM_015662 | IFT172 | −2.231 | 0.007169583 |
| A_24_P123011 | NM_006761 | YWHAE | −2.228 | 0.036298658 |
| A_23_P111995 | NM_002318 | LOXL2 | −2.226 | 0.01057123 |
| A_23_P420361 | NM_018462 | C3orf10 | −2.226 | 0.04170378 |
| A_24_P413437 | NM_007363 | NONO | −2.223 | 0.008965359 |
| A_23_P1331 | NM_005203 | COL13A1 | −2.223 | 0.008436327 |
| A_32_P131998 | THC2530075 | THC2530075 | −2.221 | 0.027908535 |
| A_23_P49391 | NM_016212 | TP53TG3 | −2.219 | 0.014364395 |
| A_23_P128728 | NM_001172 | ARG2 | −2.209 | 0.035923608 |
| A_23_P72050 | NM_153831 | PTK2 | −2.207 | 0.049650459 |
| A_23_P95672 | NM_006548 | IGF2BP2 | −2.206 | 0.005067234 |
| A_23_P255263 | NM_198194 | STOM | −2.205 | 0.001200766 |
| A_24_P926709 | A_24_P926709 | A_24_P926709 | −2.2 | 0.037137856 |
| A_23_P214168 | NM_004370 | COL12A1 | −2.199 | 0.005170733 |
| A_23_P256618 | AK024611 | CCDC90A | −2.197 | 0.002058726 |
| A_23_P18539 | NM_007351 | MMRN1 | −2.195 | 0.008183054 |
| A_23_P202881 | NM_005103 | FEZ1 | −2.195 | 0.001209863 |
| A_23_P433758 | NM_001030288 | SPN | −2.192 | 0.000764183 |
| A_32_P110472 | NM_203395 | IYD | −2.188 | 0.003147478 |
| A_32_P32463 | THC2753543 | THC2753543 | −2.183 | 0.002135648 |
| A_23_P82523 | NM_000927 | ABCB1 | −2.183 | 0.042253994 |
| A_32_P187277 | BC063570 | BC063570 | −2.183 | 0.020509156 |
| A_23_P89249 | NM_001005862 | ERBB2 | −2.177 | 0.00120887 |
| A_24_P291133 | NM_175866 | UHMK1 | −2.175 | 0.027386147 |
| A_23_P1331 | NM_005203 | COL13A1 | −2.174 | 0.011082274 |
| A_23_P7761 | NM_018356 | C5orf22 | −2.171 | 0.018649828 |
| A_23_P119337 | NM_012068 | ATF5 | −2.171 | 0.033262063 |
| A_24_P371628 | NM_054027 | ANKH | −2.169 | 0.024003161 |
| A_23_P23584 | NM_020248 | CTNNBIP1 | −2.163 | 0.002162971 |
| A_32_P133916 | NM_017637 | BNC2 | −2.159 | 0.03156881 |
| A_24_P302406 | AF086286 | AF086286 | −2.157 | 0.02151458 |
| A_32_P44987 | A_32_P44987 | A_32_P44987 | −2.156 | 0.033629531 |
| A_24_P838448 | AK022865 | FLJ22536 | −2.156 | 0.025116635 |
| A_32_P44961 | BE551634 | BE551634 | −2.154 | 0.008765175 |
| A_23_P92543 | NM_152540 | SCFD2 | −2.152 | 0.005484255 |
| A_23_P71037 | NM_000600 | IL6 | −2.149 | 0.008273309 |
| A_32_P163386 | BU679017 | BU679017 | −2.149 | 0.022765416 |
| A_32_P123743 | NR_002728 | KCNQ1OT1 | −2.147 | 8.69E−05 |
| A_23_P217326 | NM_001449 | FHL1 | −2.142 | 0.011482853 |
| A_23_P80032 | NM_005225 | E2F1 | −2.141 | 0.018655944 |
| A_23_P500333 | NM_001009955 | SSBP3 | −2.14 | 0.004482185 |
| A_23_P74920 | NM_014357 | LCE2B | −2.137 | 0.041825265 |
| A_24_P943095 | NM_001033553 | SPECC1 | −2.13 | 0.020729825 |
| A_23_P142950 | NM_015575 | GIGYF2 | −2.128 | 0.016017351 |
| A_23_P363769 | NM_002284 | KRT86 | −2.128 | 0.014998405 |
| A_24_P197537 | NM_003719 | PDE8B | −2.127 | 0.008593032 |
| A_23_P216949 | NM_005388 | PDCL | −2.121 | 0.006675504 |
| A_32_P189324 | A_32_P189324 | A_32_P189324 | −2.115 | 0.01521589 |
| A_23_P89249 | NM_001005862 | ERBB2 | −2.11 | 0.002262419 |
| A_24_P321068 | NM_001859 | SLC31A1 | −2.106 | 0.015940611 |
| A_24_P501520 | AK098185 | MCART1 | −2.104 | 0.014749838 |
| A_23_P152995 | NM_001045 | SLC6A4 | −2.104 | 0.001565029 |
| A_23_P166526 | NM_015653 | RIBC2 | −2.103 | 0.028907098 |
| A_23_P66117 | NM_032039 | ITFG3 | −2.102 | 0.009912241 |
| A_32_P104825 | NM_080678 | UBE2F | −2.098 | 0.000482675 |
| A_23_P80032 | NM_005225 | E2F1 | −2.083 | 0.031672475 |
| A_24_P128354 | AF130069 | AF130069 | −2.083 | 0.021067889 |
| A_23_P151376 | A_23_P151376 | A_23_P151376 | −2.081 | 0.009750578 |
| A_24_P110780 | AK075118 | C1orf118 | −2.081 | 0.026690118 |
| A_24_P90005 | NM_005203 | COL13A1 | −2.08 | 0.002601811 |
| A_23_P400887 | NM_012279 | ZNF346 | −2.079 | 0.043136135 |
| A_23_P20502 | NM_017767 | SLC39A4 | −2.067 | 0.003606546 |
| A_24_P20292 | ENST00000287590 | B3GNT7 | −2.067 | 0.039281294 |
| A_24_P342127 | NM_014997 | KIAA0265 | −2.065 | 0.017809849 |
| A_23_P141044 | NM_145271 | ZNF688 | −2.062 | 0.025864665 |
| A_23_P382835 | NM_002563 | P2RY1 | −2.061 | 0.013824291 |
| A_24_P230965 | NM_020931 | KIAA1586 | −2.061 | 0.020058124 |
| A_32_P39216 | NM_006761 | YWHAE | −2.058 | 0.036266732 |
| A_23_P96965 | NM_030786 | SYNC1 | −2.052 | 0.036445896 |
| A_23_P328740 | NM_001080535 | LINCR | −2.049 | 0.016331796 |
| A_23_P89249 | NM_001005862 | ERBB2 | −2.048 | 0.002099049 |
| A_23_P368794 | NM_174937 | TCERG1L | −2.046 | 0.029581886 |
| A_23_P113212 | NM_018004 | TMEM45A | −2.035 | 0.048950522 |
| A_32_P147651 | CR616314 | CR616314 | −2.033 | 0.049947719 |
| A_23_P39561 | NM_080678 | UBE2F | −2.025 | 0.001288292 |
| A_24_P375453 | NM_001009955 | SSBP3 | −2.021 | 0.01271782 |
| A_23_P152995 | NM_001045 | SLC6A4 | −2.014 | 0.003489041 |
| A_32_P106646 | NM_198076 | FAM36A | −2.014 | 0.004614169 |
| A_24_P9833 | NM_020229 | PRDM11 | −2.011 | 0.045491453 |
| A_23_P82523 | NM_000927 | ABCB1 | −2.011 | 0.039050044 |
| A_23_P152995 | NM_001045 | SLC6A4 | −2.006 | 0.002972946 |
| A_24_P494230 | AK123750 | AK123750 | −2.002 | 0.028678972 |
| A_23_P121564 | NM_000857 | GUCY1B3 | −2.001 | 0.002851862 |
| A_23_P202004 | NM_020200 | PRTFDC1 | −2 | 0.024158469 |
| A_32_P72351 | AK026140 | AK026140 | −2 | 0.039146839 |
| A_32_P188752 | AK129584 | AK129584 | −1.997 | 0.017654524 |
| A_24_P333445 | NM_012286 | MORF4L2 | −1.997 | 0.039768515 |
| A_24_P118231 | NM_001003803 | ATP5S | −1.992 | 0.00082353 |
| A_23_P407090 | NM_152995 | NFXL1 | −1.992 | 0.006412219 |
| A_24_P356015 | NM_004094 | EIF2S1 | −1.99 | 0.032371089 |
| A_23_P69310 | NM_003965 | CCRL2 | −1.988 | 0.046562901 |
| A_23_P162199 | NM_024779 | PIP4K2C | −1.984 | 0.012708383 |
| A_23_P89249 | NM_001005862 | ERBB2 | −1.98 | 0.002273174 |
| A_23_P89249 | NM_001005862 | ERBB2 | −1.98 | 0.002494749 |
| A_23_P408239 | AL117400 | PASK | −1.975 | 0.044926962 |
| A_24_P589028 | AK026750 | AK026750 | −1.971 | 3.22E−05 |
| A_32_P75141 | A_32_P75141 | A_32_P75141 | −1.967 | 0.006764815 |
| A_24_P410797 | ENST00000393496 | ENST00000393496 | −1.966 | 0.041681589 |
| A_23_P89249 | NM_001005862 | ERBB2 | −1.965 | 0.001971125 |
| A_23_P165343 | NM_002830 | PTPN4 | −1.964 | 0.003898266 |
| A_24_P927444 | ENST00000395522 | SPECC1 | −1.962 | 0.008053288 |
| A_23_P63190 | NM_002524 | NRAS | −1.961 | 0.025384513 |
| A_23_P89249 | NM_001005862 | ERBB2 | −1.96 | 0.002899256 |
| A_23_P208779 | NM_133180 | EPS8L1 | −1.96 | 0.033827985 |
| A_24_P341538 | NM_201286 | USP51 | −1.958 | 0.012769616 |
| A_23_P89249 | NM_001005862 | ERBB2 | −1.956 | 0.002932868 |
| A_24_P928522 | AK025142 | DST | −1.956 | 0.044733475 |
| A_23_P56314 | NM_006830 | UQCR | −1.954 | 0.036664463 |
| A_23_P11341 | BC006406 | FAM104B | −1.951 | 0.000253058 |
| A_23_P138706 | NM_000681 | ADRA2A | −1.949 | 0.04860168 |
| A_23_P37296 | NM_015684 | ATP5S | −1.948 | 0.000450367 |
| A_32_P99804 | A_32_P99804 | A_32_P99804 | −1.946 | 0.036790436 |
| A_32_P194095 | THC2617292 | THC2617292 | −1.94 | 4.02E−05 |
| A_24_P263310 | NM_182562 | FLJ39743 | −1.939 | 0.008807733 |
| A_23_P352879 | M90656 | GCLC | −1.934 | 0.036058456 |
| A_23_P15101 | NM_024780 | TMC5 | −1.933 | 0.001270505 |
| A_23_P89249 | NM_001005862 | ERBB2 | −1.932 | 0.002929782 |
| A_23_P104624 | NM_015036 | ENDOD1 | −1.932 | 0.000369079 |
| A_32_P40327 | BE184907 | BE184907 | −1.929 | 0.000908157 |
| A_24_P273666 | NM_001077489 | GNAS | −1.929 | 0.004550454 |
| A_24_P162211 | NM_001001438 | LSS | −1.921 | 0.024931396 |
| A_24_P128563 | NM_012316 | KPNA6 | −1.916 | 0.018652492 |
| A_32_P105311 | THC2676737 | THC2676737 | −1.913 | 0.030533797 |
| A_24_P234391 | NM_025202 | EFHD1 | −1.913 | 0.016514245 |
| A_23_P168556 | NM_004603 | STX1A | −1.91 | 0.003014378 |
| A_23_P210060 | NM_001042519 | MGC13057 | −1.907 | 0.018213183 |
| A_23_P32233 | NM_004235 | KLF4 | −1.905 | 0.036247071 |
| A_24_P942481 | NM_180989 | GPR180 | −1.898 | 0.023373189 |
| A_23_P54816 | NM_001694 | ATP6V0C | −1.897 | 0.002120646 |
| A_32_P53049 | NM_005801 | EIF1 | −1.896 | 0.004059212 |
| A_23_P152995 | NM_001045 | SLC6A4 | −1.896 | 0.004380552 |
| A_23_P18082 | NM_032806 | C3orf39 | −1.896 | 0.001799854 |
| A_23_P91430 | NM_007238 | PXMP4 | −1.894 | 0.033421888 |
| A_23_P27784 | NM_016363 | GP6 | −1.887 | 0.021993647 |
| A_24_P175435 | NM_014580 | SLC2A8 | −1.885 | 0.008988399 |
| A_23_P1331 | NM_005203 | COL13A1 | −1.883 | 0.024398062 |
| A_24_P929413 | A_24_P929413 | A_24_P929413 | −1.88 | 0.032953251 |
| A_24_P248083 | BC033233 | SNIP | −1.88 | 0.02259809 |
| A_24_P937405 | NM_007173 | PRSS23 | −1.875 | 0.001139569 |
| A_24_P100266 | NM_003932 | ST13 | −1.874 | 0.014628341 |
| A_23_P4551 | NM_015559 | SETBP1 | −1.869 | 0.000910367 |
| A_23_P213562 | NM_001992 | F2R | −1.864 | 0.003739193 |
| A_32_P181297 | NR_002330 | ST7OT1 | −1.857 | 0.00991493 |
| A_32_P224498 | THC2537367 | THC2537367 | −1.852 | 0.024181447 |
| A_23_P106497 | NM_004136 | IREB2 | −1.845 | 0.028073896 |
| A_23_P89249 | NM_001005862 | ERBB2 | −1.844 | 0.003269254 |
| A_24_P218006 | NM_032317 | WBSCR18 | −1.84 | 0.016731785 |
| A_23_P111995 | NM_002318 | LOXL2 | −1.828 | 0.043181955 |
| A_23_P20743 | NM_032342 | C9orf125 | −1.828 | 0.036404376 |
| A_24_P148503 | NM_003468 | FZD5 | −1.826 | 0.017655815 |
| A_24_P79403 | NM_002619 | PF4 | −1.825 | 7.62E−05 |
| A_24_P367397 | A_24_P367397 | A_24_P367397 | −1.824 | 0.004037516 |
| A_24_P189533 | NM_015036 | ENDOD1 | −1.822 | 0.002202659 |
| A_32_P95502 | A_32_P95502 | A_32_P95502 | −1.818 | 0.003581621 |
| A_24_P213396 | A_24_P213396 | A_24_P213396 | −1.816 | 0.012510399 |
| A_23_P103897 | NM_001081472 | SPATA1 | −1.816 | 0.036074087 |
| A_23_P159237 | NM_005293 | GPR20 | −1.814 | 0.000191112 |
| A_23_P257057 | NM_016647 | C8orf55 | −1.812 | 0.003537713 |
| A_23_P1331 | NM_005203 | COL13A1 | −1.809 | 0.016218072 |
| A_23_P127978 | NM_138706 | B3GNT6 | −1.809 | 0.035013368 |
| A_23_P86434 | BC007866 | MGC14425 | −1.807 | 0.048298481 |
| A_24_P849628 | NM_002904 | RDBP | −1.807 | 0.021886901 |
| A_23_P311818 | NM_021081 | GHRH | −1.803 | 0.042927255 |
| A_32_P171903 | BQ233868 | BQ233868 | −1.802 | 0.016913301 |
| A_23_P155666 | NM_001042402 | NAAA | −1.802 | 0.004497911 |
| A_24_P209113 | NM_030571 | NDFIP1 | −1.8 | 0.02527872 |
| A_23_P147874 | NM_023947 | CHID1 | −1.799 | 0.00462139 |
| A_23_P163697 | NM_016524 | SYT17 | −1.798 | 0.049370612 |
| A_23_P100278 | NM_000833 | GRIN2A | −1.789 | 0.008682866 |
| A_23_P45945 | NM_005801 | EIF1 | −1.789 | 0.003259467 |
| A_23_P136347 | NM_004447 | EPS8 | −1.788 | 0.01053456 |
| A_32_P56726 | A_32_P56726 | A_32_P56726 | −1.787 | 0.00719584 |
| A_23_P39074 | NM_006270 | RRAS | −1.787 | 0.000302389 |
| A_23_P4522 | NM_004786 | TXNL1 | −1.785 | 0.007716608 |
| A_23_P70701 | NM_017519 | ARID1B | −1.782 | 0.048223525 |
| A_32_P211683 | THC2506246 | THC2506246 | −1.782 | 0.041502752 |
| A_23_P322562 | NM_004210 | NEURL | −1.777 | 0.009820561 |
| A_32_P191417 | AW276186 | AW276186 | −1.773 | 0.030151312 |
| A_32_P194025 | BC006406 | FAM104B | −1.773 | 0.00580998 |
| A_32_P137408 | THC2560357 | THC2560357 | −1.773 | 0.009227372 |
| A_24_P391960 | XM_001131332 | LOC730144 | −1.77 | 0.010346384 |
| A_24_P764519 | BM666433 | BM666433 | −1.77 | 0.025556114 |
| A_23_P362712 | NM_148912 | ABHD11 | −1.765 | 0.015289015 |
| A_23_P321511 | NM_178450 | 3-Mar | −1.764 | 0.017521379 |
| A_23_P57137 | NM_018347 | C20orf29 | −1.764 | 0.014337755 |
| A_32_P167723 | A_32_P167723 | A_32_P167723 | −1.764 | 0.020731395 |
| A_23_P152087 | NM_018145 | FAM82C | −1.763 | 0.028070496 |
| A_23_P162879 | NM_032374 | C14orf153 | −1.762 | 0.020066441 |
| A_32_P13371 | BQ434958 | BQ434958 | −1.762 | 0.039916105 |
| A_23_P102950 | NM_080860 | RSPH1 | −1.762 | 0.014563301 |
| A_23_P10211 | NM_052885 | SLC2A13 | −1.76 | 0.036502539 |
| A_23_P139965 | NM_001010897 | SERP2 | −1.759 | 0.00486794 |
| A_32_P1381 | CR617556 | CR617556 | −1.759 | 0.003489157 |
| A_23_P309779 | NM_018177 | N4BP2 | −1.757 | 0.028363273 |
| A_32_P429876 | AK092378 | AK092378 | −1.754 | 0.025802162 |
| A_24_P341674 | NM_013989 | DIO2 | −1.754 | 0.031217757 |
| A_24_P462899 | NM_001012507 | C6orf173 | −1.751 | 0.017497655 |
| A_23_P114582 | THC2532155 | THC2532155 | −1.75 | 0.000282052 |
| A_23_P103149 | NM_001098 | ACO2 | −1.749 | 0.028684573 |
| A_23_P111995 | NM_002318 | LOXL2 | −1.749 | 0.016599647 |
| A_32_P201417 | NM_001039948 | SGSM1 | −1.743 | 0.038387427 |
| A_23_P74740 | NM_007262 | PARK7 | −1.738 | 0.046403749 |
| A_23_P120667 | NM_021219 | JAM2 | −1.735 | 0.022051599 |
| A_23_P1461 | NM_001008211 | OPTN | −1.735 | 8.93E−05 |
| A_23_P1691 | NM_002421 | MMP1 | −1.734 | 0.014941204 |
| A_23_P209527 | ENST00000392114 | VIL1 | −1.733 | 0.000253132 |
| A_23_P205265 | NM_001969 | EIF5 | −1.729 | 0.012487953 |
| A_23_P381714 | NM_198584 | CA13 | −1.728 | 0.001933836 |
| A_24_P298179 | A_24_P298179 | A_24_P298179 | −1.719 | 0.047791429 |
| A_32_P435367 | NM_002810 | PSMD4 | −1.713 | 0.003208996 |
| A_23_P7642 | NM_003118 | SPARC | −1.713 | 0.000410043 |
| A_23_P69573 | NM_000856 | GUCY1A3 | −1.712 | 0.002539921 |
| A_32_P98496 | BE564275 | BE564275 | −1.711 | 0.007392453 |
| A_23_P88893 | NM_017702 | DEF8 | −1.708 | 0.020046798 |
| A_32_P107833 | THC2619545 | THC2619545 | −1.707 | 0.027899683 |
| A_23_P61569 | NM_003762 | VAMP4 | −1.704 | 0.028471467 |
| A_32_P4814 | NM_032508 | TMEM185A | −1.701 | 0.002095863 |
| A_32_P85835 | NM_194318 | B3GALTL | −1.701 | 0.026217722 |
| A_32_P974 | THC2676436 | THC2676436 | −1.7 | 0.004350777 |
| A_24_P88763 | NM_032603 | LOXL3 | −1.693 | 0.009739442 |
| A_23_P252783 | NM_014580 | SLC2A8 | −1.693 | 0.028529903 |
| A_32_P155030 | AK096483 | AK096483 | −1.693 | 0.006258247 |
| A_32_P38093 | THC2632909 | THC2632909 | −1.692 | 0.033977589 |
| A_23_P308603 | NM_005417 | SRC | −1.692 | 0.004940928 |
| A_23_P59855 | NM_006524 | ZNF138 | −1.69 | 0.034854646 |
| A_23_P257945 | NM_005333 | HCCS | −1.69 | 0.024799426 |
| A_24_P918706 | THC2534144 | THC2534144 | −1.687 | 0.023614634 |
| A_23_P86171 | NM_004474 | FOXD2 | −1.685 | 0.023201005 |
| A_24_P345314 | NM_003189 | TAL1 | −1.683 | 0.007273393 |
| A_24_P633902 | NM_014455 | ZNF364 | −1.682 | 0.021729467 |
| A_23_P102965 | NM_015367 | BCL2L13 | −1.681 | 0.008030048 |
| A_23_P47464 | NM_001002925 | OR5AP2 | −1.679 | 0.007944748 |
| A_24_P342511 | ENST00000324654 | ENST00000324654 | −1.679 | 0.036823709 |
| A_23_P397999 | NM_003468 | FZD5 | −1.675 | 0.000255049 |
| A_23_P119714 | NM_001033549 | C19orf62 | −1.674 | 0.007493177 |
| A_23_P47135 | NM_005693 | NR1H3 | −1.673 | 0.034923253 |
| A_23_P382506 | NR_002169 | OR1F2P | −1.671 | 0.009118273 |
| A_32_P160206 | THC2706921 | THC2706921 | −1.668 | 0.025347317 |
| A_24_P44462 | NM_000366 | TPM1 | −1.666 | 0.00148473 |
| A_24_P703462 | CD518305 | CD518305 | −1.665 | 0.017612685 |
| A_23_P161064 | NM_014455 | ZNF364 | −1.664 | 0.041817619 |
| A_24_P176409 | NM_133458 | ZFP90 | −1.663 | 0.049136254 |
| A_23_P71037 | NM_000600 | IL6 | −1.659 | 0.028366319 |
| A_23_P354187 | BC014983 | TBC1D20 | −1.652 | 0.001482093 |
| A_32_P70891 | AK098629 | AK098629 | −1.65 | 0.000165324 |
| A_23_P140256 | NM_000270 | NP | −1.649 | 0.009106972 |
| A_23_P111995 | NM_002318 | LOXL2 | −1.648 | 0.033784889 |
| A_24_P340286 | BC068590 | CHADL | −1.647 | 0.006156064 |
| A_23_P142631 | NM_054033 | FKBP1B | −1.647 | 0.005421005 |
| A_24_P889962 | THC2701869 | THC2701869 | −1.646 | 0.037549521 |
| A_23_P102258 | NM_053050 | MRPL53 | −1.642 | 0.002661597 |
| A_23_P13852 | NM_007178 | STRAP | −1.642 | 0.007924895 |
| A_23_P71037 | NM_000600 | IL6 | −1.641 | 0.0419512 |
| A_24_P148657 | NM_018385 | LSG1 | −1.638 | 0.005756502 |
| A_32_P130577 | BC066989 | BC066989 | −1.634 | 0.044519637 |
| A_24_P160104 | NM_018943 | TUBA8 | −1.633 | 0.000416079 |
| A_23_P401106 | NM_002599 | PDE2A | −1.631 | 0.002390845 |
| A_23_P163467 | NM_207380 | C15orf52 | −1.626 | 0.0017186 |
| A_23_P91814 | NM_017875 | SLC25A38 | −1.626 | 0.010987927 |
| A_32_P210572 | NM_001024594 | C1orf53 | −1.624 | 0.03574353 |
| A_24_P407323 | NM_177951 | PPM1A | −1.621 | 0.001237573 |
| A_24_P918762 | THC2609092 | THC2609092 | −1.621 | 0.027089334 |
| A_23_P151662 | NM_197957 | MAX | −1.62 | 0.016987339 |
| A_23_P212696 | NM_007085 | FSTL1 | −1.62 | 0.000255814 |
| A_32_P218989 | NM_004559 | YBX1 | −1.617 | 0.001097902 |
| A_24_P226008 | NM_007283 | MGLL | −1.615 | 0.000568183 |
| A_24_P416079 | NM_016359 | NUSAP1 | −1.612 | 0.002253193 |
| A_23_P327426 | NM_152902 | TIPRL | −1.611 | 0.043883775 |
| A_23_P158925 | NM_145290 | GPR125 | −1.611 | 0.024202401 |
| A_32_P25639 | AK002042 | BET3L | −1.61 | 0.011957836 |
| A_23_P59630 | NM_005395 | PMS2L3 | −1.605 | 0.020156672 |
| A_32_P203500 | NM_052937 | PCMTD1 | −1.603 | 0.008356926 |
| A_24_P246552 | NM_006370 | VTI1B | −1.602 | 0.003507538 |
| A_23_P17095 | NM_006287 | TFPI | −1.6 | 0.011167862 |
| A_23_P103824 | THC2539706 | THC2539706 | −1.599 | 0.019956524 |
| A_23_P117602 | NM_004131 | GZMB | −1.598 | 0.000232475 |
| A_23_P92132 | NM_006764 | IFRD2 | −1.591 | 0.009690707 |
| A_23_P328069 | NM_000195 | HPS1 | −1.589 | 0.005310403 |
| A_23_P83939 | NM_032796 | SYAP1 | −1.585 | 0.003241346 |
| A_32_P73045 | NM_018686 | CMAS | −1.584 | 0.007957947 |
| A_23_P360354 | NM_152548 | FAM81B | −1.584 | 0.008830086 |
| A_23_P76538 | NM_017899 | TESC | −1.581 | 0.000648985 |
| A_23_P63371 | NM_003189 | TAL1gstol | −1.581 | 0.002076314 |
| A_23_P127426 | NM_003273 | TM7SF2 | −1.58 | 0.011026479 |
| A_32_P86466 | AK057142 | C17orf87 | −1.579 | 0.047487784 |
| A_23_P67960 | NM_021176 | G6PC2 | −1.579 | 0.04301164 |
| A_23_P3946 | NM_020201 | NT5M | −1.576 | 0.000160461 |
| A_23_P41390 | NM_018986 | SH3TC1 | −1.576 | 0.010509319 |
| A_32_P126832 | CD511705 | CD511705 | −1.575 | 0.009197677 |
| A_23_P1691 | NM_002421 | MMP1 | −1.575 | 0.019301206 |
| A_23_P501831 | NM_032385 | C5orf4 | −1.575 | 1.17E−05 |
| A_32_P105281 | AW771919 | AW771919 | −1.572 | 0.043481796 |
| A_32_P171061 | NM_005170 | ASCL2 | −1.572 | 0.00869792 |
| A_32_P107859 | BQ638334 | BQ638334 | −1.571 | 0.034559043 |
| A_23_P129279 | NM_018602 | DNAJA4 | −1.57 | 0.001643114 |
| A_24_P911718 | BC037919 | BC037919 | −1.569 | 0.009565317 |
| A_23_P80336 | NM_020243 | TOMM22 | −1.563 | 0.046214013 |
| A_24_P357037 | NM_182688 | UBE2G2 | −1.56 | 0.001099833 |
| A_24_P912941 | AB290171 | DNHD1 | −1.559 | 0.019915119 |
| A_23_P15123 | NM_019116 | UBFD1 | −1.556 | 0.007513103 |
| A_23_P38519 | NM_000212 | ITGB3 | −1.556 | 0.002002578 |
| A_23_P154526 | NM_004490 | GRB14 | −1.554 | 0.001364706 |
| A_24_P840343 | CA428801 | CA428801 | −1.553 | 0.029450763 |
| A_23_P255317 | NM_004290 | RNF14 | −1.553 | 0.029889295 |
| A_23_P135494 | NM_013943 | CLIC4 | −1.55 | 0.003566385 |
| A_24_P119482 | NM_024077 | SECISBP2 | −1.547 | 0.004920088 |
| A_24_P921823 | THC2499809 | THC2499809 | −1.546 | 0.000629711 |
| A_24_P920904 | BC032347 | C8orf59 | −1.544 | 0.047185589 |
| A_23_P259561 | AB362555 | AB362555 | −1.539 | 0.004673948 |
| A_23_P259189 | NM_013943 | CLIC4 | −1.539 | 0.033874944 |
| A_23_P250619 | NM_153746 | ZDHHC14 | −1.538 | 0.016116843 |
| A_23_P146654 | NM_004323 | BAG1 | −1.537 | 0.009469616 |
| A_23_P138492 | NM_004210 | NEURL | −1.534 | 0.000522538 |
| A_23_P107214 | NM_201434 | RAB5C | −1.531 | 0.021719884 |
| A_32_P173177 | BG284526 | BG284526 | −1.531 | 0.014280673 |
| A_23_P14026 | NM_001310 | CREBL2 | −1.53 | 0.004617768 |
| A_23_P390006 | NM_138325 | PCSK6 | −1.529 | 0.016341698 |
| A_24_P605233 | THC2620595 | THC2620595 | −1.529 | 0.032550936 |
| A_24_P65779 | NM_001033930 | UBA52 | −1.528 | 0.013329693 |
| A_24_P62505 | NM_015101 | GLT25D2 | −1.528 | 0.021015706 |
| A_24_P318457 | NM_001950 | E2F4 | −1.525 | 0.003255763 |
| A_24_P100277 | NM_007326 | CYB5R3 | −1.524 | 0.034624909 |
| A_23_P149664 | NM_001079809 | TMEM183B | −1.522 | 0.016092498 |
| A_24_P924862 | NM_213589 | RAPH1 | −1.521 | 0.005644918 |
| A_23_P207507 | NM_003786 | ABCC3 | −1.52 | 0.000577884 |
| A_23_P38519 | NM_000212 | ITGB3 | −1.517 | 0.001763015 |
| A_32_P134968 | NM_001024858 | SPTB | −1.513 | 0.001992059 |
| A_32_P875758 | AB046850 | AB046850 | −1.512 | 0.005822662 |
| A_23_P335920 | NM_021135 | RPS6KA2 | −1.512 | 0.002012158 |
| A_24_P304051 | NM_004832 | GSTO1 | −1.511 | 0.032049768 |
| A_23_P38519 | NM_000212 | ITGB3 | −1.511 | 0.001528973 |
| A_24_P226322 | NM_031469 | SH3BGRL2 | −1.509 | 0.041900231 |
| A_32_P207789 | BQ017638 | BQ017638 | −1.506 | 0.023922157 |
| A_24_P560909 | THC2532996 | THC2532996 | −1.505 | 0.032252872 |
| A_23_P38519 | NM_000212 | ITGB3 | −1.505 | 0.002068826 |
| A_23_P157736 | NM_032728 | PPAPDC3 | −1.504 | 0.01125016 |
| A_24_P567952 | CR606587 | HCG18 | −1.502 | 0.005734042 |
| A_32_P215100 | ENST00000377006 | ENST00000377006 | −1.502 | 0.049160506 |
| A_32_P64263 | BQ029628 | BQ029628 | −1.501 | 0.038947068 |
| A_24_P119141 | NM_000313 | PROS1 | −1.5 | 0.026149861 |
| A_24_P338145 | NM_013442 | STOML2 | −1.498 | 0.035735411 |
| A_23_P30315 | NM_033342 | TRIM7 | −1.498 | 0.02151062 |
| A_23_P413788 | NM_173540 | FUT11 | −1.495 | 0.003164761 |
| A_23_P258088 | NM_020804 | PACSIN1 | −1.494 | 0.001409194 |
| A_23_P150789 | NM_007173 | PRSS23 | −1.494 | 0.000597416 |
| A_23_P351148 | NM_053282 | SH2D1B | −1.494 | 0.000265955 |
| A_23_P163079 | NM_000161 | GCH1 | −1.49 | 0.029292145 |
| A_32_P129269 | NM_052909 | PLEKHG4B | −1.489 | 0.03694613 |
| A_23_P1691 | NM_002421 | MMP1 | −1.488 | 0.028489467 |
| A_23_P43810 | NM_206943 | LTBP1 | −1.485 | 0.002593141 |
| A_23_P312181 | NM_207013 | TCEB2 | −1.484 | 0.017335196 |
| A_32_P26464 | ENST00000329015 | ENST00000329015 | −1.483 | 0.035988035 |
| A_23_P360215 | NM_021173 | POLD4 | −1.481 | 0.015832449 |
| A_23_P19987 | NM_006547 | IGF2BP3 | −1.48 | 0.002207092 |
| A_23_P18903 | NM_032782 | HAVCR2 | −1.477 | 0.002790818 |
| A_24_P371194 | NM_053050 | MRPL53 | −1.477 | 0.004976756 |
| A_23_P213102 | NM_016081 | PALLD | −1.477 | 0.003411129 |
| A_23_P89199 | NM_003653 | COPS3 | −1.475 | 0.00806153 |
| A_24_P316364 | NM_015523 | REXO2 | −1.475 | 0.00701764 |
| A_23_P78543 | NM_005498 | AP1M2 | −1.473 | 0.013518513 |
| A_32_P125177 | THC2630830 | THC2630830 | −1.472 | 0.040173538 |
| A_24_P176079 | NM_006646 | WASF3 | −1.472 | 0.034834101 |
| A_23_P120488 | NM_024663 | NPEPL1 | −1.472 | 0.023113274 |
| A_23_P168610 | NM_014399 | TSPAN13 | −1.471 | 0.020894758 |
| A_23_P323930 | NM_005723 | TSPAN5 | −1.471 | 0.000204094 |
| A_23_P54488 | NM_015162 | ACSBG1 | −1.47 | 0.016093246 |
| A_23_P421843 | ENST00000285206 | ENST00000285206 | −1.469 | 0.007728422 |
| A_24_P62800 | NM_018203 | KLHDC8A | −1.466 | 0.006755477 |
| A_24_P378928 | NM_005160 | ADRBK2 | −1.464 | 0.033380989 |
| A_32_P73821 | NM_001007553 | CSDE1 | −1.463 | 0.044988398 |
| A_23_P416048 | AK024437 | MAPK8IP3 | −1.46 | 0.009622958 |
| A_23_P85742 | NM_003851 | CREG1 | −1.46 | 0.000653779 |
| A_23_P409516 | NM_138333 | FAM122A | −1.46 | 0.004328959 |
| A_23_P118300 | NM_031208 | FAHD1 | −1.459 | 0.015614674 |
| A_23_P123071 | NM_001233 | CAV2 | −1.457 | 0.00988223 |
| A_24_P8350 | NM_006031 | PCNT | −1.455 | 0.033413253 |
| A_23_P38519 | NM_000212 | ITGB3 | −1.455 | 0.002286853 |
| A_23_P1936 | NM_025080 | ASRGL1 | −1.453 | 0.008964099 |
| A_23_P29495 | NM_001904 | CTNNB1 | −1.452 | 0.021528978 |
| A_24_P288954 | NM_001013694 | SRRD | −1.447 | 0.001886248 |
| A_24_P409500 | BC000986 | BC000986 | −1.445 | 0.000899485 |
| A_32_P233950 | NM_001025290 | DPPA5 | −1.445 | 0.006844868 |
| A_24_P532653 | AA242987 | AA242987 | −1.445 | 0.040551503 |
| A_24_P151121 | ENST00000263100 | A1BG | −1.445 | 0.027153534 |
| A_32_P108592 | A_32_P108592 | A_32_P108592 | −1.445 | 0.01832988 |
| A_23_P16451 | NM_025241 | UBXD1 | −1.444 | 0.002466646 |
| A_32_P93187 | AL517609 | AL517609 | −1.444 | 0.026883986 |
| A_23_P29495 | NM_001904 | CTNNB1 | −1.443 | 0.019967525 |
| A_23_P1691 | NM_002421 | MMP1 | −1.442 | 0.046261894 |
| A_32_P5148 | THC2682672 | THC2682672 | −1.439 | 0.033107777 |
| A_23_P393777 | NM_000953 | PTGDR | −1.437 | 0.008197075 |
| A_23_P206103 | NM_001099436 | ULK3 | −1.437 | 0.035400962 |
| A_23_P46627 | NM_015999 | ADIPOR1 | −1.435 | 0.000431813 |
| A_23_P29495 | NM_001904 | CTNNB1 | −1.435 | 0.020535414 |
| A_23_P59210 | NM_000389 | CDKN1A | −1.435 | 0.004770811 |
| A_24_P205268 | NM_015299 | KIAA0323 | −1.43 | 0.029201413 |
| A_23_P145330 | NM_019052 | CCHCR1 | −1.429 | 0.046105827 |
| A_23_P38519 | NM_000212 | ITGB3 | −1.429 | 0.002347474 |
| A_24_P147263 | NM_020718 | USP31 | −1.429 | 0.034443705 |
| A_32_P135043 | THC2636523 | THC2636523 | −1.429 | 0.008377161 |
| A_23_P40548 | NM_013313 | YPEL1 | −1.427 | 0.021271228 |
| A_23_P423074 | NM_015566 | KIAA0888 | −1.426 | 0.026286395 |
| A_24_P173754 | NM_030806 | C1orf21 | −1.424 | 0.01916069 |
| A_24_P23245 | NM_002490 | NDUFA6 | −1.423 | 0.023238914 |
| A_23_P90357 | NM_001060 | TBXA2R | −1.421 | 0.001436169 |
| A_23_P78330 | NM_006566 | CD226 | −1.419 | 0.044778829 |
| A_32_P135385 | NM_001013694 | SRRD | −1.418 | 0.001546157 |
| A_32_P186725 | AL546498 | AL546498 | −1.417 | 0.001018687 |
| A_32_P188178 | NM_004238 | TRIP12 | −1.416 | 0.031037034 |
| A_23_P1691 | NM_002421 | MMP1 | −1.414 | 0.034145041 |
| A_23_P38519 | NM_000212 | ITGB3 | −1.413 | 0.002751524 |
| A_23_P13907 | NM_000618 | IGF1 | −1.41 | 0.023710263 |
| A_32_P69803 | NM_018566 | YOD1 | −1.409 | 0.001943653 |
| A_32_P36005 | THC2489591 | THC2489591 | −1.408 | 0.045302421 |
| A_24_P402847 | NM_001829 | CLCN3 | −1.408 | 0.044752026 |
| A_23_P75453 | NM_130803 | MEN1 | −1.405 | 0.045638493 |
| A_23_P59210 | NM_000389 | CDKN1A | −1.402 | 0.013686788 |
| A_23_P59210 | NM_000389 | CDKN1A | −1.4 | 0.014300991 |
| A_32_P214860 | A_32_P214860 | A_32_P214860 | −1.4 | 0.048228772 |
| A_23_P363344 | NM_000366 | TPM1 | −1.399 | 0.004598972 |
| A_24_P682199 | CR749500 | LOC390595 | −1.398 | 0.022893538 |
| A_23_P38519 | NM_000212 | ITGB3 | −1.396 | 0.00343016 |
| A_24_P135444 | NM_001144 | AMFR | −1.396 | 0.014627501 |
| A_23_P39076 | NM_006270 | RRAS | −1.395 | 0.020871915 |
| A_24_P836315 | A_24_P836315 | A_24_P836315 | −1.395 | 0.022469524 |
| A_23_P2601 | NM_003299 | HSP90B1 | −1.395 | 0.005222929 |
| A_24_P929003 | S70348 | ITGB3 | −1.394 | 0.002757181 |
| A_24_P209171 | NM_031469 | SH3BGRL2 | −1.393 | 0.002821556 |
| A_23_P53439 | NM_138432 | SDSL | −1.392 | 0.02918421 |
| A_23_P204998 | NM_005766 | FARP1 | −1.392 | 0.034098002 |
| A_24_P388622 | NM_016930 | STX18 | −1.391 | 0.025397882 |
| A_23_P214789 | NM_016224 | SNX9 | −1.39 | 0.006605681 |
| A_23_P34915 | NM_001040619 | ATF3 | −1.39 | 0.040531436 |
| A_24_P29885 | NM_002514 | NOV | −1.39 | 0.027862795 |
| A_32_P185149 | NM_006913 | RNF5 | −1.389 | 0.014894246 |
| A_32_P21459 | THC2664391 | THC2664391 | −1.386 | 0.045051979 |
| A_23_P300220 | NM_145008 | YPEL4 | −1.385 | 0.001379838 |
| A_23_P29495 | NM_001904 | CTNNB1 | −1.384 | 0.028998264 |
| A_23_P208389 | NM_021913 | AXL | −1.383 | 0.015477209 |
| A_23_P19894 | NM_198098 | AQP1 | −1.382 | 0.023679359 |
| A_24_P357056 | NM_152906 | C22orf25 | −1.38 | 0.00687387 |
| A_23_P132121 | NM_173354 | SNF1LK | −1.378 | 0.00837809 |
| A_23_P13232 | A_23_P13232 | A_23_P13232 | −1.378 | 0.045068576 |
| A_23_P132138 | ENST00000397683 | ENST00000397683 | −1.378 | 0.0059675 |
| A_23_P14284 | NM_006370 | VTI1B | −1.378 | 0.002989176 |
| A_23_P215096 | NM_003344 | UBE2H | −1.377 | 0.003549624 |
| A_24_P229669 | ENST00000283760 | ENST00000283760 | −1.377 | 0.016077859 |
| A_23_P162787 | NM_006704 | SUGT1 | −1.377 | 0.022728898 |
| A_23_P152909 | NM_000697 | ALOX12 | −1.376 | 0.012405347 |
| A_23_P230 | NM_004623 | TTC4 | −1.372 | 0.04345693 |
| A_32_P9575 | NM_032351 | MRPL45 | −1.372 | 0.01640871 |
| A_32_P155026 | THC2507792 | THC2507792 | −1.372 | 0.035524709 |
| A_23_P59388 | NM_001723 | DST | −1.369 | 0.005547073 |
| A_23_P257743 | NM_003028 | SHB | −1.366 | 0.043686427 |
| A_24_P942933 | AK022802 | APOBEC3G | −1.366 | 0.00402701 |
| A_23_P150609 | NM_000612 | IGF2 | −1.365 | 8.23E−05 |
| A_24_P219920 | NM_018708 | FEM1A | −1.362 | 0.003807985 |
| A_32_P96043 | THC2534106 | THC2534106 | −1.362 | 0.021859261 |
| A_23_P206697 | NM_030579 | CYB5B | −1.362 | 0.013602613 |
| A_24_P187954 | NM_213654 | ARMC8 | −1.359 | 0.020480591 |
| A_23_P211252 | NM_001001438 | LSS | −1.359 | 0.046172576 |
| A_32_P25397 | AK022962 | AK022962 | −1.359 | 0.008370729 |
| A_23_P128230 | NM_002135 | NR4A1 | −1.358 | 0.000207788 |
| A_32_P69930 | NM_153376 | CCDC96 | −1.358 | 0.037432388 |
| A_24_P755069 | A_24_P755069 | A_24_P755069 | −1.356 | 0.022338746 |
| A_23_P97457 | NM_022831 | C1orf80 | −1.355 | 0.041697281 |
| A_23_P122662 | NM_018988 | GFOD1 | −1.355 | 0.001704455 |
| A_23_P29495 | NM_001904 | CTNNB1 | −1.355 | 0.026434591 |
| A_23_P138117 | AY037153 | CAMTA1 | −1.354 | 0.040749699 |
| A_23_P64019 | NM_201278 | MTMR2 | −1.354 | 0.032041799 |
| A_23_P346900 | NM_001005505 | CACNA2D2 | −1.351 | 0.032305764 |
| A_23_P218685 | NM_001032288 | UBE2V1 | −1.351 | 0.013896684 |
| A_23_P353574 | NM_133494 | NEK7 | −1.35 | 0.049323959 |
| A_32_P2605 | AV756170 | AV756170 | −1.349 | 0.0225174 |
| A_24_P115450 | NM_018688 | BIN3 | −1.348 | 0.007824918 |
| A_23_P252758 | NM_000420 | KEL | −1.347 | 0.032965656 |
| A_23_P107744 | NM_030760 | S1PR5 | −1.345 | 0.001890705 |
| A_23_P252721 | NM_182643 | DLC1 | −1.344 | 0.031039569 |
| A_24_P673786 | NM_005028 | PIP4K2A | −1.342 | 0.001709883 |
| A_23_P13907 | NM_000618 | IGF1 | −1.34 | 0.025413227 |
| A_23_P118552 | NM_006460 | HEXIM1 | −1.34 | 0.00957893 |
| A_24_P164815 | A_24_P164815 | A_24_P164815 | −1.34 | 0.023798719 |
| A_32_P213306 | THC2579654 | THC2579654 | −1.34 | 0.007111351 |
| A_24_P299562 | AF116618 | AF116618 | −1.339 | 0.025473972 |
| A_23_P325562 | NM_006671 | SLC1A7 | −1.338 | 0.000206122 |
| A_23_P156957 | NM_181782 | NCOA7 | −1.338 | 0.043885439 |
| A_23_P389118 | NM_001025356 | TMEM16F | −1.336 | 0.003927556 |
| A_23_P48175 | NM_024056 | TMEM106C | −1.336 | 0.007786205 |
| A_24_P367079 | NM_205854 | SFTPG | −1.335 | 0.021600695 |
| A_23_P38519 | NM_000212 | ITGB3 | −1.329 | 0.004161954 |
| A_32_P222060 | A_32_P222060 | A_32_P222060 | −1.329 | 0.00954536 |
| A_24_P167377 | NM_206899 | OR10P1 | −1.328 | 0.047611944 |
| A_23_P89030 | AK026130 | FBXO31 | −1.328 | 0.012640701 |
| A_23_P49849 | NM_178860 | SEZ6 | −1.327 | 0.005624643 |
| A_24_P24332 | A_24_P24332 | A_24_P24332 | −1.326 | 0.03436009 |
| A_23_P308603 | NM_005417 | SRC | −1.323 | 0.011894952 |
| A_23_P366366 | NM_014766 | SCRN1 | −1.322 | 0.006153499 |
| A_32_P11181 | NM_006191 | PA2G4 | −1.32 | 0.016809171 |
| A_24_P127491 | ENST00000330710 | ENST00000330710 | −1.32 | 0.01587455 |
| A_23_P255111 | THC2545097 | THC2545097 | −1.319 | 0.011398533 |
| A_23_P217570 | NM_014289 | CAPN6 | −1.317 | 0.028124089 |
| A_32_P83803 | NR_004382 | MESTIT1 | −1.317 | 0.029387311 |
| A_24_P158536 | NM_014836 | RHOBTB1 | −1.316 | 0.01115731 |
| A_24_P205589 | NM_007274 | ACOT7 | −1.316 | 0.036735297 |
| A_23_P85664 | NM_152376 | UBXD3 | −1.314 | 0.040961198 |
| A_23_P171296 | NM_002436 | MPP1 | −1.313 | 0.001549252 |
| A_23_P41128 | NM_003884 | PCAF | −1.311 | 0.002965833 |
| A_23_P171314 | NM_023934 | FUNDC2 | −1.311 | 0.004285256 |
| A_23_P53081 | NM_020896 | OSBPL5 | −1.308 | 0.006968867 |
| A_32_P74778 | A_32_P74778 | A_32_P74778 | −1.308 | 0.035574456 |
| A_23_P500844 | NM_001083 | PDE5A | −1.308 | 0.021312078 |
| A_23_P152926 | J02940 | GP1BA | −1.307 | 0.001215857 |
| A_23_P395534 | NM_006511 | RSC1A1 | −1.305 | 0.032301519 |
| A_32_P231641 | THC2671299 | THC2671299 | −1.304 | 0.008809705 |
| A_32_P141724 | NM_053041 | COMMD7 | −1.303 | 0.027848298 |
| A_23_P387649 | AF116663 | DNAJA4 | −1.302 | 0.015984144 |
| A_23_P25974 | NM_001010854 | TTC7B | −1.301 | 0.002217922 |
| A_32_P169521 | H45075 | H45075 | −1.301 | 0.037234243 |
| A_23_P83368 | NM_016373 | WWOX | −1.3 | 0.033456216 |
| A_23_P86421 | NM_005437 | NCOA4 | −1.3 | 0.019118014 |
| A_23_P369815 | NM_000639 | FASLG | −1.298 | 0.01310483 |
| A_23_P62634 | NM_020485 | RHCE | −1.298 | 0.028740993 |
| A_24_P32790 | NM_018566 | YOD1 | −1.298 | 0.00705535 |
| A_23_P61127 | NM_024122 | APOO | −1.297 | 0.043723219 |
| A_32_P31165 | BC000986 | BC000986 | −1.297 | 0.002770819 |
| A_32_P192594 | CR624390 | LOC400099 | −1.295 | 0.047576156 |
| A_23_P359277 | NM_024930 | ELOVL7 | −1.294 | 0.022846907 |
| A_24_P9809 | NM_017580 | ZRANB1 | −1.29 | 0.040975117 |
| A_23_P12082 | NM_001025199 | CHI3L2 | −1.29 | 0.036117238 |
| A_23_P75220 | NM_031212 | SLC25A28 | −1.289 | 0.011187312 |
| A_23_P29495 | NM_001904 | CTNNB1 | −1.287 | 0.035481589 |
| A_23_P29495 | NM_001904 | CTNNB1 | −1.286 | 0.040163241 |
| A_23_P38519 | NM_000212 | ITGB3 | −1.285 | 0.004802208 |
| A_23_P149992 | NM_020992 | PDLIM1 | −1.283 | 0.032727583 |
| A_24_P348173 | AK001784 | AK001784 | −1.283 | 0.017720948 |
| A_32_P130968 | BC035377 | BC035377 | −1.281 | 0.046573589 |
| A_23_P10291 | NM_001910 | CTSE | −1.279 | 0.012522939 |
| A_23_P361448 | NM_144665 | SESN3 | −1.278 | 0.000805002 |
| A_23_P29495 | NM_001904 | CTNNB1 | −1.277 | 0.045480637 |
| A_23_P7074 | NM_032313 | C4orf14 | −1.272 | 0.043057497 |
| A_24_P290314 | E01827 | E01827 | −1.272 | 0.019655527 |
| A_23_P69695 | NM_001008388 | CISD2 | −1.271 | 0.008913573 |
| A_24_P173823 | BC044624 | BC044624 | −1.267 | 0.004355294 |
| A_23_P119295 | NM_016581 | ECSIT | −1.267 | 0.020302055 |
| A_24_P399680 | NM_080821 | C20orf108 | −1.266 | 0.000690282 |
| A_23_P34767 | NM_004559 | YBX1 | −1.266 | 0.009768226 |
| A_23_P58293 | NM_181886 | UBE2D3 | −1.265 | 0.009615451 |
| A_23_P216966 | NM_000962 | PTGS1 | −1.265 | 0.010355191 |
| A_23_P139486 | NM_004642 | CDK2AP1 | −1.263 | 0.037256927 |
| A_23_P147984 | NM_004238 | TRIP12 | −1.262 | 0.017970538 |
| A_23_P138541 | NM_003739 | AKR1C3 | −1.261 | 0.002718504 |
| A_23_P371266 | NM_015569 | DNM3 | −1.259 | 0.011934158 |
| A_23_P205808 | NM_012388 | PLDN | −1.254 | 0.010110833 |
| A_23_P431319 | NM_173834 | YIPF6 | −1.254 | 0.004692627 |
| A_23_P320159 | ENST00000314720 | LOC170082 | −1.252 | 0.011538816 |
| A_32_P63324 | THC2700145 | THC2700145 | −1.251 | 0.016103868 |
| A_23_P115922 | NM_004096 | EIF4EBP2 | −1.251 | 0.046396943 |
| A_23_P125408 | NM_031304 | DOHH | −1.251 | 0.018356039 |
| A_32_P96752 | AW946823 | AW946823 | −1.251 | 0.007136219 |
| A_32_P42574 | NM_032800 | C1orf198 | −1.25 | 0.00614054 |
| A_23_P156550 | AY358357 | TREML1 | −1.25 | 0.002574953 |
| A_23_P73150 | NM_031421 | TTC25 | −1.249 | 0.000593981 |
| A_23_P369919 | NM_004578 | RAB4A | −1.249 | 0.031014172 |
| A_23_P253281 | NM_001102426 | TBC1D8 | −1.248 | 0.001253289 |
| A_23_P152655 | NM_000873 | ICAM2 | −1.247 | 0.031116139 |
| A_32_P195401 | NM_173511 | ALS2CR13 | −1.245 | 0.012258104 |
| A_24_P346762 | NM_025182 | KIAA1539 | −1.245 | 0.009010161 |
| A_24_P409494 | NM_015367 | BCL2L13 | −1.242 | 0.042390432 |
| A_23_P97914 | AK021432 | AK021432 | −1.242 | 0.012461477 |
| A_32_P17743 | THC2670504 | THC2670504 | −1.242 | 0.004488486 |
| A_23_P10685 | NM_012267 | HSPBP1 | −1.242 | 0.04454452 |
| A_23_P92362 | NM_002494 | NDUFC1 | −1.24 | 0.017352479 |
| A_24_P16997 | NM_198794 | MAP4K5 | −1.24 | 0.004267742 |
| A_24_P165259 | NM_013328 | PYCR2 | −1.239 | 0.031952123 |
| A_23_P373100 | BC020879 | MGC24103 | −1.239 | 0.011157401 |
| A_23_P29495 | NM_001904 | CTNNB1 | −1.238 | 0.049802227 |
| A_23_P6034 | NM_030773 | TUBB1 | −1.237 | 0.015036076 |
| A_23_P12282 | NM_001004023 | DYRK3 | −1.237 | 0.006395023 |
| A_23_P99747 | NM_004196 | CDKL1 | −1.234 | 0.042201772 |
| A_24_P281264 | A_24_P281264 | A_24_P281264 | −1.233 | 0.012051638 |
| A_24_P277955 | NM_016068 | FIS1 | −1.231 | 0.007030677 |
| A_23_P109322 | NM_006198 | PCP4 | −1.231 | 0.036180458 |
| A_23_P86424 | NM_005437 | NCOA4 | −1.23 | 0.019141681 |
| A_23_P208582 | A_23_P208582 | A_23_P208582 | −1.229 | 0.038151572 |
| A_24_P466102 | AL390143 | AL390143 | −1.229 | 0.048710519 |
| A_23_P67708 | NM_003200 | TCF3 | −1.227 | 0.004711255 |
| A_23_P433760 | NM_001030288 | SPN | −1.227 | 0.028429855 |
| A_23_P369733 | NM_020850 | RANBP10 | −1.227 | 0.012015233 |
| A_23_P351 | NM_203342 | EPB41 | −1.226 | 0.009493062 |
| A_23_P210658 | NM_016045 | SLMO2 | −1.226 | 0.035048705 |
| A_23_P59210 | NM_000389 | CDKN1A | −1.226 | 0.014289663 |
| A_24_P168574 | ENST00000371081 | ENST00000371081 | −1.225 | 0.011356453 |
| A_23_P20622 | NM_033331 | CDC14B | −1.221 | 0.015041129 |
| A_23_P98431 | NM_000190 | HMBS | −1.22 | 0.003920833 |
| A_23_P119794 | CR608907 | CR608907 | −1.218 | 0.018656465 |
| A_32_P140139 | NM_000129 | F13A1 | −1.217 | 0.002713248 |
| A_24_P320033 | NM_005191 | CD80 | −1.217 | 0.03062659 |
| A_23_P98431 | NM_000190 | HMBS | −1.217 | 0.003774837 |
| A_24_P309594 | BC026344 | FLJ20489 | −1.216 | 0.045969735 |
| A_24_P7330 | A_24_P7330 | A_24_P7330 | −1.213 | 0.000770685 |
| A_23_P353035 | NM_001553 | IGFBP7 | −1.213 | 0.002785147 |
| A_24_P91238 | NM_032837 | FAM104A | −1.211 | 0.011729306 |
| A_24_P936537 | AL036373 | AL036373 | −1.21 | 0.041951845 |
| A_32_P17439 | THC2537721 | THC2537721 | −1.21 | 0.01191789 |
| A_24_P102981 | NM_006736 | DNAJB2 | −1.208 | 0.01066099 |
| A_23_P54376 | NM_004809 | STOML1 | −1.205 | 0.047219098 |
| A_23_P98431 | NM_000190 | HMBS | −1.205 | 0.003908706 |
| A_32_P166693 | NM_020733 | HEG1 | −1.204 | 0.024398048 |
| A_32_P226869 | BC002470 | BC002470 | −1.203 | 0.009827673 |
| A_24_P113131 | NM_004758 | BZRAP1 | −1.203 | 0.012760033 |
| A_24_P703642 | A_24_P703642 | A_24_P703642 | −1.202 | 0.015031367 |
| A_23_P69468 | NM_004547 | NDUFB4 | −1.201 | 0.035233736 |
| A_23_P204208 | NM_002262 | KLRD1 | −1.201 | 0.008144812 |
| A_23_P128808 | NM_013345 | GPR132 | −1.2 | 0.013318678 |
| A_32_P42780 | NM_001112663 | ZNF271 | −1.199 | 0.020870867 |
| A_23_P13907 | NM_000618 | IGF1 | −1.199 | 0.043374755 |
| A_23_P111981 | NM_177457 | LYNX1 | −1.199 | 0.003105978 |
| A_24_P58247 | NR_002169 | OR1F2P | −1.198 | 0.044496236 |
| A_23_P59210 | NM_000389 | CDKN1A | −1.197 | 0.028533615 |
| A_32_P167791 | NM_003932 | ST13 | −1.196 | 0.016746467 |
| A_23_P110022 | NM_032638 | GATA2 | −1.196 | 0.021911713 |
| A_23_P18713 | NM_004827 | ABCG2 | −1.195 | 0.002342884 |
| A_24_P930597 | BG259864 | BG259864 | −1.194 | 0.013101999 |
| A_23_P143817 | NM_053025 | MYLK | −1.193 | 0.010224591 |
| A_24_P826646 | NM_001037738 | NPM1 | −1.193 | 0.016027548 |
| A_23_P128147 | NM_006082 | TUBA1B | −1.193 | 0.043252366 |
| A_23_P89073 | NM_145911 | ZNF23 | −1.193 | 0.044682778 |
| A_24_P333525 | NM_014857 | RABGAP1L | −1.193 | 0.009960941 |
| A_23_P108042 | BC064806 | NCR1 | −1.192 | 0.010082878 |
| A_23_P55616 | ENST00000321925 | SLC14A1 | −1.189 | 5.75E−05 |
| A_24_P118531 | BP290435 | BP290435 | −1.188 | 0.005639706 |
| A_23_P98431 | NM_000190 | HMBS | −1.188 | 0.004709564 |
| A_23_P81441 | NM_130848 | C5orf20 | −1.187 | 0.044403174 |
| A_23_P428129 | NM_000076 | CDKN1C | −1.187 | 9.13E−05 |
| A_23_P210358 | NM_004987 | LIMS1 | −1.186 | 0.008706487 |
| A_23_P19886 | NM_207173 | NPSR1 | −1.186 | 0.024439913 |
| A_24_P148043 | NM_014864 | FAM20B | −1.186 | 0.020381927 |
| A_23_P143120 | NM_003183 | ADAM17 | −1.185 | 0.040449138 |
| A_23_P13907 | NM_000618 | IGF1 | −1.184 | 0.042184946 |
| A_23_P414771 | A_23_P414771 | A_23_P414771 | −1.183 | 0.032178235 |
| A_24_P208809 | NM_004162 | RAB5A | −1.183 | 0.013162431 |
| A_24_P281844 | THC2526432 | THC2526432 | −1.182 | 0.008160459 |
| A_23_P343221 | NM_006669 | LILRB1 | −1.181 | 0.012752846 |
| A_23_P983 | NM_004905 | PRDX6 | −1.181 | 0.000762612 |
| A_23_P93302 | NM_001470 | GABBR1 | −1.18 | 0.04621842 |
| A_23_P422922 | NM_031923 | TAF3 | −1.179 | 0.046124797 |
| A_23_P206284 | NM_201525 | GPR56 | −1.179 | 0.003006508 |
| A_23_P121596 | NM_002704 | PPBP | −1.177 | 0.000360208 |
| A_23_P397376 | NM_001031804 | MAF | −1.177 | 0.020222754 |
| A_23_P308603 | NM_005417 | SRC | −1.175 | 0.011138063 |
| A_24_P212997 | XR_019070 | XR_019070 | −1.175 | 0.020031842 |
| A_24_P415615 | NM_000738 | CHRM1 | −1.173 | 0.015457519 |
| A_23_P401361 | NM_020845 | PITPNM2 | −1.173 | 0.011776408 |
| A_23_P83388 | NM_031308 | EPPK1 | −1.171 | 0.033455481 |
| A_23_P126613 | NM_080429 | AQP10 | −1.17 | 0.001369868 |
| A_23_P103034 | NM_001886 | CRYBA4 | −1.17 | 0.017776849 |
| A_23_P369815 | NM_000639 | FASLG | −1.169 | 0.022343115 |
| A_32_P215078 | BQ364306 | BQ364306 | −1.169 | 0.007375063 |
| A_23_P425073 | NM_002898 | RBMS2 | −1.168 | 0.005027856 |
| A_23_P108522 | BC064950 | FAM134A | −1.168 | 0.011299809 |
| A_23_P89708 | NM_018129 | PNPO | −1.168 | 0.036874541 |
| A_24_P482009 | XR_038151 | LOC100131707 | −1.168 | 0.010966818 |
| A_23_P98431 | NM_000190 | HMBS | −1.167 | 0.005315258 |
| A_23_P145761 | NM_005738 | ARL4A | −1.167 | 0.020427132 |
| A_23_P143143 | NM_002166 | ID2 | −1.167 | 0.033007847 |
| A_23_P30755 | NM_138296 | PTCRA | −1.166 | 0.001103766 |
| A_23_P73114 | NM_000313 | PROS1 | −1.166 | 0.011960939 |
| A_23_P85682 | NM_005595 | NFIA | −1.163 | 0.010397376 |
| A_23_P98431 | NM_000190 | HMBS | −1.161 | 0.005429583 |
| A_24_P365807 | NM_004429 | EFNB1 | −1.16 | 0.021570365 |
| A_23_P98431 | NM_000190 | HMBS | −1.159 | 0.005296501 |
| A_23_P25253 | NM_015954 | DERA | −1.158 | 0.028544104 |
| A_23_P55107 | NM_014683 | ULK2 | −1.158 | 0.046415451 |
| A_23_P22744 | NM_130467 | PAGE5 | −1.157 | 0.001309657 |
| A_23_P152955 | NM_017622 | C17orf59 | −1.156 | 0.039130704 |
| A_23_P49597 | NM_020360 | PLSCR3 | −1.155 | 0.002592446 |
| A_24_P110967 | NM_052937 | PCMTD1 | −1.154 | 0.033987554 |
| A_23_P62932 | NM_001677 | ATP1B1 | −1.154 | 0.020000378 |
| A_24_P38572 | NM_022917 | NOL6 | −1.152 | 0.028880362 |
| A_32_P93584 | A_32_P93584 | A_32_P93584 | −1.152 | 0.008498803 |
| A_23_P79911 | NM_006814 | PSMF1 | −1.151 | 0.009013196 |
| A_23_P77562 | NM_194280 | LOC124446 | −1.15 | 0.044291661 |
| A_23_P216610 | NM_022486 | SUSD1 | −1.149 | 0.031381099 |
| A_23_P108331 | NM_003021 | SGTA | −1.149 | 0.017573236 |
| A_23_P111267 | NM_031469 | SH3BGRL2 | −1.148 | 0.005363486 |
| A_24_P648880 | NR_002211 | MEIS3P1 | −1.148 | 0.026809681 |
| A_23_P253081 | NM_002183 | IL3RA | −1.147 | 0.034683268 |
| A_24_P369232 | NM_031455 | CCDC3 | −1.147 | 0.034263644 |
| A_23_P55584 | NM_003831 | RIOK3 | −1.144 | 0.003184772 |
| A_24_P210343 | NM_002355 | M6PR | −1.144 | 0.041204399 |
| A_23_P213137 | NM_032622 | LNX1 | −1.144 | 0.041525444 |
| A_32_P37247 | AK096908 | LOC642826 | −1.144 | 0.014731434 |
| A_24_P119685 | NM_052843 | OBSCN | −1.142 | 0.001231052 |
| A_24_P196188 | NM_007224 | NXPH4 | −1.142 | 0.042076234 |
| A_23_P124522 | NM_024819 | DCAKD | −1.142 | 0.03637202 |
| A_23_P19142 | NM_004137 | KCNMB1 | −1.14 | 0.018438751 |
| A_23_P364517 | NM_021237 | SELK | −1.14 | 0.016506528 |
| A_23_P111701 | NM_004126 | GNG11 | −1.14 | 0.002790991 |
| A_23_P98431 | NM_000190 | HMBS | −1.14 | 0.00535897 |
| A_32_P77252 | THC2608799 | THC2608799 | −1.14 | 0.04111324 |
| A_32_P118397 | NM_001039372 | LOC253012 | −1.138 | 0.033776374 |
| A_24_P525144 | A_24_P525144 | A_24_P525144 | −1.138 | 0.014247305 |
| A_23_P29225 | NM_012179 | FBXO7 | −1.138 | 0.012369069 |
| A_24_P567944 | CR606587 | HCG18 | −1.136 | 0.005334899 |
| A_23_P407565 | NM_001337 | CX3CR1 | −1.135 | 0.001479874 |
| A_23_P63128 | AK024186 | OBSCN | −1.135 | 0.030619372 |
| A_23_P391586 | NM_001018004 | TPM1 | −1.134 | 0.004349566 |
| A_23_P98431 | NM_000190 | HMBS | −1.133 | 0.006575661 |
| A_23_P145114 | NM_001498 | GCLC | −1.133 | 0.004245408 |
| A_23_P372962 | NM_144670 | A2ML1 | −1.133 | 0.011295288 |
| A_23_P104346 | NM_005028 | PIP4K2A | −1.132 | 0.002088047 |
| A_24_P344711 | NM_020132 | AGPAT3 | −1.132 | 0.032223398 |
| A_23_P72668 | NM_004657 | SDPR | −1.132 | 0.007143627 |
| A_23_P44195 | NM_138962 | MSI2 | −1.132 | 0.024894993 |
| A_24_P205008 | AK094466 | TSEN54 | −1.131 | 0.014452914 |
| A_24_P149124 | NM_004772 | C5orf13 | −1.131 | 0.007383558 |
| A_24_P928243 | T15787 | T15787 | −1.13 | 0.012849713 |
| A_23_P86682 | NM_013451 | FER1L3 | −1.13 | 0.008803486 |
| A_23_P42498 | NM_003795 | SNX3 | −1.128 | 0.034382089 |
| A_23_P338495 | NM_001002265 | 8-Mar | −1.127 | 0.004516648 |
| A_23_P69513 | NM_198229 | RGS12 | −1.127 | 0.013678121 |
| A_23_P214080 | NM_001964 | EGR1 | −1.127 | 0.041905573 |
| A_32_P327750 | XR_019306 | XR_019306 | −1.126 | 0.005654669 |
| A_24_P14485 | NM_017495 | RBM38 | −1.125 | 0.001704552 |
| A_32_P22654 | NM_000032 | ALAS2 | −1.124 | 0.000327984 |
| A_23_P63547 | THC2663297 | THC2663297 | −1.123 | 0.00102733 |
| A_23_P23346 | NM_006818 | MLLT11 | −1.122 | 0.016540046 |
| A_23_P121722 | NM_001080477 | ODZ3 | −1.121 | 0.042925631 |
| A_23_P421401 | NM_002609 | PDGFRB | −1.118 | 0.039490091 |
| A_24_P180654 | BC063666 | CREB3L2 | −1.118 | 0.044595536 |
| A_23_P17130 | NM_001042519 | MGC13057 | −1.118 | 0.009524191 |
| A_23_P399579 | XR_018040 | XR_018040 | −1.118 | 0.016610407 |
| A_23_P137806 | NM_005373 | MPL | −1.117 | 0.004090377 |
| A_23_P105571 | NM_020244 | CHPT1 | −1.117 | 0.004018266 |
| A_24_P362540 | NM_003887 | DDEF2 | −1.114 | 0.015401702 |
| A_23_P202280 | NM_014000 | VCL | −1.114 | 0.041746427 |
| A_24_P916423 | AI337612 | AI337612 | −1.114 | 0.011028379 |
| A_24_P128068 | BC007822 | UBE2O | −1.114 | 0.042368758 |
| A_23_P2661 | NM_015646 | RAP1B | −1.112 | 0.042790736 |
| A_23_P84587 | NM_018979 | WNK1 | −1.112 | 0.001748678 |
| A_24_P414183 | NM_016819 | OGG1 | −1.112 | 0.032167001 |
| A_23_P68949 | NM_003932 | ST13 | −1.11 | 0.02944635 |
| A_32_P5514 | A_32_P5514 | A_32_P5514 | −1.11 | 0.006781297 |
| A_32_P39866 | BC040412 | BC040412 | −1.109 | 0.008842662 |
| A_23_P217938 | NM_006542 | SPHAR | −1.107 | 0.038324963 |
| A_24_P230907 | BC015394 | FAM149B1 | −1.107 | 0.022818155 |
| A_23_P59210 | NM_000389 | CDKN1A | −1.107 | 0.037131241 |
| A_24_P816384 | NR_003661 | FLJ43276 | −1.107 | 0.044142227 |
| A_23_P363896 | NM_152350 | C17orf45 | −1.106 | 0.016064093 |
| A_24_P168416 | NM_181738 | PRDX2 | −1.105 | 0.01986185 |
| A_23_P118266 | NM_006885 | ZFHX3 | −1.105 | 0.038463727 |
| A_32_P55462 | CR593500 | LOC388494 | −1.104 | 0.016298294 |
| A_23_P160920 | NM_005764 | PDZK1IP1 | −1.103 | 0.000993152 |
| A_23_P105461 | NM_004072 | CMKLR1 | −1.103 | 0.035322916 |
| A_24_P583549 | NM_024345 | WDR32 | −1.101 | 0.013971902 |
| A_24_P397928 | NM_147780 | CTSB | −1.098 | 0.013158962 |
| A_23_P157795 | NM_003798 | CTNNAL1 | −1.097 | 0.001791138 |
| A_23_P95029 | NM_021021 | SNTB1 | −1.097 | 0.017595387 |
| A_23_P217054 | NM_024345 | WDR32 | −1.096 | 0.008223352 |
| A_23_P165840 | NM_002539 | ODC1 | −1.096 | 0.01436092 |
| A_23_P3038 | NM_002083 | GPX2 | −1.096 | 0.039738486 |
| A_32_P109922 | NM_020863 | ZFAT | −1.094 | 0.030649239 |
| A_32_P331700 | A_32_P331700 | A_32_P331700 | −1.093 | 0.011134037 |
| A_32_P385587 | NM_000032 | ALAS2 | −1.091 | 0.000275764 |
| A_23_P8913 | NM_000067 | CA2 | −1.09 | 0.00783723 |
| A_24_P125283 | NM_001015053 | HDAC5 | −1.09 | 0.007985256 |
| A_24_P222114 | NM_018177 | N4BP2 | −1.09 | 0.021325314 |
| A_24_P88505 | NM_016505 | ZCCHC17 | −1.089 | 0.025217107 |
| A_23_P33207 | AF319957 | AF319957 | −1.088 | 0.026381744 |
| A_23_P98402 | NM_001040455 | SIDT2 | −1.088 | 0.025677396 |
| A_32_P29954 | NM_001098830 | C10orf109 | −1.087 | 0.009383877 |
| A_23_P112159 | NM_012154 | EIF2C2 | −1.086 | 0.022921317 |
| A_23_P121533 | NM_012445 | SPON2 | −1.086 | 0.000540672 |
| A_24_P188071 | NM_032704 | TUBA1C | −1.085 | 0.005062177 |
| A_23_P254193 | NM_017931 | LL22NC03-5H6.5 | −1.084 | 0.022728998 |
| A_23_P116797 | NM_021640 | C12orf10 | −1.082 | 0.026720782 |
| A_23_P213468 | A_23_P213468 | A_23_P213468 | −1.081 | 0.008034999 |
| A_23_P123866 | NM_016525 | UBAP1 | −1.08 | 0.009940877 |
| A_23_P98431 | NM_000190 | HMBS | −1.079 | 0.007187066 |
| A_32_P128258 | NR_002804 | SIGLECP3 | −1.079 | 0.002110451 |
| A_23_P206441 | NM_000135 | FANCA | −1.077 | 0.01833531 |
| A_23_P117082 | NM_015987 | HEBP1 | −1.077 | 0.000139916 |
| A_23_P30913 | NM_033554 | HLA-DPA1 | −1.073 | 0.007047572 |
| A_23_P254111 | NM_033480 | FBX09 | −1.072 | 0.004569201 |
| A_23_P210482 | NM_000022 | ADA | −1.07 | 0.032035378 |
| A_23_P76983 | NM_025057 | C14orf45 | −1.07 | 0.004049613 |
| A_23_P95790 | NM_017625 | ITLN1 | −1.069 | 1.99E−05 |
| A_23_P124132 | NM_024648 | FLJ22222 | −1.069 | 0.021107464 |
| A_23_P396765 | NM_173582 | PGM2L1 | −1.067 | 0.026469812 |
| A_24_P32560 | NM_198573 | C9orf165 | −1.067 | 0.016800795 |
| A_23_P56328 | NM_031310 | PLVAP | −1.066 | 0.041626264 |
| A_23_P25224 | NM_003651 | CSDA | −1.066 | 0.00373079 |
| A_24_P792130 | EG328448 | EG328448 | −1.065 | 0.021552093 |
| A_32_P136450 | THC2656784 | THC2656784 | −1.064 | 0.024248558 |
| A_23_P354341 | NM_007053 | CD160 | −1.063 | 0.04096449 |
| A_24_P64167 | NM_000962 | PTGS1 | −1.063 | 0.011617693 |
| A_23_P167521 | NM_033549 | TRIM41 | −1.063 | 0.022347151 |
| A_23_P145584 | NM_003344 | UBE2H | −1.062 | 0.017123014 |
| A_24_P296698 | NM_145109 | MAP2K3 | −1.062 | 0.005559596 |
| A_23_P117862 | NM_138704 | NDNL2 | −1.059 | 0.040525366 |
| A_32_P151470 | A_32_P151470 | A_32_P151470 | −1.058 | 0.019199371 |
| A_23_P40847 | NM_004267 | CHST2 | −1.056 | 0.008761836 |
| A_23_P348063 | NM_004711 | SYGR1 | −1.056 | 0.028724935 |
| A_23_P209700 | NM_006056 | NMUR1 | −1.05 | 0.001304216 |
| A_24_P887805 | A_24_P887805 | A_24_P887805 | −1.048 | 0.036884224 |
| A_23_P114275 | NM_006667 | PGRMC1 | −1.048 | 0.006378052 |
| A_24_P170726 | AK094407 | LOC645427 | −1.048 | 0.022279376 |
| A_23_P206441 | NM_000135 | FANCA | −1.048 | 0.023017146 |
| A_24_P144104 | ENST00000320010 | ENST00000320010 | −1.047 | 0.033546159 |
| A_24_P924125 | A_24_P924125 | A_24_P924125 | −1.046 | 0.012453694 |
| A_23_P320095 | NM_014460 | CSDC2 | −1.044 | 0.008706198 |
| A_32_P71437 | THC2583762 | THC2583762 | −1.044 | 0.019167809 |
| A_32_P175183 | NM_014155 | ZBTB44 | −1.043 | 0.043207247 |
| A_24_P38895 | NM_002105 | H2AFX | −1.042 | 0.040625382 |
| A_24_P308506 | NM_016347 | NAT8B | −1.042 | 0.047171467 |
| A_23_P110234 | NM_001890 | CSN1S1 | −1.041 | 0.032049379 |
| A_23_P308603 | NM_005417 | SRC | −1.041 | 0.02130036 |
| A_24_P896373 | A_24_P896373 | A_24_P896373 | −1.04 | 0.039749596 |
| A_23_P1594 | NM_003377 | VEGFB | −1.039 | 0.043050852 |
| A_32_P18251 | NM_023934 | FUNDC2 | −1.039 | 0.004328221 |
| A_32_P731227 | XR_017079 | XR_017079 | −1.036 | 0.02614295 |
| A_24_P819419 | THC2532711 | THC2532711 | −1.034 | 0.020550047 |
| A_23_P89798 | NM_001012515 | FECH | −1.034 | 0.000593173 |
| A_24_P117942 | NM_207377 | UNQ9438 | −1.032 | 0.002915529 |
| A_23_P26815 | NM_031430 | RILP | −1.031 | 0.008943676 |
| A_32_P224234 | AK123450 | LOC645195 | −1.03 | 0.009532074 |
| A_24_P719081 | AA704712 | AA704712 | −1.029 | 0.038640831 |
| A_32_P429872 | NM_016097 | IER3IP1 | −1.029 | 0.04098143 |
| A_23_P141555 | NM_013351 | TBX21 | −1.029 | 0.00351051 |
| A_32_P208078 | NM_005957 | MTHFR | −1.028 | 0.020672544 |
| A_23_P145724 | NM_006658 | C7orf16 | −1.025 | 0.010045617 |
| A_23_P68007 | NM_001679 | ATP1B3 | −1.024 | 0.021850037 |
| A_32_P63426 | THC2733814 | THC2733814 | −1.024 | 0.005688242 |
| A_32_P42236 | ENST00000342314 | ENST00000342314 | −1.023 | 0.019005987 |
| A_23_P206280 | NM_201525 | GPR56 | −1.021 | 0.001709852 |
| A_24_P316019 | ENST00000333156 | ENST00000333156 | −1.019 | 0.017941024 |
| A_32_P46214 | NM_173653 | SLC9A9 | −1.018 | 0.043167978 |
| A_32_P191859 | NM_020382 | SETD8 | −1.013 | 0.0333565 |
| A_23_P59210 | NM_000389 | CDKN1A | −1.012 | 0.028457215 |
| A_23_P354387 | NM_013451 | FER1L3 | −1.011 | 0.002820762 |
| A_23_P118693 | NM_003593 | FOXN1 | −1.01 | 0.039700315 |
| A_32_P184937 | THC2680668 | THC2680668 | −1.009 | 0.045021522 |
| A_23_P308603 | NM_005417 | SRC | −1.008 | 0.041686861 |
| A_24_P100130 | NM_001191 | BCL2L1 | −1.008 | 0.030571207 |
| A_23_P24234 | NM_001030015 | OPN4 | −1.007 | 0.046244056 |
| A_23_P50946 | NM_005855 | RAMP1 | −1.005 | 0.024372442 |
| A_23_P97860 | NM_000235 | LIPA | −1.004 | 0.008894293 |
| A_23_P165657 | NM_005415 | SLC20A1 | −1.004 | 0.025795767 |
| A_23_P137434 | NM_014372 | RNF11 | −1.004 | 0.028107812 |
| A_23_P250701 | NM_018979 | WNK1 | −1.002 | 0.014160379 |
| A_23_P110791 | NM_005211 | CSF1R | −1.002 | 0.031324006 |
| A_23_P398515 | NM_004203 | PKMYT1 | −1.002 | 0.041281396 |
| A_23_P17773 | NM_031444 | C22orf13 | −1.001 | 0.023552323 |
| A_23_P20615 | NM_006401 | ANP32B | −1 | 0.048463903 |
| A_23_P380614 | NM_006045 | ATP9A | −0.999 | 0.018149159 |
| A_24_P29703 | NM_013446 | MKRN1 | −0.998 | 0.004025003 |
| A_23_P146554 | NM_000954 | PTGDS | −0.997 | 0.001893177 |
| A_23_P141715 | NM_015476 | C18orf10 | −0.997 | 0.003096586 |
| A_23_P215944 | NM_147780 | CTSB | −0.996 | 0.022264681 |
| A_23_P1029 | NM_017459 | MFAP2 | −0.996 | 0.04906068 |
| A_23_P144151 | AF292100 | DCUN1D1 | −0.995 | 0.01814325 |
| A_32_P109683 | NM_001015038 | PAGE2B | −0.995 | 0.016103577 |
| A_32_P169754 | NM_004559 | YBX1 | −0.995 | 0.002936214 |
| A_32_P168349 | ENST00000375806 | C6orf25 | −0.993 | 0.002484017 |
| A_23_P45524 | NM_014380 | NGFRAP1 | −0.992 | 0.019617309 |
| A_24_P251534 | NM_001008392 | CTDSPL | −0.991 | 0.025329154 |
| A_23_P17430 | NM_017495 | RBM38 | −0.991 | 0.009187967 |
| A_24_P13190 | NM_138961 | ESAM | −0.99 | 0.001039624 |
| A_23_P151679 | NM_021003 | PPM1A | −0.99 | 0.032223315 |
| A_23_P156431 | NM_005907 | MAN1A1 | −0.989 | 0.016728965 |
| A_32_P139414 | AW804491 | AW804491 | −0.988 | 0.033045185 |
| A_23_P217079 | NM_003863 | DPM2 | −0.987 | 0.004109967 |
| A_23_P156708 | NM_032470 | TNXB | −0.986 | 0.009516239 |
| A_24_P753476 | NR_002942 | LOC340508 | −0.986 | 0.038417194 |
| A_23_P86330 | NM_016545 | IER5 | −0.986 | 0.009804412 |
| A_24_P353289 | NM_031444 | C22orf13 | −0.985 | 0.038492288 |
| A_23_P137630 | NM_016287 | HP1BP3 | −0.985 | 0.024799685 |
| A_23_P259272 | NM_018639 | WSB2 | −0.984 | 0.019666248 |
| A_32_P178099 | EB386378 | EB386378 | −0.984 | 0.044977336 |
| A_23_P81219 | NM_016619 | PLAC8 | −0.983 | 0.040922281 |
| A_23_P143774 | NM_018995 | MOV10L1 | −0.982 | 0.014205566 |
| A_32_P89269 | THC2670311 | THC2670311 | −0.98 | 0.048641801 |
| A_23_P408095 | NM_001011546 | DSTN | −0.979 | 0.029116309 |
| A_32_P25243 | THC2656826 | THC2656826 | −0.979 | 0.046376759 |
| A_23_P393656 | AK055893 | NPHP3 | −0.978 | 0.015790132 |
| A_24_P203000 | NM_000878 | IL2RB | −0.977 | 0.011117994 |
| A_23_P417951 | NM_033512 | TSPYL5 | −0.976 | 0.046704674 |
| A_24_P935893 | NM_017709 | FAM46C | −0.976 | 0.002022435 |
| A_24_P186354 | A_24_P186354 | A_24_P186354 | −0.974 | 0.004538597 |
| A_23_P142325 | ENST00000395095 | ENST00000395095 | −0.972 | 0.044577504 |
| A_23_P502710 | NM_152237 | GAS2L1 | −0.972 | 0.014937717 |
| A_23_P73457 | NM_025158 | RUFY1 | −0.971 | 0.022818329 |
| A_23_P200073 | NM_020362 | C1orf128 | −0.97 | 0.002923371 |
| A_24_P393151 | AF025529 | LILRA1 | −0.97 | 0.031455378 |
| A_24_P218151 | NM_016352 | CPA4 | −0.968 | 0.007992622 |
| A_23_P24843 | NM_014632 | MICAL2 | −0.968 | 0.016444503 |
| A_23_P153351 | NM_000713 | BLVRB | −0.968 | 0.006460537 |
| A_23_P205646 | NM_198794 | MAP4K5 | −0.966 | 0.042568608 |
| A_23_P206441 | NM_000135 | FANCA | −0.965 | 0.036189325 |
| A_23_P206441 | NM_000135 | FANCA | −0.964 | 0.029232983 |
| A_24_P383704 | THC2625585 | THC2625585 | −0.964 | 0.01106912 |
| A_23_P206441 | NM_000135 | FANCA | −0.964 | 0.031775657 |
| A_24_P293114 | NM_020971 | SPTBN4 | −0.963 | 0.004554433 |
| A_24_P139901 | NM_002101 | GYPC | −0.961 | 0.005786791 |
| A_24_P1545 | NM_015633 | FGFR1OP2 | −0.961 | 0.005567299 |
| A_24_P336276 | NM_013272 | SLCO3A1 | −0.96 | 0.035324369 |
| A_23_P250274 | NM_019594 | LRRC8A | −0.959 | 0.039822844 |
| A_32_P180971 | BC090920 | BC090920 | −0.958 | 0.019744858 |
| A_24_P347880 | NM_021628 | ALOXE3 | −0.957 | 0.013990144 |
| A_23_P118392 | NM_016084 | RASD1 | −0.957 | 0.014422242 |
| A_23_P212436 | NM_001008392 | CTDSPL | −0.956 | 0.045020244 |
| A_23_P65370 | NM_016417 | GLRX5 | −0.956 | 0.004529116 |
| A_23_P250212 | NM_001080826 | PRAGMIN | −0.956 | 0.044641062 |
| A_24_P176445 | NM_001040427 | HAGH | −0.955 | 0.009145834 |
| A_23_P19936 | NM_006854 | KDELR2 | −0.954 | 0.037053822 |
| A_23_P162739 | NM_183422 | TSC22D1 | −0.952 | 0.043662926 |
| A_23_P254507 | NM_139211 | HOPX | −0.952 | 0.013144674 |
| A_23_P257753 | NM_022780 | RMND5A | −0.951 | 0.038402379 |
| A_23_P59210 | NM_000389 | CDKN1A | −0.95 | 0.045494125 |
| A_23_P82169 | NM_003107 | SOX4 | −0.949 | 0.031197923 |
| A_24_P913489 | M28219 | LDLR | −0.948 | 0.015836584 |
| A_23_P204511 | NM_017842 | FLJ20489 | −0.946 | 0.019961078 |
| A_32_P70927 | NM_207339 | PAGE2 | −0.945 | 0.009281631 |
| A_24_P910818 | THC2786662 | THC2786662 | −0.945 | 0.018308564 |
| A_23_P94533 | NM_001912 | CTSL1 | −0.944 | 0.027436717 |
| A_23_P206290 | NM_153837 | GPR114 | −0.944 | 0.036899568 |
| A_23_P100660 | NM_002615 | SERPINF1 | −0.942 | 0.037508295 |
| A_23_P133949 | A_23_P133949 | A_23_P133949 | −0.942 | 0.046398654 |
| A_32_P112910 | NM_203412 | UBL4B | −0.941 | 0.00824716 |
| A_23_P97123 | BC007072 | RP4-747L4.3 | −0.941 | 0.043258544 |
| A_23_P26386 | NM_016140 | TPPP3 | −0.941 | 0.04784671 |
| A_32_P89709 | NM_001018004 | TPM1 | −0.94 | 0.004639675 |
| A_23_P307525 | NM_152326 | ANKRD9 | −0.94 | 0.014652346 |
| A_24_P80135 | NM_014369 | PTPN18 | −0.94 | 0.016325115 |
| A_24_P502660 | A_24_P502660 | A_24_P502660 | −0.939 | 0.026513951 |
| A_23_P118427 | NM_145109 | MAP2K3 | −0.939 | 0.007612662 |
| A_23_P6252 | AJ783421 | DSCR8 | −0.938 | 0.046148052 |
| A_23_P107454 | NM_031958 | KRTAP3-1 | −0.937 | 0.034844698 |
| A_24_P374244 | NM_002049 | GATA1 | −0.936 | 0.010383484 |
| A_23_P29551 | NM_018306 | TMEM40 | −0.935 | 0.011038904 |
| A_23_P80594 | NM_015184 | PLCL2 | −0.935 | 0.0476128 |
| A_23_P131825 | NM_003279 | TNNC2 | −0.934 | 0.027353442 |
| A_23_P143811 | A_23_P143811 | A_23_P143811 | −0.932 | 0.031325291 |
| A_23_P146631 | NM_006336 | ZER1 | −0.932 | 0.022970031 |
| A_23_P204937 | NM_014059 | C13orf15 | −0.931 | 0.02388524 |
| A_23_P160481 | NM_018442 | IQWD1 | −0.93 | 0.02431942 |
| A_24_P22887 | NM_005053 | RAD23A | −0.93 | 0.007339641 |
| A_23_P216004 | AY032628 | SLC25A37 | −0.93 | 0.021784563 |
| A_23_P72961 | NM_002764 | PRPS1 | −0.93 | 0.014360234 |
| A_23_P57227 | NM_003098 | SNTA1 | −0.929 | 0.024396898 |
| A_23_P209251 | THC2741486 | THC2741486 | −0.929 | 0.046237472 |
| A_32_P69368 | NM_002166 | ID2 | −0.929 | 0.042580391 |
| A_24_P181998 | ENST00000255896 | ENST00000255896 | −0.929 | 0.036159465 |
| A_24_P414187 | NM_014869 | IQSEC1 | −0.928 | 0.023304278 |
| A_23_P210886 | NM_138578 | BCL2L1 | −0.928 | 0.005764794 |
| A_24_P71938 | NM_005900 | SMAD1 | −0.928 | 0.039301763 |
| A_23_P166633 | NM_002213 | ITGB5 | −0.928 | 0.015720253 |
| A_24_P246926 | NM_001302 | CORT | −0.927 | 0.012612682 |
| A_23_P136870 | NM_175868 | MAGEA6 | −0.926 | 0.021687261 |
| A_23_P365610 | X56681 | JUD | −0.925 | 0.041036031 |
| A_24_P248251 | NM_139048 | HLTF | −0.923 | 0.032343997 |
| A_24_P187365 | A_24_P187365 | A_24_P187365 | −0.922 | 0.045062579 |
| A_32_P173122 | A_32_P173122 | A_32_P173122 | −0.921 | 0.048382249 |
| A_23_P128919 | NM_002306 | LGALS3 | −0.92 | 0.009391517 |
| A_24_P25668 | NM_000797 | DRD4 | −0.92 | 0.037246263 |
| A_23_P133107 | AK027833 | FRAS1 | −0.919 | 0.037794184 |
| A_23_P20035 | NM_138445 | GPR146 | −0.917 | 0.01106072 |
| A_23_P41267 | BC010526 | LOC401127 | −0.915 | 0.009322662 |
| A_23_P345118 | NM_002648 | PIM1 | −0.915 | 0.015122906 |
| A_23_P119042 | NM_005601 | NKG7 | −0.913 | 0.018093694 |
| A_24_P886515 | THC2654231 | THC2654231 | −0.913 | 0.004358116 |
| A_24_P38276 | NM_003505 | FZD1 | −0.912 | 0.030144914 |
| A_23_P210886 | NM_138578 | BCL2L1 | −0.91 | 0.008270324 |
| A_23_P161399 | NM_130439 | MXI1 | −0.909 | 0.008737222 |
| A_23_P134454 | NM_001753 | CAV1 | −0.908 | 0.011501773 |
| A_24_P858698 | NM_002501 | NFIX | −0.908 | 0.000844457 |
| A_23_P372834 | NM_198098 | AQP1 | −0.907 | 0.008616887 |
| A_23_P52286 | NM_015448 | RP11-529I10.4 | −0.907 | 0.014917153 |
| A_23_P136870 | NM_175868 | MAGEA6 | −0.907 | 0.038067568 |
| A_23_P308603 | NM_005417 | SRC | −0.907 | 0.045398471 |
| A_23_P166797 | NM_022147 | RTP4 | −0.907 | 0.040360762 |
| A_23_P206441 | NM_000135 | FANCA | −0.904 | 0.048294437 |
| A_23_P206441 | NM_000135 | FANCA | −0.901 | 0.048062397 |
| A_23_P414308 | NM_144606 | FLCN | −0.901 | 0.00187956 |
| A_23_P136870 | NM_175868 | MAGEA6 | −0.9 | 0.026229263 |
| A_23_P345575 | NM_001455 | FOXO3 | −0.9 | 0.010310405 |
| A_23_P352435 | NM_002926 | RGS12 | −0.9 | 0.036435682 |
| A_32_P103776 | THC2734788 | THC2734788 | −0.9 | 0.013337258 |
| A_24_P935203 | THC2500587 | THC2500587 | −0.899 | 0.031083139 |
| A_24_P485742 | THC2689427 | THC2689427 | −0.899 | 0.048158934 |
| A_24_P318656 | NM_000212 | ITGB3 | −0.897 | 0.003631432 |
| A_32_P46238 | NR_001443 | LOC339240 | −0.897 | 0.049601213 |
| A_23_P206018 | NM_001018004 | TPM1 | −0.896 | 0.036805537 |
| A_23_P78289 | NM_032837 | FAM104A | −0.894 | 0.014699225 |
| A_24_P289865 | A_24_P289865 | A_24_P289865 | −0.894 | 0.024212912 |
| A_24_P919330 | NM_002032 | FTH1 | −0.894 | 0.011432889 |
| A_23_P152906 | NM_000697 | ALOX12 | −0.892 | 0.022462007 |
| A_24_P752518 | NM_005961 | MUC6 | −0.891 | 0.043791418 |
| A_24_P73669 | NM_002094 | GSPT1 | −0.89 | 0.007954744 |
| A_24_P366787 | BC029785 | FLJ42875 | −0.89 | 0.025260698 |
| A_23_P33072 | NM_032272 | MAF1 | −0.889 | 0.018451497 |
| A_23_P120364 | NM_024299 | C20orf149 | −0.888 | 0.020101092 |
| A_24_P401150 | A_24_P401150 | A_24_P401150 | −0.887 | 0.005915351 |
| A_23_P372910 | NM_005060 | RORC | −0.887 | 0.013773819 |
| A_24_P148590 | NM_015727 | TACR1 | −0.885 | 0.026326369 |
| A_23_P62429 | NM_001078172 | FAM127B | −0.883 | 0.003838268 |
| A_23_P75430 | NM_020179 | C11orf75 | −0.882 | 0.040992364 |
| A_23_P256059 | NM_203477 | MGC70863 | −0.881 | 0.028605665 |
| A_24_P201089 | NM_016612 | SLC25A37 | −0.881 | 0.003906411 |
| A_24_P744148 | BC009800 | BC009800 | −0.878 | 0.018810939 |
| A_24_P266466 | NM_005293 | GPR20 | −0.877 | 0.031648015 |
| A_23_P200001 | NM_144573 | NEXN | −0.877 | 0.039308654 |
| A_24_P159845 | NM_007145 | ZNF146 | −0.877 | 0.023356958 |
| A_24_P172993 | NM_022066 | UBE2O | −0.876 | 0.005368728 |
| A_24_P649735 | BY796363 | BY796363 | −0.876 | 0.018706305 |
| A_23_P203351 | NM_021201 | MS4A7 | −0.872 | 0.005827151 |
| A_24_P385190 | NM_000342 | SLC4A1 | −0.872 | 0.013740393 |
| A_23_P77117 | NM_023003 | TM6SF1 | −0.871 | 0.035404496 |
| A_23_P41528 | NM_031950 | FGFBP2 | −0.87 | 0.003165653 |
| A_24_P130792 | NM_199044 | NSUN4 | −0.87 | 0.009518779 |
| A_24_P401670 | XR_015583 | XR_015583 | −0.87 | 0.013423217 |
| A_23_P26143 | BU568037 | BU568037 | −0.868 | 0.013618301 |
| A_23_P385851 | NM_133486 | MBNL3 | −0.868 | 0.045014898 |
| A_24_P128727 | ENST00000306515 | ENST00000306515 | −0.868 | 0.013502375 |
| A_23_P200015 | NM_174858 | AK5 | −0.867 | 0.013241102 |
| A_23_P78053 | NM_030802 | FAM117A | −0.867 | 0.010940632 |
| A_23_P408708 | NM_004331 | BNIP3L | −0.866 | 0.033082772 |
| A_23_P421032 | NM_174977 | SEC14L4 | −0.865 | 0.016444532 |
| A_23_P1833 | NM_054025 | B3GAT1 | −0.865 | 0.01851404 |
| A_24_P115007 | NM_170740 | ALDH5A1 | −0.865 | 0.017766601 |
| A_23_P210886 | NM_138578 | BCL2L1 | −0.863 | 0.010306287 |
| A_32_P168342 | ENST00000375806 | C6orf25 | −0.862 | 0.002760058 |
| A_24_P164718 | NM_016496 | 2-Mar | −0.862 | 0.005503335 |
| A_23_P393627 | NM_138693 | KLF14 | −0.862 | 0.001454426 |
| A_23_P325726 | NM_015547 | ACOT11 | −0.861 | 0.038505496 |
| A_23_P75260 | NM_032023 | RASSF4 | −0.86 | 0.01928567 |
| A_32_P30004 | AF086044 | AF086044 | −0.859 | 0.049159541 |
| A_23_P143374 | NM_025176 | RP4-691N24.1 | −0.858 | 0.049322269 |
| A_24_P359205 | BC010094 | PIGQ | −0.858 | 0.049451763 |
| A_32_P35751 | THC2704459 | THC2704459 | −0.858 | 0.024232202 |
| A_32_P148047 | CD239565 | CD239565 | −0.857 | 0.008908719 |
| A_23_P151506 | NM_016445 | PLEK2 | −0.856 | 0.00810997 |
| A_23_P2041 | NM_032867 | MICALCL | −0.855 | 0.007866741 |
| A_24_P51127 | BC094862 | WNK1 | −0.855 | 0.012386376 |
| A_23_P80382 | NM_015366 | PRR5 | −0.855 | 0.004157962 |
| A_24_P336853 | NM_020143 | PNO1 | −0.854 | 0.008769356 |
| A_23_P82959 | NM_003923 | FOXH1 | −0.851 | 0.002074859 |
| A_24_P59247 | XM_001720677 | LOC390282 | −0.851 | 0.025203696 |
| A_32_P25253 | NM_030940 | ISCA1 | −0.851 | 0.012341357 |
| A_23_P350059 | NR_003242 | C1orf152 | −0.85 | 0.029775313 |
| A_23_P207967 | NM_014772 | KIAA0427 | −0.85 | 0.010930804 |
| A_23_P379630 | NM_138570 | SLC38A10 | −0.85 | 0.011862072 |
| A_23_P102731 | NM_175839 | SMOX | −0.849 | 0.021129481 |
| A_23_P106559 | ENST00000397462 | ENST00000397462 | −0.848 | 0.046801596 |
| A_23_P139198 | NM_033101 | LGALS12 | −0.848 | 0.038894278 |
| A_23_P210886 | NM_138578 | BCL2L1 | −0.845 | 0.012426389 |
| A_24_P204257 | ENST00000391373 | ENST00000391373 | −0.845 | 0.009472109 |
| A_23_P308603 | NM_005417 | SRC | −0.844 | 0.042938017 |
| A_23_P210886 | NM_138578 | BCL2L1 | −0.843 | 0.010479679 |
| A_23_P254271 | NM_032525 | TUBB6 | −0.843 | 0.008035822 |
| A_24_P222054 | AF271776 | AF271776 | −0.843 | 0.046473222 |
| A_32_P122402 | BC031344 | LOC441455 | −0.842 | 0.011264687 |
| A_23_P48212 | NM_016509 | CLEC1B | −0.842 | 0.030134552 |
| A_24_P599496 | THC2613527 | THC2613527 | −0.842 | 0.017088261 |
| A_23_P210886 | NM_138578 | BCL2L1 | −0.841 | 0.011183527 |
| A_32_P95082 | NM_017738 | CNTLN | −0.841 | 0.036294859 |
| A_23_P68486 | NM_080821 | C20orf108 | −0.838 | 0.018387489 |
| A_32_P4228 | ENST00000358396 | LOC728450 | −0.837 | 0.011047822 |
| A_23_P42241 | NM_030876 | OR5V1 | −0.837 | 0.027550271 |
| A_24_P718503 | AK127768 | LOC100130152 | −0.837 | 0.044405315 |
| A_24_P595717 | NM_199280 | LOC165186 | −0.836 | 0.024643475 |
| A_32_P113742 | BC104478 | RPL21 | −0.836 | 0.033300318 |
| A_23_P257111 | NM_000507 | FBP1 | −0.836 | 0.043029992 |
| A_32_P159770 | CF139200 | CF139200 | −0.835 | 0.048391598 |
| A_23_P103131 | NM_019008 | SMCR7L | −0.835 | 0.046753169 |
| A_24_P99963 | NM_001319 | CSNK1G2 | −0.834 | 0.001126569 |
| A_24_P59297 | ENST00000391607 | ENST00000391607 | −0.833 | 0.049739973 |
| A_23_P140675 | NM_000119 | EPB42 | −0.833 | 0.003153707 |
| A_23_P210886 | NM_138578 | BCL2L1 | −0.831 | 0.011312463 |
| A_23_P420196 | NM_003745 | SOCS1 | −0.831 | 0.047693542 |
| A_32_P19466 | A_32_P19466 | A_32_P19466 | −0.831 | 0.04050838 |
| A_23_P210886 | NM_138578 | BCL2L1 | −0.83 | 0.011773337 |
| A_23_P206140 | NM_018602 | DNAJA4 | −0.83 | 0.017751665 |
| A_24_P101391 | NM_004559 | YBX1 | −0.829 | 0.016826692 |
| A_24_P355267 | NM_001006641 | SLC25A25 | −0.829 | 0.024690665 |
| A_23_P13057 | NM_020229 | PRDM11 | −0.828 | 0.01068724 |
| A_23_P77971 | NM_000419 | ITGA2B | −0.827 | 0.004955564 |
| A_32_P188841 | A_32_P188841 | A_32_P188841 | −0.826 | 0.018791933 |
| A_23_P101908 | NM_005883 | APC2 | −0.826 | 0.010376856 |
| A_23_P100326 | NM_001039476 | C16orf35 | −0.826 | 0.012568956 |
| A_23_P1473 | NM_005041 | PRF1 | −0.825 | 0.008965126 |
| A_23_P31984 | NM_006687 | ACTL7A | −0.825 | 0.026202476 |
| A_23_P35796 | NM_006244 | PPP2R5B | −0.824 | 0.02153886 |
| A_23_P432056 | NM_178568 | RTN4RL1 | −0.82 | 0.00993646 |
| A_23_P209426 | NM_015049 | TRAK2 | −0.819 | 0.038392342 |
| A_23_P5221 | NM_032433 | ZNF333 | −0.819 | 0.003499427 |
| A_24_P237117 | NM_002237 | KCNG1 | −0.818 | 0.014212163 |
| A_24_P921366 | NM_033138 | CALD1 | −0.817 | 0.032377341 |
| A_24_P682601 | XR_019446 | XR_019446 | −0.815 | 0.008822215 |
| A_23_P210886 | NM_138578 | BCL2L1 | −0.814 | 0.015207437 |
| A_23_P33683 | NM_016496 | 2-Mar | −0.814 | 0.008634299 |
| A_23_P318800 | NM_001050 | SSTR2 | −0.813 | 0.013041064 |
| A_23_P73780 | NM_001569 | IRAK1 | −0.813 | 0.00877921 |
| A_23_P48088 | NM_001242 | CD27 | −0.811 | 0.025621192 |
| A_23_P430818 | NM_014181 | HSPC159 | −0.811 | 0.014252924 |
| A_24_P922378 | NM_004449 | ERG | −0.81 | 0.024735679 |
| A_23_P49559 | NM_181790 | GPR142 | −0.808 | 0.007844983 |
| A_23_P210886 | NM_138578 | BCL2L1 | −0.808 | 0.013895752 |
| A_23_P251173 | NM_014413 | EIF2AK1 | −0.807 | 0.020667132 |
| A_23_P136870 | NM_175868 | MAGEA6 | −0.805 | 0.038562877 |
| A_23_P368366 | NM_207332 | ERICH1 | −0.805 | 0.031645458 |
| A_23_P321261 | NM_002396 | ME2 | −0.805 | 0.036538761 |
| A_23_P254120 | NM_033480 | FBXO9 | −0.803 | 0.027653021 |
| A_24_P385942 | NM_133371 | MYOZ3 | −0.802 | 0.015125662 |
| A_23_P125204 | NM_001004464 | OR10G8 | −0.802 | 0.04262958 |
| A_23_P144126 | NM_014375 | FETUB | −0.801 | 0.046119906 |
| A_24_P246626 | ENST00000308384 | ENST00000308384 | −0.8 | 0.03949341 |
| A_23_P104413 | NM_033178 | DUX4 | −0.799 | 0.040081012 |
| A_24_P47467 | NM_024092 | TMEM109 | −0.799 | 0.032354833 |
| A_23_P69121 | NM_005067 | SIAH2 | −0.797 | 0.01601201 |
| A_24_P206305 | NM_182924 | MICALL2 | −0.795 | 0.035105181 |
| A_23_P216108 | NM_000037 | ANK1 | −0.792 | 0.005501967 |
| A_23_P58729 | NM_003052 | SLC34A1 | −0.791 | 0.009203365 |
| A_23_P75149 | NM_213649 | SFXN4 | −0.791 | 0.038292358 |
| A_32_P178503 | NM_001012984 | C16orf86 | −0.791 | 0.015151804 |
| A_24_P156769 | NM_005373 | MPL | −0.79 | 0.048426697 |
| A_24_P11436 | NM_017904 | TTC22 | −0.79 | 0.033041142 |
| A_23_P116259 | NM_018400 | SCN3B | −0.789 | 0.006781432 |
| A_32_P16975 | NM_133450 | ANKS3 | −0.787 | 0.011138264 |
| A_23_P217554 | NM_005364 | MAGEA8 | −0.786 | 0.033532412 |
| A_32_P102062 | NM_001455 | FOXO3 | −0.785 | 0.01783253 |
| A_24_P323682 | A_24_P323682 | A_24_P323682 | −0.785 | 0.007378948 |
| A_32_P213624 | BU537444 | BU537444 | −0.785 | 0.025583076 |
| A_23_P160359 | NM_004437 | EPB41 | −0.783 | 0.012499004 |
| A_24_P409971 | NM_144573 | NEXN | −0.783 | 0.037400632 |
| A_23_P332937 | NM_152480 | C19orf23 | −0.78 | 0.006912274 |
| A_24_P272713 | XR_018980 | XR_018980 | −0.779 | 0.022895332 |
| A_23_P397120 | ENST00000211092 | ENST00000211092 | −0.779 | 0.016700375 |
| A_24_P709949 | BM351936 | BM351936 | −0.779 | 0.013355373 |
| A_23_P16096 | NM_020230 | PPAN | −0.778 | 0.005164182 |
| A_24_P367571 | NM_017794 | KIAA1797 | −0.778 | 0.015883237 |
| A_23_P433588 | NM_001406 | EFNB3 | −0.776 | 0.033076045 |
| A_23_P259901 | NM_012253 | TKTL1 | −0.776 | 0.028829566 |
| A_24_P910050 | CD242823 | CD242823 | −0.775 | 0.008232932 |
| A_32_P163089 | ENST00000310995 | ENST00000310995 | −0.774 | 0.043162695 |
| A_23_P50389 | NM_020378 | NAT14 | −0.774 | 0.005351012 |
| A_23_P215913 | NM_203339 | CLU | −0.773 | 0.016302179 |
| A_24_P556883 | AI732974 | AI732974 | −0.773 | 0.013573178 |
| A_23_P87329 | NM_024662 | NAT10 | −0.772 | 0.003027403 |
| A_32_P209624 | THC2522223 | THC2522223 | −0.772 | 0.016674144 |
| A_24_P334640 | NM_133367 | PAQR8 | −0.77 | 0.045427676 |
| A_23_P369899 | NM_015444 | TMEM158 | −0.77 | 0.010285347 |
| A_23_P113701 | NM_002607 | PDGFA | −0.77 | 0.029266863 |
| A_23_P257583 | NM_015689 | DENND2A | −0.768 | 0.025049083 |
| A_24_P600036 | A_24_P600036 | A_24_P600036 | −0.768 | 0.035753682 |
| A_24_P87931 | NM_145343 | APOL1 | −0.768 | 0.032442685 |
| A_32_P209960 | NM_000246 | CIITA | −0.767 | 0.032989305 |
| A_23_P79978 | NM_020689 | SLC24A3 | −0.767 | 0.011006821 |
| A_23_P100408 | NM_006181 | NTN2L | −0.766 | 0.022874366 |
| A_23_P8452 | NM_001040167 | LFNG | −0.764 | 0.014747178 |
| A_24_P341593 | XR_015535 | XR_015535 | −0.764 | 0.036571567 |
| A_23_P405088 | NM_005602 | CLDN11 | −0.763 | 0.04903843 |
| A_32_P192474 | NM_030651 | PRRT1 | −0.763 | 0.02633235 |
| A_23_P71649 | NM_005592 | MUSK | −0.762 | 0.024983032 |
| A_24_P818268 | ENST00000339692 | ENST00000339692 | −0.762 | 0.020906026 |
| A_23_P129382 | NM_005425 | TNP2 | −0.762 | 0.036207578 |
| A_23_P151106 | NM_007273 | PHB2 | −0.76 | 0.002332462 |
| A_23_P416581 | NM_002073 | GNAZ | −0.758 | 0.015814234 |
| A_24_P8220 | NM_004807 | HS6ST1 | −0.756 | 0.049495269 |
| A_23_P89799 | NM_006111 | ACAA2 | −0.754 | 0.028752976 |
| A_24_P133475 | NM_138452 | DHRS1 | −0.754 | 0.011009747 |
| A_23_P51986 | NM_181873 | MTMR11 | −0.754 | 0.010877511 |
| A_24_P356930 | NM_000782 | CYP24A1 | −0.754 | 0.03354862 |
| A_32_P204740 | BC065754 | BC065754 | −0.753 | 0.00890976 |
| A_24_P931804 | A_24_P931804 | A_24_P931804 | −0.752 | 0.045035887 |
| A_32_P184260 | AL516445 | AL516445 | −0.752 | 0.007042534 |
| A_32_P102300 | THC2687538 | THC2687538 | −0.751 | 0.022593225 |
| A_24_P330385 | NM_016609 | SLC22A17 | −0.75 | 0.019781267 |
| A_23_P152984 | NM_005782 | THOC4 | −0.749 | 0.024798857 |
| A_32_P191120 | THC2744687 | THC2744687 | −0.749 | 0.047231303 |
| A_24_P499481 | A_24_P499481 | A_24_P499481 | −0.748 | 0.027237843 |
| A_23_P134454 | NM_001753 | CAV1 | −0.747 | 0.018780675 |
| A_23_P150064 | NM_024756 | MMRN2 | −0.747 | 0.037154932 |
| A_23_P142045 | NM_005809 | PRDX2 | −0.745 | 0.014601301 |
| A_23_P26890 | NM_024302 | MMP28 | −0.745 | 0.022789107 |
| A_23_P148641 | NR_001551 | TTTY12 | −0.744 | 0.046602585 |
| A_24_P341019 | NM_001009924 | C20orf30 | −0.742 | 0.006769506 |
| A_23_P15369 | NM_174892 | CD300LB | −0.742 | 0.034082289 |
| A_23_P333129 | ENST00000391334 | ENST00000391334 | −0.74 | 0.046662458 |
| A_24_P169073 | NM_182623 | FAM131C | −0.74 | 0.008326521 |
| A_23_P129829 | NM_139280 | ORMDL3 | −0.739 | 0.00170473 |
| A_23_P134454 | NM_001753 | CAV1 | −0.738 | 0.019305087 |
| A_32_P150040 | CR606969 | CR606969 | −0.737 | 0.008290353 |
| A_23_P29897 | NM_005935 | AFF1 | −0.735 | 0.023497008 |
| A_24_P353638 | NM_021181 | SLAMF7 | −0.734 | 0.046919623 |
| A_32_P89523 | T12590 | T12590 | −0.734 | 0.040848352 |
| A_23_P1998 | NM_001164 | APBB1 | −0.733 | 0.047873402 |
| A_24_P827738 | A_24_P827738 | A_24_P827738 | −0.732 | 0.040287801 |
| A_23_P117125 | NM_000966 | RARG | −0.731 | 0.024220226 |
| A_23_P14302 | BC008299 | C14orf139 | −0.729 | 0.005187909 |
| A_23_P6321 | NM_003277 | CLDN5 | −0.729 | 0.028013153 |
| A_23_P48109 | NM_016533 | NINJ2 | −0.728 | 0.043108116 |
| A_24_P136453 | NM_022648 | TNS1 | −0.728 | 0.01476612 |
| A_24_P104512 | NM_001988 | EVPL | −0.727 | 0.006799588 |
| A_23_P134454 | NM_001753 | CAV1 | −0.727 | 0.0167663 |
| A_23_P137751 | NM_017709 | FAM46C | −0.726 | 0.010376714 |
| A_23_P206498 | NM_032366 | C16orf13 | −0.725 | 0.007369062 |
| A_23_P394304 | NM_005764 | PDZK1IP1 | −0.724 | 0.018325141 |
| A_24_P341853 | NM_031454 | RP3-402G11.5 | −0.721 | 0.046548815 |
| A_24_P911676 | NM_003107 | SOX4 | −0.721 | 0.032326734 |
| A_23_P29124 | NM_002688 | 5-SeP | −0.72 | 0.046900487 |
| A_23_P86021 | NM_003944 | SELENBP1 | −0.718 | 0.005891389 |
| A_24_P30034 | NM_032818 | C9orf100 | −0.717 | 0.039141595 |
| A_23_P61886 | AK055659 | TSPAN5 | −0.716 | 0.016939902 |
| A_23_P114670 | NM_014448 | ARHGEF16 | −0.716 | 0.010184664 |
| A_23_P7005 | NM_005787 | ALG3 | −0.716 | 0.023679765 |
| A_23_P77000 | NM_014909 | VASH1 | −0.715 | 0.014648936 |
| A_23_P218892 | NM_182917 | EIF4G1 | −0.714 | 0.018908824 |
| A_23_P106042 | NM_001037288 | CMTM5 | −0.713 | 0.031548447 |
| A_23_P144627 | NM_018933 | PCDHB13 | −0.713 | 0.012502211 |
| A_24_P243396 | NM_001008657 | TCOF1 | −0.711 | 0.023088999 |
| A_24_P247536 | NM_001040057 | FAM133B | −0.711 | 0.042720244 |
| A_23_P113005 | NM_004428 | EFNA1 | −0.711 | 0.033325538 |
| A_23_P164258 | NM_016518 | PIPOX | −0.709 | 0.046973457 |
| A_24_P305933 | NM_020698 | TMCC3 | −0.709 | 0.028175115 |
| A_24_P919256 | A_24_P919256 | A_24_P919256 | −0.707 | 0.010240398 |
| A_24_P937744 | THC2553569 | THC2553569 | −0.706 | 0.005126359 |
| A_24_P373885 | NM_152482 | C19orf25 | −0.705 | 0.020388416 |
| A_23_P73348 | NM_024335 | IRX6 | −0.704 | 0.037552773 |
| A_23_P346301 | NM_144723 | ZMAT2 | −0.704 | 0.049513416 |
| A_23_P78158 | NM_004703 | RABEP1 | −0.703 | 0.0115642 |
| A_23_P255257 | NM_015397 | WDR40A | −0.702 | 0.011709955 |
| A_24_P91094 | NM_006695 | RUNDC3A | −0.702 | 0.027923528 |
| A_32_P17133 | NM_006241 | PPP1R2 | −0.702 | 0.028590369 |
| A_23_P90679 | NM_018571 | ALS2CR2 | −0.7 | 0.021615997 |
| A_24_P936758 | AK074614 | IGF2 | −0.699 | 0.005132236 |
| A_32_P56890 | THC2749298 | THC2749298 | −0.699 | 0.04325359 |
| A_32_P219942 | NM_033132 | ZIC5 | −0.698 | 0.027056637 |
| A_24_P222139 | AK025047 | AK025047 | −0.698 | 0.041878527 |
| A_24_P405190 | NM_201626 | USP33 | −0.697 | 0.048202195 |
| A_23_P53630 | NM_014868 | RNF10 | −0.697 | 0.044159941 |
| A_23_P142255 | NM_020209 | SHD | −0.697 | 0.008250883 |
| A_32_P213755 | AL555100 | AL555100 | −0.695 | 0.006014738 |
| A_23_P138049 | THC2730708 | THC2730708 | −0.695 | 0.017740811 |
| A_24_P356218 | NM_015161 | ARL6IP1 | −0.694 | 0.039761476 |
| A_24_P7470 | A_24_P7470 | A_24_P7470 | −0.693 | 0.024922731 |
| A_32_P71888 | BX089493 | BX089493 | −0.693 | 0.009726081 |
| A_24_P319923 | NM_053025 | MYLK | −0.692 | 0.048980082 |
| A_24_P879428 | BC070363 | BC070363 | −0.69 | 0.046890295 |
| A_24_P226069 | NM_031950 | FGFBP2 | −0.689 | 0.020323466 |
| A_23_P13294 | NM_001004459 | OR1S2 | −0.687 | 0.032415377 |
| A_32_P143377 | NM_001017970 | TMEM30B | −0.686 | 0.048545328 |
| A_23_P16110 | NM_001079935 | OR7E24 | −0.685 | 0.0350984 |
| A_32_P56415 | THC2557134 | THC2557134 | −0.684 | 0.026489516 |
| A_23_P90997 | NM_148961 | OTOS | −0.684 | 0.045146354 |
| A_24_P365767 | NM_000397 | CYBB | −0.683 | 0.031955661 |
| A_23_P52986 | NM_152718 | VWCE | −0.683 | 0.013226474 |
| A_23_P157205 | NM_016116 | ASB4 | −0.683 | 0.040049631 |
| A_23_P168105 | NM_030652 | EGFL8 | −0.682 | 0.006884944 |
| A_24_P867111 | AK095081 | LOC283177 | −0.679 | 0.023808175 |
| A_32_P173744 | ENST00000339845 | ENST00000339845 | −0.679 | 0.043049615 |
| A_32_P168812 | AK094525 | AK094525 | −0.677 | 0.038417261 |
| A_24_P410627 | AF117233 | MKRN1 | −0.674 | 0.022874347 |
| A_23_P112452 | NR_003191 | GGTA1 | −0.674 | 0.013535995 |
| A_23_P55578 | NM_003831 | RIOK3 | −0.671 | 0.044732728 |
| A_23_P129188 | NM_033429 | CALML4 | −0.67 | 0.014139315 |
| A_32_P72887 | A_32_P72887 | A_32_P72887 | −0.67 | 0.012066811 |
| A_24_P153734 | BC009106 | SEC16B | −0.67 | 0.001234386 |
| A_24_P143032 | NM_152243 | CDC42EP1 | −0.67 | 0.030616493 |
| A_24_P19054 | NM_012121 | CDC42EP4 | −0.669 | 0.038205818 |
| A_23_P56288 | NM_052925 | LENG8 | −0.668 | 0.004429062 |
| A_23_P167674 | NM_000505 | F12 | −0.667 | 0.026058402 |
| A_32_P200787 | NM_145702 | TIGD1 | −0.664 | 0.042995888 |
| A_23_P134454 | NM_001753 | CAV1 | −0.664 | 0.028439939 |
| A_24_P625382 | NM_003651 | CSDA | −0.661 | 0.032727321 |
| A_23_P66497 | NM_014404 | CACNG5 | −0.661 | 0.014197915 |
| A_23_P50799 | NM_013939 | OR10H2 | −0.66 | 0.006389662 |
| A_32_P93807 | A_32_P93807 | A_32_P93807 | −0.66 | 0.012420698 |
| A_23_P314052 | NM_015431 | TRIM58 | −0.658 | 0.017514409 |
| A_24_P189739 | NM_030640 | DUSP16 | −0.658 | 0.041373048 |
| A_24_P918739 | BC028378 | ABHD1 | −0.655 | 0.008517081 |
| A_23_P20075 | NM_013389 | NPC1L1 | −0.655 | 0.00495875 |
| A_23_P109864 | NM_004122 | GHSR | −0.655 | 0.047388327 |
| A_32_P109532 | AB019568 | AB019568 | −0.654 | 0.040743514 |
| A_24_P269327 | AL136837 | DKFZp434F142 | −0.654 | 0.035212459 |
| A_24_P65373 | NM_000419 | ITGA2B | −0.653 | 0.036773124 |
| A_32_P209206 | BC035471 | PLEKHA3 | −0.649 | 0.005225456 |
| A_23_P74619 | NM_003944 | SELENBP1 | −0.648 | 0.015690431 |
| A_24_P161382 | AK096952 | LOC389833 | −0.648 | 0.017343392 |
| A_23_P86504 | NM_024541 | C10orf76 | −0.648 | 0.010315837 |
| A_23_P134454 | NM_001753 | CAV1 | −0.648 | 0.036180719 |
| A_24_P375002 | NM_004559 | YBX1 | −0.645 | 0.029824478 |
| A_23_P87432 | NM_052924 | RHPN1 | −0.643 | 0.034648588 |
| A_23_P123393 | NM_004519 | KCNQ3 | −0.643 | 0.003506958 |
| A_23_P76992 | NM_002632 | PGF | −0.642 | 0.006158092 |
| A_24_P247039 | AK094167 | FLJ36848 | −0.642 | 0.041258606 |
| A_32_P216809 | BX108033 | BX108033 | −0.642 | 0.028742512 |
| A_23_P109934 | NM_016173 | HEMK1 | −0.641 | 0.04771832 |
| A_23_P170667 | NM_181718 | ASPHD1 | −0.641 | 0.024809053 |
| A_23_P431388 | NM_144569 | SPOCD1 | −0.641 | 0.03796151 |
| A_23_P420831 | NM_052828 | TRIM10 | −0.641 | 0.038347589 |
| A_24_P342591 | NM_012102 | RERE | −0.64 | 0.034382806 |
| A_23_P4400 | NM_033059 | KRTAP4-11 | −0.637 | 0.014933719 |
| A_23_P104741 | ENST00000278934 | KIRREL3 | −0.637 | 0.006126195 |
| A_23_P8142 | NM_001832 | CLPS | −0.636 | 0.045158283 |
| A_24_P256155 | XM_372331 | NKX1-2 | −0.636 | 0.035979168 |
| A_32_P82623 | AK023045 | AGBL3 | −0.636 | 0.039703301 |
| A_23_P252491 | NM_016484 | PDZD11 | −0.634 | 0.018459153 |
| A_23_P351724 | NM_022648 | TNS1 | −0.633 | 0.014069811 |
| A_24_P923704 | ENST00000391481 | ENST00000391481 | −0.633 | 0.039810935 |
| A_23_P70843 | NM_199186 | BPGM | −0.633 | 0.015878593 |
| A_23_P94647 | NM_001005234 | OR1L3 | −0.632 | 0.032881959 |
| A_24_P25040 | A_24_P25040 | A_24_P25040 | −0.632 | 0.027680049 |
| A_24_P23636 | NM_014510 | PCLO | −0.63 | 0.007809277 |
| A_23_P160849 | NM_004106 | FCER1G | −0.629 | 0.014045321 |
| A_23_P42768 | NM_001110199 | FLJ37078 | −0.629 | 0.015012701 |
| A_23_P218269 | NM_016151 | TAOK2 | −0.629 | 0.002608445 |
| A_32_P234753 | BU674437 | BU674437 | −0.627 | 0.006791409 |
| A_23_P118086 | NM_152339 | SPATA2L | −0.627 | 0.01401342 |
| A_24_P76313 | NM_207334 | FAM43B | −0.627 | 0.035648655 |
| A_23_P39453 | NM_203304 | MEX3D | −0.626 | 0.002018765 |
| A_23_P139162 | NM_006725 | CD6 | −0.626 | 0.007820262 |
| A_24_P282288 | BC032944 | KIAA0317 | −0.625 | 0.038229525 |
| A_24_P929745 | AK021842 | AK021842 | −0.623 | 0.048167879 |
| A_24_P162485 | NM_000037 | ANK1 | −0.62 | 0.022961204 |
| A_23_P254970 | AK057799 | RNPC3 | −0.62 | 0.013664403 |
| A_23_P378514 | NM_145178 | ATOH7 | −0.619 | 0.021832676 |
| A_32_P177750 | NM_018571 | ALS2CR2 | −0.618 | 0.037774783 |
| A_24_P833097 | BE243669 | BE243669 | −0.618 | 0.041425989 |
| A_24_P945293 | NM_016079 | VPS24 | −0.618 | 0.011938834 |
| A_32_P79152 | THC2539923 | THC2539923 | −0.616 | 0.010277226 |
| A_23_P212954 | NM_006798 | UGT2A1 | −0.614 | 0.014833183 |
| A_32_P515971 | NM_173503 | EFCAB3 | −0.613 | 0.015232466 |
| A_24_P759955 | THC2548256 | THC2548256 | −0.611 | 0.007454691 |
| A_32_P6062 | A_32_P6062 | A_32_P6062 | −0.61 | 0.009382433 |
| A_23_P27424 | NM_133460 | ZNF418 | −0.608 | 0.006777366 |
| A_32_P136376 | CR594735 | CR594735 | −0.607 | 0.047247245 |
| A_23_P408955 | NM_004091 | E2F2 | −0.607 | 0.028556652 |
| A_23_P14216 | ENST00000379864 | ENST00000379864 | −0.606 | 0.015744691 |
| A_23_P17330 | NM_017859 | UCKL1 | −0.606 | 0.005824126 |
| A_23_P137046 | NM_022567 | NYX | −0.605 | 0.003418936 |
| A_32_P234570 | THC2668853 | THC2668853 | −0.605 | 0.025607284 |
| A_24_P910121 | THC2649401 | THC2649401 | −0.604 | 0.016411126 |
| A_24_P219094 | CR936651 | SIPA1L1 | −0.604 | 0.008413411 |
| A_24_P136454 | NM_022648 | TNS1 | −0.604 | 0.033368448 |
| A_23_P127721 | NM_002559 | P2RX3 | −0.603 | 0.023441929 |
| A_24_P35109 | NM_001306 | CLDN3 | −0.603 | 0.038141908 |
| A_24_P930551 | THC2585049 | THC2585049 | −0.603 | 0.002341435 |
| A_24_P42330 | AK090827 | LOC100127980 | −0.603 | 0.018226011 |
| A_23_P168993 | NM_000025 | ADRB3 | −0.602 | 0.040836245 |
| A_32_P27535 | BC045676 | C17orf72 | −0.602 | 0.014968754 |
| A_23_P128337 | NM_002824 | PTMS | −0.602 | 0.005307964 |
| A_23_P202769 | NM_005528 | DNAJC4 | −0.601 | 0.029304049 |
| A_24_P789033 | AW452819 | AW452819 | −0.601 | 0.035779749 |
| A_24_P153680 | NM_018660 | ZNF395 | −0.599 | 0.030818306 |
| A_32_P218228 | NM_001002034 | FAM109B | −0.597 | 0.00868393 |
| A_23_P32229 | NM_012127 | CIZ1 | −0.597 | 0.038757828 |
| A_23_P158485 | NM_022117 | TSPYL2 | −0.597 | 0.043887384 |
| A_23_P28595 | NM_004405 | DLX2 | −0.596 | 0.004237445 |
| A_23_P41365 | NM_012390 | SMR3A | −0.596 | 0.005162452 |
| A_32_P197026 | R96556 | R96556 | −0.595 | 0.028703029 |
| A_24_P215445 | NM_005169 | PHOX2A | −0.595 | 0.046722119 |
| A_23_P15798 | NM_031854 | KRTAP4-12 | −0.595 | 0.025708063 |
| A_24_P63347 | NM_002620 | PF4V1 | −0.595 | 0.00183578 |
| A_32_P6769 | BC071598 | D2HGDH | −0.594 | 0.024774164 |
| A_23_P209129 | NM_002288 | LAIR2 | −0.593 | 0.024602194 |
| A_23_P164112 | NM_014798 | PLEKHM1 | −0.593 | 0.021393506 |
| A_32_P232617 | AK023827 | RUNDC2B | −0.592 | 0.023636276 |
| A_23_P106945 | NM_001114 | ADCY7 | −0.591 | 0.043976536 |
| A_24_P917492 | BC009479 | TTLL3 | −0.589 | 0.025453943 |
| A_23_P11071 | NM_203473 | PORCN | −0.584 | 0.020567074 |
| A_23_P29499 | NM_001904 | CTNNB1 | −0.584 | 0.018087197 |
| A_24_P42071 | A_24_P42071 | A_24_P42071 | −0.584 | 0.046808378 |
| A_32_P156017 | THC2753719 | THC2753719 | −0.583 | 0.010821281 |
| A_24_P892790 | AL359650 | AL359650 | −0.582 | 0.006803886 |
| A_32_P163215 | THC2628099 | THC2628099 | −0.581 | 0.003142308 |
| A_23_P46740 | NM_032517 | LYZL1 | −0.579 | 0.029649709 |
| A_24_P936692 | BC033528 | BC033528 | −0.578 | 0.014871192 |
| A_24_P168033 | NM_001454 | FOXJ1 | −0.577 | 0.028887804 |
| A_23_P107465 | NM_002277 | KRT31 | −0.576 | 0.011252206 |
| A_24_P75324 | AK131217 | TTBK1 | −0.575 | 0.040956609 |
| A_23_P257144 | NM_012293 | PXDN | −0.575 | 0.037633416 |
| A_23_P2366 | NM_199040 | NUDT4 | −0.574 | 0.012914905 |
| A_23_P56239 | NM_005305 | GPR42 | −0.573 | 0.014758198 |
| A_24_P184445 | NM_002429 | MMP19 | −0.573 | 0.0265724 |
| A_24_P931583 | A_24_P931583 | A_24_P931583 | −0.573 | 0.020598822 |
| A_23_P201156 | NM_021189 | CADM3 | −0.573 | 0.039732893 |
| A_23_P149448 | NM_021170 | HES4 | −0.571 | 0.015791366 |
| A_24_P363896 | NM_032888 | COL27A1 | −0.57 | 0.030374958 |
| A_23_P74330 | BC068047 | MGC4473 | −0.569 | 0.00936323 |
| A_24_P373768 | NM_000164 | GIPR | −0.569 | 0.011167886 |
| A_23_P207049 | A_23_P207049 | A_23_P207049 | −0.569 | 0.007957544 |
| A_23_P109026 | NM_022358 | KCNK15 | −0.569 | 0.018477283 |
| A_23_P138567 | NM_032797 | AIFM2 | −0.569 | 0.022592326 |
| A_23_P17624 | NM_003253 | TIAM1 | −0.566 | 0.024079062 |
| A_24_P257259 | ENST00000354668 | ENST00000354668 | −0.565 | 0.014433993 |
| A_24_P112160 | NM_030570 | UPK3B | −0.565 | 0.020732491 |
| A_23_P75192 | A_23_P75192 | A_23_P75192 | −0.565 | 0.042419148 |
| A_24_P131580 | NM_031313 | ALPPL2 | −0.565 | 0.021404008 |
| A_24_P941404 | A_24_P941404 | A_24_P941404 | −0.564 | 0.028179164 |
| A_32_P18523 | AI064892 | AI064892 | −0.564 | 0.006011231 |
| A_23_P363313 | NM_153357 | SLC16A11 | −0.563 | 0.025117406 |
| A_23_P258769 | NM_002121 | HLA-DPB1 | −0.561 | 0.048498231 |
| A_23_P71258 | NM_057096 | CYP3A43 | −0.561 | 0.01503372 |
| A_23_P23839 | NM_001017403 | LGR6 | −0.561 | 0.012691275 |
| A_32_P80781 | THC2752167 | THC2752167 | −0.56 | 0.004304229 |
| A_24_P85143 | NM_022370 | ROBO3 | −0.56 | 0.046519025 |
| A_23_P96285 | NM_022912 | REEP1 | −0.558 | 0.009516805 |
| A_23_P106258 | NM_207117 | C14orf68 | −0.557 | 0.042795797 |
| A_24_P341972 | AK090466 | AK090466 | −0.557 | 0.022534514 |
| A_23_P202361 | NM_182639 | HPS1 | −0.557 | 0.02317859 |
| A_23_P217712 | NM_009589 | ARSD | −0.556 | 0.022853294 |
| A_23_P130169 | NM_014726 | TBKBP1 | −0.555 | 0.049260231 |
| A_32_P149967 | BX105952 | BX105952 | −0.554 | 0.012018636 |
| A_23_P422131 | NM_152375 | KLHDC7A | −0.552 | 0.02634389 |
| A_24_P163574 | NM_022574 | GIGYF1 | −0.551 | 0.011808024 |
| A_23_P365081 | NM_005250 | FOXL1 | −0.55 | 0.027252353 |
| A_23_P117380 | NM_138344 | C14orf152 | −0.549 | 0.016039903 |
| A_23_P101893 | NM_032447 | FBN3 | −0.547 | 0.049482991 |
| A_24_P919727 | A_24_P919727 | A_24_P919727 | −0.547 | 0.02189773 |
| A_23_P387552 | NM_000966 | RARG | −0.546 | 0.038407821 |
| A_24_P173294 | NM_031918 | KLF16 | −0.545 | 0.043154903 |
| A_23_P209578 | NM_013445 | GAD1 | −0.544 | 0.029568141 |
| A_23_P117882 | NM_004378 | CRABP1 | −0.544 | 0.016846985 |
| A_23_P87461 | NM_001478 | B4GALNT1 | −0.543 | 0.021040642 |
| A_32_P13348 | NP186315 | NP186315 | −0.543 | 0.021869662 |
| A_32_P141338 | THC2565151 | THC2565151 | −0.543 | 0.049944042 |
| A_32_P175282 | BF149382 | BF149382 | −0.543 | 0.01271058 |
| A_23_P20804 | NM_147202 | C9orf25 | −0.542 | 0.010429242 |
| A_24_P642328 | AA420998 | AA420998 | −0.542 | 0.008421302 |
| A_32_P200820 | A_32_P200820 | A_32_P200820 | −0.542 | 0.024598529 |
| A_23_P166207 | NR_003087 | ABCC13 | −0.541 | 0.043866268 |
| A_23_P168584 | NM_152992 | POMZP3 | −0.541 | 0.022421414 |
| A_24_P697573 | AI942297 | AI942297 | −0.541 | 0.045044626 |
| A_23_P82002 | NM_003214 | TEAD3 | −0.541 | 0.017080702 |
| A_24_P809964 | AA465699 | AA465699 | −0.541 | 0.009591289 |
| A_32_P75888 | CD243286 | CD243286 | −0.541 | 0.01046487 |
| A_23_P12858 | NM_152710 | C10orf27 | −0.541 | 0.047306196 |
| A_32_P336690 | NM_013373 | ZDHHC8 | −0.54 | 0.028534859 |
| A_23_P131909 | BC021178 | C20orf141 | −0.54 | 0.009251326 |
| A_23_P10135 | NM_001001349 | NKIRAS2 | −0.539 | 0.046392912 |
| A_32_P58407 | ENST00000369697 | KCND3 | −0.539 | 0.040374489 |
| A_32_P129288 | NM_030981 | RAB1B | −0.539 | 0.038621841 |
| A_24_P374962 | NM_018991 | STAG3L1 | −0.539 | 0.006602685 |
| A_24_P256570 | BC014215 | BC014215 | −0.537 | 0.039632239 |
| A_32_P9518 | BX090181 | BX090181 | −0.535 | 0.030121378 |
| A_32_P159445 | NM_015075 | IQSEC2 | −0.534 | 0.0409097 |
| A_23_P209954 | NM_006433 | GNLY | −0.534 | 0.023420275 |
| A_23_P206741 | A_23_P206741 | A_23_P206741 | −0.533 | 0.027930486 |
| A_24_P278192 | NM_000476 | AK1 | −0.533 | 0.046711537 |
| A_32_P220666 | THC2558776 | THC2558776 | −0.533 | 0.013318461 |
| A_24_P913598 | S66917 | S66917 | −0.532 | 0.03949616 |
| A_23_P25187 | NM_002281 | KRT81 | −0.532 | 0.029879733 |
| A_24_P64524 | ENST00000333139 | C11orf72 | −0.53 | 0.014175802 |
| A_23_P355471 | NM_201636 | TBXA2R | −0.53 | 0.034249461 |
| A_32_P151437 | NM_020825 | CRAMP1L | −0.53 | 0.033138259 |
| A_32_P131090 | A_32_P131090 | A_32_P131090 | −0.528 | 0.047849343 |
| A_24_P308096 | NM_000215 | JAK3 | −0.528 | 0.012341432 |
| A_32_P516342 | NM_182608 | ANKRD33 | −0.527 | 0.033055075 |
| A_23_P116765 | NM_002289 | LALBA | −0.527 | 0.014252314 |
| A_23_P87346 | NM_000519 | HBD | −0.526 | 0.024515213 |
| A_32_P233155 | BX105253 | BX105253 | −0.525 | 0.049011568 |
| A_23_P210361 | NM_022082 | C20orf59 | −0.525 | 0.037849129 |
| A_32_P192480 | CR627122 | CR627122 | −0.525 | 0.039918578 |
| A_23_P17880 | NM_005740 | DNAL4 | −0.524 | 0.011699418 |
| A_24_P105332 | NM_016382 | CD244 | −0.524 | 0.020241114 |
| A_24_P76008 | A_24_P76008 | A_24_P76008 | −0.524 | 0.037924028 |
| A_23_P435407 | NM_001448 | GPC4 | −0.522 | 0.012279437 |
| A_23_P319557 | AF426268 | C21orf89 | −0.521 | 0.020152106 |
| A_23_P101783 | NM_005535 | IL12RB1 | −0.52 | 0.035613509 |
| A_32_P214611 | THC2690528 | THC2690528 | −0.52 | 0.014105266 |
| A_23_P170901 | NM_152410 | PACRG | −0.52 | 0.0374655 |
| A_23_P371794 | NM_000725 | CACNB3 | −0.52 | 0.017441476 |
| A_23_P118095 | NM_005061 | RPL3L | −0.52 | 0.012654872 |
| A_23_P130639 | A_23_P130639 | A_23_P130639 | −0.52 | 0.032294096 |
| A_23_P309292 | NM_001109662 | C12orf51 | −0.519 | 0.040524136 |
| A_24_P416055 | NM_031452 | FAM103A1 | −0.519 | 0.020628284 |
| A_32_P106925 | THC2625851 | THC2625851 | −0.516 | 0.008243908 |
| A_24_P239183 | NM_018406 | MUC4 | −0.515 | 0.019009967 |
| A_24_P242036 | NR_002184 | dJ222E13.2 | −0.514 | 0.023917136 |
| A_23_P165952 | NM_024855 | ACTR5 | −0.512 | 0.038173666 |
| A_32_P75563 | THC2688821 | THC2688821 | −0.512 | 0.007348724 |
| A_23_P160890 | NM_138697 | TAS1R1 | −0.511 | 0.031378718 |
| A_23_P76622 | NM_001922 | DCT | −0.51 | 0.018241062 |
| A_24_P522678 | AF117899 | AF117899 | −0.509 | 0.030988363 |
| A_23_P258164 | NM_001302 | CORT | −0.509 | 0.011227047 |
| A_24_P930442 | NM_001048194 | RCC1 | −0.509 | 0.019683173 |
| A_23_P334186 | NM_005920 | MEF2D | −0.505 | 0.019249167 |
| A_24_P917783 | X90978 | RUNX1 | −0.504 | 0.008928 |
| A_32_P161836 | THC2588392 | THC2588392 | −0.504 | 0.011559569 |
| A_23_P163814 | XM_928464 | ATXN1L | −0.504 | 0.04512481 |
| A_32_P109653 | THC2669092 | THC2669092 | −0.503 | 0.018779902 |
| A_23_P45304 | NM_021083 | XK | −0.503 | 0.038811938 |
| A_24_P84781 | AK021933 | AK021933 | −0.501 | 0.031091894 |
| A_24_P106728 | NM_019042 | PUS7 | −0.501 | 0.013165379 |
| A_32_P220881 | THC2754464 | THC2754464 | −0.501 | 0.004682787 |
| A_23_P502808 | NM_178013 | PRIMA1 | −0.5 | 0.02461482 |
| A_32_P10432 | THC2679040 | THC2679040 | −0.5 | 0.015171471 |
| A_32_P19101 | THC2699069 | THC2699069 | −0.499 | 0.021731331 |
| A_32_P218493 | THC2649348 | THC2649348 | −0.499 | 0.032081813 |
| A_24_P903715 | A_24_P903715 | A_24_P903715 | −0.498 | 0.03448528 |
| A_24_P401990 | NM_000400 | ERCC2 | −0.498 | 0.019729652 |
| A_32_P191665 | BF088423 | BF088423 | −0.497 | 0.030617635 |
| A_24_P260325 | NM_004744 | LRAT | −0.497 | 0.036100945 |
| A_23_P500410 | NM_130463 | ATP6V1G2 | −0.496 | 0.039672889 |
| A_23_P21862 | A_23_P21862 | A_23_P21862 | −0.495 | 0.01284155 |
| A_23_P21425 | NM_178471 | GPR119 | −0.493 | 0.026388435 |
| A_24_P288298 | NM_002255 | KIR2DL4 | −0.491 | 0.023204677 |
| A_24_P322771 | NM_003225 | TFF1 | −0.49 | 0.037951312 |
| A_32_P48536 | THC2529969 | THC2529969 | −0.487 | 0.021136658 |
| A_32_P52076 | BX537551 | BX537551 | −0.486 | 0.012192815 |
| A_23_P47102 | NM_080658 | ACY3 | −0.484 | 0.045132865 |
| A_23_P336034 | NM_148962 | OXER1 | −0.483 | 0.032715048 |
| A_32_P174385 | A_32_P174385 | A_32_P174385 | −0.481 | 0.027595771 |
| A_23_P114707 | NM_004285 | H6PD | −0.48 | 0.049462975 |
| A_23_P89380 | NM_000342 | SLC4A1 | −0.48 | 0.027492203 |
| A_23_P211326 | NM_031413 | CECR2 | −0.478 | 0.03008958 |
| A_23_P86874 | NM_033347 | KCNK7 | −0.476 | 0.021710063 |
| A_32_P82907 | BX427435 | BX427435 | −0.476 | 0.022172374 |
| A_24_P221551 | AK094477 | LOC100129113 | −0.472 | 0.025642991 |
| A_24_P219418 | BC029861 | MGC34800 | −0.47 | 0.019959583 |
| A_32_P448360 | NM_178542 | C17orf65 | −0.47 | 0.019310341 |
| A_24_P375168 | BC034236 | MGC39821 | −0.469 | 0.035720066 |
| A_24_P82358 | NM_005251 | FOXC2 | −0.467 | 0.021847847 |
| A_32_P33294 | BM808621 | BM808621 | −0.466 | 0.020039429 |
| A_23_P82088 | NM_016588 | NRN1 | −0.465 | 0.0041099 |
| A_23_P332392 | AK054935 | TTC9B | −0.462 | 0.043380784 |
| A_23_P128235 | NM_006121 | KRT1 | −0.462 | 0.008094033 |
| A_23_P136961 | A_23_P136961 | A_23_P136961 | −0.461 | 0.023092786 |
| A_24_P576591 | AL137705 | AL137705 | −0.46 | 0.019191382 |
| A_23_P301995 | NM_173083 | LIN9 | −0.46 | 0.012104849 |
| A_32_P173862 | BC039385 | LOC494558 | −0.456 | 0.025699421 |
| A_24_P920525 | AK022468 | SORBS1 | −0.452 | 0.040448485 |
| A_23_P502343 | NM_025220 | ADAM33 | −0.449 | 0.046450625 |
| A_24_P64407 | NM_005519 | HMX2 | −0.445 | 0.027877298 |
| A_24_P490746 | THC2642612 | THC2642612 | −0.445 | 0.035385784 |
| A_24_P400832 | BC029580 | LOC100130950 | −0.443 | 0.030939258 |
| A_24_P931711 | A_24_P931711 | A_24_P931711 | −0.442 | 0.040034514 |
| A_23_P75867 | NM_207186 | OR10A4 | −0.442 | 0.031968334 |
| A_24_P919502 | A_24_P919502 | A_24_P919502 | −0.441 | 0.026881048 |
| A_23_P107735 | NM_001783 | CD79A | −0.441 | 0.032417699 |
| A_23_P67127 | NM_173633 | TMEM145 | −0.437 | 0.046892332 |
| A_23_P21747 | NM_019855 | CABP5 | −0.434 | 0.032578907 |
| A_23_P79086 | NM_002503 | NFKBIB | −0.434 | 0.029888669 |
| A_23_P51679 | NM_005920 | MEF2D | −0.434 | 0.032643378 |
| A_24_P150428 | NM_145019 | FAM124A | −0.433 | 0.029648161 |
| A_24_P355609 | NM_145015 | MRGPRF | −0.432 | 0.037150607 |
| A_24_P337104 | NM_000916 | OXTR | −0.424 | 0.032219762 |
| A_23_P345564 | NM_182647 | OPRL1 | −0.422 | 0.036116398 |
| A_32_P160438 | AF085874 | AF085874 | −0.422 | 0.030633047 |
| A_23_P160438 | NM_002479 | MYOG | −0.421 | 0.0313622 |
| A_24_P153732 | BC007572 | BC007572 | −0.421 | 0.049722426 |
| A_32_P30075 | THC2689579 | THC2689579 | −0.42 | 0.029093636 |
| A_24_P852756 | NM_020056 | HLA-DQA2 | −0.419 | 0.011712794 |
| A_32_P105217 | THC2673062 | THC2673062 | −0.419 | 0.043162876 |
| A_23_P435444 | NM_032087 | PCDHGA7 | −0.418 | 0.042805065 |
| A_24_P136744 | BC036431 | BC036431 | −0.416 | 0.025087548 |
| A_32_P40547 | NR_002766 | MEG3 | −0.415 | 0.046235799 |
| A_23_P363196 | NM_014418 | TCL6 | −0.415 | 0.024778623 |
| A_23_P377717 | NM_002516 | NOVA2 | −0.414 | 0.022110824 |
| A_32_P138366 | THC2714978 | THC2714978 | −0.413 | 0.018809112 |
| A_23_P335988 | NM_014235 | UBL4A | −0.412 | 0.025838038 |
| A_23_P62361 | NM_014235 | UBL4A | −0.41 | 0.032544385 |
| A_23_P207842 | NM_001024809 | RARA | −0.409 | 0.038200377 |
| A_23_P142835 | NM_004082 | DCTN1 | −0.407 | 0.020375696 |
| A_23_P168928 | NM_000497 | CYP11B1 | −0.405 | 0.037946548 |
| A_24_P7873 | XM_374386 | ELFN1 | −0.405 | 0.027459957 |
| A_32_P120014 | A_32_P120014 | A_32_P120014 | −0.4 | 0.032852717 |
| A_32_P215404 | AI915259 | AI915259 | −0.399 | 0.022942708 |
| A_24_P388593 | NM_024871 | MAP6D1 | −0.399 | 0.04321487 |
| A_23_P254797 | NM_025227 | BPIL1 | −0.393 | 0.01464561 |
| A_23_P132139 | NM_058180 | C21orf58 | −0.39 | 0.020209941 |
| A_24_P341897 | NM_004302 | ACVR1B | −0.389 | 0.044678074 |
| A_23_P141005 | NM_001144 | AMFR | −0.388 | 0.036973831 |
| A_23_P404685 | NM_178348 | LCE1A | −0.386 | 0.040671551 |
| A_23_P120513 | NM_080739 | C20orf141 | −0.386 | 0.046672909 |
| A_23_P44533 | NM_153268 | PLCXD2 | −0.385 | 0.019910185 |
| A_23_P113204 | NM_005247 | FGF3 | −0.38 | 0.033074767 |
| A_24_P50281 | XR_019167 | XR_019167 | −0.376 | 0.040608323 |
| A_23_P167537 | NM_006650 | CPLX2 | −0.375 | 0.030161615 |
| A_24_P311063 | BC018095 | hCG_20426 | −0.372 | 0.013430592 |
| A_24_P281636 | ENST00000398290 | ENST00000398290 | −0.37 | 0.040402051 |
| A_24_P384397 | NM_133452 | RAVER1 | −0.363 | 0.029518443 |
| A_24_P928250 | A_24_P928250 | A_24_P928250 | −0.36 | 0.015057449 |
| A_23_P130761 | U06936 | DBP | −0.357 | 0.021370949 |
| A_23_P89691 | BC066363 | SDK2 | −0.354 | 0.021952811 |
| A_23_P218505 | NM_000894 | LHB | −0.351 | 0.044693116 |
| A_23_P113682 | NM_080877 | SLC34A3 | −0.345 | 0.043101529 |
| A_23_P398574 | NM_005581 | BCAM | −0.343 | 0.028416218 |
| A_32_P185089 | A_32_P185089 | A_32_P185089 | −0.338 | 0.040170948 |
| A_24_P372643 | AF037066 | SLC22A18AS | −0.328 | 0.048525087 |
| A_23_P84670 | NM_007327 | GRIN1 | −0.327 | 0.04690795 |
| A_23_P325862 | AF116661 | AF116661 | −0.325 | 0.04411835 |
| A_23_P36364 | NM_006288 | THY1 | −0.309 | 0.048270102 |
| A_32_P23748 | BC037911 | BC037911 | −0.307 | 0.021070792 |
| A_24_P707860 | XM_001714359 | LOC100132738 | 0.3843 | 0.045006765 |
| A_23_P136026 | AK128476 | IGHA1 | 0.4421 | 0.015264773 |
| A_24_P40775 | NM_020862 | LRFN1 | 0.4543 | 0.033796609 |
| A_32_P108544 | NM_001078170 | RGPD2 | 0.4664 | 0.027980085 |
| A_23_P169494 | NM_000607 | ORM1 | 0.4824 | 0.038181406 |
| A_23_P258493 | NM_005573 | LMNB1 | 0.4946 | 0.028716403 |
| A_24_P169873 | ENST00000390539 | ENST00000390539 | 0.51 | 0.001197881 |
| A_32_P8351 | CR627362 | LOC595101 | 0.5122 | 0.045175384 |
| A_24_P605563 | AY172962 | AY172962 | 0.5159 | 0.016533244 |
| A_23_P9485 | NM_000608 | ORM2 | 0.5505 | 0.03644601 |
| A_23_P78248 | NM_015515 | KRT23 | 0.5528 | 0.033868198 |
| A_24_P636974 | XR_039576 | LOC100132640 | 0.5546 | 0.030686942 |
| A_32_P51524 | CR627362 | LOC595101 | 0.5568 | 0.041712074 |
| A_23_P208706 | NM_138764 | BAX | 0.5577 | 0.02965597 |
| A_23_P208706 | NM_138764 | BAX | 0.5628 | 0.029584102 |
| A_23_P208706 | NM_138764 | BAX | 0.563 | 0.029175998 |
| A_24_P384210 | A_24_P384210 | A_24_P384210 | 0.5679 | 0.041110032 |
| A_23_P208706 | NM_138764 | BAX | 0.5696 | 0.027622888 |
| A_32_P129660 | NM_181453 | GCC2 | 0.5752 | 0.026419417 |
| A_24_P8151 | AK096606 | LOC100131170 | 0.5792 | 0.035287525 |
| A_23_P208706 | NM_138764 | BAX | 0.5869 | 0.02378101 |
| A_24_P83102 | NM_020070 | IGLL1 | 0.5938 | 0.012754748 |
| A_23_P25235 | NM_080387 | CLEC4D | 0.5942 | 0.012690235 |
| A_23_P208706 | NM_138764 | BAX | 0.5947 | 0.02164913 |
| A_23_P502320 | NM_001138 | AGRP | 0.595 | 0.029306098 |
| A_23_P208706 | NM_138764 | BAX | 0.5951 | 0.02019631 |
| A_23_P151820 | NM_024832 | RIN3 | 0.5956 | 0.046721445 |
| A_23_P390116 | NM_153023 | SPATA13 | 0.5973 | 0.040614105 |
| A_24_P239076 | NM_001013618 | IGLL3 | 0.6086 | 0.011200969 |
| A_23_P54781 | BC051317 | RBBP6 | 0.6155 | 0.007825062 |
| A_24_P67948 | AK001116 | AK001116 | 0.6165 | 0.038907699 |
| A_24_P88690 | NM_000578 | SLC11A1 | 0.6176 | 0.01274634 |
| A_23_P158868 | A_23_P158868 | A_23_P158868 | 0.6185 | 0.048291666 |
| A_23_P208706 | NM_138764 | BAX | 0.623 | 0.018755943 |
| A_24_P510357 | ENST00000390312 | ENST00000390312 | 0.6261 | 0.049830556 |
| A_23_P122563 | NM_014260 | PFDN6 | 0.6261 | 0.018027253 |
| A_24_P324074 | A_24_P324074 | A_24_P324074 | 0.6273 | 0.030748216 |
| A_23_P208706 | NM_138764 | BAX | 0.6287 | 0.018728598 |
| A_32_P8666 | XR_018217 | LOC644063 | 0.629 | 0.036145519 |
| A_24_P144163 | XR_037026 | LOC100128140 | 0.6364 | 0.04128856 |
| A_23_P78975 | A_23_P78975 | A_23_P78975 | 0.6399 | 0.022122809 |
| A_23_P435390 | A_23_P435390 | A_23_P435390 | 0.6415 | 0.012362732 |
| A_32_P78395 | A_32_P78395 | A_32_P78395 | 0.6422 | 0.048976721 |
| A_23_P103011 | NM_004914 | RAB36 | 0.6479 | 0.017052015 |
| A_23_P140760 | NM_170776 | GPR97 | 0.6505 | 0.018497652 |
| A_23_P208706 | NM_138764 | BAX | 0.6539 | 0.015369461 |
| A_24_P7181 | XR_037686 | LOC100128548 | 0.6548 | 0.022247115 |
| A_23_P39665 | NM_000578 | SLC11A1 | 0.6588 | 0.007231669 |
| A_24_P854492 | NR_003491 | MIAT | 0.6614 | 0.028714015 |
| A_24_P362904 | NM_004567 | PFKFB4 | 0.6647 | 0.031875134 |
| A_23_P165624 | NM_007115 | TNFAIP6 | 0.6664 | 0.016418204 |
| A_23_P330561 | NM_174918 | C19orf59 | 0.6678 | 0.015758321 |
| A_23_P209116 | AB002454 | CYP4F3 | 0.6704 | 0.049927365 |
| A_24_P253827 | NM_001030006 | AP2B1 | 0.677 | 0.044520963 |
| A_24_P260101 | NM_007289 | MME | 0.6775 | 0.019449679 |
| A_32_P38989 | NR_002712 | IL8RBP | 0.6782 | 0.022112595 |
| A_24_P359859 | NM_006037 | HDAC4 | 0.6829 | 0.032753007 |
| A_23_P39931 | NM_003494 | DYSF | 0.6861 | 0.01057674 |
| A_23_P61042 | ENST00000390539 | ENST00000390539 | 0.6919 | 0.000230911 |
| A_32_P79396 | NM_005746 | NAMPT | 0.6939 | 0.032739662 |
| A_24_P283341 | NM_022765 | MICAL1 | 0.6978 | 0.049729417 |
| A_23_P34597 | NM_001785 | CDA | 0.6979 | 0.026570283 |
| A_23_P117971 | M15530 | BCGF1 | 0.7011 | 0.032029442 |
| A_24_P10233 | NM_014326 | DAPK2 | 0.7058 | 0.039960562 |
| A_24_P238996 | A_24_P238996 | A_24_P238996 | 0.708 | 0.028050546 |
| A_23_P119583 | NM_000923 | PDE4C | 0.7081 | 0.038837695 |
| A_24_P392790 | A_24_P392790 | A_24_P392790 | 0.709 | 0.041182692 |
| A_23_P92499 | NM_003264 | TLR2 | 0.7161 | 0.049597719 |
| A_23_P152002 | NM_004049 | BCL2A1 | 0.7199 | 0.035667608 |
| A_23_P15621 | NM_032391 | C17orf92 | 0.7211 | 0.019178372 |
| A_24_P195145 | ENST00000399221 | ENST00000399221 | 0.7216 | 0.047348342 |
| A_23_P119583 | NM_000923 | PDE4C | 0.7236 | 0.034348694 |
| A_23_P406330 | NM_022733 | SMAP2 | 0.7274 | 0.02757474 |
| A_23_P92499 | NM_003264 | TLR2 | 0.7279 | 0.044422211 |
| A_23_P122724 | NM_004665 | VNN2 | 0.7306 | 0.031798796 |
| A_32_P112452 | BI026064 | BI026064 | 0.7313 | 0.007483406 |
| A_24_P353619 | NM_000478 | ALPL | 0.7344 | 0.01352007 |
| A_23_P119583 | NM_000923 | PDE4C | 0.7351 | 0.034204723 |
| A_24_P248255 | AF116642 | AF116642 | 0.7352 | 0.043347454 |
| A_32_P206308 | CD556746 | CD556746 | 0.736 | 0.048950361 |
| A_23_P39481 | NM_019112 | ABCA7 | 0.7367 | 0.033638024 |
| A_23_P92499 | NM_003264 | TLR2 | 0.7379 | 0.044739178 |
| A_23_P119583 | NM_000923 | PDE4C | 0.7386 | 0.036199247 |
| A_23_P214935 | NM_078625 | VNN3 | 0.7415 | 0.032111952 |
| A_23_P92499 | NM_003264 | TLR2 | 0.7418 | 0.040982613 |
| A_23_P130113 | NM_080912 | ASGR2 | 0.7424 | 0.031867033 |
| A_23_P115534 | NM_024874 | KIAA0319L | 0.7435 | 0.026645335 |
| A_23_P92499 | NM_003264 | TLR2 | 0.7439 | 0.038906489 |
| A_24_P396375 | NM_001113347 | ECE1 | 0.7441 | 0.025859815 |
| A_24_P868905 | XR_018907 | XR_018907 | 0.7463 | 0.003645997 |
| A_23_P119583 | NM_000923 | PDE4C | 0.7468 | 0.03248631 |
| A_23_P92499 | NM_003264 | TLR2 | 0.7486 | 0.039923781 |
| A_23_P333218 | NM_001031711 | ERGIC1 | 0.7492 | 0.025623398 |
| A_23_P119583 | NM_000923 | PDE4C | 0.7506 | 0.032538967 |
| A_23_P91230 | NM_003064 | SLPI | 0.7509 | 0.006074256 |
| A_23_P92499 | NM_003264 | TLR2 | 0.7513 | 0.039657294 |
| A_24_P666795 | A_24_P666795 | A_24_P666795 | 0.7531 | 0.027775147 |
| A_23_P119583 | NM_000923 | PDE4C | 0.7539 | 0.031292297 |
| A_23_P143662 | NM_014338 | PISD | 0.7573 | 0.032739601 |
| A_23_P21072 | AF258583 | RAB24 | 0.7583 | 0.048023011 |
| A_23_P128974 | NM_006399 | BATF | 0.7591 | 0.042795059 |
| A_23_P119583 | NM_000923 | PDE4C | 0.7618 | 0.031337991 |
| A_23_P431933 | NM_032294 | CAMKK1 | 0.7638 | 0.015277939 |
| A_23_P251387 | NM_004726 | REPS2 | 0.7645 | 0.049992857 |
| A_23_P68240 | NM_207328 | LOC150763 | 0.7654 | 0.018845719 |
| A_23_P119583 | NM_000923 | PDE4C | 0.7666 | 0.03083285 |
| A_24_P127442 | A_24_P127442 | A_24_P127442 | 0.7692 | 0.044774407 |
| A_23_P211603 | A_23_P211603 | A_23_P211603 | 0.7709 | 0.021947311 |
| A_23_P164022 | NM_033375 | MYO1C | 0.7714 | 0.040678048 |
| A_23_P57856 | NM_001706 | BCL6 | 0.7745 | 0.030764347 |
| A_23_P32500 | NM_015136 | STAB1 | 0.7807 | 0.015343157 |
| A_23_P170186 | NM_017570 | OPLAH | 0.7817 | 0.043477386 |
| A_23_P140190 | AF161538 | FAM30A | 0.7822 | 0.038765016 |
| A_23_P66774 | NM_153338 | GGT6 | 0.7828 | 0.036234441 |
| A_24_P7040 | A_24_P7040 | A_24_P7040 | 0.7873 | 0.025903621 |
| A_32_P47754 | BC060766 | SLC2A14 | 0.7896 | 0.0373637 |
| A_23_P53198 | NM_032564 | DGAT2 | 0.7907 | 0.006911737 |
| A_24_P239731 | NM_004776 | B4GALT5 | 0.7911 | 0.010877373 |
| A_32_P118010 | THC2657593 | THC2657593 | 0.792 | 0.02531932 |
| A_32_P7916 | BX096603 | BX096603 | 0.7939 | 0.034013667 |
| A_23_P314526 | NM_005803 | FLOT1 | 0.7944 | 0.046735411 |
| A_23_P18294 | NM_001034996 | RPL14 | 0.7948 | 0.047234519 |
| A_23_P40347 | NM_178580 | HM13 | 0.795 | 0.043852587 |
| A_23_P207138 | NM_203351 | MAP3K3 | 0.7953 | 0.048317331 |
| A_24_P322395 | NM_080732 | EGLN2 | 0.797 | 0.032016006 |
| A_23_P15146 | NM_001012631 | IL32 | 0.7974 | 0.021841809 |
| A_23_P14649 | AK021784 | C15orf28 | 0.8006 | 0.047086782 |
| A_24_P452293 | A_24_P452293 | A_24_P452293 | 0.8015 | 0.039679475 |
| A_23_P200728 | NM_000569 | FCGR3A | 0.8058 | 0.021583438 |
| A_24_P340886 | A_24_P340886 | A_24_P340886 | 0.8066 | 0.016432407 |
| A_24_P763243 | NM_001402 | EEF1A1 | 0.8077 | 0.042514224 |
| A_24_P117294 | NM_002463 | MX2 | 0.8086 | 0.027522638 |
| A_24_P228717 | NM_002872 | RAC2 | 0.8094 | 0.030131608 |
| A_24_P418044 | BC062324 | BC062324 | 0.8098 | 0.041386489 |
| A_23_P126593 | NM_005620 | S100A11 | 0.8101 | 0.03914557 |
| A_23_P109508 | NM_000631 | NCF4 | 0.8139 | 0.045006678 |
| A_23_P134176 | NM_001024465 | SOD2 | 0.814 | 0.046196993 |
| A_24_P322215 | NM_144683 | DHRS13 | 0.8147 | 0.042349876 |
| A_23_P135184 | NM_001042368 | RALGDS | 0.8154 | 0.020900468 |
| A_23_P255436 | NM_000487 | ARSA | 0.8154 | 0.037320222 |
| A_23_P358394 | NM_015864 | C6orf32 | 0.8157 | 0.045322155 |
| A_23_P74001 | NM_005621 | S100A12 | 0.8165 | 0.002926622 |
| A_24_P418044 | BC062324 | BC062324 | 0.8189 | 0.043784147 |
| A_23_P95130 | NM_207113 | SLC37A3 | 0.82 | 0.026934854 |
| A_23_P121885 | NM_031916 | ROPN1L | 0.8206 | 0.034270497 |
| A_23_P86653 | NM_002727 | SRGN | 0.8247 | 0.030058969 |
| A_23_P259868 | NM_020406 | CD177 | 0.8254 | 0.022975885 |
| A_23_P125109 | A_23_P125109 | A_23_P125109 | 0.8266 | 0.043023119 |
| A_23_P94230 | NM_015364 | LY96 | 0.8284 | 0.04966147 |
| A_24_P263144 | NM_001721 | BMX | 0.8291 | 0.014046054 |
| A_24_P84808 | XR_015548 | XR_015548 | 0.8301 | 0.042309212 |
| A_24_P202581 | NM_199002 | ARHGEF1 | 0.8302 | 0.046055163 |
| A_23_P200056 | NM_023018 | NADK | 0.8305 | 0.040860236 |
| A_23_P256724 | NM_003841 | TNFRSF10C | 0.8327 | 0.03733118 |
| A_23_P109907 | NM_175924 | ILDR1 | 0.8339 | 0.011664777 |
| A_23_P19543 | NM_003137 | SRPK1 | 0.8368 | 0.030990394 |
| A_32_P148914 | BC037255 | LOC389634 | 0.8377 | 0.021885624 |
| A_24_P3704 | DQ786246 | DQ786246 | 0.8377 | 0.047316838 |
| A_23_P17655 | NM_170736 | KCNJ15 | 0.839 | 0.013549313 |
| A_23_P134980 | NM_033086 | FGD3 | 0.8394 | 0.040526139 |
| A_23_P77049 | NM_001039355 | SLC25A29 | 0.8401 | 0.031912542 |
| A_23_P142447 | NM_012335 | MYO1F | 0.8409 | 0.04698973 |
| A_23_P138985 | NM_000732 | CD3D | 0.8412 | 0.044791239 |
| A_23_P68807 | BC021857 | FLJ23185 | 0.8424 | 0.046173768 |
| A_24_P297078 | NM_020531 | C20orf3 | 0.8442 | 0.011885659 |
| A_23_P47304 | NM_004347 | CASP5 | 0.8459 | 0.01071847 |
| A_32_P118013 | THC2657593 | THC2657593 | 0.8463 | 0.020011122 |
| A_24_P89257 | NM_001031711 | ERGIC1 | 0.8474 | 0.040782285 |
| A_23_P426989 | NM_030645 | SH3BP5L | 0.8492 | 0.007790487 |
| A_23_P258108 | XR_015767 | LOC727835 | 0.8495 | 0.027799161 |
| A_24_P174924 | NM_003537 | HIST1H3B | 0.8503 | 0.046244483 |
| A_23_P98686 | NM_025092 | ATHL1 | 0.8511 | 0.0082581 |
| A_32_P14544 | NM_002952 | RPS2 | 0.8514 | 0.049815387 |
| A_24_P295791 | NM_032564 | DGAT2 | 0.8526 | 0.017383747 |
| A_32_P145790 | XR_015746 | LOC728732 | 0.8533 | 0.040922987 |
| A_23_P251232 | NR_001543 | TTTY14 | 0.8536 | 0.048182023 |
| A_23_P321703 | NM_004049 | BCL2A1 | 0.8544 | 0.040439823 |
| A_23_P218549 | NM_032571 | EMR3 | 0.8557 | 0.040765493 |
| A_23_P208516 | NM_024298 | MBOAT7 | 0.8558 | 0.023017277 |
| A_23_P68121 | NM_012455 | PSD4 | 0.8567 | 0.044355105 |
| A_23_P207837 | NM_001304 | CPD | 0.8568 | 0.030799345 |
| A_23_P214603 | NM_005803 | FLOT1 | 0.8569 | 0.026262198 |
| A_32_P55438 | A_32_P55438 | A_32_P55438 | 0.8574 | 0.01618447 |
| A_23_P135755 | NM_001557 | IL8RB | 0.8583 | 0.010353364 |
| A_24_P70480 | NM_001817 | CEACAM4 | 0.8586 | 0.010527009 |
| A_23_P132438 | NM_018133 | MSL2L1 | 0.8601 | 0.036924804 |
| A_23_P305060 | NM_005746 | NAMPT | 0.8604 | 0.035791769 |
| A_32_P79115 | XR_039086 | LOC100133075 | 0.8607 | 0.033477373 |
| A_24_P192914 | NM_153206 | AMICA1 | 0.8611 | 0.03846877 |
| A_32_P49392 | XR_036968 | LOC100129742 | 0.862 | 0.036314914 |
| A_23_P13899 | NM_002046 | GAPDH | 0.8624 | 0.03925237 |
| A_24_P252497 | NM_025195 | TRIB1 | 0.863 | 0.035531755 |
| A_24_P929621 | NP111770 | NP111770 | 0.8637 | 0.026027454 |
| A_23_P305711 | NM_012234 | RYBP | 0.8646 | 0.019555591 |
| A_24_P295245 | NM_032467 | ASPH | 0.8653 | 0.025590872 |
| A_23_P118406 | NM_001033046 | C17orf62 | 0.8665 | 0.0470983 |
| A_23_P250353 | NM_017912 | HERC6 | 0.8674 | 0.022614255 |
| A_23_P371215 | NM_012092 | ICOS | 0.8682 | 0.045455438 |
| A_24_P105648 | DB304731 | DB304731 | 0.8712 | 0.047403585 |
| A_23_P16538 | NM_006532 | ELL | 0.8713 | 0.036889445 |
| A_24_P659122 | AK125790 | LOC401357 | 0.8716 | 0.017456523 |
| A_23_P111037 | NM_003529 | HIST1H3A | 0.8729 | 0.04996939 |
| A_23_P61637 | NM_002108 | HAL | 0.8732 | 0.018523372 |
| A_32_P41375 | AK093729 | tcag7.907 | 0.8735 | 0.025382355 |
| A_23_P145863 | NM_005620 | S100A11 | 0.8741 | 0.026676096 |
| A_23_P384405 | NM_001440 | EXTL3 | 0.8756 | 0.046613991 |
| A_32_P168375 | THC2659236 | THC2659236 | 0.8773 | 0.009842734 |
| A_23_P148121 | NM_001099409 | EHBP1L1 | 0.8792 | 0.026705901 |
| A_24_P204574 | A_24_P204574 | A_24_P204574 | 0.8805 | 0.023366346 |
| A_32_P213002 | THC2526402 | THC2526402 | 0.8809 | 0.04048581 |
| A_23_P66694 | NM_006495 | EVI2B | 0.8824 | 0.020071531 |
| A_32_P221641 | A_32_P221641 | A_32_P221641 | 0.8833 | 0.048534092 |
| A_24_P190472 | NM_003064 | SLPI | 0.8836 | 0.003157991 |
| A_23_P148768 | NM_000130 | F5 | 0.8852 | 0.021402064 |
| A_24_P358760 | AK090481 | AK090481 | 0.8862 | 0.021442957 |
| A_23_P209625 | NM_000104 | CYP1B1 | 0.887 | 0.010289808 |
| A_24_P120352 | NM_020773 | TBC1D14 | 0.887 | 0.0489837 |
| A_32_P228438 | THC2637028 | THC2637028 | 0.8875 | 0.015795695 |
| A_23_P218111 | NM_001002236 | SERPINA1 | 0.889 | 0.04052717 |
| A_23_P148768 | NM_000130 | F5 | 0.8896 | 0.022241863 |
| A_23_P144054 | NM_006254 | PRKCD | 0.8929 | 0.049030972 |
| A_23_P58266 | NM_005980 | S100P | 0.893 | 0.002082629 |
| A_24_P341058 | XR_018572 | XR_018572 | 0.8931 | 0.023453644 |
| A_23_P111583 | NM_001001547 | CD36 | 0.8936 | 0.034872045 |
| A_32_P62137 | A_32_P62137 | A_32_P62137 | 0.8948 | 0.031221611 |
| A_23_P101434 | NM_033297 | NLRP12 | 0.8964 | 0.034995041 |
| A_23_P4096 | NM_000717 | CA4 | 0.8971 | 0.004791835 |
| A_32_P135818 | NM_001006 | RPS3A | 0.8996 | 0.041087446 |
| A_23_P408353 | NM_002116 | HLA-A | 0.9 | 0.025674928 |
| A_32_P105469 | AK074696 | AK074696 | 0.9003 | 0.033751929 |
| A_23_P148768 | NM_000130 | F5 | 0.9014 | 0.021209283 |
| A_24_P369898 | ENST00000293201 | ENST00000293201 | 0.9024 | 0.02801005 |
| A_32_P116088 | A_32_P116088 | A_32_P116088 | 0.9034 | 0.017811475 |
| A_23_P7066 | NM_001024921 | RPL9 | 0.9076 | 0.027320127 |
| A_23_P143845 | NM_015508 | TIPARP | 0.908 | 0.048993741 |
| A_32_P209350 | NM_001033853 | RPL3 | 0.9103 | 0.038849285 |
| A_32_P66881 | NM_138554 | TLR4 | 0.9112 | 0.018735538 |
| A_24_P90097 | NM_016824 | ADD3 | 0.9131 | 0.036848159 |
| A_24_P351906 | NM_024636 | STEAP4 | 0.9141 | 0.033618479 |
| A_23_P16537 | NM_006532 | ELL | 0.9159 | 0.041738435 |
| A_24_P55391 | BC082970 | BC082970 | 0.9176 | 0.007643549 |
| A_23_P49616 | NM_020198 | CCDC47 | 0.9199 | 0.017567685 |
| A_23_P148768 | NM_000130 | F5 | 0.9202 | 0.018681797 |
| A_23_P218817 | NM_152246 | CPT1B | 0.9216 | 0.030525101 |
| A_24_P314597 | NM_182686 | KIAA0319L | 0.9243 | 0.012850333 |
| A_23_P436336 | ENST00000340536 | ENST00000340536 | 0.9254 | 0.005466423 |
| A_24_P273561 | NM_018169 | C12orf35 | 0.9259 | 0.025723931 |
| A_23_P201521 | A_23_P201521 | A_23_P201521 | 0.926 | 0.045947704 |
| A_23_P144054 | NM_006254 | PRKCD | 0.9264 | 0.047015308 |
| A_24_P280933 | XR_018187 | XR_018187 | 0.9267 | 0.013426548 |
| A_24_P81900 | NM_006931 | SLC2A3 | 0.9281 | 0.046046267 |
| A_23_P376557 | NM_022468 | MMP25 | 0.9288 | 0.005317262 |
| A_23_P13899 | NM_002046 | GAPDH | 0.9291 | 0.032907072 |
| A_32_P164758 | AK128836 | LOC730286 | 0.9301 | 0.011907248 |
| A_23_P163492 | NM_003933 | BAIAP3 | 0.9306 | 0.015023548 |
| A_23_P377616 | AK128423 | CIRBP | 0.9325 | 0.017324879 |
| A_23_P155828 | NM_020773 | TBC1D14 | 0.9325 | 0.042849493 |
| A_23_P408353 | NM_002116 | HLA-A | 0.9327 | 0.022528948 |
| A_23_P148768 | NM_000130 | F5 | 0.9328 | 0.014214073 |
| A_32_P19294 | NM_144669 | GLT1D1 | 0.933 | 0.024472203 |
| A_24_P349636 | XR_016879 | LOC388401 | 0.9334 | 0.049552408 |
| A_23_P408353 | NM_002116 | HLA-A | 0.9338 | 0.022265768 |
| A_23_P101829 | NM_004720 | LPAR2 | 0.9339 | 0.008490402 |
| A_23_P352266 | NM_000633 | BCL2 | 0.934 | 0.04657783 |
| A_23_P126623 | NM_002631 | PGD | 0.9346 | 0.013848734 |
| A_24_P298238 | A_24_P298238 | A_24_P298238 | 0.9348 | 0.036334931 |
| A_23_P213863 | NM_019057 | FLJ10404 | 0.9349 | 0.038006647 |
| A_23_P259506 | NM_032412 | C5orf32 | 0.9372 | 0.014809113 |
| A_23_P148768 | NM_000130 | F5 | 0.9382 | 0.014645962 |
| A_23_P96556 | NM_203391 | GK | 0.9401 | 0.043723764 |
| A_23_P352266 | NM_000633 | BCL2 | 0.9403 | 0.041362192 |
| A_32_P64016 | NR_002939 | RUNDC2C | 0.9404 | 0.01902399 |
| A_24_P267814 | BI521983 | BI521983 | 0.9404 | 0.007688423 |
| A_23_P154488 | NM_033109 | PNPT1 | 0.9414 | 0.015516471 |
| A_24_P15502 | ENST00000399573 | ENST00000399573 | 0.9422 | 0.007154632 |
| A_24_P375949 | XR_019375 | XR_019375 | 0.9429 | 0.034109029 |
| A_24_P172768 | NM_004124 | GMFB | 0.944 | 0.039136709 |
| A_32_P25809 | A_32_P25809 | A_32_P25809 | 0.9447 | 0.046488461 |
| A_23_P217866 | NM_005531 | IFI16 | 0.9477 | 0.011320951 |
| A_32_P58074 | NM_001006 | RPS3A | 0.9487 | 0.049977324 |
| A_24_P228796 | NM_021123 | GAGE7 | 0.9496 | 0.029484007 |
| A_23_P148768 | NM_000130 | F5 | 0.95 | 0.01385847 |
| A_23_P137391 | NM_001428 | ENO1 | 0.9513 | 0.031623654 |
| A_23_P119835 | NM_021209 | NLRC4 | 0.9518 | 0.043208663 |
| A_24_P110403 | BF931515 | BF931515 | 0.9535 | 0.03751388 |
| A_24_P68631 | NM_175065 | HIST2H2AB | 0.9535 | 0.02539545 |
| A_23_P145264 | NM_018950 | HLA-F | 0.9535 | 0.036220684 |
| A_23_P24784 | NM_003282 | TNNI2 | 0.9538 | 0.031934129 |
| A_24_P789842 | A_24_P789842 | A_24_P789842 | 0.9538 | 0.029204091 |
| A_23_P148768 | NM_000130 | F5 | 0.9541 | 0.014282143 |
| A_23_P352266 | NM_000633 | BCL2 | 0.9545 | 0.039797227 |
| A_23_P60753 | THC2721409 | THC2721409 | 0.9545 | 0.027467482 |
| A_23_P54636 | NM_004691 | ATP6V0D1 | 0.9551 | 0.043835458 |
| A_24_P349743 | NM_002295 | RPSA | 0.9558 | 0.047628526 |
| A_23_P4653 | BC025318 | KLC3 | 0.9559 | 0.018998935 |
| A_32_P38821 | BF475893 | BF475893 | 0.956 | 0.044921122 |
| A_23_P426305 | NM_003734 | AOC3 | 0.9561 | 0.039606964 |
| A_23_P101380 | NM_198540 | B3GNT8 | 0.9562 | 0.033737401 |
| A_23_P334021 | NM_000876 | IGF2R | 0.9571 | 0.011587146 |
| A_32_P37089 | AK097700 | AK097700 | 0.9574 | 0.042194585 |
| A_23_P312932 | NM_175857 | KRTAP8-1 | 0.9582 | 0.015071347 |
| A_23_P413456 | AK128423 | CIRBP | 0.9592 | 0.016177216 |
| A_23_P408353 | NM_002116 | HLA-A | 0.9596 | 0.018492889 |
| A_23_P148768 | NM_000130 | F5 | 0.9596 | 0.015250859 |
| A_24_P89708 | NM_000883 | IMPDH1 | 0.9601 | 0.012814294 |
| A_23_P408353 | NM_002116 | HLA-A | 0.9607 | 0.018100349 |
| A_24_P66027 | NM_004900 | APOBEC3B | 0.9607 | 0.004001614 |
| A_32_P225604 | NM_000969 | RPL5 | 0.9636 | 0.04566378 |
| A_23_P65789 | BC041387 | MCTP2 | 0.964 | 0.034076825 |
| A_23_P7684 | NM_024565 | CCNJL | 0.9652 | 0.013246964 |
| A_23_P65174 | NM_001040443 | PHF11 | 0.9655 | 0.044603288 |
| A_23_P85903 | NM_003268 | TLR5 | 0.9658 | 0.024819021 |
| A_32_P72067 | AK130576 | ARHGAP24 | 0.9658 | 0.023301694 |
| A_23_P502312 | NM_078481 | CD97 | 0.9679 | 0.044068374 |
| A_23_P408353 | NM_002116 | HLA-A | 0.9699 | 0.018096759 |
| A_23_P30069 | NM_001012967 | DDX60L | 0.9704 | 0.005569584 |
| A_23_P36658 | NM_145791 | MGST1 | 0.9707 | 0.032292042 |
| A_23_P72503 | NM_007246 | KLHL2 | 0.9716 | 0.008122535 |
| A_32_P71744 | BG695979 | BG695979 | 0.9725 | 0.033171518 |
| A_23_P67932 | NM_000634 | IL8RA | 0.9729 | 0.00622518 |
| A_24_P222591 | NM_001077619 | LOC137886 | 0.9733 | 0.027955794 |
| A_24_P8109 | NM_001012302 | TMEM16J | 0.9735 | 0.033265056 |
| A_32_P213692 | AK025695 | TMED8 | 0.9751 | 0.013471578 |
| A_24_P229531 | NM_001031716 | OBFC2A | 0.9765 | 0.041015099 |
| A_24_P367199 | A_24_P367199 | A_24_P367199 | 0.9766 | 0.025265293 |
| A_24_P395415 | AF343666 | AF343666 | 0.9779 | 0.000408622 |
| A_24_P279121 | NM_018670 | MESP1 | 0.9779 | 0.026933581 |
| A_23_P104689 | BC110830 | RELA | 0.9792 | 0.042950253 |
| A_24_P58054 | NM_015266 | SLC9A8 | 0.9829 | 0.023919141 |
| A_23_P129492 | NM_016332 | SEPX1 | 0.9843 | 0.012015618 |
| A_32_P159347 | NM_194247 | HNRNPA3 | 0.9846 | 0.029302476 |
| A_23_P104689 | BC110830 | RELA | 0.9848 | 0.045806921 |
| A_23_P107933 | NM_016440 | VRK3 | 0.9851 | 0.041150635 |
| A_23_P408353 | NM_002116 | HLA-A | 0.9852 | 0.017767068 |
| A_23_P154367 | NM_004226 | STK17B | 0.9863 | 0.040001383 |
| A_23_P101321 | NM_199002 | ARHGEF1 | 0.9868 | 0.042226806 |
| A_23_P53467 | NM_201612 | IKIP | 0.9872 | 0.029202262 |
| A_24_P761818 | XM_928025 | LOC644937 | 0.9875 | 0.037103372 |
| A_23_P137196 | NM_001560 | IL13RA1 | 0.9884 | 0.020898753 |
| A_23_P321201 | NM_015213 | RAB6IP1 | 0.9886 | 0.046813931 |
| A_32_P776626 | NM_001024457 | RGPD1 | 0.9889 | 0.020485543 |
| A_23_P150919 | AK027372 | ORAI1 | 0.9891 | 0.021571917 |
| A_24_P89701 | NM_000883 | IMPDH1 | 0.9897 | 0.043829245 |
| A_24_P21715 | NM_004584 | RAD9A | 0.9901 | 0.037777537 |
| A_24_P389517 | NM_031262 | HNRNPK | 0.9911 | 0.007767474 |
| A_24_P336759 | NM_021960 | MCL1 | 0.9921 | 0.008405634 |
| A_23_P54929 | NM_020424 | LYRM1 | 0.9926 | 0.048635736 |
| A_24_P297798 | XR_019052 | XR_019052 | 0.9929 | 0.030703719 |
| A_23_P148768 | NM_000130 | F5 | 0.993 | 0.010570759 |
| A_23_P6746 | NM_014869 | IQSEC1 | 0.9951 | 0.025847011 |
| A_32_P74561 | ENST00000332686 | ENST00000332686 | 0.9952 | 0.023169978 |
| A_24_P59387 | AK022255 | C8orf60 | 0.9959 | 0.016098802 |
| A_24_P25252 | NM_015208 | ANKRD12 | 0.9975 | 0.049382983 |
| A_23_P120345 | NM_020651 | PELI1 | 0.9978 | 0.026222374 |
| A_24_P167825 | NM_015289 | VPS39 | 0.9999 | 0.033665098 |
| A_32_P137382 | BX089701 | BX089701 | 1.0006 | 0.04924763 |
| A_23_P23048 | NM_002965 | S100A9 | 1.0014 | 0.00667731 |
| A_32_P136662 | THC2560098 | THC2560098 | 1.0019 | 0.032059275 |
| A_24_P684328 | ENST00000339120 | ENST00000339120 | 1.0024 | 0.032358886 |
| A_23_P304237 | NM_198679 | RAPGEF1 | 1.0025 | 0.032936159 |
| A_23_P67529 | NM_002250 | KCNN4 | 1.0032 | 0.014072386 |
| A_23_P14559 | NM_006871 | RIPK3 | 1.0039 | 0.042846501 |
| A_23_P55319 | NM_004475 | FLOT2 | 1.004 | 0.00509027 |
| A_23_P86164 | NM_020765 | UBR4 | 1.005 | 0.044385661 |
| A_23_P408353 | NM_002116 | HLA-A | 1.0054 | 0.014916957 |
| A_23_P133142 | NM_025144 | ALPK1 | 1.0059 | 0.027878979 |
| A_23_P212522 | NM_014616 | ATP11B | 1.007 | 0.024686415 |
| A_23_P119502 | NM_003775 | S1PR4 | 1.0074 | 0.033969721 |
| A_24_P471121 | XR_019546 | XR_019546 | 1.0079 | 0.024044649 |
| A_23_P28485 | NM_012198 | GCA | 1.0094 | 0.007850733 |
| A_24_P50554 | XR_018405 | LOC391655 | 1.0101 | 0.030057455 |
| A_24_P58381 | NM_017780 | CHD7 | 1.0109 | 0.037115882 |
| A_24_P417706 | AK092842 | MXD3 | 1.0117 | 0.019881585 |
| A_24_P350008 | XR_038176 | LOC100129553 | 1.0124 | 0.0316389 |
| A_32_P56001 | NM_012072 | CD93 | 1.0127 | 0.026108445 |
| A_23_P132948 | NM_201999 | ELF2 | 1.0143 | 0.014614121 |
| A_23_P112162 | NM_012079 | DGAT1 | 1.0158 | 0.028192303 |
| A_24_P794833 | THC2597357 | THC2597357 | 1.0159 | 0.015723341 |
| A_23_P408353 | NM_002116 | HLA-A | 1.0164 | 0.012643005 |
| A_23_P408353 | NM_002116 | HLA-A | 1.0171 | 0.013733065 |
| A_23_P384517 | NM_004130 | GYG1 | 1.0173 | 0.011421306 |
| A_23_P19333 | NM_018643 | TREM1 | 1.0182 | 0.004430647 |
| A_24_P367069 | XR_018975 | XR_018975 | 1.0187 | 0.032768443 |
| A_24_P36898 | AL832451 | AL832451 | 1.0192 | 0.036110283 |
| A_23_P217269 | NM_007268 | VSIG4 | 1.0201 | 0.004656018 |
| A_23_P5273 | NM_014963 | SBNO2 | 1.0206 | 0.02220238 |
| A_24_P367063 | A_24_P367063 | A_24_P367063 | 1.0244 | 0.008405568 |
| A_24_P89038 | ENST00000307533 | ENST00000307533 | 1.0251 | 0.030956727 |
| A_24_P29641 | NM_148936 | NSUN5C | 1.0263 | 0.029001032 |
| A_24_P649624 | BC063684 | BC063684 | 1.0264 | 0.049396455 |
| A_23_P149075 | NM_018067 | MAP7D1 | 1.0278 | 0.018360505 |
| A_24_P928798 | AK056260 | AK056260 | 1.0282 | 0.029352322 |
| A_23_P104689 | BC110830 | RELA | 1.0298 | 0.035996146 |
| A_32_P71113 | THC2508355 | THC2508355 | 1.031 | 0.046975669 |
| A_23_P123125 | NM_001029835 | CCM2 | 1.031 | 0.011164351 |
| A_24_P754803 | NM_006013 | RPL10 | 1.0318 | 0.042783211 |
| A_23_P56150 | NM_032753 | RAX2 | 1.0322 | 0.041823042 |
| A_24_P400528 | NM_001080495 | TNRC18 | 1.0328 | 0.049267176 |
| A_23_P155162 | AF398511 | PANX2 | 1.0333 | 0.033182989 |
| A_23_P336198 | NM_138426 | GLCCI1 | 1.034 | 0.047866477 |
| A_24_P394702 | NM_001662 | ARF5 | 1.0348 | 0.034247445 |
| A_23_P154539 | NM_012469 | PRPF6 | 1.0352 | 0.043209368 |
| A_23_P160025 | NM_005531 | IFI16 | 1.0358 | 0.012232486 |
| A_23_P155830 | NM_020773 | TBC1D14 | 1.0361 | 0.025028631 |
| A_23_P63896 | NM_000043 | FAS | 1.0372 | 0.043153223 |
| A_23_P14340 | NM_014672 | KIAA0391 | 1.0378 | 0.047257322 |
| A_23_P428184 | NM_021065 | HIST1H2AD | 1.0379 | 0.03945727 |
| A_24_P451992 | XR_018393 | XR_018393 | 1.0395 | 0.021569505 |
| A_23_P165007 | NM_170604 | RASGRP4 | 1.0396 | 0.046299221 |
| A_24_P48078 | NM_015247 | CYLD | 1.0397 | 0.037254286 |
| A_23_P112481 | NM_004925 | AQP3 | 1.04 | 0.006753951 |
| A_23_P207896 | NM_001893 | CSNK1D | 1.0414 | 0.024172376 |
| A_23_P39925 | NM_003494 | DYSF | 1.0421 | 0.005468302 |
| A_23_P205686 | NM_000021 | PSEN1 | 1.0421 | 0.046913271 |
| A_24_P719096 | THC2729213 | THC2729213 | 1.0433 | 0.0303925 |
| A_32_P31992 | XR_017310 | LOC343515 | 1.0437 | 0.023492185 |
| A_23_P102109 | NM_006000 | TUBA4A | 1.0439 | 0.009658599 |
| A_32_P22401 | NM_018067 | MAP7D1 | 1.044 | 0.020374815 |
| A_23_P1199 | NM_014142 | NUDT5 | 1.0446 | 0.040092716 |
| A_32_P203320 | BC041849 | ROCK1 | 1.0455 | 0.042157244 |
| A_32_P142652 | AK091931 | LOC220433 | 1.046 | 0.048798907 |
| A_23_P151791 | NM_181657 | LTB4R | 1.0462 | 0.024006252 |
| A_23_P121253 | NM_003810 | TNFSF10 | 1.0463 | 0.009925026 |
| A_23_P124559 | NM_031300 | MXD3 | 1.0473 | 0.016269251 |
| A_23_P128447 | NM_198578 | LRRK2 | 1.0493 | 0.027571386 |
| A_23_P2801 | NM_172373 | ELF1 | 1.0495 | 0.02774426 |
| A_24_P318544 | AB209463 | CSNK1D | 1.0507 | 0.0162617 |
| A_23_P56127 | NM_019108 | C19orf61 | 1.051 | 0.031813506 |
| A_23_P27846 | NM_001080400 | KIAA1881 | 1.0529 | 0.025337183 |
| A_24_P306355 | XR_018781 | XR_018781 | 1.0534 | 0.033976836 |
| A_23_P68730 | NM_003681 | PDXK | 1.0535 | 0.048532166 |
| A_24_P170147 | A_24_P170147 | A_24_P170147 | 1.0575 | 0.017882458 |
| A_24_P49533 | NM_024646 | ZYG11B | 1.0587 | 0.041881297 |
| A_24_P213284 | XR_019258 | XR_019258 | 1.0602 | 0.04304303 |
| A_24_P107336 | NM_052875 | VPS26B | 1.0609 | 0.041256046 |
| A_23_P4060 | AK026312 | FLJ22659 | 1.0613 | 0.030646989 |
| A_23_P35349 | NM_021738 | SVIL | 1.064 | 0.032853758 |
| A_23_P251825 | NM_001007245 | IFRD1 | 1.065 | 0.031259331 |
| A_23_P363968 | NM_016546 | C1RL | 1.0657 | 0.033506866 |
| A_24_P168456 | NM_001001975 | ATP5D | 1.0662 | 0.022777002 |
| A_23_P170534 | NM_004479 | FUT7 | 1.0668 | 0.010602402 |
| A_23_P3823 | NM_001122957 | BCKDK | 1.0679 | 0.029836349 |
| A_24_P24831 | NM_182905 | WASH1 | 1.0692 | 0.046430949 |
| A_23_P47991 | NM_015335 | MED13L | 1.0694 | 0.03308744 |
| A_23_P121253 | NM_003810 | TNFSF10 | 1.0708 | 0.009464275 |
| A_23_P104689 | BC110830 | RELA | 1.0714 | 0.030862098 |
| A_23_P348587 | A_23_P348587 | A_23_P348587 | 1.0716 | 0.045566774 |
| A_24_P354412 | AK091335 | AK091335 | 1.072 | 0.038512287 |
| A_24_P516728 | AL547890 | AL547890 | 1.0725 | 0.042966235 |
| A_23_P90014 | NM_020171 | GPR108 | 1.0738 | 0.029965378 |
| A_23_P75097 | NM_024895 | PDZD7 | 1.074 | 0.034786747 |
| A_32_P209472 | NM_170606 | MLL3 | 1.0753 | 0.044499794 |
| A_23_P200489 | NM_014698 | TMEM63A | 1.0753 | 0.016355182 |
| A_32_P212058 | NM_032898 | C3orf34 | 1.0755 | 0.006109245 |
| A_24_P223124 | NM_022763 | FNDC3B | 1.0761 | 0.039116861 |
| A_32_P19752 | NM_144664 | FAM76B | 1.0767 | 0.034005683 |
| A_23_P89283 | NM_006224 | PITPNA | 1.0773 | 0.045968231 |
| A_23_P201778 | NM_080588 | PTPN7 | 1.0781 | 0.034813888 |
| A_32_P308040 | NR_002775 | RPLP0P2 | 1.0792 | 0.031193884 |
| A_24_P278367 | NM_005736 | ACTR1A | 1.0819 | 0.026604737 |
| A_24_P370372 | NM_014292 | CBX6 | 1.0824 | 0.042259224 |
| A_23_P87545 | NM_021034 | IFITM3 | 1.0829 | 0.005002961 |
| A_24_P96709 | CR606630 | CR606630 | 1.0844 | 0.036421842 |
| A_24_P74559 | NM_013385 | PSCD4 | 1.0851 | 0.027696795 |
| A_24_P307443 | XR_018808 | XR_018808 | 1.0854 | 0.020418543 |
| A_23_P125117 | NM_173546 | KLHDC8B | 1.0867 | 0.00716958 |
| A_24_P7494 | A_24_P7494 | A_24_P7494 | 1.0868 | 0.046074674 |
| A_23_P85598 | NM_020247 | CABC1 | 1.0871 | 0.013276833 |
| A_23_P254415 | NM_002295 | RPSA | 1.0871 | 0.028323968 |
| A_24_P252718 | BC036529 | EPC1 | 1.0892 | 0.007159496 |
| A_23_P29378 | NM_007282 | RNF13 | 1.0898 | 0.022249867 |
| A_24_P303420 | ENST00000338759 | ENST00000338759 | 1.0899 | 0.049474658 |
| A_24_P127082 | A_24_P127082 | A_24_P127082 | 1.0904 | 0.021956799 |
| A_24_P289795 | ENST00000323057 | ENST00000323057 | 1.0906 | 0.02939697 |
| A_32_P100109 | NM_004726 | REPS2 | 1.0913 | 0.007550959 |
| A_23_P258393 | NM_144641 | PPM1M | 1.0953 | 0.043914711 |
| A_23_P162211 | NM_018050 | MANSC1 | 1.0971 | 0.008836528 |
| A_24_P344053 | NM_015556 | SIPA1L1 | 1.0971 | 0.016441153 |
| A_23_P205686 | NM_000021 | PSEN1 | 1.0975 | 0.040768798 |
| A_32_P204019 | NM_000992 | RPL29 | 1.0987 | 0.03840544 |
| A_23_P251196 | A_23_P251196 | A_23_P251196 | 1.0993 | 0.048201901 |
| A_24_P8721 | NM_003517 | HIST2H2AC | 1.1011 | 0.049334763 |
| A_24_P354468 | NM_002106 | H2AFZ | 1.1012 | 0.044511111 |
| A_23_P121253 | NM_003810 | TNFSF10 | 1.1015 | 0.007802416 |
| A_23_P253012 | NM_017577 | GRAMD1C | 1.1019 | 0.011815258 |
| A_23_P201287 | NM_015074 | KIF1B | 1.1029 | 0.034149029 |
| A_24_P392201 | NM_198517 | TBC1D10C | 1.1032 | 0.040815594 |
| A_23_P9903 | NM_178326 | ATG4B | 1.1035 | 0.044711119 |
| A_23_P212354 | NM_000647 | CCR2 | 1.1038 | 0.012727628 |
| A_23_P121253 | NM_003810 | TNFSF10 | 1.1039 | 0.007374725 |
| A_24_P820302 | THC2594845 | THC2594845 | 1.1039 | 0.032678521 |
| A_24_P230100 | ENST00000397494 | ENST00000397494 | 1.1049 | 0.049461022 |
| A_23_P8281 | NM_000416 | IFNGR1 | 1.107 | 0.028720456 |
| A_24_P941441 | NM_006572 | GNA13 | 1.1071 | 0.044916968 |
| A_24_P314179 | NM_005239 | ETS2 | 1.1073 | 0.035218777 |
| A_23_P161194 | NM_003380 | VIM | 1.1077 | 0.028049693 |
| A_23_P48637 | BG108194 | BG108194 | 1.1079 | 0.048601071 |
| A_23_P152234 | NM_144673 | CMTM2 | 1.1094 | 0.005146288 |
| A_23_P154070 | NM_006000 | TUBA4A | 1.1101 | 0.036553436 |
| A_23_P210379 | NM_175609 | ARFGAP1 | 1.1105 | 0.041152762 |
| A_24_P309415 | NM_052932 | TMEM123 | 1.1111 | 0.01518838 |
| A_24_P229766 | A_24_P229766 | A_24_P229766 | 1.1117 | 0.04897584 |
| A_23_P77552 | NM_198490 | RAB43 | 1.1125 | 0.040387818 |
| A_23_P2582 | NM_015401 | HDAC7 | 1.113 | 0.027449348 |
| A_24_P245838 | NM_002409 | MGAT3 | 1.1134 | 0.015368992 |
| A_23_P51926 | NM_000952 | PTAFR | 1.1135 | 0.0117525 |
| A_23_P93629 | NM_015905 | TRIM24 | 1.1139 | 0.039627217 |
| A_23_P214766 | NM_006734 | HIVEP2 | 1.1148 | 0.034348837 |
| A_32_P10856 | THC2689623 | THC2689623 | 1.1154 | 0.026339677 |
| A_23_P144896 | NM_005451 | PDLIM7 | 1.1173 | 0.009772048 |
| A_23_P121253 | NM_003810 | TNFSF10 | 1.1173 | 0.006973012 |
| A_24_P144275 | A_24_P144275 | A_24_P144275 | 1.1175 | 0.016829991 |
| A_23_P125519 | NM_001007 | RPS4X | 1.1185 | 0.022041596 |
| A_24_P274640 | NM_001042490 | LOC730394 | 1.119 | 0.009242862 |
| A_32_P179837 | NM_174903 | RNF151 | 1.1191 | 0.014215552 |
| A_32_P226786 | BC045174 | BC045174 | 1.1193 | 0.007072604 |
| A_23_P67339 | NM_020650 | RCN3 | 1.1197 | 0.011268647 |
| A_23_P207301 | AK001891 | PRR11 | 1.1198 | 0.013301839 |
| A_23_P328652 | NM_153240 | NPHP3 | 1.1204 | 0.039484383 |
| A_24_P519504 | X57802 | X57802 | 1.1212 | 0.001152039 |
| A_23_P119168 | A_23_P119168 | A_23_P119168 | 1.1221 | 0.037682852 |
| A_23_P26878 | NM_001025579 | NDEL1 | 1.1228 | 0.024885223 |
| A_23_P160631 | NM_005998 | CCT3 | 1.1233 | 0.040105488 |
| A_24_P629900 | AK024602 | AK024602 | 1.1234 | 0.043655166 |
| A_23_P18490 | NM_001017405 | MAEA | 1.1248 | 0.040947211 |
| A_23_P389708 | NM_032378 | EEF1D | 1.1257 | 0.0354915 |
| A_23_P127793 | NM_153265 | EML3 | 1.1259 | 0.037367389 |
| A_23_P114947 | NM_002923 | RGS2 | 1.1259 | 0.005213497 |
| A_24_P330971 | NM_013234 | EIF3K | 1.1274 | 0.037524913 |
| A_24_P13041 | NM_145307 | RTKN2 | 1.1281 | 0.013838728 |
| A_23_P63816 | NM_030759 | NRBF2 | 1.1298 | 0.027235233 |
| A_23_P350895 | AK022408 | RABGAP1 | 1.1299 | 0.041329074 |
| A_23_P157580 | NM_005625 | SDCBP | 1.13 | 0.008797712 |
| A_32_P64936 | X05126 | X05126 | 1.1301 | 0.015026729 |
| A_24_P97526 | NM_017801 | CMTM6 | 1.1301 | 0.021386292 |
| A_23_P67971 | NM_138801 | GALM | 1.1301 | 0.048712016 |
| A_23_P327069 | NM_014743 | KIAA0232 | 1.1305 | 0.04524267 |
| A_24_P538459 | AF076205 | AF076205 | 1.1316 | 0.024141263 |
| A_32_P219657 | NM_002295 | RPSA | 1.1318 | 0.025433338 |
| A_23_P35912 | NM_033306 | CASP4 | 1.1329 | 0.030759431 |
| A_23_P38427 | NM_032932 | RAB11FIP4 | 1.1365 | 0.031396439 |
| A_23_P3221 | NM_021199 | SQRDL | 1.1369 | 0.032202205 |
| A_23_P34628 | NM_001514 | GTF2B | 1.1371 | 0.022111826 |
| A_23_P143551 | NM_001696 | ATP6V1E1 | 1.1377 | 0.030329249 |
| A_23_P250866 | NM_030935 | TSC22D4 | 1.1377 | 0.025798841 |
| A_23_P23141 | NM_002107 | H3F3A | 1.1382 | 0.02848718 |
| A_24_P254933 | ENST00000270031 | ENST00000270031 | 1.1392 | 0.005285936 |
| A_23_P16673 | NM_004368 | CNN2 | 1.1394 | 0.001997125 |
| A_23_P121253 | NM_003810 | TNFSF10 | 1.1394 | 0.005199365 |
| A_23_P48637 | BG108194 | BG108194 | 1.1397 | 0.044795063 |
| A_24_P511877 | NR_003955 | LOC647121 | 1.1415 | 0.025520194 |
| A_24_P650893 | A_24_P650893 | A_24_P650893 | 1.1423 | 0.021414041 |
| A_32_P31123 | THC2690780 | THC2690780 | 1.1427 | 0.022585323 |
| A_24_P450285 | NM_001033658 | LOC283152 | 1.1427 | 0.022445353 |
| A_24_P252739 | THC2621222 | THC2621222 | 1.1431 | 0.029748661 |
| A_23_P353905 | AK093617 | AK093617 | 1.1431 | 0.011023742 |
| A_23_P211299 | NM_004571 | PKNOX1 | 1.1432 | 0.049788456 |
| A_24_P417267 | AK097861 | C22orf37 | 1.1434 | 0.039281955 |
| A_24_P673209 | A_24_P673209 | A_24_P673209 | 1.1436 | 0.002836327 |
| A_32_P198371 | CR617352 | CR617352 | 1.1438 | 0.038003681 |
| A_24_P341958 | NM_178862 | STT3B | 1.1451 | 0.04234756 |
| A_23_P121253 | NM_003810 | TNFSF10 | 1.1459 | 0.005758017 |
| A_23_P144497 | NM_001006 | RPS3A | 1.1467 | 0.021226121 |
| A_24_P323835 | NM_002107 | H3F3A | 1.1468 | 0.019681745 |
| A_23_P157316 | NM_178829 | C7orf34 | 1.147 | 0.025440132 |
| A_23_P11922 | AK024925 | FLJ21272 | 1.148 | 0.022815935 |
| A_23_P121253 | NM_003810 | TNFSF10 | 1.1483 | 0.005612487 |
| A_23_P251937 | NM_030627 | CPEB4 | 1.1486 | 0.009593819 |
| A_23_P20285 | NM_021630 | PDLIM2 | 1.1488 | 0.016885329 |
| A_23_P119907 | NM_018089 | ANKZF1 | 1.1489 | 0.037334745 |
| A_24_P96403 | NM_001001890 | RUNX1 | 1.1502 | 0.017347498 |
| A_23_P11744 | NM_182905 | WASH1 | 1.1507 | 0.034149181 |
| A_32_P146508 | NM_002295 | RPSA | 1.1521 | 0.020092662 |
| A_24_P49421 | A_24_P49421 | A_24_P49421 | 1.1528 | 0.027243376 |
| A_23_P140907 | NM_021259 | TMEM8 | 1.1539 | 0.011159546 |
| A_24_P681218 | THC2544911 | THC2544911 | 1.154 | 0.026120861 |
| A_23_P213385 | NM_006317 | BASP1 | 1.1546 | 0.002845972 |
| A_23_P99320 | NM_000224 | KRT18 | 1.1554 | 0.048074248 |
| A_23_P211673 | AY358828 | SLITRK2 | 1.157 | 0.006961828 |
| A_24_P46689 | NM_015278 | SASH1 | 1.1577 | 0.010956803 |
| A_23_P76480 | BF213738 | BF213738 | 1.1578 | 0.018368705 |
| A_23_P121253 | NM_003810 | TNFSF10 | 1.1597 | 0.005349006 |
| A_23_P155057 | NM_013385 | PSCD4 | 1.1606 | 0.026225086 |
| A_32_P162004 | NM_000992 | RPL29 | 1.1634 | 0.031457351 |
| A_23_P46928 | NM_002627 | PFKP | 1.1645 | 0.033223718 |
| A_23_P360874 | NM_152892 | LRWD1 | 1.1645 | 0.009707128 |
| A_23_P137209 | NM_003334 | UBA1 | 1.1652 | 0.016472417 |
| A_23_P252825 | NM_012235 | SCAP | 1.1656 | 0.015147599 |
| A_23_P137865 | NM_198883 | MTX1 | 1.1659 | 0.03430755 |
| A_23_P358195 | NR_003246 | FLJ40113 | 1.1662 | 0.018553227 |
| A_23_P152804 | NM_198175 | NME1 | 1.1667 | 0.041721796 |
| A_23_P215233 | NM_020246 | SLC12A9 | 1.1678 | 0.040754821 |
| A_23_P207999 | NM_021127 | PMAIP1 | 1.1678 | 0.048352165 |
| A_24_P397150 | NM_005255 | GAK | 1.168 | 0.020364594 |
| A_23_P48637 | BG108194 | BG108194 | 1.1688 | 0.038053722 |
| A_24_P126741 | NM_001123388 | GLRXL | 1.1691 | 0.028035015 |
| A_24_P36847 | NM_001357 | DHX9 | 1.1693 | 0.032964423 |
| A_24_P40978 | ENST00000292543 | ENST00000292543 | 1.1703 | 0.01168164 |
| A_23_P80778 | NM_032839 | DIRC2 | 1.1705 | 0.032566889 |
| A_24_P381945 | NM_002134 | HMOX2 | 1.171 | 0.016734022 |
| A_24_P7934 | XM_373079 | LOC391769 | 1.1726 | 0.04609419 |
| A_23_P251505 | NM_148956 | NSUN5 | 1.1727 | 0.034727588 |
| A_23_P129486 | NM_016332 | SEPX1 | 1.1731 | 0.006258325 |
| A_23_P204929 | NM_016248 | AKAP11 | 1.1735 | 0.038008246 |
| A_24_P75230 | NM_033546 | MRLC2 | 1.1739 | 0.017412658 |
| A_24_P73370 | NM_003565 | ULK1 | 1.1744 | 0.015877975 |
| A_23_P56213 | NM_020895 | GRAMD1A | 1.1744 | 0.008010589 |
| A_24_P42495 | BC010544 | BC010544 | 1.1749 | 0.025842869 |
| A_24_P272710 | NM_006013 | RPL10 | 1.175 | 0.014830322 |
| A_23_P121253 | NM_003810 | TNFSF10 | 1.175 | 0.004371467 |
| A_23_P27936 | NM_006532 | ELL | 1.1751 | 0.030799076 |
| A_23_P207999 | NM_021127 | PMAIP1 | 1.1757 | 0.049358602 |
| A_32_P26092 | NM_003327 | TNFRSF4 | 1.1762 | 0.030758475 |
| A_23_P98092 | NM_000274 | OAT | 1.1784 | 0.041775445 |
| A_23_P22957 | NM_016009 | SH3GLB1 | 1.1785 | 0.017164106 |
| A_32_P459533 | NM_015122 | FCHO1 | 1.1795 | 0.043315128 |
| A_23_P122582 | NM_024294 | C6orf106 | 1.1797 | 0.04091184 |
| A_23_P139929 | NM_006817 | ERP29 | 1.1801 | 0.017364886 |
| A_23_P64721 | NM_006018 | GPR109B | 1.1811 | 0.003747385 |
| A_23_P34093 | NM_000402 | G6PD | 1.1816 | 0.01068437 |
| A_32_P38003 | ENST00000379156 | EIF2AK2 | 1.1836 | 0.016314266 |
| A_23_P20832 | NM_003127 | SPTAN1 | 1.1836 | 0.028844384 |
| A_23_P151805 | NM_006329 | FBLN5 | 1.1855 | 0.015202815 |
| A_32_P133564 | THC2663970 | THC2663970 | 1.1865 | 0.001762871 |
| A_23_P257155 | NM_000333 | ATXN7 | 1.1869 | 0.026009084 |
| A_24_P508410 | NM_002295 | RPSA | 1.187 | 0.025757384 |
| A_24_P303480 | NM_006834 | RAB32 | 1.1881 | 0.049135144 |
| A_23_P48637 | BG108194 | BG108194 | 1.1885 | 0.036870937 |
| A_32_P10003 | NM_080386 | TUBA3D | 1.1885 | 0.007347141 |
| A_32_P26895 | AB046820 | KIAA1600 | 1.1891 | 0.027125075 |
| A_23_P23279 | NM_052862 | RCSD1 | 1.1892 | 0.031067242 |
| A_23_P24104 | NM_002658 | PLAU | 1.1893 | 0.04922515 |
| A_32_P132317 | NM_001033045 | GPR155 | 1.1897 | 0.032459365 |
| A_23_P25605 | NM_032138 | KBTBD7 | 1.1912 | 0.014000761 |
| A_23_P53763 | NM_025113 | C13orf18 | 1.1913 | 0.017701753 |
| A_32_P930953 | AK024147 | AK024147 | 1.1914 | 0.024329936 |
| A_24_P178784 | A_24_P178784 | A_24_P178784 | 1.1915 | 0.017825172 |
| A_24_P766208 | NM_000967 | RPL3 | 1.1923 | 0.027588349 |
| A_23_P112004 | NM_012472 | LRRC6 | 1.1925 | 0.005083908 |
| A_23_P112774 | NM_032611 | PTP4A3 | 1.194 | 0.024114585 |
| A_24_P485105 | THC2632823 | THC2632823 | 1.1941 | 0.014922249 |
| A_23_P167983 | ENST00000314088 | HIST1H2AC | 1.1959 | 0.032644075 |
| A_23_P153383 | NM_015414 | RPL36 | 1.1959 | 0.022431932 |
| A_23_P114662 | NM_001889 | CRYZ | 1.1961 | 0.017978039 |
| A_32_P167592 | XR_015343 | XR_015343 | 1.1963 | 0.001614725 |
| A_24_P16124 | NR_001590 | IFITM4P | 1.1977 | 0.000850561 |
| A_24_P538403 | NM_005406 | ROCK1 | 1.1985 | 0.039328281 |
| A_24_P358328 | NR_002187 | tcag7.1015 | 1.1989 | 0.014914518 |
| A_23_P13344 | NM_001404 | EEF1G | 1.2001 | 0.01726049 |
| A_32_P325680 | AK074162 | AK074162 | 1.2002 | 0.019248605 |
| A_23_P107684 | NM_014347 | ZNF324 | 1.2009 | 0.047469567 |
| A_23_P120660 | NM_001024 | RPS21 | 1.2012 | 0.045496577 |
| A_23_P147805 | BC047030 | UPP1 | 1.2013 | 0.043239705 |
| A_24_P52111 | NM_001031677 | RAB24 | 1.2013 | 0.025777001 |
| A_23_P404678 | NM_004283 | RAB3D | 1.2019 | 0.04199538 |
| A_24_P41391 | NM_001031617 | COX19 | 1.2027 | 0.039303252 |
| A_23_P138514 | NM_012071 | COMMD3 | 1.2029 | 0.048906536 |
| A_32_P208424 | NM_002107 | H3F3A | 1.2034 | 0.034017997 |
| A_24_P593120 | THC2631229 | THC2631229 | 1.2036 | 0.024733662 |
| A_23_P35650 | NM_032905 | RBM17 | 1.2037 | 0.037010777 |
| A_23_P203376 | NM_152852 | MS4A6A | 1.2043 | 0.014600661 |
| A_24_P154080 | NM_001397 | ECE1 | 1.2045 | 0.007000934 |
| A_23_P125348 | NM_021210 | TRAPPC1 | 1.2063 | 0.048256339 |
| A_32_P21384 | NM_000985 | RPL17 | 1.207 | 0.012861472 |
| A_23_P15857 | NM_001042388 | PPP4R1 | 1.2071 | 0.015709152 |
| A_23_P207999 | NM_021127 | PMAIP1 | 1.2075 | 0.042783196 |
| A_24_P601695 | AK026194 | AK026194 | 1.2089 | 0.015015398 |
| A_23_P36647 | NM_177441 | TMUB2 | 1.209 | 0.0159301 |
| A_23_P77174 | NM_004855 | PIGB | 1.2096 | 0.029908239 |
| A_23_P312652 | AK128423 | CIRBP | 1.2103 | 0.037412067 |
| A_32_P201868 | CD359823 | CD359823 | 1.2108 | 0.009535279 |
| A_24_P940426 | NM_206855 | QKI | 1.2119 | 0.007916129 |
| A_24_P311926 | NM_002127 | HLA-G | 1.2123 | 0.033443759 |
| A_32_P111507 | XR_016779 | LOC440311 | 1.2123 | 0.018103231 |
| A_23_P319808 | NM_015054 | UHRF1BP1L | 1.214 | 0.036689946 |
| A_23_P5626 | A_23_P5626 | A_23_P5626 | 1.2141 | 0.024426117 |
| A_24_P390833 | NM_023075 | MPPE1 | 1.2154 | 0.036139243 |
| A_23_P140967 | NM_000243 | MEFV | 1.2155 | 0.043382645 |
| A_24_P53215 | NM_199242 | UNC13D | 1.2173 | 0.014364528 |
| A_24_P287198 | NM_001404 | EEF1G | 1.2174 | 0.04323264 |
| A_23_P102391 | NM_014585 | SLC40A1 | 1.2178 | 0.022925435 |
| A_24_P259799 | NM_024041 | SCNM1 | 1.2179 | 0.040603766 |
| A_23_P39704 | NM_001031684 | SFRS7 | 1.2181 | 0.047641089 |
| A_23_P124927 | NM_006480 | RGS14 | 1.2184 | 0.004795033 |
| A_23_P394836 | NM_022489 | INF2 | 1.2188 | 0.038369951 |
| A_32_P30710 | NM_000978 | RPL23 | 1.2195 | 0.041490537 |
| A_23_P201035 | NM_001005749 | GBA | 1.2213 | 0.022593862 |
| A_24_P899020 | THC2645960 | THC2645960 | 1.2215 | 0.028998639 |
| A_23_P101297 | NM_012155 | EML2 | 1.2216 | 0.019804737 |
| A_23_P49686 | AF090926 | AF090926 | 1.2217 | 0.035572969 |
| A_24_P653354 | THC2543840 | THC2543840 | 1.2219 | 0.037430962 |
| A_24_P192338 | CR608275 | CR608275 | 1.2222 | 0.015536175 |
| A_23_P71867 | NM_147162 | IL11RA | 1.2226 | 0.023970606 |
| A_24_P63290 | NM_198562 | C3orf62 | 1.2226 | 0.046232739 |
| A_23_P209619 | ENST00000378954 | ENST00000378954 | 1.2229 | 0.040552912 |
| A_24_P50738 | AF035020 | AF035020 | 1.2238 | 0.02957378 |
| A_24_P565496 | A_24_P565496 | A_24_P565496 | 1.2247 | 0.017011129 |
| A_32_P128980 | BC062780 | BC062780 | 1.2251 | 0.006328154 |
| A_23_P40952 | NM_002880 | RAF1 | 1.2291 | 0.024091472 |
| A_23_P217737 | L06133 | ATP7A | 1.2291 | 0.044038984 |
| A_24_P50767 | AF161342 | AF161342 | 1.2291 | 0.021944672 |
| A_24_P677608 | AK024171 | AK024171 | 1.2292 | 0.044905171 |
| A_23_P108501 | NM_004438 | EPHA4 | 1.2293 | 0.030327844 |
| A_23_P207999 | NM_021127 | PMAIP1 | 1.2294 | 0.043401195 |
| A_32_P129894 | NM_001080497 | MEGF9 | 1.2303 | 0.004479904 |
| A_32_P10396 | NM_014991 | WDFY3 | 1.2309 | 0.011440827 |
| A_23_P74278 | NM_001037341 | PDE4B | 1.2311 | 0.042601882 |
| A_24_P111737 | NM_014616 | ATP11B | 1.2316 | 0.03906361 |
| A_24_P41570 | NM_002106 | H2AFZ | 1.2326 | 0.036942124 |
| A_24_P137897 | NM_001007245 | IFRD1 | 1.2329 | 0.014131378 |
| A_23_P7101 | NM_006527 | SLBP | 1.2346 | 0.027557155 |
| A_23_P109254 | NM_184234 | RBM39 | 1.2359 | 0.040629656 |
| A_23_P257609 | NM_000992 | RPL29 | 1.2369 | 0.034918204 |
| A_32_P43436 | NM_000992 | RPL29 | 1.237 | 0.044527936 |
| A_24_P417162 | NM_005647 | TBL1X | 1.2378 | 0.025134339 |
| A_24_P367139 | A_24_P367139 | A_24_P367139 | 1.238 | 0.017804152 |
| A_23_P23616 | NM_032129 | PLEKHN1 | 1.2384 | 0.038149101 |
| A_23_P355067 | NM_019026 | TMCO1 | 1.2388 | 0.047255946 |
| A_24_P406132 | NM_002754 | MAPK13 | 1.239 | 0.008050136 |
| A_24_P639441 | NM_203330 | CD59 | 1.239 | 0.030298914 |
| A_23_P313640 | NM_006609 | MAP3K2 | 1.2401 | 0.015407549 |
| A_23_P156531 | NM_002793 | PSMB1 | 1.2413 | 0.036572896 |
| A_23_P332042 | NM_004259 | RECQL5 | 1.2421 | 0.047210102 |
| A_23_P163258 | NM_020214 | PARP6 | 1.2429 | 0.03267538 |
| A_24_P237624 | NM_182563 | C16orf79 | 1.2451 | 0.038390091 |
| A_23_P168882 | NM_033285 | TP53INP1 | 1.2457 | 0.01427629 |
| A_24_P641406 | A_24_P641406 | A_24_P641406 | 1.2461 | 0.003382495 |
| A_24_P544661 | BC068044 | BC068044 | 1.2461 | 0.03749834 |
| A_23_P44149 | NM_005730 | CTDSP2 | 1.2463 | 0.011624131 |
| A_24_P287043 | NM_006435 | IFITM2 | 1.2492 | 0.001204585 |
| A_24_P942517 | ENST00000246024 | TXNDC13 | 1.2498 | 0.015601315 |
| A_23_P16817 | NM_004071 | CLK1 | 1.2503 | 0.019560603 |
| A_23_P206396 | NM_001040138 | CKLF | 1.2506 | 0.023236548 |
| A_23_P92065 | AF116626 | AF116626 | 1.2515 | 0.035635404 |
| A_23_P146572 | NM_015392 | NPDC1 | 1.2534 | 0.011620566 |
| A_24_P76078 | XM_001724177 | LOC100130794 | 1.2542 | 0.045145789 |
| A_23_P204472 | NM_053275 | RPLP0 | 1.2549 | 0.015776528 |
| A_23_P204472 | NM_053275 | RPLP0 | 1.255 | 0.018223568 |
| A_24_P157415 | NM_014616 | ATP11B | 1.255 | 0.019686968 |
| A_23_P75889 | NM_175932 | PSMD13 | 1.2576 | 0.030591651 |
| A_24_P290293 | ENST00000399198 | ENST00000399198 | 1.2579 | 0.01643912 |
| A_24_P296347 | NM_018427 | RRN3 | 1.2588 | 0.00110157 |
| A_24_P6975 | NM_001111266 | LOC342994 | 1.2596 | 0.019729664 |
| A_23_P68942 | NM_000967 | RPL3 | 1.2598 | 0.01359095 |
| A_23_P204472 | NM_053275 | RPLP0 | 1.2598 | 0.016807062 |
| A_24_P85200 | NM_194278 | C14orf43 | 1.2607 | 0.042950807 |
| A_24_P251688 | NM_018358 | ABCF3 | 1.2609 | 0.029483023 |
| A_24_P67308 | XR_037795 | LOC100129424 | 1.2614 | 0.013455673 |
| A_32_P464568 | NM_020944 | GBA2 | 1.2643 | 0.037582508 |
| A_23_P170574 | NM_178310 | SNAI3 | 1.2646 | 0.042493372 |
| A_23_P204472 | NM_053275 | RPLP0 | 1.2648 | 0.016107645 |
| A_23_P402081 | NM_003520 | HIST1H2BN | 1.265 | 0.047188033 |
| A_24_P80915 | NM_014739 | BCLAF1 | 1.2653 | 0.025835869 |
| A_24_P73178 | NM_001005354 | PRR13 | 1.2657 | 0.030486491 |
| A_24_P161733 | A_24_P161733 | A_24_P161733 | 1.2658 | 0.030212644 |
| A_23_P207999 | NM_021127 | PMAIP1 | 1.2659 | 0.036716056 |
| A_23_P38723 | NM_015295 | SMCHD1 | 1.2665 | 0.012011423 |
| A_32_P212373 | XM_933693 | LOC440731 | 1.2695 | 0.010908217 |
| A_23_P133284 | NM_018343 | RIOK2 | 1.2695 | 0.033526772 |
| A_24_P395042 | NM_003754 | EIF3F | 1.2697 | 0.035870152 |
| A_32_P220912 | AA132288 | AA132288 | 1.2698 | 0.015725948 |
| A_23_P359647 | NM_138714 | NFAT5 | 1.2702 | 0.018588781 |
| A_24_P813550 | ENST00000390630 | ENST00000390630 | 1.2709 | 0.033364181 |
| A_23_P48495 | NM_199206 | TCL1B | 1.2714 | 0.02541552 |
| A_23_P131435 | NM_014880 | CD302 | 1.2725 | 0.037117814 |
| A_23_P204472 | NM_053275 | RPLP0 | 1.273 | 0.015704644 |
| A_24_P360269 | NM_003730 | RNASET2 | 1.2744 | 0.008667057 |
| A_23_P2967 | NM_003917 | AP1G2 | 1.2744 | 0.026196707 |
| A_32_P214503 | BC038512 | BC038512 | 1.2745 | 0.006739337 |
| A_23_P125656 | NM_001356 | DDX3X | 1.2759 | 0.036353168 |
| A_32_P91107 | BC089156 | BC089156 | 1.2763 | 0.036756339 |
| A_24_P204084 | THC2494410 | THC2494410 | 1.277 | 0.036727686 |
| A_23_P82412 | NM_001039575 | NSUN5B | 1.2771 | 0.015570806 |
| A_23_P105625 | NM_032314 | COQ5 | 1.2782 | 0.030968997 |
| A_32_P8604 | AK093691 | AK093691 | 1.2789 | 0.046829958 |
| A_23_P106562 | NM_000512 | GALNS | 1.279 | 0.020269537 |
| A_23_P254601 | NM_001662 | ARF5 | 1.2796 | 0.022536421 |
| A_32_P515088 | NM_002035 | KDSR | 1.2806 | 0.047489088 |
| A_23_P48416 | NM_014953 | DIS3 | 1.2806 | 0.016596228 |
| A_23_P316601 | NM_006912 | RIT1 | 1.2813 | 0.034082262 |
| A_24_P66270 | NR_001444 | LYPLA2P1 | 1.2821 | 0.043336763 |
| A_32_P430996 | AK001120 | AK001120 | 1.2838 | 0.037559435 |
| A_23_P131626 | NM_014014 | ASCC3L1 | 1.2842 | 0.024289318 |
| A_23_P143199 | NM_006282 | STK4 | 1.2852 | 0.012450478 |
| A_23_P90143 | NM_012423 | RPL13A | 1.2858 | 0.009743806 |
| A_23_P71241 | NM_014302 | SEC61G | 1.2865 | 0.047565225 |
| A_23_P102122 | NM_152862 | ARPC2 | 1.2869 | 0.028322836 |
| A_23_P8513 | NM_013322 | SNX10 | 1.2874 | 0.032981408 |
| A_24_P867201 | CR613944 | CR613944 | 1.2874 | 0.007938061 |
| A_23_P204472 | NM_053275 | RPLP0 | 1.2877 | 0.015296349 |
| A_23_P389676 | AK131103 | EXOC3 | 1.2889 | 0.017695529 |
| A_23_P48676 | NM_002863 | PYGL | 1.2895 | 0.002744909 |
| A_23_P204472 | NM_053275 | RPLP0 | 1.2902 | 0.014098331 |
| A_23_P215175 | NM_005692 | ABCF2 | 1.2903 | 0.049879476 |
| A_24_P590560 | NM_018427 | RRN3 | 1.2915 | 0.02270862 |
| A_23_P120744 | NM_003906 | MCM3AP | 1.2921 | 0.040358778 |
| A_32_P52609 | NM_145693 | LPIN1 | 1.2921 | 0.008757845 |
| A_23_P887 | NM_014002 | IKBKE | 1.2927 | 0.024894424 |
| A_23_P434809 | NM_002964 | S100A8 | 1.2934 | 0.003581141 |
| A_24_P67638 | A_24_P67638 | A_24_P67638 | 1.2955 | 0.047844664 |
| A_23_P99163 | NM_018370 | DRAM | 1.2973 | 0.020986275 |
| A_24_P42066 | NR_003267 | GGT3P | 1.2977 | 0.033508589 |
| A_24_P329065 | NM_007048 | BTN3A1 | 1.3018 | 0.038753605 |
| A_32_P136204 | BQ548663 | BQ548663 | 1.3024 | 0.031257851 |
| A_24_P214556 | A_24_P214556 | A_24_P214556 | 1.3025 | 0.036461169 |
| A_23_P63584 | NM_015446 | AHCTF1 | 1.3026 | 0.010778019 |
| A_32_P20367 | NM_001011 | RPS7 | 1.3044 | 0.018671304 |
| A_24_P479645 | AK093640 | AK093640 | 1.3048 | 0.049366878 |
| A_23_P407628 | NM_006766 | MYST3 | 1.3056 | 0.028368976 |
| A_24_P160920 | A_24_P160920 | A_24_P160920 | 1.3058 | 0.016348772 |
| A_24_P333019 | NM_007219 | RNF24 | 1.3059 | 0.002037004 |
| A_23_P84154 | NM_018460 | ARHGAP15 | 1.306 | 0.021845297 |
| A_23_P70785 | NM_001624 | AIM1 | 1.3062 | 0.021095067 |
| A_23_P117175 | NM_006437 | PARP4 | 1.3063 | 0.022855919 |
| A_24_P236091 | NM_001975 | ENO2 | 1.3063 | 0.029452744 |
| A_24_P375340 | A_24_P375340 | A_24_P375340 | 1.3066 | 0.019898324 |
| A_23_P138308 | NM_001779 | CD58 | 1.307 | 0.03824284 |
| A_23_P115998 | NM_003120 | SPI1 | 1.3074 | 0.014292893 |
| A_24_P916718 | NM_207336 | ZNF467 | 1.3079 | 0.040240496 |
| A_24_P924697 | AK055915 | AK055915 | 1.308 | 0.009700483 |
| A_23_P97932 | NM_012228 | MSRB2 | 1.3082 | 0.007811231 |
| A_23_P61050 | NM_152649 | MLKL | 1.3085 | 0.036741323 |
| A_23_P76851 | NM_001039619 | PRMT5 | 1.3092 | 0.008727603 |
| A_23_P253221 | NM_032995 | ARHGEF4 | 1.3098 | 0.012011184 |
| A_32_P58029 | A_32_P58029 | A_32_P58029 | 1.311 | 0.021310281 |
| A_23_P142421 | NM_014727 | MLL4 | 1.3113 | 0.006766245 |
| A_23_P148015 | NM_004655 | AXIN2 | 1.3114 | 0.019696448 |
| A_32_P38404 | BC015133 | BC015133 | 1.3118 | 0.048914877 |
| A_32_P218251 | BC019599 | BC019599 | 1.3132 | 0.041306653 |
| A_24_P755505 | A_24_P755505 | A_24_P755505 | 1.3134 | 0.012493891 |
| A_23_P150718 | NM_001665 | RHOG | 1.3139 | 0.004449196 |
| A_23_P68072 | NM_032118 | WDR54 | 1.3144 | 0.031698728 |
| A_23_P28084 | CR601641 | STX10 | 1.3174 | 0.025408392 |
| A_23_P49033 | NM_003099 | SNX1 | 1.3176 | 0.030147045 |
| A_23_P40952 | NM_002880 | RAF1 | 1.3195 | 0.017547559 |
| A_23_P150147 | NM_017857 | SSH3 | 1.3196 | 0.029898658 |
| A_32_P208178 | NM_001006 | RPS3A | 1.3198 | 0.013351018 |
| A_24_P941505 | NM_014612 | FAM120A | 1.3214 | 0.02753233 |
| A_24_P169013 | NR_001298 | HLA-DRB6 | 1.3219 | 0.023204403 |
| A_24_P181149 | NM_024685 | BBS10 | 1.3221 | 0.023973971 |
| A_23_P92410 | NM_004346 | CASP3 | 1.3234 | 0.037164173 |
| A_23_P213592 | NM_014901 | RNF44 | 1.3239 | 0.038447297 |
| A_24_P850428 | BC070327 | BC070327 | 1.3239 | 0.006417593 |
| A_23_P40952 | NM_002880 | RAF1 | 1.3242 | 0.01854739 |
| A_24_P153840 | NM_033086 | FGD3 | 1.3245 | 0.006898747 |
| A_23_P9823 | NM_014938 | MLXIP | 1.3248 | 0.0061622 |
| A_23_P157569 | NM_144650 | ADHFE1 | 1.3249 | 0.046425714 |
| A_23_P39602 | NM_147233 | NCOA1 | 1.3252 | 0.028946245 |
| A_32_P213946 | A_32_P213946 | A_32_P213946 | 1.3259 | 0.008879441 |
| A_24_P101960 | A_24_P101960 | A_24_P101960 | 1.3267 | 0.022268934 |
| A_24_P250922 | NM_000963 | PTGS2 | 1.327 | 0.007194093 |
| A_24_P396105 | NM_153273 | IHPK1 | 1.3272 | 0.033893435 |
| A_24_P349356 | NM_207354 | ANKRD13D | 1.3278 | 0.003585557 |
| A_24_P57837 | THC2567891 | THC2567891 | 1.3285 | 0.007312619 |
| A_24_P926960 | NM_001409 | MEGF6 | 1.3285 | 0.009288802 |
| A_23_P60376 | NM_153200 | EDF1 | 1.3297 | 0.023885199 |
| A_24_P216087 | NM_019088 | PAF1 | 1.3298 | 0.044469046 |
| A_24_P76868 | X01147 | X01147 | 1.3299 | 0.013093863 |
| A_23_P341065 | NM_031454 | RP3-402G11.5 | 1.3308 | 0.017010758 |
| A_24_P780052 | NM_001089588 | hCG_1984468 | 1.3311 | 0.007917302 |
| A_23_P361569 | NM_025181 | SLC35F5 | 1.3315 | 0.041041311 |
| A_23_P204472 | NM_053275 | RPLP0 | 1.3315 | 0.013168155 |
| A_24_P217834 | NM_003530 | HIST1H3D | 1.3362 | 0.003887422 |
| A_23_P155417 | NM_032750 | ABHD14B | 1.3362 | 0.035136118 |
| A_23_P138881 | NM_001104 | ACTN3 | 1.3364 | 0.01564035 |
| A_23_P90752 | NM_015909 | NAG | 1.3365 | 0.039063351 |
| A_23_P163942 | NM_003168 | SUPT4H1 | 1.3368 | 0.019601649 |
| A_24_P103686 | NM_178819 | AGPAT6 | 1.3375 | 0.017875026 |
| A_23_P117546 | NM_006939 | SOS2 | 1.3379 | 0.016872286 |
| A_23_P408473 | NM_032450 | KIAA1833 | 1.3385 | 0.03884339 |
| A_23_P62583 | NM_001409 | MEGF6 | 1.3394 | 0.019111377 |
| A_24_P303080 | A_24_P303080 | A_24_P303080 | 1.3399 | 0.04991547 |
| A_23_P140515 | NM_078474 | TM2D3 | 1.3404 | 0.040455767 |
| A_24_P349756 | A_24_P349756 | A_24_P349756 | 1.3405 | 0.010443499 |
| A_23_P408271 | NM_016245 | HSD17B11 | 1.3415 | 0.014952321 |
| A_32_P184488 | NM_198850 | PHLDB3 | 1.3417 | 0.010957782 |
| A_23_P40952 | NM_002880 | RAF1 | 1.3443 | 0.016346403 |
| A_24_P340976 | XR_018155 | XR_018155 | 1.3445 | 0.016202847 |
| A_24_P168760 | NM_004286 | GTPBP1 | 1.3453 | 0.040270947 |
| A_24_P128361 | AF289562 | LOC100130339 | 1.3459 | 0.005115464 |
| A_23_P43086 | NM_018688 | BIN3 | 1.3467 | 0.0189685 |
| A_32_P76399 | NM_016091 | EIF3EIP | 1.347 | 0.022925089 |
| A_23_P141100 | NM_012091 | ADAT1 | 1.347 | 0.049642073 |
| A_24_P419211 | NM_004685 | MTMR6 | 1.3478 | 0.048418052 |
| A_23_P58747 | NM_015111 | N4BP3 | 1.348 | 0.02246531 |
| A_24_P403501 | BC043198 | CECR7 | 1.3494 | 0.010548103 |
| A_24_P7085 | NR_002778 | RPL29P2 | 1.3495 | 0.014368276 |
| A_23_P390190 | NM_014089 | NUPL1 | 1.3499 | 0.019515664 |
| A_24_P185044 | NM_152649 | MLKL | 1.3501 | 0.034418892 |
| A_23_P317800 | NM_013367 | ANAPC4 | 1.3514 | 0.046280778 |
| A_23_P7221 | NM_033625 | RPL34 | 1.3514 | 0.022229979 |
| A_32_P184796 | NM_053275 | RPLP0 | 1.3515 | 0.013154512 |
| A_23_P106898 | NM_152288 | ORAI3 | 1.3518 | 0.035533115 |
| A_23_P156198 | NM_015288 | PHF15 | 1.3521 | 0.028318803 |
| A_23_P216630 | NM_080546 | SLC44A1 | 1.3532 | 0.030343973 |
| A_23_P118105 | NM_004548 | NDUFB10 | 1.3564 | 0.032122236 |
| A_24_P8049 | NM_014515 | CNOT2 | 1.3582 | 0.016964342 |
| A_23_P93881 | NM_182715 | SYPL1 | 1.3592 | 0.017903907 |
| A_24_P27229 | NM_015989 | CSAD | 1.3599 | 0.015589769 |
| A_23_P54243 | NM_003485 | GPR68 | 1.3602 | 0.028428343 |
| A_23_P31840 | NM_032378 | EEF1D | 1.3603 | 0.043831756 |
| A_23_P209962 | NM_024624 | SMC6 | 1.3621 | 0.042071798 |
| A_23_P110571 | NM_015183 | MAST4 | 1.3623 | 0.02791112 |
| A_24_P925455 | A_24_P925455 | A_24_P925455 | 1.3626 | 0.039984886 |
| A_23_P207999 | NM_021127 | PMAIP1 | 1.3629 | 0.025568831 |
| A_23_P2998 | NM_005015 | OXA1L | 1.3633 | 0.039278544 |
| A_23_P335958 | NM_001033045 | GPR155 | 1.3637 | 0.014285343 |
| A_23_P61823 | NM_001031677 | RAB24 | 1.3644 | 0.009751222 |
| A_32_P56618 | XM_001726460 | LOC100130600 | 1.3648 | 0.019654907 |
| A_23_P148919 | NM_000098 | CPT2 | 1.3658 | 0.020225834 |
| A_23_P48550 | NM_015005 | KIAA0284 | 1.3661 | 0.043893785 |
| A_23_P217564 | NM_004458 | ACSL4 | 1.3662 | 0.006203388 |
| A_24_P383788 | NM_138368 | DKFZp761E198 | 1.3664 | 0.022304926 |
| A_24_P271014 | NM_001042388 | PPP4R1 | 1.3683 | 0.013654976 |
| A_23_P27515 | NM_012268 | PLD3 | 1.3696 | 0.021743859 |
| A_23_P9603 | NM_006904 | PRKDC | 1.3697 | 0.00949145 |
| A_23_P209904 | NM_002081 | GPC1 | 1.3702 | 0.015385967 |
| A_24_P450172 | AK095151 | AK095151 | 1.3705 | 0.001733626 |
| A_24_P40795 | XM_497352 | LOC283412 | 1.3706 | 0.008488065 |
| A_32_P4403 | NM_014117 | C16orf72 | 1.3707 | 0.033419341 |
| A_32_P177040 | NM_001099435 | LOC442590 | 1.3708 | 0.009973596 |
| A_24_P295601 | NM_014741 | HARBI1 | 1.371 | 0.018516177 |
| A_23_P21409 | NM_001042384 | CEP63 | 1.3713 | 0.016927439 |
| A_23_P214330 | NM_030666 | SERPINB1 | 1.3719 | 0.00469849 |
| A_23_P92410 | NM_004346 | CASP3 | 1.372 | 0.027871686 |
| A_32_P15156 | BX648207 | BX648207 | 1.3727 | 0.028821475 |
| A_23_P35399 | NM_145869 | ANXA11 | 1.375 | 0.006212661 |
| A_24_P303974 | NM_006023 | CDC123 | 1.375 | 0.029162513 |
| A_32_P38145 | AL136845 | PACSIN2 | 1.3753 | 0.004740403 |
| A_23_P215931 | NM_015344 | LEPROTL1 | 1.3773 | 0.025272413 |
| A_23_P64129 | NM_006410 | HTATIP2 | 1.3773 | 0.009201545 |
| A_23_P134835 | NM_018371 | CSGALNACT1 | 1.3773 | 0.000161715 |
| A_23_P204472 | NM_053275 | RPLP0 | 1.3795 | 0.010413651 |
| A_23_P329198 | NM_001031716 | OBFC2A | 1.3799 | 0.012960888 |
| A_24_P367109 | XR_019503 | LOC728533 | 1.3802 | 0.035241396 |
| A_24_P940551 | ENST00000299125 | ENST00000299125 | 1.3803 | 0.032681154 |
| A_24_P250922 | NM_000963 | PTGS2 | 1.3808 | 0.005223947 |
| A_23_P257164 | NM_000481 | AMT | 1.3816 | 0.038223703 |
| A_23_P256561 | NM_006068 | TLR6 | 1.3819 | 0.003132212 |
| A_23_P40952 | NM_002880 | RAF1 | 1.3824 | 0.01279998 |
| A_23_P210708 | NM_001040022 | SIRPA | 1.3829 | 0.012038201 |
| A_23_P209962 | NM_024624 | SMC6 | 1.3835 | 0.039695985 |
| A_24_P96171 | NM_030578 | B9D2 | 1.3838 | 0.013843529 |
| A_32_P184367 | NM_006013 | RPL10 | 1.3857 | 0.035176935 |
| A_24_P250922 | NM_000963 | PTGS2 | 1.3858 | 0.005053515 |
| A_24_P235429 | NM_005502 | ABCA1 | 1.3863 | 0.001929929 |
| A_23_P87853 | NM_020698 | TMCC3 | 1.3866 | 0.00368456 |
| A_23_P202750 | NM_014039 | C11orf54 | 1.3872 | 0.035387521 |
| A_23_P16683 | NM_017722 | TRMT1 | 1.388 | 0.036648149 |
| A_23_P308305 | ENST00000317571 | ENST00000317571 | 1.3883 | 0.040255265 |
| A_23_P99540 | NM_004926 | ZFP36L1 | 1.3891 | 0.018595299 |
| A_23_P61674 | NM_020666 | CLK4 | 1.3891 | 0.008364492 |
| A_23_P94216 | NM_152271 | LONRF1 | 1.3892 | 0.018945517 |
| A_24_P506977 | NR_003697 | C7orf40 | 1.3904 | 0.039848771 |
| A_24_P266880 | NM_021009 | UBC | 1.3916 | 0.048991854 |
| A_24_P250922 | NM_000963 | PTGS2 | 1.3928 | 0.004348978 |
| A_32_P167176 | THC2701422 | THC2701422 | 1.3929 | 0.025746291 |
| A_23_P45875 | NM_003292 | TPR | 1.3939 | 0.036646739 |
| A_23_P95594 | NM_000662 | NAT1 | 1.3944 | 0.048617865 |
| A_23_P69141 | NM_016141 | DYNC1LI1 | 1.3949 | 0.02434401 |
| A_32_P115749 | CD104030 | CD104030 | 1.3951 | 0.003205023 |
| A_23_P1682 | NM_138788 | TMEM45B | 1.3953 | 0.000725906 |
| A_23_P13978 | NM_016123 | IRAK4 | 1.3969 | 0.016600966 |
| A_23_P165548 | NM_022460 | HS1BP3 | 1.3971 | 0.01703597 |
| A_32_P115518 | AI207522 | AI207522 | 1.3972 | 0.032105585 |
| A_23_P217797 | AF000984 | DDX3Y | 1.3975 | 0.020704049 |
| A_32_P65303 | THC2672675 | THC2672675 | 1.3975 | 0.019188554 |
| A_23_P428219 | NM_001991 | EZH1 | 1.398 | 0.014540679 |
| A_24_P419087 | NM_006576 | AVIL | 1.3985 | 0.01564608 |
| A_24_P250922 | NM_000963 | PTGS2 | 1.3987 | 0.00452308 |
| A_24_P761386 | A_24_P761386 | A_24_P761386 | 1.3999 | 0.031032967 |
| A_23_P31097 | NM_014028 | OSTM1 | 1.4019 | 0.047899293 |
| A_24_P114617 | NM_014043 | CHMP2B | 1.4034 | 0.029549368 |
| A_24_P204454 | A_24_P204454 | A_24_P204454 | 1.4045 | 0.03156366 |
| A_23_P8561 | NM_001040457 | RHBDD2 | 1.4047 | 0.039342637 |
| A_24_P650011 | A_24_P650011 | A_24_P650011 | 1.405 | 0.016695904 |
| A_24_P126060 | NM_001356 | DDX3X | 1.4051 | 0.009245085 |
| A_23_P204472 | NM_053275 | RPLP0 | 1.4056 | 0.008979128 |
| A_24_P565503 | A_24_P565503 | A_24_P565503 | 1.4057 | 0.011406734 |
| A_23_P398770 | AK055921 | PWWP2A | 1.4057 | 0.029227969 |
| A_23_P159907 | NM_030801 | MAGED4B | 1.4064 | 0.025989545 |
| A_24_P358164 | NM_001089588 | hCG_1984468 | 1.4065 | 0.01161647 |
| A_23_P62652 | NM_003037 | SLAMF1 | 1.4065 | 0.034490111 |
| A_23_P10463 | NM_004552 | NDUFS5 | 1.4067 | 0.042429404 |
| A_24_P289188 | XM_001133762 | LOC729611 | 1.4067 | 0.035975479 |
| A_23_P26583 | NM_032206 | NLRC5 | 1.407 | 0.004488797 |
| A_23_P161501 | NM_024954 | UBTD1 | 1.4073 | 0.012063776 |
| A_23_P217507 | NM_004729 | ZBED1 | 1.4077 | 0.007028993 |
| A_23_P80062 | NM_003185 | TAF4 | 1.4081 | 0.015537769 |
| A_32_P158746 | NM_000985 | RPL17 | 1.4081 | 0.006426148 |
| A_24_P28165 | NM_015071 | ARHGAP26 | 1.4081 | 0.002834246 |
| A_32_P114739 | DB518505 | DB518505 | 1.4083 | 0.038699826 |
| A_23_P396299 | ENST00000324928 | ENST00000324928 | 1.4102 | 0.02774058 |
| A_23_P206347 | NM_016025 | METTL9 | 1.4102 | 0.033256351 |
| A_32_P75867 | THC2691419 | THC2691419 | 1.4102 | 0.001580288 |
| A_24_P230691 | AK025303 | KIF13A | 1.4108 | 0.00846651 |
| A_24_P48069 | NM_018110 | DOK4 | 1.4118 | 0.017388028 |
| A_23_P17382 | NM_022095 | ZNF335 | 1.4119 | 0.019547446 |
| A_23_P28857 | NM_018556 | SIRPG | 1.4121 | 0.036215702 |
| A_23_P200199 | NM_015913 | TXNDC12 | 1.4129 | 0.01591757 |
| A_23_P87973 | NM_213590 | TRIM13 | 1.4134 | 0.048776167 |
| A_24_P919304 | BC012204 | FNDC3B | 1.4138 | 0.025594413 |
| A_23_P93258 | NM_003537 | HIST1H3B | 1.4141 | 0.012442362 |
| A_24_P250922 | NM_000963 | PTGS2 | 1.4154 | 0.004266148 |
| A_23_P258698 | NM_005908 | MANBA | 1.4155 | 0.034927226 |
| A_23_P362759 | NM_018699 | PRDM5 | 1.4157 | 0.036454286 |
| A_23_P140328 | NM_004713 | SDCCAG1 | 1.4175 | 0.046917422 |
| A_24_P187023 | XM_001718967 | LOC390876 | 1.4179 | 0.021063173 |
| A_23_P40952 | NM_002880 | RAF1 | 1.419 | 0.012262561 |
| A_23_P165171 | NM_017814 | TMEM161A | 1.419 | 0.040957875 |
| A_23_P86884 | NM_005146 | SART1 | 1.4194 | 0.014199098 |
| A_23_P51187 | NM_002744 | PRKCZ | 1.4199 | 0.022076999 |
| A_23_P98876 | NM_173596 | SLC39A5 | 1.4217 | 0.029013252 |
| A_24_P373174 | NM_004580 | RAB27A | 1.4219 | 0.015328208 |
| A_23_P101007 | NM_178493 | NOTUM | 1.4233 | 0.047219016 |
| A_24_P406034 | NM_006416 | SLC35A1 | 1.4234 | 0.016502191 |
| A_24_P67148 | AF318328 | AF318328 | 1.4239 | 0.027165247 |
| A_32_P112279 | NM_001039690 | CTF8 | 1.4239 | 0.00923095 |
| A_23_P157452 | NM_005034 | POLR2K | 1.4245 | 0.032204367 |
| A_23_P254472 | NM_024573 | C6orf211 | 1.4247 | 0.029336126 |
| A_24_P250922 | NM_000963 | PTGS2 | 1.4249 | 0.004467371 |
| A_23_P212728 | BC020955 | TBC1D23 | 1.4255 | 0.033956909 |
| A_23_P90804 | NM_145686 | MAP4K4 | 1.4255 | 0.023728333 |
| A_23_P36345 | NM_006645 | STARD10 | 1.426 | 0.013803646 |
| A_23_P142776 | NM_003754 | EIF3F | 1.4271 | 0.041109331 |
| A_32_P50066 | NM_001039580 | MAP9 | 1.4278 | 0.030881494 |
| A_32_P105195 | NM_014829 | DDX46 | 1.4278 | 0.031165847 |
| A_23_P39356 | NM_005303 | FFAR1 | 1.43 | 0.025857672 |
| A_24_P379750 | NM_002357 | MXD1 | 1.4311 | 0.00375457 |
| A_23_P149509 | NM_005341 | ZBTB48 | 1.4311 | 0.0133703 |
| A_24_P75708 | XR_018526 | XR_018526 | 1.4315 | 0.007892469 |
| A_24_P266880 | NM_021009 | UBC | 1.4321 | 0.039566584 |
| A_23_P71464 | NM_001359 | DECR1 | 1.4322 | 0.042584862 |
| A_23_P49467 | NM_001114600 | C1orf144 | 1.4331 | 0.039002817 |
| A_23_P151075 | NM_001175 | ARHGDIB | 1.4342 | 0.031744525 |
| A_23_P170986 | NM_001017395 | TMCC1 | 1.4345 | 0.039844088 |
| A_23_P40952 | NM_002880 | RAF1 | 1.436 | 0.011144452 |
| A_23_P218476 | NM_030578 | B9D2 | 1.4368 | 0.023649996 |
| A_24_P81740 | NM_006755 | TALDO1 | 1.4389 | 0.022299508 |
| A_23_P40952 | NM_002880 | RAF1 | 1.4407 | 0.010140166 |
| A_23_P2901 | AK000041 | C14orf113 | 1.4408 | 0.02742528 |
| A_32_P137035 | NM_006510 | TRIM27 | 1.4413 | 0.004166967 |
| A_24_P332780 | A_24_P332780 | A_24_P332780 | 1.4415 | 0.045154692 |
| A_32_P8857 | A_32_P8857 | A_32_P8857 | 1.443 | 0.026133307 |
| A_23_P385267 | NM_173618 | CCDC95 | 1.4436 | 0.041171701 |
| A_23_P202939 | NM_001642 | APLP2 | 1.4445 | 0.042611645 |
| A_23_P105307 | NM_201444 | DGKA | 1.4458 | 0.01439333 |
| A_24_P350126 | A_24_P350126 | A_24_P350126 | 1.446 | 0.024374281 |
| A_23_P92410 | NM_004346 | CASP3 | 1.4473 | 0.034327585 |
| A_23_P157449 | NM_005034 | POLR2K | 1.4494 | 0.008829461 |
| A_23_P117163 | NM_018191 | RCBTB1 | 1.4496 | 0.027730588 |
| A_32_P14894 | NM_001014 | RPS10 | 1.4498 | 0.035741101 |
| A_23_P81660 | NM_018368 | LMBRD1 | 1.4501 | 0.023610097 |
| A_32_P126375 | NM_198270 | NHS | 1.4504 | 0.011061648 |
| A_23_P65877 | NM_001042496 | SLC12A6 | 1.4509 | 0.024406524 |
| A_24_P245976 | NM_013452 | VCX | 1.4509 | 0.019662584 |
| A_23_P317001 | U15173 | BNIP2 | 1.4511 | 0.019679263 |
| A_23_P74547 | NM_001040033 | CD53 | 1.4511 | 0.024848836 |
| A_23_P74843 | NM_018060 | IARS2 | 1.4521 | 0.049338874 |
| A_32_P180435 | NM_175064 | WBSCR19 | 1.4538 | 0.012109191 |
| A_23_P251342 | NM_002093 | GSK3B | 1.4538 | 0.043355695 |
| A_24_P12435 | NM_181782 | NCOA7 | 1.4541 | 0.0172043 |
| A_23_P347131 | AK094181 | TTC15 | 1.4542 | 0.020486122 |
| A_24_P50472 | XM_938903 | LOC649839 | 1.4546 | 0.040088223 |
| A_32_P33083 | NM_016378 | VCX2 | 1.4555 | 0.012893502 |
| A_24_P85181 | NM_022340 | ZFYVE20 | 1.456 | 0.027196844 |
| A_24_P109921 | ENST00000390541 | ENST00000390541 | 1.4577 | 0.017720847 |
| A_23_P209962 | NM_024624 | SMC6 | 1.4581 | 0.035003176 |
| A_23_P104351 | NM_207009 | FAM45A | 1.4587 | 0.027225941 |
| A_23_P204736 | NM_005276 | GPD1 | 1.4594 | 0.027187288 |
| A_23_P10815 | NM_001020658 | PUM1 | 1.4594 | 0.022754989 |
| A_23_P154986 | NM_005265 | GGT1 | 1.4595 | 0.020181243 |
| A_23_P43079 | NM_017864 | INTS8 | 1.4599 | 0.036771553 |
| A_23_P86222 | NM_138794 | LYPLAL1 | 1.4605 | 0.029724989 |
| A_23_P382043 | NM_152729 | NT5DC1 | 1.4612 | 0.036815972 |
| A_24_P139943 | NM_022460 | HS1BP3 | 1.4628 | 0.013997247 |
| A_23_P384085 | NM_181453 | GCC2 | 1.463 | 0.026102134 |
| A_23_P107166 | NM_024722 | ACBD4 | 1.4642 | 0.037304364 |
| A_24_P532232 | NM_182898 | CREB5 | 1.4648 | 0.001613599 |
| A_23_P401380 | NM_173602 | DIP2B | 1.465 | 0.008603351 |
| A_24_P336113 | NM_012295 | CABIN1 | 1.4654 | 0.033425492 |
| A_23_P156609 | A_23_P156609 | A_23_P156609 | 1.4666 | 0.004921304 |
| A_24_P54178 | NM_016040 | TMED5 | 1.4693 | 0.006949059 |
| A_23_P31195 | NM_031434 | TMUB1 | 1.4698 | 0.023777852 |
| A_23_P431543 | NM_182612 | PDDC1 | 1.4704 | 0.03983858 |
| A_23_P100141 | NM_023076 | UNKL | 1.4707 | 0.008175681 |
| A_24_P2584 | BC065040 | C1orf63 | 1.4717 | 0.025674503 |
| A_24_P58647 | XR_018731 | LOC441131 | 1.4718 | 0.002464993 |
| A_23_P61987 | NM_025268 | TMEM121 | 1.4727 | 0.001690954 |
| A_23_P135778 | NM_018461 | PPP2R2D | 1.4729 | 0.041723404 |
| A_24_P250922 | NM_000963 | PTGS2 | 1.4731 | 0.00342908 |
| A_24_P12539 | NM_015483 | KBTBD2 | 1.4736 | 0.024991822 |
| A_23_P55564 | BC006340 | ZCCHC2 | 1.4739 | 0.034709988 |
| A_32_P41065 | NM_001017395 | TMCC1 | 1.4743 | 0.007208469 |
| A_23_P14716 | NM_031284 | ADPGK | 1.4745 | 0.042004497 |
| A_24_P404245 | NM_002861 | PCYT2 | 1.4747 | 0.033213129 |
| A_23_P218637 | NM_005054 | RGPD5 | 1.4749 | 0.024077635 |
| A_23_P11543 | NM_000147 | FUCA1 | 1.4755 | 0.000941246 |
| A_23_P52589 | NM_003475 | RASSF7 | 1.4758 | 0.049747488 |
| A_23_P6447 | NM_006860 | RABL4 | 1.4765 | 0.046818287 |
| A_24_P255954 | XR_019330 | KRT18P33 | 1.4767 | 0.026480861 |
| A_23_P75369 | NM_006019 | TCIRG1 | 1.4769 | 0.009053404 |
| A_23_P215406 | NM_198829 | RAC1 | 1.4775 | 0.010430086 |
| A_23_P54900 | NM_016936 | UBN1 | 1.4791 | 0.002214617 |
| A_23_P391164 | NM_032434 | ZNF512 | 1.4793 | 0.044484394 |
| A_24_P132787 | NM_021252 | RAB18 | 1.4796 | 0.024490751 |
| A_23_P376661 | NM_005600 | NIT1 | 1.4798 | 0.033030122 |
| A_23_P40952 | NM_002880 | RAFl | 1.4804 | 0.008915304 |
| A_24_P612656 | XR_018574 | XR_018574 | 1.4804 | 0.010520117 |
| A_23_P202199 | NM_014720 | SLK | 1.4806 | 0.027153425 |
| A_24_P465772 | BC107865 | LOC204010 | 1.482 | 0.005827629 |
| A_23_P143559 | NM_007098 | CLTCL1 | 1.4821 | 0.036330445 |
| A_23_P380181 | NM_006769 | LMO4 | 1.4842 | 0.008045633 |
| A_24_P350060 | XR_018193 | KRT18P42 | 1.4847 | 0.016663793 |
| A_23_P9591 | NM_006816 | LMAN2 | 1.485 | 0.018540463 |
| A_24_P50437 | BC065737 | BC065737 | 1.4868 | 0.018473491 |
| A_23_P129246 | NM_025201 | PLEKHO2 | 1.487 | 0.007286003 |
| A_24_P286054 | NM_001105251 | ZFYVE16 | 1.4877 | 0.008587419 |
| A_32_P124461 | AK129743 | AK129743 | 1.4881 | 0.037037777 |
| A_23_P133227 | NM_002109 | HARS | 1.4884 | 0.048455609 |
| A_23_P87917 | NM_012284 | KCNH3 | 1.4905 | 0.037196829 |
| A_23_P164797 | NM_016202 | ZNF580 | 1.4909 | 0.01921845 |
| A_32_P22078 | NM_001013 | RPS9 | 1.4917 | 0.028374043 |
| A_23_P167464 | AK022140 | FLJ12078 | 1.4927 | 0.034003522 |
| A_23_P48897 | AF011794 | CCPG1 | 1.4928 | 0.015442478 |
| A_23_P306500 | NM_033360 | KRAS | 1.4945 | 0.019475867 |
| A_24_P134526 | NM_000714 | TSPO | 1.4957 | 0.007963878 |
| A_24_P322369 | NM_004869 | VPS4B | 1.4961 | 0.011676985 |
| A_24_P250922 | NM_000963 | PTGS2 | 1.4973 | 0.002883912 |
| A_23_P93348 | NM_002341 | LTB | 1.498 | 0.00355036 |
| A_23_P145068 | NM_012123 | MTO1 | 1.4982 | 0.029796158 |
| A_23_P45517 | NM_013239 | PPP2R3B | 1.4984 | 0.004993513 |
| A_23_P105747 | NM_018171 | APPL2 | 1.4985 | 0.049795216 |
| A_23_P208866 | NM_004877 | GMFG | 1.4987 | 0.003986124 |
| A_23_P133506 | NM_024872 | DOK3 | 1.4988 | 0.015546663 |
| A_24_P250922 | NM_000963 | PTGS2 | 1.4997 | 0.002916895 |
| A_24_P23979 | NM_016379 | VCX3A | 1.5012 | 0.002941747 |
| A_23_P139687 | NM_152321 | ERP27 | 1.5013 | 0.037398249 |
| A_23_P251342 | NM_002093 | GSK3B | 1.5016 | 0.039461212 |
| A_23_P30870 | ENST00000383267 | ENST00000383267 | 1.5025 | 0.016518883 |
| A_23_P48897 | AF011794 | CCPG1 | 1.5039 | 0.014523932 |
| A_24_P19337 | NM_015338 | ASXL1 | 1.506 | 0.025313879 |
| A_24_P207727 | NM_001003891 | MED15 | 1.5065 | 0.006068636 |
| A_23_P214139 | NM_002912 | REV3L | 1.5069 | 0.046809712 |
| A_23_P250379 | NM_015274 | MAN2B2 | 1.5069 | 0.029921193 |
| A_23_P163428 | NM_021823 | PPCDC | 1.5073 | 0.008047626 |
| A_23_P366216 | NM_003524 | HIST1H2BH | 1.508 | 0.015340347 |
| A_24_P266880 | NM_021009 | UBC | 1.5087 | 0.042637244 |
| A_23_P21644 | NM_016245 | HSD17B11 | 1.5087 | 0.005490619 |
| A_23_P145865 | NM_006869 | CENTA1 | 1.5101 | 0.017087037 |
| A_24_P392231 | AW302767 | AW302767 | 1.5137 | 0.035661702 |
| A_24_P191971 | AK027248 | LOC56757 | 1.5139 | 0.045423909 |
| A_23_P120002 | NM_004510 | SP110 | 1.5146 | 0.004074607 |
| A_23_P202602 | NM_007190 | SEC23IP | 1.5146 | 0.025036503 |
| A_23_P133095 | NM_014247 | RAPGEF2 | 1.5148 | 0.014478037 |
| A_24_P281374 | XR_018462 | XR_018462 | 1.5153 | 0.036708734 |
| A_24_P211565 | NM_031910 | C1QTNF6 | 1.5156 | 0.018878072 |
| A_24_P332837 | BC039449 | MSL-1 | 1.5161 | 0.005917555 |
| A_24_P168232 | NM_030576 | LIMD2 | 1.5163 | 0.024154285 |
| A_24_P242299 | NM_005455 | ZRANB2 | 1.5166 | 0.011142748 |
| A_23_P105368 | AK001057 | MGC16384 | 1.5172 | 0.004076665 |
| A_23_P215406 | NM_198829 | RAC1 | 1.5188 | 0.008087221 |
| A_23_P256158 | NM_000683 | ADRA2C | 1.5204 | 0.038211453 |
| A_23_P30776 | NM_003523 | HIST1H2BE | 1.5205 | 0.034320193 |
| A_23_P215406 | NM_198829 | RAC1 | 1.5206 | 0.009476368 |
| A_24_P322741 | NM_000628 | IL10RB | 1.5209 | 0.00642504 |
| A_23_P306507 | NM_033360 | KRAS | 1.5213 | 0.015844145 |
| A_24_P414371 | NM_000944 | PPP3CA | 1.5217 | 0.009910111 |
| A_23_P258124 | NM_012279 | ZNF346 | 1.5231 | 0.042365757 |
| A_24_P3140 | NM_014497 | ZNF638 | 1.5235 | 0.043515787 |
| A_23_P135611 | NM_018114 | DALRD3 | 1.5248 | 0.017486635 |
| A_24_P266880 | NM_021009 | UBC | 1.526 | 0.042599114 |
| A_32_P91902 | A_32_P91902 | A_32_P91902 | 1.526 | 0.014147323 |
| A_23_P61057 | NM_004513 | IL16 | 1.5262 | 0.012368632 |
| A_32_P128097 | A_32_P128097 | A_32_P128097 | 1.5264 | 0.025058659 |
| A_23_P92899 | NM_031908 | C1QTNF2 | 1.5278 | 0.031048212 |
| A_24_P93111 | A_24_P93111 | A_24_P93111 | 1.5296 | 0.027772594 |
| A_23_P48897 | AF011794 | CCPG1 | 1.5299 | 0.011946536 |
| A_23_P501460 | NM_033489 | CDC2L1 | 1.5301 | 0.008338956 |
| A_23_P215787 | NM_012257 | HBP1 | 1.5304 | 0.011150735 |
| A_24_P87498 | NM_177441 | TMUB2 | 1.5309 | 0.028434016 |
| A_23_P86283 | NM_006762 | LAPTM5 | 1.531 | 0.004521776 |
| A_32_P36192 | NM_004640 | BAT1 | 1.5312 | 0.026186875 |
| A_23_P329727 | NM_015133 | MAPK8IP3 | 1.5317 | 0.031665598 |
| A_23_P204342 | NM_213594 | RFX4 | 1.5318 | 0.043750686 |
| A_23_P92410 | NM_004346 | CASP3 | 1.5318 | 0.025322199 |
| A_23_P155123 | NM_000106 | CYP2D6 | 1.5321 | 0.024657481 |
| A_23_P123916 | NM_138361 | LRSAM1 | 1.5325 | 0.049342718 |
| A_23_P205997 | NM_031301 | APH1B | 1.5328 | 0.0086324 |
| A_23_P113245 | D14041 | RBPJ | 1.5332 | 0.005605777 |
| A_23_P169189 | NM_003640 | IKBKAP | 1.534 | 0.038055469 |
| A_23_P397635 | NM_007131 | ZNF75 | 1.5343 | 0.023564473 |
| A_23_P82402 | NM_138426 | GLCCI1 | 1.5349 | 0.041407367 |
| A_23_P53891 | NM_001730 | KLF5 | 1.5358 | 0.012165928 |
| A_23_P215406 | NM_198829 | RAC1 | 1.5359 | 0.009635592 |
| A_32_P163125 | NM_147156 | SGMS1 | 1.536 | 0.029046404 |
| A_32_P35734 | NR_001443 | LOC339240 | 1.5372 | 0.025329727 |
| A_23_P138376 | NM_021252 | RAB18 | 1.5375 | 0.02226834 |
| A_23_P26511 | NM_024307 | GDPD3 | 1.5389 | 0.003229591 |
| A_23_P202067 | NM_012311 | KIN | 1.5392 | 0.039267135 |
| A_23_P166431 | NM_015372 | C22orf24 | 1.541 | 0.031613452 |
| A_23_P48897 | AF011794 | CCPG1 | 1.5412 | 0.01279472 |
| A_23_P11286 | NM_019597 | HNRNPH2 | 1.5421 | 0.027108021 |
| A_23_P164341 | NM_014232 | VAMP2 | 1.5428 | 0.041734704 |
| A_23_P122174 | NM_022550 | XRCC4 | 1.543 | 0.019741313 |
| A_23_P306105 | NM_020474 | GALNT1 | 1.5433 | 0.032370125 |
| A_23_P77073 | NM_032802 | SPPL2A | 1.5437 | 0.013442365 |
| A_23_P37399 | NM_014216 | ITPK1 | 1.5457 | 0.003991335 |
| A_24_P174563 | NM_025196 | GRPEL1 | 1.5458 | 0.049776549 |
| A_23_P143356 | AY007156 | AY007156 | 1.5461 | 0.006822196 |
| A_23_P41145 | NM_138805 | FAM3D | 1.5478 | 0.035095273 |
| A_24_P41021 | NM_203301 | FBXO33 | 1.548 | 0.007846376 |
| A_24_P144383 | A_24_P144383 | A_24_P144383 | 1.5489 | 0.024674739 |
| A_23_P131227 | NM_017735 | TTC27 | 1.549 | 0.018875873 |
| A_24_P142473 | NM_015343 | DULLARD | 1.5494 | 0.040152872 |
| A_32_P150632 | ENST00000372234 | ANXA11 | 1.5498 | 0.031037089 |
| A_23_P108835 | NM_016061 | YPEL5 | 1.5504 | 0.04667877 |
| A_24_P71244 | NM_005026 | PIK3CD | 1.5505 | 0.002224515 |
| A_23_P120048 | NM_013450 | BAZ2B | 1.551 | 0.002777009 |
| A_32_P161934 | THC2657931 | THC2657931 | 1.5536 | 0.029921722 |
| A_23_P104323 | NM_002412 | MGMT | 1.5539 | 0.031648942 |
| A_24_P119331 | AK054562 | AK054562 | 1.5542 | 0.029162422 |
| A_23_P66017 | NM_145239 | PRRT2 | 1.5559 | 0.020376226 |
| A_23_P251342 | NM_002093 | GSK3B | 1.5562 | 0.042192786 |
| A_23_P318604 | BC004544 | CYHR1 | 1.5565 | 0.01371172 |
| A_24_P362317 | NM_001111 | ADAR | 1.5565 | 0.006210199 |
| A_23_P92410 | NM_004346 | CASP3 | 1.5566 | 0.020850327 |
| A_23_P127824 | NM_001042780 | TNNT3 | 1.5573 | 0.02394841 |
| A_24_P8768 | NM_017971 | MRPL20 | 1.5581 | 0.039463184 |
| A_23_P40952 | NM_002880 | RAF1 | 1.5583 | 0.006531766 |
| A_23_P431310 | NM_170604 | RASGRP4 | 1.5584 | 0.029690199 |
| A_24_P322756 | NM_003307 | TRPM2 | 1.56 | 0.01565436 |
| A_23_P37441 | NM_004048 | B2M | 1.5603 | 0.008939838 |
| A_23_P156748 | NM_015245 | ANKS1A | 1.5609 | 0.023783747 |
| A_23_P93180 | NM_003526 | HIST1H2BC | 1.5614 | 0.015737784 |
| A_24_P943106 | NM_001080415 | SR140 | 1.5617 | 0.026220979 |
| A_24_P396702 | NM_014880 | CD302 | 1.5626 | 0.004062869 |
| A_23_P37441 | NM_004048 | B2M | 1.5645 | 0.012775175 |
| A_23_P118888 | NM_000430 | PAFAH1B1 | 1.5646 | 0.014652392 |
| A_23_P131149 | NM_002552 | ORC4L | 1.5651 | 0.046613373 |
| A_24_P481113 | AI971459 | AI971459 | 1.5652 | 0.031138188 |
| A_24_P244952 | NM_015092 | SMG1 | 1.5656 | 0.033950545 |
| A_23_P169197 | NM_032303 | HSDL2 | 1.5691 | 0.001870369 |
| A_23_P143047 | NM_080653 | ATP6V1E2 | 1.5695 | 0.039177776 |
| A_23_P126398 | NM_012432 | SETDB1 | 1.5707 | 0.017286575 |
| A_23_P150818 | AK093078 | MBD6 | 1.5716 | 0.000822298 |
| A_24_P940725 | AL080186 | SFRS18 | 1.5717 | 0.01242882 |
| A_23_P147826 | NM_021183 | RAP2C | 1.5724 | 0.031377489 |
| A_32_P107441 | XM_001726460 | LOC100130600 | 1.5733 | 0.001738538 |
| A_23_P20225 | NM_015713 | RRM2B | 1.574 | 0.004915772 |
| A_24_P139816 | NM_031901 | MRPS21 | 1.5749 | 0.031938696 |
| A_24_P117323 | NM_032775 | KLHL22 | 1.5755 | 0.025705938 |
| A_24_P910169 | A_24_P910169 | A_24_P910169 | 1.5764 | 0.006727314 |
| A_23_P63178 | NM_005644 | TAF12 | 1.5765 | 0.047058727 |
| A_24_P497226 | NM_003161 | RPS6KB1 | 1.5772 | 0.043072556 |
| A_24_P757154 | THC2661968 | THC2661968 | 1.5787 | 0.003383565 |
| A_23_P213141 | NM_015271 | TRIM2 | 1.5793 | 0.01386237 |
| A_23_P201619 | ENST00000367385 | NEK7 | 1.5796 | 0.012891903 |
| A_23_P252145 | NM_020156 | C1GALT1 | 1.5799 | 0.020916351 |
| A_23_P215406 | NM_198829 | RAC1 | 1.5812 | 0.008292111 |
| A_23_P255663 | NM_024641 | MANEA | 1.5813 | 0.020149147 |
| A_32_P221590 | THC2643233 | THC2643233 | 1.5819 | 0.016999837 |
| A_23_P67312 | NM_003437 | ZNF136 | 1.5819 | 0.043599833 |
| A_23_P157240 | J05272 | IMPDH1 | 1.5822 | 0.011731022 |
| A_23_P365119 | ENST00000324808 | ENST00000324808 | 1.5835 | 0.024345104 |
| A_23_P349083 | NM_138782 | FCHO2 | 1.5846 | 0.00108159 |
| A_23_P138137 | NM_145243 | OMA1 | 1.5848 | 0.018511665 |
| A_23_P96853 | NM_007051 | FAF1 | 1.5849 | 0.048378464 |
| A_23_P8297 | NM_198081 | SCML4 | 1.5856 | 0.015658533 |
| A_23_P309281 | NM_005543 | INSL3 | 1.5859 | 0.024287466 |
| A_24_P400473 | NM_014655 | SLC25A44 | 1.5866 | 0.007332643 |
| A_24_P3783 | NM_003521 | HIST1H2BM | 1.5868 | 0.002719309 |
| A_23_P63289 | NM_014188 | SSU72 | 1.5871 | 0.022276355 |
| A_24_P409681 | A_24_P409681 | A_24_P409681 | 1.5872 | 0.006480115 |
| A_24_P713668 | NM_014670 | BZW1 | 1.5886 | 0.022794936 |
| A_23_P418046 | AL137400 | AL137400 | 1.5895 | 0.021442899 |
| A_23_P37475 | NM_030800 | C15orf44 | 1.591 | 0.045518557 |
| A_23_P37311 | NM_014982 | PCNX | 1.591 | 0.002108574 |
| A_23_P215406 | NM_198829 | RAC1 | 1.5926 | 0.007840801 |
| A_23_P86182 | NM_031901 | MRPS21 | 1.5928 | 0.035888902 |
| A_23_P37441 | NM_004048 | B2M | 1.5928 | 0.00712169 |
| A_24_P272310 | NM_205853 | MUSTN1 | 1.5932 | 0.038217919 |
| A_32_P134090 | THC2719547 | THC2719547 | 1.5938 | 0.023169491 |
| A_32_P99097 | NM_002270 | TNPO1 | 1.5949 | 0.024092537 |
| A_32_P3127 | AF038199 | AF038199 | 1.5957 | 0.037596393 |
| A_23_P30799 | NM_021018 | HIST1H3F | 1.5961 | 0.006351076 |
| A_23_P332316 | NM_152285 | ARRDC1 | 1.5964 | 0.042630278 |
| A_23_P35591 | NM_016046 | EXOSC1 | 1.5971 | 0.013496663 |
| A_23_P109774 | NM_014415 | ZBTB11 | 1.5993 | 0.015152956 |
| A_23_P124837 | NM_002332 | LRP1 | 1.5997 | 0.029558421 |
| A_23_P166698 | NM_016305 | SS18L2 | 1.6007 | 0.039076833 |
| A_23_P161488 | NM_001033925 | TIAL1 | 1.6007 | 0.035080425 |
| A_23_P130149 | NM_001976 | ENO3 | 1.6019 | 0.02649298 |
| A_23_P54770 | NM_018690 | APOB48R | 1.6024 | 0.006624785 |
| A_23_P202427 | NM_025130 | HKDC1 | 1.6025 | 0.006623116 |
| A_23_P39237 | NM_003407 | ZFP36 | 1.6029 | 0.010997148 |
| A_23_P215675 | NM_018224 | C7orf44 | 1.6031 | 0.03210394 |
| A_24_P916853 | A_24_P916853 | A_24_P916853 | 1.6033 | 0.024236422 |
| A_23_P23960 | NM_001001342 | BLOC1S2 | 1.6036 | 0.029886805 |
| A_23_P73589 | NM_002444 | MSN | 1.6037 | 0.023458374 |
| A_23_P53530 | NM_001033050 | MTERFD3 | 1.6041 | 0.0167841 |
| A_23_P24375 | NM_017670 | OTUB1 | 1.6046 | 0.009394326 |
| A_23_P93898 | NM_020369 | FSCN3 | 1.6046 | 0.047532324 |
| A_23_P146990 | NM_007013 | WWP1 | 1.6056 | 0.047261202 |
| A_24_P323715 | XM_001717499 | LOC642076 | 1.6064 | 0.012100724 |
| A_24_P76740 | NM_032449 | CC2D1B | 1.6074 | 0.015758525 |
| A_23_P23234 | BC000988 | C1orf217 | 1.6086 | 0.020608677 |
| A_23_P393425 | NM_173797 | PAPD4 | 1.6087 | 0.008697172 |
| A_24_P913760 | NM_012141 | INTS6 | 1.609 | 0.021002494 |
| A_23_P140362 | NM_018228 | C14orf115 | 1.6095 | 0.039093429 |
| A_23_P92410 | NM_004346 | CASP3 | 1.6097 | 0.018722181 |
| A_23_P109895 | NM_001040454 | SLC26A6 | 1.6102 | 0.011899226 |
| A_23_P77401 | NM_018340 | FLJ11151 | 1.6105 | 0.005634889 |
| A_24_P796652 | U69195 | U69195 | 1.6119 | 0.006605083 |
| A_32_P95739 | NM_000365 | TPI1 | 1.6119 | 0.032243725 |
| A_24_P98975 | NM_033121 | ANKRD13A | 1.6123 | 0.019720844 |
| A_23_P80409 | NM_020839 | WDR48 | 1.6126 | 0.048336061 |
| A_24_P361167 | NM_020920 | CHD8 | 1.6131 | 0.012194326 |
| A_32_P75792 | NM_001014980 | FAM132A | 1.6134 | 0.048862935 |
| A_23_P24260 | NM_001776 | ENTPD1 | 1.6145 | 0.003624646 |
| A_23_P349203 | AK027172 | RNH1 | 1.6154 | 0.023673194 |
| A_23_P168951 | NM_014943 | ZHX2 | 1.6155 | 0.026516029 |
| A_23_P111041 | NM_003525 | HIST1H2BI | 1.6155 | 0.003513069 |
| A_24_P66522 | NM_014984 | AZI1 | 1.6163 | 0.010577704 |
| A_23_P120710 | NM_003316 | TTC3 | 1.6164 | 0.032430523 |
| A_24_P25470 | BC027922 | BC027922 | 1.6171 | 0.036607586 |
| A_23_P213908 | NM_032177 | RNUXA | 1.6175 | 0.047221724 |
| A_23_P92410 | NM_004346 | CASP3 | 1.6184 | 0.019186196 |
| A_32_P138004 | NM_207009 | FAM45A | 1.6192 | 0.012268192 |
| A_24_P148836 | NM_173546 | KLHDC8B | 1.6195 | 0.007131927 |
| A_23_P396353 | NM_020921 | NIN | 1.6198 | 0.009949222 |
| A_23_P16143 | NM_002096 | GTF2F1 | 1.6199 | 0.034384971 |
| A_23_P92410 | NM_004346 | CASP3 | 1.62 | 0.017751647 |
| A_24_P409891 | XR_018524 | XR_018524 | 1.6209 | 0.015313844 |
| A_24_P818005 | NM_177441 | TMUB2 | 1.621 | 0.003096871 |
| A_23_P416705 | NM_147129 | ALS2CL | 1.6211 | 0.031154043 |
| A_23_P215406 | NM_198829 | RAC1 | 1.6215 | 0.00675055 |
| A_32_P108420 | A_32_P108420 | A_32_P108420 | 1.6219 | 0.04376406 |
| A_23_P251342 | NM_002093 | GSK3B | 1.623 | 0.032514811 |
| A_24_P937582 | AK027610 | AK027610 | 1.6254 | 0.01360597 |
| A_23_P112825 | NM_016309 | LCMT1 | 1.626 | 0.027041053 |
| A_24_P143574 | NM_006065 | SIRPB1 | 1.6266 | 0.007055899 |
| A_23_P429689 | NM_148894 | FAM44A | 1.627 | 0.004598426 |
| A_23_P78444 | NM_018255 | ELP2 | 1.6271 | 0.021707298 |
| A_32_P92295 | NM_012423 | RPL13A | 1.6281 | 0.015417503 |
| A_24_P186174 | NM_014678 | SAPS2 | 1.6284 | 0.03850274 |
| A_24_P266880 | NM_021009 | UBC | 1.6289 | 0.027823601 |
| A_32_P104075 | NM_002577 | PAK2 | 1.6294 | 0.004790919 |
| A_23_P430800 | NM_001114938 | CCDC17 | 1.6297 | 0.004389476 |
| A_24_P345131 | ENST00000330947 | LRRC8B | 1.6301 | 0.030465515 |
| A_23_P48070 | NM_016162 | ING4 | 1.6307 | 0.038455503 |
| A_23_P94800 | NM_002961 | S100A4 | 1.6307 | 0.006938147 |
| A_32_P115451 | THC2651395 | THC2651395 | 1.6312 | 0.026865211 |
| A_23_P37441 | NM_004048 | B2M | 1.6315 | 0.006682618 |
| A_23_P52657 | NM_005609 | PYGM | 1.6315 | 0.049139851 |
| A_23_P70888 | NM_033506 | FBXO24 | 1.6316 | 0.030492058 |
| A_23_P48897 | AF011794 | CCPG1 | 1.633 | 0.009745348 |
| A_23_P48897 | AF011794 | CCPG1 | 1.6333 | 0.008900072 |
| A_23_P133245 | NM_006083 | IK | 1.6335 | 0.032296919 |
| A_23_P215227 | NM_005494 | DNAJB6 | 1.6339 | 0.027914442 |
| A_32_P30813 | NM_006601 | PTGES3 | 1.634 | 0.028625789 |
| A_23_P110445 | NM_006051 | APBB3 | 1.634 | 0.003806877 |
| A_32_P87809 | BC041636 | BC041636 | 1.6351 | 0.024498312 |
| A_23_P32320 | NM_024083 | ASPSCR1 | 1.6354 | 0.041972395 |
| A_23_P213486 | THC2504035 | THC2504035 | 1.6355 | 0.006925462 |
| A_32_P527371 | NM_001039948 | SGSM1 | 1.6358 | 0.036165075 |
| A_23_P61222 | AJ002425 | HSAJ2425 | 1.6358 | 0.006940436 |
| A_23_P309599 | AK026760 | PABPC1L | 1.637 | 0.012582069 |
| A_32_P36313 | NM_145206 | VTI1A | 1.6375 | 0.036400761 |
| A_23_P346206 | NM_001015885 | RAE1 | 1.638 | 0.042677687 |
| A_24_P251221 | NM_006243 | PPP2R5A | 1.6389 | 0.011640527 |
| A_24_P27977 | NM_003307 | TRPM2 | 1.6402 | 0.003623992 |
| A_23_P37441 | NM_004048 | B2M | 1.6411 | 0.006520631 |
| A_24_P195327 | XR_019253 | XR_019253 | 1.6415 | 0.029054198 |
| A_24_P290799 | NM_024419 | PGS1 | 1.6427 | 0.038640981 |
| A_24_P213144 | A_24_P213144 | A_24_P213144 | 1.6431 | 0.019823213 |
| A_23_P215109 | NM_080385 | CPA5 | 1.6437 | 0.049723729 |
| A_23_P337753 | NM_138402 | LOC93349 | 1.6442 | 0.046824645 |
| A_23_P37441 | NM_004048 | B2M | 1.6446 | 0.007068492 |
| A_23_P106362 | NM_020980 | AQP9 | 1.6481 | 0.000321533 |
| A_24_P382765 | NM_005008 | NHP2L1 | 1.6484 | 0.02659071 |
| A_32_P144421 | NM_053042 | ZNF518B | 1.6488 | 0.033216773 |
| A_23_P71433 | NM_001001481 | UBE2W | 1.6493 | 0.008629069 |
| A_24_P913115 | NM_000314 | PTEN | 1.6508 | 0.003094429 |
| A_23_P311632 | NM_138329 | NLRP6 | 1.6512 | 0.032118582 |
| A_23_P83277 | NM_004512 | IL11RA | 1.6519 | 0.00420962 |
| A_32_P100464 | THC2691500 | THC2691500 | 1.6522 | 0.042963366 |
| A_23_P251548 | NM_144582 | TEX261 | 1.6525 | 0.049842952 |
| A_23_P29036 | NM_005534 | IFNGR2 | 1.6528 | 0.009390021 |
| A_24_P390403 | NM_015138 | RTF1 | 1.653 | 0.029597706 |
| A_23_P212447 | NM_018262 | IFT122 | 1.655 | 0.031786323 |
| A_23_P311616 | NM_015167 | JMJD6 | 1.6551 | 0.015017025 |
| A_23_P202170 | NM_012215 | MGEA5 | 1.6553 | 0.007439858 |
| A_32_P63162 | THC2557053 | THC2557053 | 1.6555 | 0.005096555 |
| A_24_P919510 | AF107493 | RBM5 | 1.6558 | 0.039221417 |
| A_32_P153833 | AK098597 | AK098597 | 1.6559 | 0.035846878 |
| A_23_P70571 | NM_006979 | SLC39A7 | 1.6562 | 0.015775173 |
| A_23_P23421 | NM_198236 | ARHGEF11 | 1.6566 | 0.033266618 |
| A_23_P207927 | NM_017941 | C17orf80 | 1.657 | 0.017269248 |
| A_23_P40821 | NM_032383 | HPS3 | 1.659 | 0.039327278 |
| A_23_P155907 | NM_015143 | METAP1 | 1.6595 | 0.017854808 |
| A_32_P63729 | BX117044 | BX117044 | 1.66 | 0.047823379 |
| A_24_P171268 | NM_182663 | RASSF5 | 1.6604 | 0.016081382 |
| A_23_P325075 | NM_003800 | RNGTT | 1.6632 | 0.019198814 |
| A_24_P339416 | NM_014960 | ARSG | 1.6645 | 0.030402332 |
| A_24_P945147 | NM_004703 | RABEP1 | 1.6648 | 0.018430149 |
| A_23_P59069 | NM_003527 | HIST1H2BO | 1.6674 | 0.011305786 |
| A_32_P232851 | THC2645586 | THC2645586 | 1.6686 | 0.021545982 |
| A_23_P353726 | NM_153810 | C10orf46 | 1.6686 | 0.024025704 |
| A_23_P48897 | AF011794 | CCPG1 | 1.6716 | 0.00724153 |
| A_24_P111054 | BC035878 | SLC2A5 | 1.6718 | 0.018783519 |
| A_23_P96658 | ENST00000382832 | CYorf15B | 1.672 | 0.033845992 |
| A_24_P208081 | NM_022819 | PLA2G2F | 1.6729 | 0.049928338 |
| A_32_P113682 | AK025056 | AK025056 | 1.673 | 0.018239841 |
| A_23_P92410 | NM_004346 | CASP3 | 1.6734 | 0.018363983 |
| A_24_P13083 | NM_130783 | TSPAN18 | 1.6738 | 0.014096641 |
| A_23_P256231 | NM_032145 | FBXO30 | 1.674 | 0.009108553 |
| A_24_P556049 | A_24_P556049 | A_24_P556049 | 1.6745 | 0.045408262 |
| A_23_P429670 | NM_032440 | LCOR | 1.6746 | 0.029220564 |
| A_32_P148407 | A_32_P148407 | A_32_P148407 | 1.6747 | 0.029408778 |
| A_24_P165082 | NM_013360 | ZNF222 | 1.6755 | 0.027487537 |
| A_32_P58937 | NM_017643 | MBTD1 | 1.6762 | 0.034151743 |
| A_24_P348892 | NM_052849 | CCDC32 | 1.6765 | 0.047425226 |
| A_24_P201702 | NM_005127 | CLEC2B | 1.6766 | 0.002590308 |
| A_24_P225477 | NM_018281 | ECHDC2 | 1.6767 | 0.006696062 |
| A_23_P215406 | NM_198829 | RAC1 | 1.6771 | 0.006389039 |
| A_24_P277747 | NM_006586 | CNPY3 | 1.6785 | 0.011986213 |
| A_23_P170978 | NM_017865 | ZNF692 | 1.6787 | 0.01807041 |
| A_23_P356122 | NM_001031623 | ZNF451 | 1.6796 | 0.032747655 |
| A_23_P99253 | NM_004664 | LIN7A | 1.6803 | 0.001048841 |
| A_32_P7502 | NM_198196 | CD96 | 1.6808 | 0.0175441 |
| A_23_P217899 | NM_001039577 | CCNL2 | 1.681 | 0.013359233 |
| A_23_P146798 | NM_012248 | SEPHS2 | 1.6816 | 0.025560346 |
| A_32_P141948 | THC2564562 | THC2564562 | 1.6816 | 0.045295226 |
| A_23_P142974 | NM_001007231 | ARHGAP25 | 1.6816 | 0.004818907 |
| A_23_P38115 | NM_017748 | CCDC49 | 1.682 | 0.023466315 |
| A_23_P168324 | NM_014809 | KIAA0319 | 1.6821 | 0.009154452 |
| A_24_P301557 | NM_014646 | LPIN2 | 1.6822 | 0.01184063 |
| A_23_P89460 | NM_012138 | AATF | 1.6825 | 0.01424282 |
| A_24_P255303 | XR_018489 | LOC391160 | 1.683 | 0.00408922 |
| A_24_P406525 | NM_018371 | CSGALNACT1 | 1.683 | 0.005977639 |
| A_23_P163347 | NM_016166 | PIAS1 | 1.6831 | 0.006898461 |
| A_24_P301954 | AK001057 | MGC16384 | 1.6835 | 0.003613947 |
| A_23_P20648 | NM_003172 | SURF1 | 1.6844 | 0.010475234 |
| A_23_P118025 | NM_022355 | DPEP2 | 1.6844 | 0.001970743 |
| A_32_P86494 | A_32_P86494 | A_32_P86494 | 1.6848 | 0.00850066 |
| A_24_P934861 | A_24_P934861 | A_24_P934861 | 1.6849 | 0.019436579 |
| A_24_P15083 | A_24_P15083 | A_24_P15083 | 1.685 | 0.00664804 |
| A_23_P48897 | AF011794 | CCPG1 | 1.686 | 0.008299614 |
| A_23_P207400 | NM_007295 | BRCA1 | 1.6863 | 0.00788412 |
| A_23_P104054 | NM_016227 | C1orf9 | 1.6866 | 0.016225366 |
| A_23_P170399 | NM_021942 | C4orf41 | 1.6873 | 0.035934373 |
| A_24_P419120 | NM_015250 | BICD2 | 1.6876 | 0.024214917 |
| A_24_P544882 | NM_003144 | SSR1 | 1.6882 | 0.02148697 |
| A_23_P159071 | NM_024515 | WDR25 | 1.6884 | 0.02702457 |
| A_23_P59349 | NM_016217 | HECA | 1.6888 | 0.019096465 |
| A_23_P37441 | NM_004048 | B2M | 1.6891 | 0.008693986 |
| A_23_P167789 | NM_016605 | FAM53C | 1.6893 | 0.002039462 |
| A_23_P65129 | NM_032840 | SPRYD3 | 1.6909 | 0.008594213 |
| A_32_P167122 | NM_018254 | RCOR3 | 1.6915 | 0.01262458 |
| A_24_P281443 | A_24_P281443 | A_24_P281443 | 1.6924 | 0.030732051 |
| A_32_P63013 | AF086052 | AF086052 | 1.6938 | 0.00036073 |
| A_23_P251342 | NM_002093 | GSK3B | 1.6946 | 0.024089443 |
| A_23_P66900 | NM_176096 | CDK5RAP3 | 1.6951 | 0.008919807 |
| A_23_P201047 | NM_007112 | THBS3 | 1.6959 | 0.015890676 |
| A_23_P8013 | NM_003519 | HIST1H2BL | 1.6973 | 0.015220765 |
| A_32_P58999 | XR_015267 | LOC728003 | 1.6982 | 0.008743495 |
| A_23_P88234 | NM_016049 | C14orf122 | 1.6989 | 0.049945958 |
| A_23_P324873 | NR_000011 | SNORA70 | 1.6992 | 0.00950336 |
| A_23_P2032 | A_23_P2032 | A_23_P2032 | 1.6994 | 0.024808576 |
| A_23_P65823 | AF180519 | GABARAPL3 | 1.7003 | 0.007447596 |
| A_32_P200600 | THC2591088 | THC2591088 | 1.7008 | 0.022382122 |
| A_23_P219045 | NM_003530 | HIST1H3D | 1.7017 | 0.001685553 |
| A_23_P99632 | NM_017999 | RNF31 | 1.7036 | 0.007265721 |
| A_23_P125829 | NM_000291 | PGK1 | 1.704 | 0.046177025 |
| A_23_P77669 | NM_017530 | ZNF821 | 1.7042 | 0.024066133 |
| A_23_P120414 | NM_003404 | YWHAB | 1.7054 | 0.010606671 |
| A_24_P727868 | XR_015267 | LOC728003 | 1.7068 | 0.003123728 |
| A_24_P17870 | NM_006674 | HCP5 | 1.7078 | 0.004792213 |
| A_23_P5435 | AY358993 | KRTCAP3 | 1.708 | 0.026399845 |
| A_23_P28797 | THC2525955 | THC2525955 | 1.7095 | 0.013348389 |
| A_23_P166159 | NM_030815 | PDRG1 | 1.7096 | 0.024923443 |
| A_32_P169574 | NM_017602 | OTUD5 | 1.7098 | 0.030814104 |
| A_24_P99838 | NM_013361 | ZNF223 | 1.7098 | 0.045995621 |
| A_23_P332992 | NM_175055 | HIST3H2BB | 1.7106 | 0.007994474 |
| A_32_P141257 | ENST00000382909 | ENST00000382909 | 1.7109 | 0.045594418 |
| A_23_P91350 | NM_019593 | RP5-1022P6.2 | 1.7114 | 0.008552396 |
| A_24_P942694 | NM_018017 | C10orf118 | 1.7121 | 0.020243392 |
| A_32_P66148 | AI359141 | AI359141 | 1.714 | 0.014350454 |
| A_24_P124325 | CR749800 | IKZF5 | 1.7153 | 0.007637766 |
| A_23_P79836 | NM_006811 | SERINC3 | 1.7161 | 0.026136893 |
| A_32_P130217 | A_32_P130217 | A_32_P130217 | 1.7162 | 0.049506801 |
| A_24_P465799 | AF063695 | AF063695 | 1.7167 | 0.04717475 |
| A_23_P368278 | NM_014824 | FCHSD2 | 1.7177 | 0.040701566 |
| A_23_P259207 | NM_018271 | THNSL2 | 1.7201 | 0.02053448 |
| A_24_P584463 | XR_018311 | XR_018311 | 1.7217 | 0.008703132 |
| A_23_P5392 | NM_004881 | TP53I3 | 1.7241 | 0.004832646 |
| A_23_P250735 | NM_175709 | CBX7 | 1.7249 | 0.009165172 |
| A_23_P105184 | NM_001040118 | CENTD2 | 1.7256 | 0.001760445 |
| A_23_P215406 | NM_198829 | RAC1 | 1.7271 | 0.004776521 |
| A_23_P26777 | NM_024844 | NUP85 | 1.7279 | 0.031172084 |
| A_23_P206759 | NM_014761 | KIAA0174 | 1.7279 | 0.022013092 |
| A_24_P852082 | AK125829 | LOC100130175 | 1.7286 | 0.018885741 |
| A_23_P88163 | NM_018036 | ATG2B | 1.7288 | 0.02799864 |
| A_24_P600603 | A_24_P600603 | A_24_P600603 | 1.729 | 0.029911455 |
| A_23_P103885 | NM_017900 | AURKAIP1 | 1.7299 | 0.00778736 |
| A_23_P69242 | NM_182760 | SUMF1 | 1.7306 | 0.007460093 |
| A_23_P215406 | NM_198829 | RAC1 | 1.7314 | 0.004385328 |
| A_24_P327011 | NM_021823 | PPCDC | 1.7317 | 0.043903449 |
| A_23_P313734 | NM_033395 | KIAA1731 | 1.732 | 0.045391054 |
| A_23_P59988 | NM_054028 | AMAC1L2 | 1.7335 | 0.042487956 |
| A_24_P295379 | NM_020960 | GPR107 | 1.7345 | 0.010990628 |
| A_23_P132654 | NM_017442 | TLR9 | 1.7347 | 0.049273854 |
| A_23_P71752 | NM_006007 | ZFAND5 | 1.7359 | 0.015390152 |
| A_23_P323749 | NM_025049 | PIF1 | 1.7364 | 0.028035993 |
| A_32_P114235 | THC2505819 | THC2505819 | 1.7382 | 0.030829725 |
| A_23_P158007 | NM_032799 | ZDHHC12 | 1.7406 | 0.011424708 |
| A_23_P23266 | ENST00000367808 | BLZF1 | 1.7412 | 0.039624141 |
| A_24_P52801 | NM_017670 | OTUB1 | 1.7426 | 0.041912521 |
| A_32_P223140 | NM_145313 | RASGEF1A | 1.7443 | 0.016064965 |
| A_24_P153399 | NM_015705 | SGSM3 | 1.7444 | 0.014476127 |
| A_23_P332509 | NM_015909 | NAG | 1.7455 | 0.017103092 |
| A_32_P216734 | NM_001004351 | tcag7.1017 | 1.7458 | 0.045575345 |
| A_23_P362637 | NM_015255 | UBR2 | 1.7479 | 0.010975352 |
| A_24_P942469 | AK129652 | SPAG9 | 1.7488 | 0.003404313 |
| A_24_P229756 | XM_001726996 | LOC644950 | 1.7495 | 0.003095762 |
| A_23_P37441 | NM_004048 | B2M | 1.7504 | 0.004910731 |
| A_24_P336728 | NM_014873 | LPGAT1 | 1.751 | 0.012957481 |
| A_23_P37441 | NM_004048 | B2M | 1.7515 | 0.006420381 |
| A_23_P139143 | NM_004177 | STX3 | 1.7525 | 0.00167876 |
| A_24_P305678 | NM_012399 | PITPNB | 1.7542 | 0.032081451 |
| A_23_P46618 | NM_025179 | PLXNA2 | 1.7544 | 0.032391162 |
| A_24_P13475 | ENST00000382108 | SPATA13 | 1.7545 | 0.005770464 |
| A_32_P32835 | NM_145274 | TMEM99 | 1.7554 | 0.01020279 |
| A_32_P130265 | NM_014827 | ZC3H11A | 1.7558 | 0.017543863 |
| A_24_P341504 | NM_017619 | RNPC3 | 1.7574 | 0.015414014 |
| A_24_P141522 | NM_016320 | NUP98 | 1.7584 | 0.004923996 |
| A_23_P376735 | NM_153219 | ZNF524 | 1.759 | 0.035852069 |
| A_32_P192984 | AK128396 | AK128396 | 1.7597 | 0.047120558 |
| A_24_P609323 | AF098968 | AF098968 | 1.7617 | 0.028302421 |
| A_23_P48897 | AF011794 | CCPG1 | 1.762 | 0.005883486 |
| A_23_P102000 | NM_001008540 | CXCR4 | 1.7631 | 0.004271932 |
| A_23_P108376 | NM_022065 | THADA | 1.7632 | 0.026370318 |
| A_24_P357791 | NP1247838 | NP1247838 | 1.7643 | 0.038635934 |
| A_23_P124044 | NM_021008 | DEAF1 | 1.7655 | 0.040498118 |
| A_23_P61230 | NM_004712 | HGS | 1.7662 | 0.011225268 |
| A_23_P133585 | NM_001799 | CDK7 | 1.7681 | 0.034951348 |
| A_24_P578641 | A_24_P578641 | A_24_P578641 | 1.7707 | 0.038459394 |
| A_23_P28201 | AF119861 | AF119861 | 1.7723 | 0.036662969 |
| A_23_P205855 | AK000336 | AK000336 | 1.7726 | 0.015356923 |
| A_23_P372771 | NM_020447 | C15orf17 | 1.7732 | 0.021769113 |
| A_24_P186370 | NM_002444 | MSN | 1.7735 | 0.014402541 |
| A_23_P125829 | NM_000291 | PGK1 | 1.7757 | 0.031773802 |
| A_23_P201079 | NM_012231 | PRDM2 | 1.7774 | 0.007111174 |
| A_23_P169934 | NM_178314 | RILPL1 | 1.7784 | 0.007174125 |
| A_23_P204427 | NM_002710 | PPP1CC | 1.7787 | 0.043823168 |
| A_23_P31376 | NM_018334 | LRRN3 | 1.7803 | 1.87E−07 |
| A_23_P38959 | NM_005428 | VAV1 | 1.7807 | 0.001115949 |
| A_32_P6452 | A_32_P6452 | A_32_P6452 | 1.7815 | 0.017886078 |
| A_23_P254179 | NM_015339 | ADNP | 1.7827 | 0.04391773 |
| A_32_P220472 | NM_019006 | ZFAND6 | 1.7831 | 0.006996736 |
| A_23_P337550 | NM_017602 | OTUD5 | 1.7836 | 0.020766474 |
| A_24_P226278 | NM_015288 | PHF15 | 1.7869 | 0.008419241 |
| A_32_P69399 | BQ072652 | BQ072652 | 1.787 | 0.028958064 |
| A_24_P338757 | NM_005800 | USPL1 | 1.7884 | 0.015799735 |
| A_23_P74993 | AK096104 | AK096104 | 1.7886 | 0.040801306 |
| A_23_P104116 | NM_005105 | RBM8A | 1.7905 | 0.041971449 |
| A_24_P74329 | NM_001076678 | ZNF493 | 1.7914 | 0.047218743 |
| A_23_P25835 | NM_002028 | FNTB | 1.7918 | 0.003913382 |
| A_24_P36285 | NM_013398 | ZNF224 | 1.7919 | 0.033718929 |
| A_24_P598999 | BF932142 | BF932142 | 1.7929 | 0.017910391 |
| A_23_P209678 | NM_002664 | PLEK | 1.7936 | 0.020736297 |
| A_23_P92292 | NM_022457 | RFWD2 | 1.7939 | 0.015051798 |
| A_23_P58763 | NM_015946 | PELO | 1.7939 | 0.006702515 |
| A_32_P179351 | BX095281 | BX095281 | 1.7939 | 0.035818713 |
| A_32_P87769 | NM_203301 | FBXO33 | 1.7957 | 0.005462067 |
| A_23_P60387 | NM_017617 | NOTCH1 | 1.7959 | 0.001992863 |
| A_23_P202565 | NM_007373 | SHOC2 | 1.797 | 0.007822925 |
| A_32_P21646 | AK093641 | FAM120AOS | 1.797 | 0.046499252 |
| A_23_P218841 | NM_018297 | NGLY1 | 1.7983 | 0.046700144 |
| A_24_P944049 | NM_015147 | CEP68 | 1.7993 | 0.016181228 |
| A_23_P145247 | NM_001023560 | ZNF187 | 1.7999 | 0.045661318 |
| A_23_P111843 | NM_006349 | ZNHIT1 | 1.8006 | 0.040404694 |
| A_23_P37569 | NM_006122 | MAN2A2 | 1.8009 | 0.001926652 |
| A_23_P136964 | NM_000328 | RPGR | 1.8023 | 0.002351674 |
| A_24_P850336 | A_24_P850336 | A_24_P850336 | 1.8032 | 0.017773138 |
| A_23_P101237 | NM_031446 | C18orf21 | 1.8033 | 0.024975425 |
| A_23_P126349 | NM_020063 | BARHL2 | 1.8034 | 0.00793703 |
| A_23_P210619 | NM_016407 | C20orf43 | 1.8034 | 0.008284772 |
| A_23_P253317 | NM_013308 | GPR171 | 1.8037 | 0.000510803 |
| A_23_P216017 | NM_004901 | ENTPD4 | 1.8043 | 0.040637337 |
| A_24_P266880 | NM_021009 | UBC | 1.8059 | 0.01328374 |
| A_24_P95198 | NM_013318 | KIAA0515 | 1.8063 | 0.017106957 |
| A_23_P47034 | NM_002729 | HHEX | 1.8079 | 0.006211649 |
| A_23_P23705 | BC040655 | BC040655 | 1.8094 | 0.017687436 |
| A_32_P129530 | NM_018341 | C6orf70 | 1.8097 | 0.018966609 |
| A_24_P7510 | A_24_P7510 | A_24_P7510 | 1.8114 | 0.04389939 |
| A_24_P266880 | NM_021009 | UBC | 1.8122 | 0.010084838 |
| A_32_P85500 | NM_006674 | HCP5 | 1.8136 | 0.009108234 |
| A_24_P152398 | AB045831 | P53AIP1 | 1.8138 | 0.042399568 |
| A_23_P68472 | NM_003859 | DPM1 | 1.814 | 0.017778389 |
| A_23_P112798 | NM_001312 | CRIP2 | 1.8144 | 0.01102741 |
| A_24_P49383 | NM_024684 | C11orf67 | 1.8146 | 0.047408156 |
| A_23_P204847 | NM_002298 | LCP1 | 1.8146 | 0.004225215 |
| A_23_P26629 | NM_013258 | PYCARD | 1.8149 | 0.007710432 |
| A_24_P177634 | A_24_P177634 | A_24_P177634 | 1.815 | 0.049474349 |
| A_23_P366682 | NM_033513 | C19orf20 | 1.816 | 0.034492665 |
| A_24_P916816 | L79989 | SP100 | 1.8165 | 0.007593691 |
| A_32_P230828 | NR_002578 | GAS5 | 1.8165 | 0.001210563 |
| A_23_P151018 | NM_014319 | LEMD3 | 1.8168 | 0.010869557 |
| A_23_P102508 | NM_021095 | SLC5A6 | 1.8172 | 0.026627474 |
| A_23_P350187 | AB046766 | TET2 | 1.8173 | 0.003670613 |
| A_23_P96008 | NM_006785 | MALT1 | 1.8188 | 0.018613592 |
| A_23_P125829 | NM_000291 | PGK1 | 1.8195 | 0.038281749 |
| A_24_P28657 | NM_015446 | AHCTF1 | 1.8196 | 0.011445025 |
| A_23_P376488 | NM_000594 | TNF | 1.8196 | 0.01123304 |
| A_23_P169278 | NM_015239 | AGTPBP1 | 1.8213 | 0.003671977 |
| A_23_P109224 | THC2655417 | THC2655417 | 1.8217 | 0.008590945 |
| A_24_P201879 | NM_021645 | UTP14C | 1.8218 | 0.048415063 |
| A_24_P88721 | NM_013412 | RABL2A | 1.8228 | 0.004482721 |
| A_23_P26954 | NM_006373 | VAT1 | 1.8236 | 0.018042883 |
| A_24_P921232 | BC017848 | PEX14 | 1.8246 | 0.041544432 |
| A_23_P207400 | NM_007295 | BRCA1 | 1.8264 | 0.006488921 |
| A_23_P110846 | NM_004779 | CNOT8 | 1.8265 | 0.016449699 |
| A_23_P211598 | NM_002676 | PMM1 | 1.8281 | 0.032519454 |
| A_23_P15375 | NM_024585 | ARMC7 | 1.8292 | 0.009162602 |
| A_23_P87603 | NM_134323 | TARBP2 | 1.8296 | 0.039735804 |
| A_24_P134319 | ENST00000349014 | ADNP | 1.83 | 0.017128372 |
| A_23_P112950 | NM_018179 | ATF7IP | 1.8306 | 0.023751066 |
| (+)E1A_r60_a104 | E1A_r60_a104 | E1A_r60_a104 | 1.8307 | 0.033838653 |
| A_23_P133438 | NM_019018 | FAM105A | 1.8313 | 0.028416102 |
| A_23_P159172 | NM_001080395 | AATK | 1.8319 | 0.000517945 |
| A_23_P132669 | NM_001010983 | GLT8D1 | 1.8325 | 0.016225486 |
| A_23_P156697 | NM_021160 | BAT5 | 1.8336 | 0.016836848 |
| A_23_P12053 | NM_015001 | SPEN | 1.834 | 0.006401925 |
| A_32_P795813 | BC037918 | BC037918 | 1.8342 | 0.038290052 |
| A_32_P45009 | NM_005896 | IDH1 | 1.8352 | 0.023203631 |
| A_23_P164141 | NM_176863 | PSME3 | 1.8357 | 0.029682825 |
| A_23_P62270 | NM_004187 | JARID1C | 1.8369 | 0.034361674 |
| A_24_P273157 | NM_052843 | OBSCN | 1.8378 | 0.037224272 |
| A_23_P422178 | NM_003588 | CUL4B | 1.8379 | 0.02733174 |
| A_23_P209356 | NM_014946 | SPAST | 1.8381 | 0.038565549 |
| A_24_P197284 | NM_173821 | FLJ33590 | 1.8389 | 0.040082918 |
| A_23_P111712 | NM_006980 | MTERF | 1.8405 | 0.01648927 |
| A_23_P43504 | NM_001606 | ABCA2 | 1.8407 | 0.017759343 |
| A_23_P15073 | NM_001005920 | LOC339123 | 1.8411 | 0.015420694 |
| A_23_P424080 | NM_032312 | YIPF4 | 1.8412 | 0.012627335 |
| A_32_P12454 | BC041686 | BC041686 | 1.8412 | 0.031496553 |
| A_24_P124567 | NM_014182 | ORMDL2 | 1.8414 | 0.028878222 |
| A_23_P385206 | NM_177424 | STX12 | 1.8417 | 0.03295138 |
| A_23_P48897 | AF011794 | CCPG1 | 1.8417 | 0.004589907 |
| A_23_P218517 | NM_013360 | ZNF222 | 1.8418 | 0.013499357 |
| A_23_P30995 | NM_016230 | CYB5R4 | 1.8424 | 0.005391978 |
| A_24_P681301 | NM_021009 | UBC | 1.8426 | 0.004971775 |
| A_23_P24948 | NM_005472 | KCNE3 | 1.8426 | 0.000727408 |
| A_23_P102000 | NM_001008540 | CXCR4 | 1.8429 | 0.003266152 |
| A_23_P102000 | NM_001008540 | CXCR4 | 1.8431 | 0.003312136 |
| A_24_P75920 | NM_001080503 | LOC126075 | 1.8435 | 0.041607266 |
| A_24_P339157 | NM_014761 | KIAA0174 | 1.8441 | 0.023630918 |
| A_23_P8185 | NM_006519 | DYNLT1 | 1.8445 | 0.00567342 |
| A_23_P201816 | NM_014810 | CEP350 | 1.8483 | 0.023983391 |
| A_23_P24104 | NM_002658 | PLAU | 1.8487 | 0.005647777 |
| A_24_P244410 | NM_014042 | C11orf51 | 1.8492 | 0.017094636 |
| A_23_P84084 | NM_001495 | GFRA2 | 1.85 | 0.036708399 |
| A_32_P512061 | NR_002188 | GBAP | 1.8503 | 0.00183684 |
| A_23_P102000 | NM_001008540 | CXCR4 | 1.8506 | 0.00374228 |
| A_23_P257726 | NM_016481 | C9orf156 | 1.8514 | 0.012468463 |
| A_24_P57170 | NM_001039887 | C19orf55 | 1.8515 | 0.010655552 |
| A_23_P5761 | NM_006164 | NFE2L2 | 1.8522 | 0.01488471 |
| A_24_P196351 | AB208934 | PLXNC1 | 1.8528 | 0.000198597 |
| A_23_P102000 | NM_001008540 | CXCR4 | 1.8533 | 0.002736102 |
| A_23_P250283 | NM_004161 | RAB1A | 1.8559 | 0.025341586 |
| A_23_P36183 | NM_005316 | GTF2H1 | 1.8563 | 0.011639758 |
| A_24_P253713 | AK022252 | AK022252 | 1.8587 | 0.025447286 |
| A_32_P116881 | NM_053275 | RPLP0 | 1.859 | 0.003352786 |
| A_23_P84836 | NM_006310 | NPEPPS | 1.86 | 0.005570462 |
| A_23_P61551 | NM_006110 | CD2BP2 | 1.8619 | 0.023956464 |
| A_24_P134683 | NM_005745 | BCAP31 | 1.8626 | 0.009022668 |
| A_32_P11723 | ENST00000249480 | ENST00000249480 | 1.8631 | 0.002971396 |
| A_23_P353704 | NM_019593 | RP5-1022P6.2 | 1.864 | 0.003674501 |
| A_23_P139509 | NM_016570 | ERGIC2 | 1.8668 | 0.012653756 |
| A_24_P91035 | NM_005324 | H3F3B | 1.867 | 0.002586124 |
| A_23_P333498 | NM_030636 | EEPD1 | 1.8685 | 0.003732959 |
| A_24_P122862 | NM_017657 | AFTPH | 1.8692 | 0.017412351 |
| A_23_P20777 | NM_033117 | RBM18 | 1.8696 | 0.022948249 |
| A_23_P251342 | NM_002093 | GSK3B | 1.8704 | 0.014669993 |
| A_23_P159927 | NM_001666 | ARHGAP4 | 1.8713 | 0.021579082 |
| A_23_P29684 | NM_015873 | VILL | 1.8727 | 0.01258059 |
| A_24_P49775 | NM_001076678 | ZNF493 | 1.8732 | 0.04308382 |
| A_23_P128773 | NM_203356 | CTAGE5 | 1.8736 | 0.022349764 |
| A_23_P132294 | NM_001001560 | GGA1 | 1.8754 | 0.010752566 |
| A_23_P207736 | AK023077 | AK023077 | 1.8765 | 0.024017172 |
| A_23_P69188 | NM_206831 | DPH3 | 1.8767 | 0.005093234 |
| A_23_P139509 | NM_016570 | ERGIC2 | 1.8769 | 0.015145215 |
| A_23_P55242 | NM_032595 | PPP1R9B | 1.8773 | 0.046194192 |
| A_23_P98483 | ENST00000250524 | ENST00000250524 | 1.8774 | 0.003751657 |
| A_24_P904484 | AK123849 | LOC283174 | 1.8775 | 9.67E−05 |
| A_23_P18276 | NM_005778 | RBM5 | 1.8785 | 0.010907078 |
| A_23_P125829 | NM_000291 | PGK1 | 1.8793 | 0.031969959 |
| A_24_P736617 | A_24_P736617 | A_24_P736617 | 1.8798 | 0.003413684 |
| A_24_P373286 | NM_014711 | CP110 | 1.88 | 0.01860048 |
| A_32_P86400 | NM_198273 | LYSMD3 | 1.881 | 0.006708375 |
| A_32_P147324 | BC038355 | BC038355 | 1.8812 | 0.035519149 |
| A_23_P102000 | NM_001008540 | CXCR4 | 1.882 | 0.002639299 |
| A_23_P345081 | NM_001083956 | ZNF655 | 1.882 | 0.008319508 |
| A_24_P140827 | NM_005389 | PCMT1 | 1.8823 | 0.033910931 |
| A_23_P102000 | NM_001008540 | CXCR4 | 1.8835 | 0.003340305 |
| A_23_P98953 | NM_145648 | SLC15A4 | 1.884 | 0.008550275 |
| A_23_P427217 | NM_032776 | JMJD1C | 1.8846 | 0.002606957 |
| A_23_P210048 | NM_006037 | HDAC4 | 1.8851 | 0.007436007 |
| A_23_P22263 | NM_079837 | BANP | 1.8866 | 0.006246685 |
| A_23_P160729 | NM_006594 | AP4B1 | 1.8867 | 0.022797921 |
| A_24_P740705 | NM_194326 | RPS19BP1 | 1.887 | 0.004918064 |
| A_23_P139558 | NM_022771 | TBC1D15 | 1.8884 | 0.007996397 |
| A_23_P95823 | NM_145080 | NSMCE1 | 1.8896 | 0.016387554 |
| A_23_P125829 | NM_000291 | PGK1 | 1.8898 | 0.028538735 |
| A_23_P150876 | NM_024667 | VPS37B | 1.8899 | 0.003055359 |
| A_23_P70371 | NM_015153 | PHF3 | 1.8909 | 0.024031809 |
| A_23_P1387 | NM_032900 | ARHGAP19 | 1.8922 | 0.005907538 |
| A_24_P608330 | AK095242 | CTNNB1 | 1.8935 | 0.023812266 |
| A_24_P205230 | NM_001004333 | RNASEK | 1.8946 | 0.043888014 |
| A_24_P630039 | CR600369 | CR600369 | 1.8951 | 0.006977701 |
| A_23_P366312 | NM_138473 | SP1 | 1.897 | 0.02076222 |
| A_24_P43588 | NM_001003690 | MAD2L1BP | 1.9008 | 0.017131515 |
| A_23_P8055 | NM_007243 | NRM | 1.9008 | 0.030415685 |
| A_23_P48771 | NM_024952 | C14orf159 | 1.9018 | 0.001028941 |
| A_32_P144920 | NM_015509 | NECAP1 | 1.9021 | 0.045750826 |
| A_24_P406480 | NM_152271 | LONRF1 | 1.9029 | 0.04693611 |
| A_23_P78092 | NM_001003927 | EVI2A | 1.9035 | 0.00080044 |
| A_23_P31747 | NM_032847 | C8orf76 | 1.906 | 0.028041889 |
| A_24_P236799 | NM_006868 | RAB31 | 1.9065 | 0.002449804 |
| A_24_P109351 | NM_001009814 | KIAA0564 | 1.9075 | 0.046050784 |
| A_23_P102622 | NM_003833 | MATN4 | 1.9078 | 0.013017546 |
| A_23_P66767 | NM_153338 | GGT6 | 1.9094 | 0.017584505 |
| A_24_P115762 | NM_148170 | CTSC | 1.9095 | 0.015292698 |
| A_23_P48166 | NM_002822 | TWF1 | 1.9099 | 0.009301318 |
| A_23_P204702 | NM_003217 | TEGT | 1.9107 | 0.011978625 |
| A_23_P207400 | NM_007295 | BRCA1 | 1.913 | 0.004235409 |
| A_23_P125829 | NM_000291 | PGK1 | 1.9167 | 0.023777523 |
| A_23_P388900 | NM_018420 | SLC22A15 | 1.9188 | 0.001163992 |
| A_23_P203137 | NM_004788 | UBE4A | 1.9195 | 0.011981961 |
| A_23_P130293 | NM_015208 | ANKRD12 | 1.9203 | 0.034884518 |
| A_23_P140830 | NM_024712 | ELMO3 | 1.9208 | 0.0174762 |
| A_23_P60387 | NM_017617 | NOTCH1 | 1.9209 | 0.001046465 |
| A_23_P124358 | X75962 | X75962 | 1.9219 | 0.026385703 |
| A_23_P314191 | NM_015336 | ZDHHC17 | 1.9224 | 0.003521939 |
| A_23_P97221 | NM_015911 | ZNF691 | 1.9237 | 0.030542051 |
| A_23_P102364 | NM_019850 | NGEF | 1.9243 | 0.020727675 |
| A_23_P255503 | NM_002572 | PAFAH1B2 | 1.9246 | 0.037081484 |
| A_24_P614362 | NR_002806 | FAM13A1OS | 1.9253 | 0.001451212 |
| A_23_P115137 | NM_006402 | HBXIP | 1.928 | 0.010610541 |
| A_23_P50241 | NM_001294 | CLPTM1 | 1.9286 | 0.04156251 |
| A_23_P138139 | NM_145243 | OMA1 | 1.9291 | 0.019255803 |
| A_24_P108005 | NM_022818 | MAP1LC3B | 1.9293 | 0.006556775 |
| A_32_P3556 | NM_018206 | VPS35 | 1.9308 | 0.010491959 |
| A_24_P383199 | A_24_P383199 | A_24_P383199 | 1.9353 | 0.040621359 |
| A_24_P618928 | CR602022 | CR602022 | 1.9369 | 0.011292601 |
| A_23_P254648 | NM_012164 | FBXW2 | 1.9374 | 0.026894108 |
| A_24_P332902 | XR_018818 | XR_018818 | 1.9375 | 0.00533084 |
| A_24_P60217 | AK055730 | SLC23A3 | 1.9378 | 0.043518111 |
| A_23_P250800 | NM_006100 | ST3GAL6 | 1.938 | 0.004394511 |
| A_24_P125273 | NM_001661 | ARL4D | 1.9388 | 0.039385481 |
| A_24_P124831 | NM_002028 | FNTB | 1.939 | 0.011320319 |
| A_23_P218317 | NM_001038618 | NARF | 1.9395 | 0.001396518 |
| A_23_P102000 | NM_001008540 | CXCR4 | 1.9397 | 0.002787532 |
| A_32_P179746 | XM_001716343 | LOC100133781 | 1.9403 | 0.001158408 |
| A_23_P207400 | NM_007295 | BRCA1 | 1.9421 | 0.003160092 |
| A_23_P103454 | NM_032264 | NBPF3 | 1.9426 | 0.001357895 |
| A_32_P128391 | AL162073 | AL162073 | 1.9444 | 0.043125805 |
| A_23_P98930 | NM_018169 | C12orf35 | 1.9447 | 0.000307008 |
| A_24_P260440 | NM_002270 | TNPO1 | 1.9447 | 0.012586423 |
| A_24_P361457 | NM_173627 | FLJ35220 | 1.9459 | 0.04802068 |
| A_23_P322 | NM_182690 | EFNA4 | 1.9469 | 0.040758836 |
| A_24_P241330 | NM_024824 | ZC3H14 | 1.9472 | 0.038741419 |
| A_24_P381164 | BC041893 | TTC17 | 1.9477 | 0.019001223 |
| A_23_P133582 | NM_004730 | ETF1 | 1.9485 | 0.003640656 |
| A_23_P88849 | NM_004165 | RRAD | 1.9485 | 0.006748696 |
| A_23_P28169 | NM_152522 | ARL6IP6 | 1.9495 | 0.00998207 |
| A_23_P366312 | NM_138473 | SP1 | 1.9498 | 0.020149622 |
| A_23_P139509 | NM_016570 | ERGIC2 | 1.9508 | 0.011511234 |
| A_24_P307466 | A_24_P307466 | A_24_P307466 | 1.9511 | 0.00996016 |
| A_23_P435697 | NM_015608 | C10orf137 | 1.9521 | 0.035600979 |
| A_23_P218774 | NM_002872 | RAC2 | 1.9527 | 0.013584149 |
| A_23_P47116 | NM_003475 | RASSF7 | 1.9528 | 0.02727358 |
| A_23_P9056 | NM_014781 | RB1CC1 | 1.9558 | 0.003035944 |
| A_23_P250800 | NM_006100 | ST3GAL6 | 1.9567 | 0.003695244 |
| A_23_P354864 | NM_001031737 | CCDC78 | 1.9572 | 0.020280462 |
| A_32_P12639 | NM_002816 | PSMD12 | 1.9578 | 0.026249375 |
| A_24_P261052 | NM_015458 | MTMR9 | 1.9578 | 0.02697607 |
| A_32_P187245 | AA669846 | AA669846 | 1.9584 | 0.041508649 |
| A_23_P87742 | NM_001039670 | IFFO | 1.9584 | 0.011580041 |
| A_23_P62647 | NM_003037 | SLAMF1 | 1.9591 | 0.008425473 |
| A_23_P60387 | NM_017617 | NOTCH1 | 1.9599 | 0.001011355 |
| A_23_P79962 | NM_170784 | MKKS | 1.9605 | 0.006412782 |
| A_24_P409661 | XR_016019 | LOC729768 | 1.9612 | 0.025195196 |
| A_23_P64689 | NM_014871 | PAN2 | 1.9619 | 0.039702812 |
| A_23_P315386 | NM_052924 | RHPN1 | 1.962 | 0.001916462 |
| A_23_P168629 | NM_018077 | RBM28 | 1.9628 | 0.040315456 |
| A_23_P92082 | NM_001064 | TKT | 1.9628 | 0.000683607 |
| A_23_P369987 | NM_001039479 | KIAA0317 | 1.9633 | 0.005182004 |
| A_24_P345193 | NM_015001 | SPEN | 1.9639 | 0.008169442 |
| A_23_P331992 | AK056657 | STX6 | 1.9643 | 0.032264382 |
| A_23_P120812 | NM_020070 | IGLL1 | 1.9663 | 0.001298467 |
| A_24_P83848 | ENST00000249062 | ENST00000249062 | 1.9665 | 0.007077066 |
| A_23_P110704 | NM_006425 | SLU7 | 1.967 | 0.007422104 |
| A_23_P164637 | NM_024691 | ZNF419 | 1.9677 | 0.015528577 |
| A_23_P40354 | NM_012325 | MAPRE1 | 1.9681 | 0.015141783 |
| A_24_P374586 | NM_000535 | PMS2 | 1.969 | 0.022144496 |
| A_23_P25503 | NM_001079673 | FNDC3A | 1.9693 | 0.002302354 |
| A_23_P60387 | NM_017617 | NOTCH1 | 1.9703 | 0.000992723 |
| A_23_P32249 | AK001616 | FNBP1 | 1.9716 | 0.01707043 |
| A_24_P109272 | BC042998 | ADD1 | 1.9742 | 0.03037294 |
| (+)E1A_r60_a104 | E1A_r60_a104 | E1A_r60_a104 | 1.9743 | 0.033262801 |
| A_24_P916435 | AF147727 | AF147727 | 1.9745 | 0.011262422 |
| A_23_P28507 | NM_012214 | MGAT4A | 1.9753 | 0.022608697 |
| A_24_P607107 | A_24_P607107 | A_24_P607107 | 1.976 | 0.004174939 |
| A_23_P23625 | NM_030649 | CENTB5 | 1.9774 | 0.024821882 |
| A_23_P3527 | NM_032940 | POLR2C | 1.9783 | 0.000931238 |
| A_24_P307395 | A_24_P307395 | A_24_P307395 | 1.9791 | 0.005013891 |
| A_24_P49214 | AF469129 | C1orf86 | 1.9806 | 0.007041508 |
| A_32_P193322 | NM_152756 | RICTOR | 1.9812 | 0.001399752 |
| A_24_P272061 | NM_012423 | RPL13A | 1.9813 | 0.004172752 |
| A_23_P254702 | NM_003472 | DEK | 1.9817 | 0.010855096 |
| A_23_P115824 | NM_012207 | HNRNPH3 | 1.9836 | 0.004730236 |
| A_23_P139509 | NM_016570 | ERGIC2 | 1.9843 | 0.009751422 |
| A_23_P428382 | NM_203437 | AFTPH | 1.9843 | 0.014015936 |
| A_32_P218249 | NM_003211 | TDG | 1.9848 | 0.018968751 |
| A_23_P384127 | ENST00000390550 | ENST00000390550 | 1.9862 | 0.041070828 |
| A_32_P1434 | A_32_P1434 | A_32_P1434 | 1.987 | 0.002643135 |
| A_23_P148556 | NM_000033 | ABCD1 | 1.9872 | 0.0160992 |
| A_24_P247233 | XR_018420 | XR_018420 | 1.988 | 0.018653976 |
| A_24_P283635 | NM_001040272 | ADAMTSL1 | 1.9894 | 0.01481198 |
| A_24_P4054 | NM_003302 | TRIP6 | 1.9902 | 0.0043024 |
| A_24_P551028 | NM_001001664 | SPOPL | 1.9907 | 0.003072567 |
| A_23_P79545 | NM_003849 | SUCLG1 | 1.9921 | 0.009761392 |
| A_23_P368729 | NM_007162 | TFEB | 1.9924 | 0.001854203 |
| A_23_P55551 | NM_020412 | CHMP1B | 1.9929 | 0.002418458 |
| A_24_P191588 | NM_000688 | ALAS1 | 1.9944 | 0.005893642 |
| A_23_P28318 | NM_144736 | C2orf56 | 1.9945 | 0.044375928 |
| A_23_P75500 | NM_001024957 | BRMS1 | 1.9955 | 0.003355582 |
| A_23_P203586 | NM_016320 | NUP98 | 1.9964 | 0.007224259 |
| A_32_P20203 | BC035247 | BC035247 | 1.9966 | 0.029520803 |
| A_24_P25326 | NM_007167 | ZMYM6 | 1.9971 | 0.009321012 |
| A_23_P250800 | NM_006100 | ST3GAL6 | 1.9975 | 0.003301316 |
| A_24_P266880 | NM_021009 | UBC | 1.9981 | 0.004334889 |
| A_23_P140069 | NM_012158 | FBXL3 | 1.9983 | 0.002613846 |
| A_23_P78191 | NM_025099 | C17orf68 | 1.9984 | 0.004699777 |
| A_23_P366312 | NM_138473 | SP1 | 2.0002 | 0.016293712 |
| A_32_P186921 | BC032805 | ZNF616 | 2.0003 | 0.023205788 |
| A_23_P340890 | CR601567 | THAP3 | 2.001 | 0.023626235 |
| A_32_P163368 | AW076051 | AW076051 | 2.0016 | 0.012459909 |
| A_23_P56228 | NM_016573 | GMIP | 2.0043 | 0.000418838 |
| A_23_P126278 | NM_003465 | CHIT1 | 2.0044 | 0.007267957 |
| A_23_P60387 | NM_017617 | NOTCH1 | 2.0049 | 0.000778174 |
| A_23_P60387 | NM_017617 | NOTCH1 | 2.0053 | 0.000979335 |
| A_24_P294832 | NM_003463 | PTP4A1 | 2.0063 | 0.004317662 |
| A_23_P125815 | NM_005676 | RBM10 | 2.0065 | 0.006993021 |
| A_23_P44760 | NM_014159 | SETD2 | 2.0072 | 0.015788566 |
| A_24_P179489 | NM_006642 | SDCCAG8 | 2.0098 | 0.019936837 |
| A_23_P38408 | NM_024835 | GGNBP2 | 2.0102 | 0.012278876 |
| A_23_P116430 | NM_005709 | USH1C | 2.0112 | 0.027503406 |
| A_23_P308924 | AK093617 | AK093617 | 2.0115 | 0.00363063 |
| A_24_P290719 | AY047582 | AY047582 | 2.0123 | 0.030368262 |
| A_23_P90710 | NM_001927 | DES | 2.0127 | 0.028531622 |
| A_23_P60387 | NM_017617 | NOTCH1 | 2.0129 | 0.000844691 |
| A_32_P192033 | AK055918 | FLJ31356 | 2.0143 | 0.018569056 |
| A_23_P342348 | BC047869 | COX4I1 | 2.0156 | 0.0084027 |
| A_23_P24723 | NM_016464 | TMEM138 | 2.016 | 0.02802426 |
| A_23_P27638 | NM_144566 | ZNF700 | 2.0168 | 0.040970282 |
| A_32_P92171 | BC052596 | LOC388564 | 2.0192 | 0.002900582 |
| A_23_P51069 | NM_001862 | COX5B | 2.0197 | 0.007747466 |
| A_23_P110253 | NM_000222 | KIT | 2.0198 | 0.016900503 |
| A_23_P68922 | NM_033386 | MICALL1 | 2.0202 | 0.023644458 |
| A_24_P276531 | NM_024700 | SNIP1 | 2.0204 | 0.04728301 |
| A_24_P366670 | NM_002285 | AFF3 | 2.0214 | 0.028270145 |
| A_24_P937435 | AL713718 | RBED1 | 2.0221 | 0.042585538 |
| A_23_P209694 | NM_022894 | PAPOLG | 2.0228 | 0.005667753 |
| A_24_P252078 | NM_007047 | BTN3A2 | 2.0252 | 0.004241016 |
| A_23_P96775 | NM_020710 | LRRC47 | 2.0257 | 0.01321528 |
| A_24_P46953 | NM_013257 | SGK3 | 2.0257 | 0.005384422 |
| A_24_P318319 | NM_005466 | MED6 | 2.0258 | 0.037708379 |
| A_23_P139509 | NM_016570 | ERGIC2 | 2.026 | 0.008594505 |
| A_23_P396194 | NM_022457 | RFWD2 | 2.0266 | 0.002788261 |
| A_23_P117527 | NM_017943 | FBXO34 | 2.0268 | 0.046184539 |
| A_23_P81247 | NM_030571 | NDFIP1 | 2.0271 | 0.012369312 |
| A_23_P217367 | NM_052936 | ATG4A | 2.0271 | 0.03467508 |
| A_23_P366312 | NM_138473 | SP1 | 2.028 | 0.013872319 |
| A_23_P60387 | NM_017617 | NOTCH1 | 2.0296 | 0.000781277 |
| A_24_P393880 | NM_001043352 | TPM3 | 2.0296 | 0.042048421 |
| A_23_P83463 | NM_004261 | 15-Sep | 2.0296 | 0.003977991 |
| A_23_P21485 | NM_017933 | PID1 | 2.0299 | 0.000299499 |
| A_24_P278460 | NM_032228 | MLSTD2 | 2.0323 | 0.008206392 |
| A_24_P6850 | A_24_P6850 | A_24_P6850 | 2.0325 | 0.022702475 |
| A_23_P207400 | NM_007295 | BRCA1 | 2.0326 | 0.003533802 |
| A_23_P105619 | NM_138341 | TMEM116 | 2.0326 | 0.000999577 |
| A_23_P94683 | NM_002504 | NFX1 | 2.0339 | 0.027649161 |
| A_23_P54340 | NM_032445 | MEGF11 | 2.0342 | 0.032221115 |
| (+)E1A_r60_a104 | E1A_r60_a104 | E1A_r60_a104 | 2.0353 | 0.045960103 |
| A_23_P252936 | NM_015266 | SLC9A8 | 2.0368 | 0.04473795 |
| A_23_P159671 | NM_000292 | PHKA2 | 2.038 | 0.009140834 |
| A_24_P477127 | NM_000296 | PKD1 | 2.038 | 0.030987614 |
| A_23_P10873 | NM_003263 | TLR1 | 2.0382 | 0.00089009 |
| A_23_P44768 | NM_013254 | TBK1 | 2.0423 | 0.009743639 |
| A_23_P98910 | NM_006152 | LRMP | 2.0429 | 0.003708805 |
| A_23_P366312 | NM_138473 | SP1 | 2.0431 | 0.016627941 |
| A_23_P208450 | NM_024103 | SLC25A23 | 2.0459 | 0.037367748 |
| A_23_P209944 | NM_017750 | RETSAT | 2.0464 | 0.018986505 |
| A_24_P841622 | AK096589 | RPL32P3 | 2.0465 | 0.016011608 |
| A_23_P37441 | NM_004048 | B2M | 2.0466 | 0.002698998 |
| A_23_P331379 | NM_198400 | NEDD4 | 2.047 | 0.031191928 |
| A_23_P38482 | NM_024297 | PHF23 | 2.047 | 0.015698334 |
| A_23_P126008 | NM_015112 | MAST2 | 2.0472 | 0.029102501 |
| A_23_P14105 | NM_001268 | RCBTB2 | 2.0473 | 0.004335949 |
| A_23_P46356 | NM_024575 | TNFAIP8L2 | 2.0477 | 0.002798734 |
| A_23_P133146 | NM_002106 | H2AFZ | 2.0479 | 0.005360514 |
| A_23_P250800 | NM_006100 | ST3GAL6 | 2.0495 | 0.002533558 |
| A_32_P93121 | THC2650012 | THC2650012 | 2.0497 | 0.029198685 |
| A_23_P69491 | NM_001664 | RHOA | 2.0527 | 0.01229859 |
| A_23_P102000 | NM_001008540 | CXCR4 | 2.0528 | 0.001434444 |
| A_23_P165028 | NM_001348 | DAPK3 | 2.0533 | 0.001940503 |
| A_24_P452326 | A_24_P452326 | A_24_P452326 | 2.0562 | 0.038286529 |
| A_23_P34710 | CR604521 | EGLN1 | 2.0564 | 0.000348775 |
| A_23_P207400 | NM_007295 | BRCA1 | 2.0565 | 0.003153613 |
| A_23_P4798 | NM_016535 | ZNF581 | 2.0571 | 0.014090921 |
| A_23_P207400 | NM_007295 | BRCA1 | 2.0573 | 0.002109248 |
| A_23_P250800 | NM_006100 | ST3GAL6 | 2.0582 | 0.002807603 |
| A_23_P35168 | NM_013339 | ALG6 | 2.0583 | 0.004279772 |
| A_23_P250800 | NM_006100 | ST3GAL6 | 2.059 | 0.002349124 |
| A_24_P17677 | NM_006068 | TLR6 | 2.0601 | 8.34E−05 |
| A_23_P502957 | NM_021810 | CDH26 | 2.0602 | 0.012444288 |
| A_23_P79199 | NM_020548 | DBI | 2.0615 | 0.041918395 |
| A_23_P254801 | NM_002660 | PLCG1 | 2.0618 | 0.005388999 |
| A_23_P160881 | NM_001009568 | SMPDL3B | 2.0622 | 0.036458381 |
| A_32_P174398 | ENST00000391665 | ENST00000391665 | 2.0625 | 0.020560023 |
| A_32_P199263 | BC073929 | BC073929 | 2.063 | 0.03803385 |
| A_23_P60387 | NM_017617 | NOTCH1 | 2.0635 | 0.000922006 |
| A_23_P83094 | NM_007005 | TLE4 | 2.0651 | 0.006534089 |
| A_24_P205242 | BC034378 | RPL23 | 2.068 | 0.028621436 |
| A_23_P204408 | NM_005056 | JARID1A | 2.0684 | 0.01699178 |
| A_23_P125829 | NM_000291 | PGK1 | 2.0689 | 0.018988473 |
| A_23_P4494 | NM_024422 | DSC2 | 2.073 | 0.003563305 |
| A_23_P1926 | NM_017841 | C11orf79 | 2.0734 | 0.006150038 |
| A_23_P133470 | NM_014819 | PJA2 | 2.074 | 0.001094086 |
| A_24_P157698 | NM_173828 | RELL2 | 2.0747 | 0.012139291 |
| A_24_P357576 | NM_019600 | KIAA1370 | 2.0749 | 0.000985984 |
| A_24_P412512 | NM_016350 | NIN | 2.0752 | 0.009802318 |
| A_23_P100912 | NM_014030 | GIT1 | 2.0758 | 0.036066897 |
| A_24_P916614 | NM_005156 | ROD1 | 2.0791 | 0.002040968 |
| A_23_P130919 | NM_130807 | MOBKL2A | 2.0837 | 0.00103252 |
| A_23_P80739 | NM_006225 | PLCD1 | 2.085 | 0.011495653 |
| A_23_P266 | U82382 | PIN1L | 2.0852 | 0.021144867 |
| A_23_P254801 | NM_002660 | PLCG1 | 2.0854 | 0.010428513 |
| A_23_P110253 | NM_000222 | KIT | 2.0856 | 0.010239367 |
| A_23_P352358 | NM_138346 | KIAA2013 | 2.0862 | 0.001482782 |
| A_23_P90626 | NM_004288 | PSCDBP | 2.0866 | 0.005686503 |
| A_24_P684186 | NR_003955 | LOC647121 | 2.0875 | 0.000385001 |
| A_23_P1641 | NM_005133 | RCE1 | 2.0901 | 0.032211812 |
| A_23_P93613 | NM_013255 | MKLN1 | 2.0903 | 0.005510451 |
| A_23_P205724 | NM_199043 | C14orf102 | 2.0917 | 0.04915901 |
| A_23_P139509 | NM_016570 | ERGIC2 | 2.092 | 0.006032093 |
| A_23_P250800 | NM_006100 | ST3GAL6 | 2.0929 | 0.002380288 |
| A_23_P24751 | NM_173810 | TTC9C | 2.0937 | 0.035332381 |
| A_23_P110253 | NM_000222 | KIT | 2.0938 | 0.013846464 |
| A_23_P204016 | NM_000725 | CACNB3 | 2.0941 | 0.030801746 |
| A_23_P102000 | NM_001008540 | CXCR4 | 2.0943 | 0.001884718 |
| A_32_P17364 | THC2664263 | THC2664263 | 2.0955 | 0.035891713 |
| A_23_P134946 | NM_014665 | LRRC14 | 2.0973 | 0.019955514 |
| A_32_P27271 | NM_198256 | E2F6 | 2.0982 | 0.022176549 |
| A_32_P186027 | ENST00000267918 | ANP32A | 2.0986 | 0.005470216 |
| A_24_P254346 | NM_006052 | DSCR3 | 2.099 | 0.013864596 |
| A_23_P105519 | NM_016312 | WBP11 | 2.1012 | 0.010191234 |
| A_24_P181672 | NM_001009905 | B3GNTL1 | 2.1019 | 0.001120164 |
| A_23_P26697 | NM_033452 | TRIM47 | 2.1023 | 0.01472311 |
| A_24_P162128 | AK054840 | C4orf38 | 2.1026 | 0.001655313 |
| A_23_P137157 | NM_002910 | RENBP | 2.1033 | 0.022815288 |
| A_32_P90551 | NM_014230 | SRP68 | 2.104 | 0.0457735 |
| A_24_P204374 | A_24_P204374 | A_24_P204374 | 2.1048 | 0.003004131 |
| A_24_P111019 | BC028243 | BC028243 | 2.1058 | 0.010907667 |
| A_23_P203737 | NM_033547 | INTS4 | 2.1061 | 0.041010663 |
| A_24_P268210 | ENST00000358951 | RAB3GAP2 | 2.1063 | 0.018292254 |
| A_23_P431789 | NM_001660 | ARF4 | 2.1068 | 0.015192886 |
| A_23_P97218 | CR596670 | CR596670 | 2.1072 | 0.015305012 |
| A_32_P93144 | XM_001714697 | LOC653458 | 2.1094 | 0.015563665 |
| A_23_P46170 | NM_052877 | MED8 | 2.1097 | 0.010602809 |
| A_23_P12423 | NM_022089 | ATP13A2 | 2.1098 | 0.029446633 |
| A_32_P181564 | THC2606490 | THC2606490 | 2.1099 | 0.029185593 |
| A_32_P190864 | THC2718727 | THC2718727 | 2.1113 | 0.015948724 |
| A_23_P254801 | NM_002660 | PLCG1 | 2.1118 | 0.005952682 |
| A_23_P122464 | NM_006299 | ZNF193 | 2.1118 | 0.016466219 |
| A_23_P69491 | NM_001664 | RHOA | 2.1119 | 0.013641558 |
| A_23_P366455 | NM_133264 | WIPF2 | 2.1145 | 0.003947082 |
| A_23_P313476 | NM_032448 | FAM120B | 2.1148 | 0.015198928 |
| (+)E1A_r60_a104 | E1A_r60_a104 | E1A_r60_a104 | 2.1174 | 0.013745832 |
| A_23_P65262 | NM_033111 | N4BP2L2 | 2.1179 | 0.000669777 |
| A_24_P321919 | NM_003870 | IQGAP1 | 2.1195 | 0.000739426 |
| A_23_P7543 | NM_014733 | ZFYVE16 | 2.1205 | 0.005259647 |
| A_24_P211842 | NM_178128 | FADS6 | 2.1215 | 0.038171044 |
| A_23_P47790 | NM_005371 | METTL1 | 2.1215 | 0.032670425 |
| A_24_P566891 | AK023803 | ARF1 | 2.1221 | 0.001287347 |
| A_24_P367496 | AK124894 | LOC100131650 | 2.1294 | 0.01453245 |
| A_23_P374782 | NM_001024666 | SH3KBP1 | 2.1314 | 0.015917041 |
| A_23_P103282 | NM_004872 | TMEM59 | 2.1317 | 0.009953263 |
| A_24_P268893 | NM_144721 | THAP6 | 2.1318 | 0.008820978 |
| A_23_P396777 | NM_006315 | PCGF3 | 2.1319 | 0.016890278 |
| A_23_P102113 | NM_025216 | WNT10A | 2.134 | 0.003054325 |
| A_23_P94932 | NM_015702 | C2orf25 | 2.1353 | 0.002937811 |
| A_24_P886040 | NM_152624 | DCP2 | 2.138 | 0.004751221 |
| A_24_P370887 | NM_004781 | VAMP3 | 2.1384 | 0.000663233 |
| A_32_P26103 | NM_006412 | AGPAT2 | 2.1393 | 0.009115299 |
| A_24_P201404 | NM_014039 | C11orf54 | 2.1406 | 0.007543729 |
| A_23_P8664 | NM_021145 | DMTF1 | 2.1411 | 0.009224825 |
| A_24_P195544 | BC061642 | BC061642 | 2.1439 | 0.015302658 |
| A_24_P7750 | XR_038299 | LOC100128116 | 2.1462 | 0.023681168 |
| A_23_P34045 | NM_001399 | EDA | 2.1473 | 0.028285006 |
| A_23_P67583 | NM_018174 | MAP1S | 2.1497 | 0.004831118 |
| A_23_P79661 | NM_019044 | CCDC93 | 2.1499 | 0.014921252 |
| A_24_P928574 | AK023400 | AK023400 | 2.1502 | 0.011351465 |
| A_24_P916338 | A_24_P916338 | A_24_P916338 | 2.1503 | 0.030298451 |
| A_23_P91891 | NM_004766 | COPB2 | 2.1526 | 0.01329819 |
| A_32_P115220 | NM_152569 | C9orf66 | 2.1552 | 0.012831527 |
| A_23_P366312 | NM_138473 | SP1 | 2.1553 | 0.006683254 |
| A_23_P250800 | NM_006100 | ST3GAL6 | 2.1569 | 0.002242187 |
| A_23_P72850 | NM_032889 | MFSD5 | 2.1584 | 0.016213166 |
| A_23_P125829 | NM_000291 | PGK1 | 2.1591 | 0.015385762 |
| A_23_P100022 | NM_014848 | SV2B | 2.1597 | 0.029347778 |
| A_23_P395033 | NM_020892 | DTX2 | 2.1598 | 0.019057719 |
| A_23_P3856 | NM_153688 | ZFP1 | 2.1627 | 0.035647689 |
| A_23_P56709 | NM_005667 | RNF103 | 2.167 | 0.01006977 |
| A_23_P9392 | NM_016390 | C9orf114 | 2.1697 | 0.00958676 |
| A_32_P178499 | NM_001012984 | C16orf86 | 2.1699 | 0.049433555 |
| A_24_P306704 | XR_016161 | XR_016161 | 2.1702 | 0.014894949 |
| A_23_P95353 | NM_014678 | SAPS2 | 2.1705 | 0.017896483 |
| A_23_P98232 | NM_006782 | ZFPL1 | 2.1706 | 0.010430987 |
| A_23_P320478 | BC065260 | FLJ22795 | 2.1709 | 0.008973447 |
| A_23_P164946 | NM_001039885 | FKRP | 2.1736 | 0.044119285 |
| A_23_P207400 | NM_007295 | BRCA1 | 2.1737 | 0.001844391 |
| A_23_P31085 | NM_015439 | CCDC28A | 2.1738 | 0.00403391 |
| A_23_P87500 | NM_014182 | ORMDL2 | 2.174 | 0.04545291 |
| A_23_P139509 | NM_016570 | ERGIC2 | 2.1742 | 0.005739595 |
| A_23_P26824 | NM_030576 | LIMD2 | 2.1745 | 0.006997798 |
| A_24_P265539 | NM_015607 | C1orf77 | 2.1748 | 0.021434046 |
| A_24_P371303 | NM_015224 | C3orf63 | 2.1748 | 0.01361405 |
| A_24_P198629 | NM_018148 | LINS1 | 2.1754 | 0.009448194 |
| A_32_P206293 | NM_024639 | ZNF322A | 2.1766 | 0.024373464 |
| A_23_P311087 | NM_012482 | ZNF281 | 2.1788 | 0.000231406 |
| A_24_P108351 | NM_007040 | HNRNPUL1 | 2.1789 | 0.031671499 |
| A_24_P353208 | NM_018556 | SIRPG | 2.1803 | 0.002349497 |
| A_23_P305938 | NM_007131 | ZNF75 | 2.1804 | 0.012956778 |
| A_24_P346807 | NM_022079 | HERC4 | 2.1805 | 0.006981955 |
| A_24_P166397 | NM_014809 | KIAA0319 | 2.1812 | 0.009632631 |
| A_24_P86968 | NM_000733 | CD3E | 2.1823 | 0.010929209 |
| A_23_P159839 | NM_152692 | C1GALT1C1 | 2.1837 | 0.008921391 |
| A_23_P168062 | NM_003587 | DHX16 | 2.184 | 0.016551578 |
| A_24_P926666 | AK000925 | AK000925 | 2.1841 | 0.000549901 |
| A_23_P334111 | BC002482 | WDR55 | 2.1842 | 0.00939149 |
| A_23_P100486 | NM_206824 | VKORC1 | 2.1842 | 0.039988989 |
| A_24_P912404 | A_24_P912404 | A_24_P912404 | 2.1867 | 0.04507079 |
| A_23_P142708 | NM_030805 | LMAN2L | 2.1882 | 0.030679924 |
| A_23_P139509 | NM_016570 | ERGIC2 | 2.1887 | 0.004214876 |
| A_23_P11915 | NM_017686 | GDAP2 | 2.1887 | 0.014242837 |
| A_24_P583007 | BC039246 | BC039246 | 2.1908 | 0.011098991 |
| A_23_P98722 | NM_005898 | CAPRIN1 | 2.1913 | 0.015228421 |
| A_23_P141302 | NM_144680 | ZNF18 | 2.1953 | 0.025961973 |
| A_23_P50418 | NM_153358 | ZNF791 | 2.1954 | 0.011452875 |
| A_23_P205336 | NM_016472 | C14orf129 | 2.1969 | 0.003896974 |
| A_24_P932632 | CR617018 | CR617018 | 2.1999 | 0.002440445 |
| A_23_P168443 | NM_004444 | EPHB4 | 2.2011 | 0.034283426 |
| A_23_P422083 | NM_018710 | TMEM55A | 2.2024 | 0.001083352 |
| A_23_P344531 | NM_007286 | SYPO | 2.2029 | 0.024838806 |
| A_23_P7655 | NM_013978 | BNIP1 | 2.205 | 0.028103942 |
| A_23_P201567 | NM_018061 | PRPF38B | 2.2056 | 0.016489399 |
| A_23_P126129 | NM_030934 | C1orf25 | 2.2068 | 0.014370694 |
| A_24_P101181 | XR_019095 | XR_019095 | 2.2091 | 0.012324664 |
| A_23_P319895 | ENST00000267197 | SETD1B | 2.2105 | 0.007256649 |
| A_23_P151624 | NM_173527 | REM2 | 2.2117 | 0.029056801 |
| A_24_P187197 | BX648930 | LOC652005 | 2.214 | 0.00675532 |
| A_23_P34126 | NM_001711 | BGN | 2.2145 | 0.007256591 |
| A_24_P310616 | NM_001042498 | SLC35A2 | 2.2159 | 0.023827742 |
| A_23_P24577 | AK091966 | PDZD3 | 2.2169 | 0.017438277 |
| A_23_P47800 | NM_019887 | DIABLO | 2.2174 | 0.015485701 |
| A_24_P497186 | NM_182972 | IRF2BP2 | 2.2183 | 0.003485684 |
| A_23_P158897 | BE531123 | BE531123 | 2.2195 | 0.033235338 |
| A_24_P927996 | A_24_P927996 | A_24_P927996 | 2.22 | 0.04041645 |
| A_24_P341546 | XR_018415 | XR_018415 | 2.2211 | 0.04207428 |
| A_23_P84648 | NM_000202 | IDS | 2.2261 | 0.008984428 |
| A_23_P138826 | NM_172230 | SYVN1 | 2.2271 | 0.038344677 |
| A_23_P17911 | NM_145912 | NFAM1 | 2.2274 | 0.006714185 |
| A_32_P144596 | NM_003747 | TNKS | 2.2276 | 0.049859765 |
| A_32_P167856 | AF338232 | CTAGE4 | 2.2279 | 0.01025125 |
| A_24_P83952 | BC014432 | PDZRN3 | 2.2283 | 0.012467249 |
| A_23_P69339 | NM_001607 | ACAA1 | 2.2293 | 0.00148215 |
| A_32_P119949 | A_32_P119949 | A_32_P119949 | 2.2299 | 0.009064435 |
| A_24_P76911 | NM_015528 | RNF167 | 2.2305 | 0.025302808 |
| A_24_P173566 | X57818 | X57818 | 2.231 | 0.002415691 |
| A_23_P137935 | NM_002432 | MNDA | 2.2338 | 0.000423188 |
| A_24_P150874 | CR611712 | CR611712 | 2.2343 | 0.002990929 |
| A_23_P69491 | NM_001664 | RHOA | 2.2347 | 0.010826467 |
| A_23_P321855 | NM_003899 | ARHGEF7 | 2.2348 | 0.009758404 |
| A_24_P118608 | NM_032437 | EFCAB7 | 2.2351 | 0.024361887 |
| A_23_P76610 | AK001817 | PSPC1 | 2.2356 | 0.007888433 |
| A_23_P48997 | NM_003978 | PSTPIP1 | 2.2362 | 0.000456498 |
| A_23_P4144 | NM_025233 | COASY | 2.2374 | 0.001136547 |
| A_24_P940086 | AB032985 | NXPH3 | 2.2381 | 0.011427086 |
| A_23_P139509 | NM_016570 | ERGIC2 | 2.2384 | 0.004762297 |
| A_23_P60387 | NM_017617 | NOTCH1 | 2.2397 | 0.000445001 |
| A_23_P430161 | NM_175876 | EXOC8 | 2.2402 | 0.004329801 |
| A_23_P47565 | NM_005566 | LDHA | 2.2405 | 0.001740116 |
| A_24_P800824 | AK096031 | AK096031 | 2.2412 | 0.012215472 |
| A_24_P239451 | BC011648 | BC011648 | 2.242 | 0.038275102 |
| A_23_P417965 | NM_015603 | CCDC9 | 2.2432 | 0.015600356 |
| A_24_P398972 | NM_016138 | COQ7 | 2.2448 | 0.03024088 |
| A_23_P251945 | NM_016221 | DCTN4 | 2.2464 | 0.002581251 |
| A_23_P416656 | NM_033375 | MYOIC | 2.2465 | 0.037558514 |
| A_23_P98963 | AK002085 | LOC144438 | 2.2467 | 0.005156685 |
| A_32_P76627 | CR597597 | CR597597 | 2.2493 | 0.046403535 |
| A_24_P298027 | NM_004655 | AXIN2 | 2.2516 | 0.043954905 |
| A_24_P392480 | NM_001012643 | LOC339344 | 2.2529 | 0.012724728 |
| A_23_P5389 | NM_032574 | DPY30 | 2.2536 | 0.011138875 |
| A_23_P7783 | NM_016604 | JMJD1B | 2.2557 | 0.007784773 |
| A_23_P124224 | NM_004941 | DHX8 | 2.2573 | 0.002223612 |
| A_23_P31348 | THC2571921 | THC2571921 | 2.2581 | 0.024407463 |
| A_23_P131899 | NM_080489 | SDCBP2 | 2.2653 | 0.021302716 |
| A_23_P13382 | NM_001013254 | LSP1 | 2.267 | 0.000783718 |
| A_24_P218587 | NM_004268 | MED17 | 2.2679 | 0.003221888 |
| A_23_P61308 | AK023691 | CCDC71 | 2.2681 | 0.006066805 |
| A_23_P71778 | NM_203347 | RP11-216L13.5 | 2.2696 | 0.03060543 |
| A_24_P234462 | NM_147196 | TMIE | 2.2704 | 0.033967664 |
| A_24_P826578 | AK311609 | AK311609 | 2.2738 | 0.022309498 |
| A_23_P57118 | NM_003245 | TGM3 | 2.2741 | 0.001435842 |
| A_23_P157051 | NM_004722 | AP4M1 | 2.2772 | 0.011219817 |
| A_24_P565430 | BQ778202 | BQ778202 | 2.2786 | 0.043164729 |
| A_23_P75509 | NM_003626 | PPFIA1 | 2.2786 | 0.001494699 |
| A_32_P150142 | AL713776 | DKFZp434L192 | 2.2786 | 0.046896655 |
| A_24_P176255 | NM_005254 | GABPB2 | 2.2789 | 0.035546439 |
| A_24_P300379 | NM_153370 | PI16 | 2.2789 | 0.000479551 |
| A_24_P92183 | NM_001124756 | PABPC1L | 2.2805 | 0.003908202 |
| A_24_P573089 | NM_001103161 | SH2D5 | 2.2816 | 0.02083766 |
| A_23_P40039 | NM_031445 | AMMECR1L | 2.2825 | 0.03419384 |
| A_23_P37237 | NM_145165 | CHURC1 | 2.2845 | 0.011131599 |
| A_23_P69491 | NM_001664 | RHOA | 2.2857 | 0.007911372 |
| A_24_P79617 | CR602920 | KIAA0040 | 2.2863 | 0.020358965 |
| A_23_P139509 | NM_016570 | ERGIC2 | 2.2869 | 0.004591816 |
| A_24_P266728 | NM_004630 | SF1 | 2.2876 | 0.005645795 |
| A_24_P930415 | NM_001012508 | SPRN | 2.2884 | 0.017764653 |
| A_32_P226449 | THC2778545 | THC2778545 | 2.2886 | 0.025631483 |
| A_24_P593724 | A_24_P593724 | A_24_P593724 | 2.289 | 0.009701966 |
| A_23_P218770 | NM_002872 | RAC2 | 2.2894 | 0.006897721 |
| A_23_P69491 | NM_001664 | RHOA | 2.2897 | 0.007197259 |
| A_32_P58425 | NM_153812 | PHF13 | 2.291 | 0.011523705 |
| A_32_P229746 | NM_005494 | DNAJB6 | 2.2915 | 0.010217787 |
| A_23_P125829 | NM_000291 | PGK1 | 2.2917 | 0.010087967 |
| A_32_P117758 | BG208131 | BG208131 | 2.2942 | 0.033404636 |
| A_23_P145529 | NM_181795 | PKIB | 2.2943 | 0.02774305 |
| A_23_P76598 | NM_152726 | EFHA1 | 2.2949 | 0.007562223 |
| A_23_P106682 | NM_001424 | EMP2 | 2.2954 | 0.049879886 |
| A_23_P215037 | NM_152858 | WTAP | 2.2959 | 0.019833578 |
| A_23_P29566 | NM_005718 | ARPC4 | 2.2973 | 0.014702611 |
| A_23_P133648 | NM_016255 | FAM8A1 | 2.2991 | 0.000671444 |
| A_23_P66967 | NM_004066 | CETN1 | 2.3039 | 0.039360808 |
| A_23_P99853 | NM_019600 | KIAA1370 | 2.3061 | 0.001893927 |
| A_23_P56069 | NM_015578 | LSM14A | 2.3069 | 0.014873825 |
| A_23_P6869 | NM_007024 | TMEM115 | 2.3072 | 0.007007717 |
| A_23_P69853 | NM_173828 | RELL2 | 2.3093 | 0.004842324 |
| A_24_P2995 | NM_015317 | PUM2 | 2.3102 | 0.006399536 |
| A_23_P113966 | NM_001024924 | EXOC1 | 2.3105 | 0.002707254 |
| A_24_P339664 | NM_020170 | NCLN | 2.3108 | 0.00469517 |
| A_24_P358406 | A_24_P358406 | A_24_P358406 | 2.3111 | 0.0182995 |
| A_24_P929867 | A_24_P929867 | A_24_P929867 | 2.3126 | 0.027664775 |
| A_23_P415033 | NM_138795 | ARL8A | 2.3128 | 0.03690486 |
| A_23_P166910 | NM_015268 | DNAJC13 | 2.3157 | 0.047858035 |
| A_32_P160216 | BM702130 | BM702130 | 2.3171 | 0.018409209 |
| A_24_P941824 | NM_004798 | KIF3B | 2.3177 | 0.007714649 |
| A_32_P220826 | AK091162 | WBSCR22 | 2.3186 | 0.02182845 |
| A_23_P409988 | NM_024947 | PHC3 | 2.3191 | 0.023192451 |
| A_23_P43283 | NM_032777 | GPR124 | 2.32 | 0.009276624 |
| A_23_P157607 | NM_018142 | INTS10 | 2.3203 | 0.010455341 |
| A_23_P105409 | NM_006301 | MAP3K12 | 2.3229 | 0.034160969 |
| A_24_P24230 | A_24_P24230 | A_24_P24230 | 2.3231 | 0.007056155 |
| A_24_P209285 | NM_018141 | MRPS10 | 2.3235 | 0.018511469 |
| A_24_P125839 | NM_017447 | C21orf91 | 2.3235 | 0.009356931 |
| A_23_P53788 | NM_152912 | MTIF3 | 2.3279 | 0.004831984 |
| A_32_P60343 | NM_198181 | LOC440295 | 2.3279 | 0.005630058 |
| A_24_P281313 | AK095554 | LOC100129858 | 2.3301 | 0.014387061 |
| A_32_P202587 | A_32_P202587 | A_32_P202587 | 2.3338 | 0.020862761 |
| A_23_P250800 | NM_006100 | ST3GAL6 | 2.3339 | 0.000964148 |
| A_23_P254031 | NM_007344 | TTF1 | 2.3348 | 0.002259195 |
| A_24_P396660 | NM_147148 | GSTM4 | 2.3442 | 0.014473976 |
| A_23_P431981 | NM_005487 | HMG2L1 | 2.3451 | 0.032394771 |
| A_23_P2705 | NM_005767 | P2RY5 | 2.3462 | 9.99E−05 |
| A_23_P102973 | NM_022719 | DGCR14 | 2.347 | 0.006344865 |
| A_23_P207400 | NM_007295 | BRCA1 | 2.3489 | 0.00103258 |
| A_23_P38365 | NM_006852 | TLK2 | 2.3489 | 0.018851621 |
| A_23_P404667 | NM_001197 | BIK | 2.3494 | 0.00888116 |
| A_23_P30024 | NM_003998 | NFKB1 | 2.3496 | 0.012852874 |
| A_32_P198810 | ENST00000381800 | ENST00000381800 | 2.3497 | 0.043981112 |
| A_24_P96505 | NM_001495 | GFRA2 | 2.3504 | 0.020102498 |
| A_24_P380061 | NM_001025616 | ARHGAP24 | 2.3508 | 0.047422345 |
| A_23_P254801 | NM_002660 | PLCG1 | 2.3511 | 0.005920625 |
| A_23_P125033 | THC2493066 | THC2493066 | 2.3529 | 0.023505591 |
| A_24_P365129 | THC2504193 | THC2504193 | 2.3553 | 0.031229825 |
| A_24_P141995 | NM_032632 | PAPOLA | 2.3554 | 0.007267279 |
| A_24_P111134 | NM_013382 | POMT2 | 2.3577 | 0.032026661 |
| A_23_P115407 | NM_146421 | GSTM1 | 2.3594 | 0.03970805 |
| A_23_P411379 | NM_178508 | C6orf1 | 2.3616 | 0.006623628 |
| A_32_P4934 | THC2640099 | THC2640099 | 2.362 | 0.000589929 |
| A_24_P230521 | CR623296 | GPSM3 | 2.3634 | 0.034299288 |
| A_23_P200443 | NM_003029 | SHC1 | 2.3637 | 0.009395563 |
| A_24_P942002 | NM_012287 | CENTB2 | 2.3644 | 0.001181458 |
| A_24_P232696 | NM_139071 | SMARCD1 | 2.3653 | 0.023752623 |
| A_24_P117672 | NM_021228 | SCAF1 | 2.3655 | 0.033785887 |
| A_23_P160689 | NM_018372 | C1orf103 | 2.3705 | 0.001671133 |
| A_24_P46808 | CR590302 | MDH2 | 2.3744 | 0.016245059 |
| A_24_P330366 | NM_015156 | RCOR1 | 2.3747 | 0.004241911 |
| A_23_P200160 | NM_001014975 | CFH | 2.3758 | 0.000876975 |
| A_23_P203389 | NM_016499 | MGC13379 | 2.3767 | 0.0081828 |
| A_23_P335452 | NM_153367 | C10orf56 | 2.3782 | 0.049824543 |
| A_23_P145437 | NM_017934 | PHIP | 2.3796 | 0.006246569 |
| A_23_P200030 | NM_003838 | FPGT | 2.3805 | 0.00344041 |
| A_23_P30495 | NM_000859 | HMGCR | 2.3815 | 0.00170078 |
| A_24_P147407 | NM_153335 | LYK5 | 2.3846 | 0.001827682 |
| A_24_P396994 | NM_001690 | ATP6V1A | 2.3847 | 0.001127957 |
| A_32_P178339 | A_32_P178339 | A_32_P178339 | 2.3863 | 0.022073715 |
| A_23_P77286 | NM_024713 | C15orf29 | 2.3866 | 0.000325131 |
| A_23_P64743 | NM_015000 | STK38L | 2.388 | 0.00223508 |
| A_24_P485012 | BC017563 | BC017563 | 2.3895 | 0.027506417 |
| A_23_P110253 | NM_000222 | KIT | 2.3906 | 0.006817525 |
| A_23_P80008 | NM_033118 | MYLK2 | 2.3912 | 0.028459717 |
| A_24_P307580 | NM_001098523 | HTATIP2 | 2.3913 | 0.04148733 |
| A_32_P88719 | NM_001031834 | RAB40AL | 2.3934 | 0.014522958 |
| A_23_P106575 | NM_002080 | GOT2 | 2.3936 | 0.003390024 |
| A_24_P109071 | NM_017576 | KIF27 | 2.3941 | 0.036486202 |
| A_23_P213247 | NM_033535 | FBXL5 | 2.3973 | 0.000122827 |
| A_23_P140050 | NM_017569 | FAM48A | 2.3973 | 0.005131703 |
| A_23_P20463 | NM_006265 | RAD21 | 2.3981 | 0.002731659 |
| A_24_P126417 | NM_014709 | USP34 | 2.3983 | 0.000573798 |
| A_23_P380917 | NM_014300 | SEC11A | 2.4015 | 0.00628912 |
| A_23_P69491 | NM_001664 | RHOA | 2.4017 | 0.004753126 |
| A_23_P144684 | NM_032290 | ANKRD32 | 2.402 | 0.002009267 |
| A_23_P58898 | NM_012115 | CASP8AP2 | 2.4024 | 0.005362071 |
| A_24_P367576 | AK125170 | LOC100131993 | 2.405 | 0.000472311 |
| A_23_P21134 | NM_004083 | DDIT3 | 2.4057 | 0.001110723 |
| A_24_P77008 | NM_000963 | PTGS2 | 2.4062 | 0.010740898 |
| A_24_P937240 | U09197 | U09197 | 2.4066 | 0.006676618 |
| A_23_P19778 | NM_012450 | SLC13A4 | 2.4067 | 0.00684222 |
| A_24_P294719 | NM_014613 | UBXD8 | 2.4067 | 0.015375632 |
| A_23_P62901 | NM_006763 | BTG2 | 2.4069 | 0.002110378 |
| A_23_P202696 | NM_016506 | KBTBD4 | 2.408 | 0.03169192 |
| A_24_P394368 | NM_025132 | WDR19 | 2.4102 | 0.046910306 |
| A_23_P112113 | NM_014846 | KIAA0196 | 2.4117 | 0.00933422 |
| A_23_P373992 | NM_145294 | WDR90 | 2.4126 | 0.015346778 |
| A_24_P928510 | NM_053055 | THEM4 | 2.4182 | 0.005789878 |
| A_23_P321354 | NM_144649 | TMEM71 | 2.4211 | 0.000178318 |
| A_24_P920979 | AK057921 | LOC145758 | 2.4231 | 0.016415845 |
| A_23_P109547 | NM_019008 | SMCR7L | 2.4248 | 0.014260251 |
| A_32_P31633 | NM_012433 | SF3B1 | 2.4252 | 0.003225772 |
| A_23_P69491 | NM_001664 | RHOA | 2.4294 | 0.005178173 |
| A_23_P170467 | NM_006537 | USP3 | 2.4308 | 0.002758455 |
| A_23_P131449 | ENST00000290390 | ENST00000290390 | 2.4317 | 0.017663066 |
| A_23_P84892 | NM_032882 | PNMA6A | 2.4353 | 0.009740079 |
| A_23_P203629 | NM_173042 | IL18BP | 2.4359 | 0.024513437 |
| A_24_P317827 | NM_001042590 | C9orf127 | 2.436 | 0.029462825 |
| A_23_P106675 | NM_002661 | PLCG2 | 2.439 | 0.000804359 |
| A_23_P213415 | NM_003966 | SEMA5A | 2.4394 | 0.027228737 |
| A_23_P36496 | NM_016836 | RBMS1 | 2.4399 | 0.001645626 |
| A_23_P207400 | NM_007295 | BRCA1 | 2.4429 | 0.000803981 |
| A_24_P409728 | NM_015037 | KIAA0913 | 2.4452 | 0.002545249 |
| A_24_P358131 | XR_017211 | AK2P2 | 2.4474 | 0.046214609 |
| A_23_P154058 | NM_001034116 | EIF2B4 | 2.4474 | 0.021933236 |
| A_23_P164253 | NR_002181 | PPY2 | 2.4493 | 0.025037933 |
| A_23_P150379 | NM_144765 | MPZL2 | 2.4493 | 0.008123072 |
| A_24_P68819 | NM_152994 | CCDC128 | 2.4504 | 0.006851921 |
| A_23_P30024 | NM_003998 | NFKB1 | 2.4524 | 0.010561975 |
| A_24_P630875 | THC2647213 | THC2647213 | 2.4552 | 0.01113781 |
| A_24_P193570 | NM_016284 | CNOT1 | 2.456 | 0.030291265 |
| A_24_P922877 | NM_182923 | KLC1 | 2.4569 | 0.001842097 |
| A_23_P216984 | NM_016322 | RAB14 | 2.4575 | 0.016418689 |
| A_23_P77310 | NM_022473 | ZFP106 | 2.467 | 0.002241005 |
| A_23_P368896 | NM_013346 | SNX12 | 2.4706 | 0.02280198 |
| A_24_P357709 | NM_001009921 | VPS8 | 2.4723 | 0.002072996 |
| A_24_P295709 | BF446608 | BF446608 | 2.4728 | 0.017981822 |
| A_23_P93704 | NM_001004323 | LOC402573 | 2.4729 | 0.008876621 |
| A_23_P52451 | NM_025130 | HKDC1 | 2.4736 | 0.00488197 |
| A_32_P390781 | NM_020695 | REXO1 | 2.4743 | 0.024818358 |
| A_23_P157460 | NM_018660 | ZNF395 | 2.4751 | 0.020772584 |
| A_23_P207399 | NM_031858 | NBR1 | 2.4774 | 0.005090358 |
| A_24_P156576 | NM_017856 | GEMIN8 | 2.485 | 0.025193836 |
| A_23_P110253 | NM_000222 | KIT | 2.4857 | 0.004370688 |
| A_23_P54953 | NM_013260 | SAP30BP | 2.4878 | 0.029268051 |
| A_23_P68211 | NM_003124 | SPR | 2.4881 | 0.019747729 |
| A_24_P62521 | NM_000447 | PSEN2 | 2.4885 | 0.013513766 |
| A_23_P34546 | NM_006642 | SDCCAG8 | 2.4889 | 0.008064439 |
| A_24_P293120 | NM_024327 | ZNF576 | 2.4891 | 0.029101782 |
| A_23_P366312 | NM_138473 | SP1 | 2.4934 | 0.004834311 |
| A_24_P73920 | CR609948 | CR609948 | 2.4938 | 0.002793219 |
| A_23_P69491 | NM_001664 | RHOA | 2.4939 | 0.004059276 |
| A_23_P323143 | NM_024910 | ZNF767 | 2.4943 | 0.003129067 |
| A_24_P306788 | A_24_P306788 | A_24_P306788 | 2.4969 | 0.040554311 |
| A_23_P17620 | NM_052954 | CYYR1 | 2.4972 | 0.029659944 |
| A_23_P200443 | NM_003029 | SHC1 | 2.498 | 0.010270108 |
| A_23_P125475 | NM_018558 | GABRQ | 2.4996 | 0.026025679 |
| A_24_P167063 | NM_014803 | ZNF518A | 2.5014 | 0.003706909 |
| A_32_P430695 | NM_178339 | C3orf35 | 2.5022 | 0.033595373 |
| A_24_P15043 | NM_025010 | KLHL18 | 2.5055 | 0.004584483 |
| A_32_P206541 | NR_003955 | LOC647121 | 2.5056 | 0.001568644 |
| A_24_P534290 | A_24_P534290 | A_24_P534290 | 2.5061 | 0.005299771 |
| (+)E1A_r60_a104 | E1A_r60_a104 | E1A_r60_a104 | 2.5075 | 0.013222639 |
| A_23_P30024 | NM_003998 | NFKB1 | 2.5076 | 0.009400766 |
| A_23_P106080 | NM_032572 | RNASE7 | 2.5099 | 0.02747824 |
| A_23_P217872 | NM_006015 | ARID1A | 2.5109 | 0.003428852 |
| A_23_P217778 | NM_078629 | MSL3L1 | 2.5122 | 0.000138581 |
| A_24_P244470 | NM_194358 | RNF41 | 2.5132 | 0.028945934 |
| A_24_P403303 | NM_198513 | PHF20L1 | 2.5137 | 0.020786982 |
| A_23_P50020 | NM_018714 | COG1 | 2.515 | 0.009031104 |
| A_32_P180741 | NM_001010938 | TNK2 | 2.5167 | 0.048644468 |
| A_23_P30024 | NM_003998 | NFKB1 | 2.517 | 0.00679428 |
| A_32_P189952 | THC2667190 | THC2667190 | 2.52 | 0.025387734 |
| A_23_P500734 | NM_015100 | POGZ | 2.5201 | 0.003717168 |
| A_23_P396666 | NM_015079 | TBC1D2B | 2.5204 | 0.002547025 |
| A_23_P146811 | NM_205836 | FBXO38 | 2.5239 | 0.002172817 |
| A_23_P380965 | NM_016475 | C14orf100 | 2.5266 | 0.002466596 |
| A_23_P30024 | NM_003998 | NFKB1 | 2.5281 | 0.008142616 |
| A_23_P26336 | NM_014153 | ZC3H7A | 2.5289 | 0.002039322 |
| A_24_P55356 | CR596746 | CR596746 | 2.5293 | 0.003300281 |
| A_32_P174025 | NM_182704 | SELV | 2.5333 | 0.031465973 |
| A_23_P60002 | NM_014673 | TTC35 | 2.5362 | 0.009049554 |
| A_23_P25348 | NM_025247 | ACAD10 | 2.538 | 0.007291061 |
| A_24_P143138 | NM_004463 | FGD1 | 2.5387 | 0.02408119 |
| A_23_P2537 | NM_052845 | MMAB | 2.54 | 0.015033715 |
| A_23_P159382 | NM_014822 | SEC24D | 2.5404 | 0.004802744 |
| A_23_P213069 | NM_018126 | TMEM33 | 2.5417 | 0.001126903 |
| A_23_P635 | NM_007221 | PMF1 | 2.5417 | 0.001283573 |
| A_23_P129058 | NM_005154 | USP8 | 2.545 | 0.003967987 |
| A_23_P89762 | NM_194449 | PHLPP | 2.5453 | 0.005156455 |
| A_32_P74366 | ENST00000310421 | VCPIP1 | 2.546 | 0.002390637 |
| A_23_P347508 | NM_023077 | C1orf163 | 2.5494 | 0.033291698 |
| A_32_P53212 | AA045093 | AA045093 | 2.5501 | 0.048509041 |
| A_23_P75921 | NM_145803 | TRAF6 | 2.5545 | 0.048375038 |
| A_24_P334445 | NM_001017405 | MAEA | 2.5547 | 0.000353819 |
| A_23_P161237 | NM_004193 | GBF1 | 2.5572 | 0.013238083 |
| A_32_P194779 | NM_001099270 | ZBTB34 | 2.5572 | 0.000622157 |
| A_24_P936319 | BC030115 | BC030115 | 2.5582 | 0.006022274 |
| A_24_P12660 | THC2586764 | THC2586764 | 2.5612 | 0.001369747 |
| A_23_P139825 | NM_005748 | YAF2 | 2.5616 | 0.008591106 |
| A_23_P68740 | NM_000658 | AIRE | 2.5618 | 0.008804258 |
| A_23_P93442 | NM_015278 | SASH1 | 2.5629 | 2.74E−06 |
| A_24_P396662 | NM_147148 | GSTM4 | 2.564 | 0.002836917 |
| A_23_P366312 | NM_138473 | SP1 | 2.5663 | 0.003463589 |
| A_23_P81087 | NM_020395 | INTS12 | 2.5664 | 0.015134513 |
| A_32_P45875 | BC071826 | PSMD8 | 2.5683 | 0.032628926 |
| A_24_P366994 | NM_004309 | ARHGDIA | 2.5689 | 0.003839339 |
| A_32_P195387 | NR_015339 | LOC286144 | 2.5701 | 0.001858389 |
| A_24_P795283 | THC2541781 | THC2541781 | 2.5713 | 0.025695969 |
| A_23_P30024 | NM_003998 | NFKB1 | 2.5728 | 0.00933419 |
| A_32_P58464 | A_32_P58464 | A_32_P58464 | 2.5736 | 0.005804415 |
| A_23_P13998 | NM_017600 | GOLGA2L1 | 2.5749 | 0.023668942 |
| A_23_P163278 | NM_017851 | PARP16 | 2.576 | 0.000592543 |
| A_32_P4364 | NM_005745 | BCAP31 | 2.5769 | 0.010501866 |
| A_24_P350683 | BC012121 | SLC9A1 | 2.5817 | 0.048142628 |
| A_23_P254801 | NM_002660 | PLCG1 | 2.5821 | 0.003685261 |
| A_23_P320457 | NM_033088 | FAM40A | 2.5855 | 0.001463143 |
| A_32_P29784 | NM_001043352 | TPM3 | 2.5895 | 0.01356457 |
| A_23_P387031 | NM_173465 | COL23A1 | 2.5918 | 0.015589089 |
| A_24_P310667 | NM_031407 | HUWE1 | 2.5923 | 0.002267388 |
| A_24_P209765 | NM_007234 | DCTN3 | 2.5937 | 0.019270242 |
| A_23_P351232 | NM_173680 | ZNF775 | 2.5989 | 0.003107942 |
| A_24_P321752 | NM_015072 | TTLL5 | 2.5991 | 0.012129404 |
| A_23_P33420 | NM_030956 | TLR10 | 2.6008 | 0.008299618 |
| A_23_P37076 | NM_001015049 | BAG5 | 2.6054 | 0.00840999 |
| A_23_P217447 | NM_002294 | LAMP2 | 2.6098 | 8.21E−05 |
| A_23_P117387 | NM_054024 | MIA2 | 2.6119 | 0.021726242 |
| A_24_P39759 | NM_197939 | RNF135 | 2.6141 | 0.036119735 |
| A_23_P30024 | NM_003998 | NFKB1 | 2.6143 | 0.006756037 |
| A_24_P725365 | A_24_P725365 | A_24_P725365 | 2.6147 | 0.022682681 |
| A_24_P130026 | NM_001015049 | BAG5 | 2.6164 | 0.007647975 |
| A_24_P411899 | NM_183419 | RNF19A | 2.6184 | 0.001491501 |
| A_23_P105664 | NM_014167 | CCDC59 | 2.6202 | 0.016134015 |
| A_32_P145867 | NM_006188 | LOC4951 | 2.6264 | 0.032818732 |
| A_24_P254904 | NM_032236 | USP48 | 2.6306 | 0.002243573 |
| A_23_P19479 | NM_032454 | STK19 | 2.6333 | 0.01429352 |
| A_23_P30024 | NM_003998 | NFKB1 | 2.6354 | 0.006712125 |
| A_24_P199774 | NM_022164 | TINAGL1 | 2.6366 | 0.004132882 |
| A_23_P200443 | NM_003029 | SHC1 | 2.6386 | 0.006445911 |
| A_23_P200443 | NM_003029 | SHC1 | 2.6431 | 0.007097105 |
| A_23_P110253 | NM_000222 | KIT | 2.6455 | 0.004196448 |
| A_24_P337058 | NM_020307 | CCNL1 | 2.6481 | 0.000162233 |
| A_23_P200396 | NM_019118 | C1orf91 | 2.6492 | 0.001520073 |
| A_24_P941597 | AK055067 | AK055067 | 2.6523 | 0.040790898 |
| A_24_P181944 | NM_016436 | PHF20 | 2.6564 | 0.006195699 |
| A_23_P206981 | NM_007146 | VEZF1 | 2.6586 | 0.002840002 |
| A_23_P44088 | BC004179 | MGC2780 | 2.6615 | 0.032540536 |
| A_24_P391431 | NM_015975 | TAF9B | 2.6627 | 0.0110542 |
| A_23_P16242 | NM_021143 | ZNF20 | 2.6627 | 0.026782343 |
| A_23_P14915 | NM_001896 | CSNK2A2 | 2.6663 | 0.01712349 |
| A_23_P348264 | NM_144652 | LETM2 | 2.6701 | 0.026219177 |
| A_24_P641130 | NM_080686 | BAT2 | 2.6714 | 0.000298922 |
| A_23_P145016 | NM_006696 | BRD8 | 2.6718 | 0.007401314 |
| A_23_P147495 | NM_021946 | BC0RL1 | 2.6735 | 0.00063868 |
| A_23_P210300 | NM_018263 | ASXL2 | 2.6742 | 0.001710851 |
| A_24_P71661 | NM_006371 | CRTAP | 2.6798 | 0.035724793 |
| A_23_P107552 | NM_014939 | KIAA1012 | 2.6843 | 0.006180837 |
| A_32_P219011 | A_32_P219011 | A_32_P219011 | 2.6859 | 0.000812777 |
| A_23_P167559 | NM_144726 | RNF145 | 2.687 | 5.70E−05 |
| A_23_P114968 | NM_020440 | PTGFRN | 2.6904 | 0.004480088 |
| A_23_P307502 | AF211119 | C9orf53 | 2.696 | 0.002568644 |
| A_23_P91552 | NM_206965 | FTCD | 2.6962 | 0.032913558 |
| A_24_P804667 | NM_001043229 | LOC751071 | 2.6991 | 0.028889108 |
| A_23_P30024 | NM_003998 | NFKB1 | 2.7039 | 0.005305652 |
| A_23_P117447 | NM_017924 | C14orf119 | 2.7087 | 0.003338683 |
| A_24_P89413 | NM_021253 | TRIM39 | 2.709 | 0.002085786 |
| A_23_P110879 | NM_147686 | TRAF3IP2 | 2.7106 | 0.042248886 |
| A_23_P101774 | NM_000453 | SLC5A5 | 2.7153 | 0.039382172 |
| A_24_P349750 | ENST00000324414 | ENST00000324414 | 2.717 | 0.005864983 |
| A_24_P167030 | NM_207012 | AP3M1 | 2.7186 | 0.000548004 |
| A_23_P353048 | NM_198455 | SSPO | 2.7205 | 0.004201905 |
| A_23_P152970 | NM_016339 | RAPGEFL1 | 2.7211 | 0.0302114 |
| A_23_P56978 | NM_005975 | PTK6 | 2.7245 | 0.012642353 |
| A_23_P211957 | NM_001024847 | TGFBR2 | 2.725 | 0.000698698 |
| A_23_P201445 | NM_016076 | FAM152A | 2.7284 | 0.002352913 |
| A_23_P91293 | NM_004738 | VAPB | 2.7299 | 0.006961709 |
| A_23_P86242 | NM_018713 | SLC30A10 | 2.7363 | 0.035934465 |
| A_23_P325501 | NM_015358 | MORC3 | 2.7387 | 0.000678767 |
| A_23_P69491 | NM_001664 | RHOA | 2.7404 | 0.002658877 |
| A_24_P926125 | BC041879 | LOC390705 | 2.7435 | 0.000116132 |
| A_24_P250227 | NM_021724 | NR1D1 | 2.7483 | 0.013475807 |
| A_23_P51136 | NM_004040 | RHOB | 2.7503 | 0.001269037 |
| A_23_P89543 | NM_014804 | KIAA0753 | 2.7512 | 0.002915029 |
| A_23_P352226 | NM_014994 | MAPKBP1 | 2.7538 | 0.044236894 |
| A_32_P201773 | NM_015365 | AMMECR1 | 2.7553 | 0.008553896 |
| A_23_P73530 | NM_003828 | MTMR1 | 2.7561 | 0.001459754 |
| A_24_P792389 | THC2671169 | THC2671169 | 2.7564 | 0.015613921 |
| A_23_P304446 | NM_006213 | PHKG1 | 2.7688 | 0.027006438 |
| A_23_P366312 | NM_138473 | SP1 | 2.7698 | 0.002624002 |
| A_23_P259094 | NM_025231 | ZSCAN16 | 2.7759 | 0.004667783 |
| A_23_P12010 | NM_015997 | C1orf66 | 2.7776 | 0.009260444 |
| A_24_P203328 | NR_002223 | TPRXL | 2.7779 | 0.037887451 |
| A_24_P160969 | NM_001076787 | TP53I11 | 2.7802 | 0.020141659 |
| A_23_P91954 | NM_001296 | CCBP2 | 2.7814 | 0.016636335 |
| A_24_P313678 | NM_016060 | MED31 | 2.7869 | 0.005122134 |
| A_24_P68222 | AK023533 | AK023533 | 2.7881 | 0.003031697 |
| A_23_P256676 | NM_013396 | USP25 | 2.7886 | 0.004315983 |
| A_23_P30363 | NM_004199 | P4HA2 | 2.7917 | 0.047663734 |
| A_24_P316127 | AF255792 | C12orf3l | 2.7935 | 0.017149065 |
| A_23_P200443 | NM_003029 | SHC1 | 2.7943 | 0.00351583 |
| A_23_P69491 | NM_001664 | RHOA | 2.8035 | 0.002200539 |
| A_24_P72479 | NM_006409 | ARPC1A | 2.8071 | 0.003354848 |
| A_23_P405815 | NM_005609 | PYGM | 2.8153 | 0.005599658 |
| A_32_P42925 | NM_033546 | MRLC2 | 2.8161 | 0.001309487 |
| A_23_P60499 | NM_021224 | ZNF462 | 2.818 | 0.028766818 |
| A_23_P359897 | NM_030647 | JHDM1D | 2.8193 | 6.68E−05 |
| A_24_P84340 | NM_198828 | MAST4 | 2.8227 | 0.009992063 |
| A_23_P132454 | NM_020307 | CCNL1 | 2.8237 | 0.000255281 |
| A_32_P73217 | NM_020141 | C1orf119 | 2.8261 | 0.000607203 |
| A_23_P75609 | NM_014956 | CEP164 | 2.8261 | 0.011321724 |
| A_23_P5550 | NM_015317 | PUM2 | 2.8292 | 0.000398041 |
| A_24_P220548 | NM_001010845 | ACSM2A | 2.8304 | 0.02851178 |
| A_23_P203191 | NM_000039 | APOA1 | 2.8355 | 0.047396547 |
| A_23_P121898 | NM_024615 | PARP8 | 2.8369 | 7.44E−05 |
| A_24_P233878 | NM_017686 | GDAP2 | 2.838 | 0.02165042 |
| A_23_P49041 | NM_024956 | TMEM62 | 2.8383 | 0.018737009 |
| A_24_P630640 | AK126509 | CENTB1 | 2.8409 | 0.004566682 |
| A_32_P139894 | NM_007314 | ABL2 | 2.8432 | 0.012366974 |
| A_23_P111126 | L06175 | HCP5 | 2.8458 | 0.000196334 |
| A_32_P78488 | A_32_P78488 | A_32_P78488 | 2.8462 | 0.019600967 |
| A_23_P109410 | NM_030573 | THAP7 | 2.8497 | 0.006993931 |
| A_23_P163820 | NM_001080417 | ZNF629 | 2.8535 | 0.007249151 |
| A_23_P313223 | NM_138471 | LOC144097 | 2.8541 | 0.036600376 |
| A_32_P157465 | AK091904 | AK091904 | 2.8558 | 0.01242785 |
| A_23_P55880 | NM_144976 | ZNF564 | 2.856 | 0.001911352 |
| A_23_P102884 | NM_203446 | SYNJ1 | 2.8598 | 0.020845 |
| A_23_P30024 | NM_003998 | NFKB1 | 2.8732 | 0.004117531 |
| A_23_P22994 | NM_014223 | NFYC | 2.8774 | 0.002110682 |
| A_23_P95060 | NM_004443 | EPHB3 | 2.8775 | 0.03426634 |
| A_24_P787897 | NM_022166 | XYLT1 | 2.8788 | 0.004054761 |
| A_23_P110253 | NM_000222 | KIT | 2.8812 | 0.002109001 |
| A_23_P200443 | NM_003029 | SHC1 | 2.8824 | 0.00274339 |
| A_23_P91727 | NM_012407 | PICK1 | 2.8835 | 0.012749584 |
| A_23_P424597 | NM_152482 | C19orf25 | 2.8861 | 0.002884101 |
| A_23_P160638 | NM_199173 | BGLAP | 2.8871 | 0.00814298 |
| A_23_P319565 | NM_170753 | PGBD3 | 2.8878 | 0.003682373 |
| A_23_P107036 | NM_003876 | TMEM11 | 2.8883 | 0.002110026 |
| A_32_P205303 | AF070595 | AF070595 | 2.8912 | 0.007599152 |
| A_24_P346269 | THC2657554 | THC2657554 | 2.8916 | 0.025946824 |
| A_23_P54649 | NM_003789 | TRADD | 2.8951 | 0.002861939 |
| A_23_P254801 | NM_002660 | PLCG1 | 2.899 | 0.001905125 |
| A_32_P127105 | BC037740 | BC037740 | 2.9031 | 0.001224549 |
| A_23_P151209 | NM_030809 | FAM130A1 | 2.9033 | 0.008848845 |
| A_24_P150676 | BC011369 | TRAPPC2L | 2.9043 | 0.034193604 |
| A_23_P259663 | NM_014569 | ZKSCAN5 | 2.9064 | 0.031158719 |
| A_32_P212333 | BM479077 | BM479077 | 2.9089 | 0.023859081 |
| A_23_P254801 | NM_002660 | PLCG1 | 2.9095 | 0.001568479 |
| A_24_P374675 | NM_001037125 | UNKL | 2.9127 | 0.035163993 |
| A_23_P4714 | NM_006533 | MIA | 2.9176 | 0.001921027 |
| A_23_P26895 | NM_016261 | TUBD1 | 2.9192 | 0.009532721 |
| A_23_P106806 | NM_031948 | PRSS27 | 2.9195 | 0.036443459 |
| A_24_P154868 | NM_005588 | MEP1A | 2.921 | 0.038912161 |
| A_23_P93360 | NM_001136 | AGER | 2.9258 | 0.00054064 |
| A_23_P312837 | BC007366 | C9orf70 | 2.9277 | 0.022286927 |
| A_23_P254801 | NM_002660 | PLCG1 | 2.9312 | 0.00134469 |
| A_32_P67769 | THC2633447 | THC2633447 | 2.9336 | 0.032678496 |
| A_23_P54147 | NM_016350 | NIN | 2.9343 | 0.000229056 |
| A_23_P144622 | NM_005471 | GNPDA1 | 2.9356 | 0.02166529 |
| A_23_P108785 | NM_005721 | ACTR3 | 2.9356 | 0.000527032 |
| A_23_P118544 | NM_004859 | CLTC | 2.9368 | 0.003905054 |
| A_23_P306203 | NM_030754 | SAA2 | 2.9396 | 0.013544787 |
| A_24_P169773 | AF338232 | CTAGE4 | 2.9471 | 0.003627641 |
| A_23_P79155 | NM_001508 | GPR39 | 2.9471 | 0.003290552 |
| A_23_P43326 | NM_006415 | SPTLC1 | 2.9479 | 0.001934393 |
| A_24_P632160 | A_24_P632160 | A_24_P632160 | 2.9498 | 0.001710981 |
| A_23_P415533 | AK054879 | AK054879 | 2.9507 | 0.003426702 |
| A_32_P9700 | NM_002802 | PSMC1 | 2.9511 | 0.003759437 |
| A_24_P328646 | NM_005778 | RBM5 | 2.9514 | 0.000194263 |
| A_23_P254801 | NM_002660 | PLCG1 | 2.9536 | 0.001370937 |
| A_23_P104892 | NM_001418 | EIF4G2 | 2.954 | 0.000618349 |
| A_23_P157416 | NM_032164 | ZNF394 | 2.9545 | 0.001704552 |
| A_24_P376224 | AK098514 | LOC100130604 | 2.9578 | 4.08E−05 |
| A_24_P392958 | NM_144628 | TBC1D20 | 2.9592 | 0.002213388 |
| A_24_P409420 | A_24_P409420 | A_24_P409420 | 2.9601 | 0.022461015 |
| A_23_P4782 | NM_031896 | CACNG7 | 2.9617 | 0.018495018 |
| A_23_P430201 | NM_152446 | C14orf145 | 2.9665 | 0.017028664 |
| A_23_P208400 | NM_024876 | ADCK4 | 2.9725 | 0.002937315 |
| A_23_P210948 | NM_018244 | UQCC | 2.9728 | 0.008796692 |
| A_23_P71094 | NM_024523 | GCC1 | 2.9753 | 0.001107978 |
| A_23_P39616 | NM_006190 | ORC2L | 2.9757 | 0.001343016 |
| A_24_P211351 | A_24_P211351 | A_24_P211351 | 2.9776 | 0.000266657 |
| A_32_P12142 | THC2691941 | THC2691941 | 2.9781 | 0.000367769 |
| A_24_P273303 | XR_019523 | XR_019523 | 2.9853 | 0.034907657 |
| A_32_P34138 | ENST00000343959 | ENST00000343959 | 2.9872 | 0.027196325 |
| A_23_P200443 | NM_003029 | SHC1 | 2.9934 | 0.002677047 |
| A_24_P50759 | NM_000594 | TNF | 2.9934 | 0.008799074 |
| A_23_P9036 | NM_015117 | ZC3H3 | 2.9936 | 0.003237266 |
| A_23_P254801 | NM_002660 | PLCG1 | 2.9964 | 0.00103017 |
| A_23_P209215 | NM_005843 | STAM2 | 3.0058 | 0.003618269 |
| A_23_P71091 | NM_024523 | GCC1 | 3.0101 | 0.001469549 |
| A_23_P58443 | NM_020690 | ANKHD1-EIF4EBP3 | 3.0106 | 0.000277828 |
| A_23_P38100 | NM_173547 | TRIM65 | 3.0117 | 0.027125373 |
| A_23_P9621 | NM_001421 | ELF4 | 3.0129 | 0.013081871 |
| A_23_P134786 | NM_016018 | PHF20L1 | 3.0162 | 0.000282645 |
| A_24_P238040 | NM_004505 | USP6 | 3.0169 | 0.049419065 |
| A_24_P347488 | NM_002802 | PSMC1 | 3.0193 | 0.02326983 |
| A_23_P165295 | NM_002501 | NFIX | 3.0225 | 0.022782738 |
| A_32_P163147 | NM_182607 | VSIG1 | 3.0238 | 0.011973946 |
| A_32_P181131 | AW467174 | AW467174 | 3.0239 | 0.026356257 |
| A_23_P256868 | NM_152624 | DCP2 | 3.0267 | 0.001883377 |
| A_23_P154188 | NM_024545 | SAP130 | 3.0302 | 0.00015153 |
| A_24_P93012 | NR_002826 | LOC401010 | 3.0323 | 0.019845418 |
| A_23_P566 | NM_014947 | FOXJ3 | 3.0379 | 0.002285937 |
| A_24_P350644 | NM_013318 | KIAA0515 | 3.0389 | 0.000138549 |
| A_23_P71440 | NM_001002296 | GOLGA7 | 3.0542 | 0.000279784 |
| A_32_P121716 | AK057443 | AK057443 | 3.0547 | 0.044847725 |
| A_23_P110253 | NM_000222 | KIT | 3.0559 | 0.001208778 |
| A_23_P135966 | NM_003951 | SLC25A14 | 3.0592 | 0.001792551 |
| A_23_P75516 | NM_003626 | PPFIA1 | 3.0602 | 0.000566247 |
| A_23_P253368 | NM_018951 | HOXA10 | 3.0718 | 0.044038169 |
| A_23_P103256 | NM_021023 | CFHR3 | 3.0779 | 0.000862784 |
| A_23_P211047 | NM_206866 | BACH1 | 3.0781 | 0.000116558 |
| A_23_P200443 | NM_003029 | SHC1 | 3.0812 | 0.001546305 |
| A_23_P216188 | NM_001556 | IKBKB | 3.0812 | 0.004461652 |
| A_23_P256504 | NM_001633 | AMBP | 3.0812 | 0.025811232 |
| A_23_P360329 | NM_001039775 | AIM1L | 3.0835 | 0.000449941 |
| A_23_P322367 | AL050131 | AL050131 | 3.0899 | 0.004430889 |
| A_32_P68504 | NM_020923 | ZDBF2 | 3.0921 | 0.048096423 |
| A_23_P14975 | NM_032140 | C16orf48 | 3.0921 | 0.000143388 |
| A_24_P323974 | A_24_P323974 | A_24_P323974 | 3.0995 | 0.003678617 |
| A_23_P89334 | NM_003802 | MYH13 | 3.1064 | 0.026420364 |
| A_24_P230563 | NM_000417 | IL2RA | 3.1088 | 0.010921867 |
| A_24_P292831 | NM_025187 | C16orf70 | 3.1109 | 0.00631834 |
| A_32_P45297 | THC2653459 | THC2653459 | 3.1224 | 0.013354627 |
| A_32_P235159 | CR623684 | MSL3L2 | 3.1251 | 8.42E−05 |
| A_24_P476386 | THC2662279 | THC2662279 | 3.1258 | 0.000457386 |
| A_23_P425587 | NM_001001683 | MED11 | 3.1267 | 0.00109996 |
| A_23_P72187 | NM_004229 | MED14 | 3.1364 | 0.037108567 |
| A_24_P179504 | NM_144668 | WDR66 | 3.1417 | 0.020704634 |
| A_23_P140821 | NM_016948 | PARD6A | 3.1428 | 0.002962085 |
| A_23_P257250 | NM_015196 | KIAA0922 | 3.1443 | 0.001957223 |
| A_23_P114164 | NM_013444 | UBQLN2 | 3.1461 | 0.000441603 |
| A_32_P222664 | ENST00000372899 | UBR2 | 3.1491 | 0.000542843 |
| A_24_P941279 | AB002307 | SRCAP | 3.1582 | 0.00121549 |
| A_24_P205120 | NM_199367 | SPG7 | 3.1607 | 0.044630075 |
| A_23_P144807 | NM_001098811 | 8-Sep | 3.1607 | 0.010413748 |
| A_24_P554040 | BF509345 | BF509345 | 3.1709 | 0.035485381 |
| A_23_P412980 | NM_015132 | SNX13 | 3.1734 | 0.000745654 |
| A_24_P198178 | NM_001033953 | CALCA | 3.1762 | 0.049358445 |
| A_32_P70073 | AV749851 | AV749851 | 3.1802 | 0.010596862 |
| A_24_P264664 | NM_001271 | CHD2 | 3.1866 | 0.000363559 |
| A_23_P115407 | NM_146421 | GSTM1 | 3.1909 | 0.00771543 |
| A_23_P70213 | NM_000038 | APC | 3.194 | 0.027858384 |
| A_24_P347185 | NM_003717 | NPFF | 3.2058 | 0.000826159 |
| A_23_P77160 | NM_017610 | RNF111 | 3.2112 | 0.000156251 |
| A_23_P378329 | NM_003395 | WNT9A | 3.2122 | 0.008328477 |
| A_23_P88873 | NM_022041 | GAN | 3.2128 | 0.03234786 |
| A_32_P7308 | BX101927 | BX101927 | 3.214 | 0.004403436 |
| A_23_P402733 | NM_032195 | SON | 3.2159 | 0.015798246 |
| A_24_P417751 | A_24_P417751 | A_24_P417751 | 3.2295 | 0.039692007 |
| A_23_P200443 | NM_003029 | SHC1 | 3.2352 | 0.001010874 |
| (+)eQC-41 | EQC | EQC | 3.2373 | 0.040900653 |
| A_23_P431410 | BC065192 | C2orf12 | 3.2426 | 0.000464595 |
| A_23_P5131 | NM_016368 | ISYNA1 | 3.2449 | 0.002877823 |
| A_23_P351667 | NM_003812 | ADAM23 | 3.2509 | 4.16E−05 |
| A_24_P203689 | NM_198525 | KIF7 | 3.2515 | 0.030789682 |
| A_23_P142574 | NM_058165 | MOGAT1 | 3.2532 | 0.032278911 |
| A_23_P132248 | NM_019103 | ZMAT5 | 3.264 | 0.004319035 |
| A_23_P93321 | NM_007109 | TCF19 | 3.2797 | 0.040585369 |
| A_32_P76137 | ENST00000390256 | ENST00000390256 | 3.2831 | 0.049524172 |
| A_32_P225355 | NM_182485 | CPEB2 | 3.2915 | 0.007796683 |
| A_23_P30547 | NM_005565 | LCP2 | 3.2918 | 0.00048838 |
| A_24_P240137 | NM_004487 | GOLGB1 | 3.2991 | 0.000225286 |
| A_23_P12920 | NM_004584 | RAD9A | 3.3047 | 0.034400603 |
| A_24_P481314 | A_24_P481314 | A_24_P481314 | 3.3142 | 0.043451935 |
| A_32_P222096 | BX115782 | BX115782 | 3.3156 | 0.011705339 |
| A_23_P213551 | NM_001025105 | CSNK1A1 | 3.3392 | 0.002877395 |
| A_23_P116025 | NM_004268 | MED17 | 3.342 | 0.001947767 |
| A_32_P16263 | THC2557351 | THC2557351 | 3.3425 | 0.020675493 |
| A_24_P715865 | AW134708 | AW134708 | 3.3516 | 0.035265736 |
| A_24_P913482 | THC2634909 | THC2634909 | 3.3531 | 0.004442316 |
| A_23_P256973 | A_23_P256973 | A_23_P256973 | 3.3763 | 0.035148998 |
| A_23_P69493 | NM_001664 | RHOA | 3.3788 | 0.00056665 |
| A_24_P93728 | AK096488 | C1orf83 | 3.3955 | 0.012920034 |
| A_23_P44964 | NM_001010924 | C10orf38 | 3.406 | 0.009833231 |
| A_23_P152949 | NM_033413 | LRRC46 | 3.4069 | 0.024952632 |
| A_23_P213326 | NM_017747 | ANKHD1 | 3.4233 | 0.001664853 |
| A_32_P224522 | NM_024103 | SLC25A23 | 3.4258 | 0.004813068 |
| A_23_P142756 | NM_017759 | FLJ20309 | 3.4259 | 0.039044903 |
| A_24_P67280 | NM_178561 | CTAGE6 | 3.4365 | 0.001490161 |
| A_24_P366989 | THC2569387 | THC2569387 | 3.4527 | 0.011920772 |
| A_32_P537765 | NM_001083909 | GPR123 | 3.4576 | 0.041960159 |
| A_23_P159316 | NM_003571 | BFSP2 | 3.4654 | 0.024745316 |
| A_23_P343927 | NM_175065 | HIST2H2AB | 3.4722 | 0.014522416 |
| A_23_P205818 | NM_001024463 | HISPPD2A | 3.4805 | 0.02226563 |
| A_24_P942211 | NM_001110781 | RP11-345P4.4 | 3.4833 | 8.69E−05 |
| A_24_P153596 | AK092445 | LOC100132014 | 3.4859 | 0.045279373 |
| A_32_P440768 | NR_002710 | ALOX12P2 | 3.488 | 0.019026806 |
| A_23_P354326 | AK056275 | FLJ31713 | 3.4943 | 0.022807965 |
| A_23_P138089 | NM_000530 | MPZ | 3.4952 | 0.006904072 |
| A_23_P322519 | NM_005438 | FOSL1 | 3.4963 | 0.016376566 |
| A_32_P20240 | BX648857 | SP3 | 3.4984 | 0.000153042 |
| A_23_P137856 | NM_002456 | MUC1 | 3.5008 | 0.003590676 |
| A_32_P147297 | CR592318 | CR592318 | 3.5012 | 0.006328584 |
| A_24_P133933 | NM_004309 | ARHGDIA | 3.5052 | 0.009783101 |
| A_24_P935889 | NM_004035 | ACOX1 | 3.5054 | 0.045670195 |
| A_23_P207520 | Z74615 | COL1A1 | 3.5155 | 0.040856181 |
| A_23_P201887 | NM_005057 | RBBP5 | 3.5183 | 0.014480606 |
| A_23_P115785 | NM_145235 | FANK1 | 3.5314 | 5.49E−05 |
| A_23_P79416 | NM_025133 | FBXO11 | 3.5369 | 0.000726679 |
| A_24_P393611 | NM_138736 | GNAO1 | 3.5445 | 0.004657014 |
| A_24_P372932 | NM_000319 | PEX5 | 3.5536 | 0.012062076 |
| A_24_P45728 | NM_020770 | CGN | 3.5552 | 0.014142675 |
| A_23_P156209 | NM_033414 | ZNF622 | 3.5554 | 0.005703543 |
| A_24_P935252 | X90874 | FXR1 | 3.5606 | 0.015927032 |
| A_24_P230057 | XR_016590 | KRT18P49 | 3.5638 | 0.002756881 |
| ETG10_195139 | ETG10_195139 | ETG10_195139 | 3.5726 | 0.012487436 |
| A_24_P169688 | NM_005931 | MICB | 3.5842 | 0.000631128 |
| A_23_P100240 | NM_004062 | CDH16 | 3.5844 | 0.040210898 |
| A_24_P218814 | NM_002905 | RDH5 | 3.586 | 0.000154964 |
| A_23_P346390 | AK094877 | CXorf36 | 3.5874 | 0.024491844 |
| AT_nD_3 | AT_nD_3 | AT_nD_3 | 3.5912 | 0.012718213 |
| A_24_P921906 | AF229803 | AF229803 | 3.5917 | 0.034973138 |
| A_23_P38987 | NM_014516 | CNOT3 | 3.5921 | 6.56E−05 |
| A_23_P410587 | NM_024900 | PHF17 | 3.5982 | 0.000665182 |
| A_23_P58967 | NM_014827 | ZC3H11A | 3.6039 | 0.000472924 |
| A_24_P408715 | AK098811 | LOC285398 | 3.6107 | 0.03906667 |
| A_23_P118174 | NM_005030 | PLK1 | 3.6138 | 0.031389299 |
| A_23_P70547 | NM_005527 | HSPA1L | 3.6148 | 0.000332365 |
| A_24_P243560 | AK314822 | AK314822 | 3.6215 | 0.012488874 |
| A_24_P150160 | NM_004265 | FADS2 | 3.6238 | 0.002683227 |
| A_23_P156017 | NM_022130 | GOLPH3 | 3.6322 | 0.002449774 |
| A_23_P258978 | NM_002077 | GOLGA1 | 3.6607 | 0.002445471 |
| A_24_P66672 | NM_001080453 | INTS1 | 3.6663 | 0.046129256 |
| A_24_P119745 | NM_212482 | FN1 | 3.6686 | 0.035033673 |
| A_24_P350261 | ENST00000332902 | ENST00000332902 | 3.6749 | 0.022115948 |
| A_24_P70183 | NM_001040113 | MYH11 | 3.676 | 0.00668511 |
| A_23_P127467 | NM_032325 | EIF1AD | 3.6777 | 0.006057611 |
| A_32_P505133 | NM_178830 | C19orf47 | 3.6835 | 0.011774314 |
| A_24_P399694 | NM_033089 | ZCCHC3 | 3.6971 | 0.000799787 |
| A_24_P392030 | ENST00000331447 | ENST00000331447 | 3.6996 | 0.000122446 |
| A_23_P405942 | NM_015155 | LARP5 | 3.7021 | 0.000184178 |
| A_23_P115407 | NM_146421 | GSTM1 | 3.7027 | 0.004119647 |
| A_23_P11644 | NM_006945 | SPRR2D | 3.7191 | 0.008626861 |
| A_23_P4443 | NM_018488 | TBX4 | 3.7381 | 0.018824708 |
| A_24_P21752 | NM_032811 | TBRG1 | 3.7389 | 0.000365503 |
| A_23_P100676 | NM_017575 | SMG6 | 3.7835 | 0.001400395 |
| A_23_P122254 | NM_005124 | NUP153 | 3.7843 | 0.000262396 |
| A_23_P64611 | NM_176798 | P2RY6 | 3.7868 | 0.000126326 |
| A_23_P58622 | NM_181838 | UBE2D2 | 3.7878 | 0.001924184 |
| A_24_P155815 | NM_032382 | COG8 | 3.7949 | 0.001193209 |
| A_32_P14582 | NM_001013640 | LOC388503 | 3.8074 | 0.030920926 |
| A_24_P515815 | AI263220 | AI263220 | 3.8146 | 0.029206701 |
| A_24_P25137 | NM_000740 | CHRM3 | 3.8182 | 0.034501432 |
| A_23_P170238 | NM_020815 | PCDH10 | 3.8233 | 0.046162554 |
| A_24_P19993 | NM_014827 | ZC3H11A | 3.8303 | 0.000235568 |
| A_23_P157593 | NM_004198 | CHRNA6 | 3.8438 | 0.041007561 |
| A_23_P98616 | NM_153277 | SLC22A6 | 3.8463 | 0.049232352 |
| A_23_P211572 | NM_145733 | 3-Sep | 3.8594 | 0.044265304 |
| A_32_P210622 | THC2660732 | THC2660732 | 3.8854 | 0.033236752 |
| A_23_P77304 | NM_004644 | AP3B2 | 3.8858 | 0.009061602 |
| A_24_P925124 | THC2515402 | THC2515402 | 3.8877 | 0.010626054 |
| A_23_P148916 | NM_148909 | OSBPL9 | 3.8923 | 9.86E−06 |
| A_32_P70724 | NM_006618 | JARID1B | 3.8929 | 0.000160759 |
| A_32_P57870 | NM_002802 | PSMC1 | 3.9183 | 0.003220979 |
| A_32_P56584 | AW015165 | AW015165 | 3.9284 | 0.006243035 |
| A_24_P12397 | NM_018965 | TREM2 | 3.9332 | 0.043106425 |
| A_32_P4985 | NM_015215 | CAMTA1 | 3.9437 | 0.016278936 |
| A_23_P50399 | NM_138353 | LOC90379 | 3.9573 | 0.000448253 |
| A_32_P468341 | NM_173507 | C1orf127 | 3.9606 | 0.01952715 |
| A_32_P143048 | NM_004799 | ZFYVE9 | 3.9745 | 0.037809133 |
| A_32_P200608 | BC049373 | LOC151300 | 3.9762 | 0.018282854 |
| A_24_P280833 | BC014218 | LOC100129138 | 4.0081 | 0.002539551 |
| A_24_P934541 | A_24_P934541 | A_24_P934541 | 4.0092 | 0.019416248 |
| A_23_P142969 | NM_021088 | ZNF2 | 4.012 | 0.022565746 |
| A_23_P128956 | NM_021260 | ZFYVE1 | 4.0418 | 0.000196454 |
| A_24_P307712 | AK057787 | AK057787 | 4.0459 | 0.006434409 |
| A_24_P936975 | AW337952 | AW337952 | 4.0485 | 0.034883007 |
| A_24_P84021 | XM_001714867 | LOC441016 | 4.0609 | 0.000851673 |
| A_24_P7021 | XR_016847 | XR_016847 | 4.0651 | 0.042772863 |
| A_32_P87972 | AA579646 | AA579646 | 4.0844 | 0.043619789 |
| A_32_P151875 | NM_020781 | ZNF398 | 4.1079 | 0.000933171 |
| A_24_P132813 | NM_001040103 | OPALIN | 4.1197 | 0.026066745 |
| A_23_P28761 | NM_001030004 | HNF4A | 4.1247 | 0.014610959 |
| A_23_P26965 | NM_005408 | CCL13 | 4.1266 | 0.032409804 |
| A_23_P424637 | AK098143 | LOC283861 | 4.1582 | 0.033859974 |
| A_23_P55286 | NM_002544 | OMG | 4.1708 | 0.0223399 |
| A_24_P291074 | AF305819 | AF305819 | 4.1753 | 0.0132317 |
| A_23_P61524 | NM_022903 | CCDC71 | 4.1822 | 0.01863663 |
| A_23_P3643 | NM_001374 | DNASE1L2 | 4.1913 | 0.004989844 |
| A_23_P615 | NM_014215 | INSRR | 4.1993 | 0.034678415 |
| A_23_P337875 | NM_001040202 | PAQR3 | 4.2023 | 0.027431599 |
| A_23_P401098 | NM_153211 | C18orf17 | 4.2033 | 0.000461193 |
| A_23_P502750 | NM_002392 | MDM2 | 4.2119 | 0.027956312 |
| A_24_P920102 | BC015824 | SYF2 | 4.2555 | 0.000361384 |
| A_32_P30421 | AA975908 | AA975908 | 4.2634 | 0.019788356 |
| A_24_P109524 | NM_018984 | SSH1 | 4.293 | 0.000156212 |
| A_32_P186121 | NM_002637 | PHKAl | 4.3158 | 0.024425909 |
| A_23_P133799 | NM_201523 | KLC4 | 4.3379 | 0.00104076 |
| A_24_P23921 | NM_001080465 | LOC388381 | 4.3414 | 0.012486676 |
| A_23_P409449 | NM_005421 | TAL2 | 4.3415 | 0.008086388 |
| A_24_P214043 | AF289552 | LOC100133287 | 4.3493 | 0.032575309 |
| A_23_P123539 | NM_002717 | PPP2R2A | 4.3566 | 8.23E−05 |
| A_23_P82762 | NM_001042416 | ZNF596 | 4.367 | 0.031160842 |
| A_24_P658584 | NM_015278 | SASH1 | 4.37 | 5.43E−05 |
| A_23_P9875 | NM_007170 | TESK2 | 4.3732 | 0.000198176 |
| A_23_P321846 | NM_002251 | KCNS1 | 4.3792 | 0.014351179 |
| A_23_P19020 | NM_005460 | SNCAIP | 4.4289 | 0.049608023 |
| A_23_P108216 | NM_005031 | FXYD1 | 4.4471 | 0.028424095 |
| A_24_P942954 | A_24_P942954 | A_24_P942954 | 4.4475 | 0.009293485 |
| A_24_P315674 | XR_018514 | XR_018514 | 4.485 | 0.036048833 |
| A_23_P110253 | NM_000222 | KIT | 4.4858 | 3.15E−05 |
| A_23_P72651 | NM_001077693 | ECSM2 | 4.4937 | 0.040137791 |
| A_23_P53417 | NM_006741 | PPP1R1A | 4.4947 | 0.021488538 |
| A_32_P155035 | AK096500 | AK096500 | 4.5086 | 0.044729502 |
| A_24_P340390 | NM_182485 | CPEB2 | 4.5351 | 0.001141092 |
| A_32_P35839 | A_32_P35839 | A_32_P35839 | 4.5424 | 0.016249227 |
| A_23_P213020 | NM_001085382 | PSAPL1 | 4.5464 | 0.004434361 |
| A_23_P88831 | NM_001076785 | SLC7A6 | 4.5479 | 0.00270926 |
| A_24_P478350 | NM_001017924 | LRIT2 | 4.5547 | 0.009932076 |
| A_24_P392947 | A_24_P392947 | A_24_P392947 | 4.5594 | 0.028952923 |
| A_23_P41588 | NM_012208 | HARS2 | 4.5695 | 4.23E−05 |
| A_24_P286465 | NM_033224 | PURB | 4.5909 | 0.000301802 |
| A_23_P70566 | NM_022110 | FKBPL | 4.6553 | 0.020545319 |
| A_23_P357724 | NM_002246 | KCNK3 | 4.6596 | 0.025460194 |
| A_23_P81018 | NM_005390 | PDHA2 | 4.6773 | 0.032950797 |
| A_23_P208085 | NM_001025101 | MBP | 4.683 | 0.000847089 |
| A_24_P247269 | AL834492 | C8orf66 | 4.7132 | 0.041882919 |
| A_23_P259135 | NM_014071 | NCOA6 | 4.73 | 7.44E−05 |
| (+)eQC-41 | EQC | EQC | 4.7674 | 0.012273616 |
| A_24_P230358 | XR_018215 | XR_018215 | 4.7708 | 0.012150623 |
| A_24_P56330 | NM_152324 | C13orf16 | 4.781 | 0.00161551 |
| A_32_P235840 | AL526449 | AL526449 | 4.8157 | 0.000464925 |
| A_23_P70213 | NM_000038 | APC | 4.8232 | 0.030514271 |
| A_24_P917316 | AF035790 | AF035790 | 4.8341 | 0.0106655 |
| A_23_P30582 | NM_004174 | SLC9A3 | 4.8358 | 0.005519457 |
| A_23_P1912 | NM_207341 | ZP1 | 4.8359 | 0.003379707 |
| A_32_P886589 | NM_175895 | C12orf61 | 4.9056 | 0.032848976 |
| A_24_P179569 | NM_000227 | LAMA3 | 4.9272 | 0.047524839 |
| A_23_P436048 | NM_012152 | LPAR3 | 4.9383 | 0.033927086 |
| A_23_P152462 | NM_052874 | STX1B | 4.9454 | 0.015038062 |
| A_24_P926895 | AF007190 | AF007190 | 4.9669 | 0.033577258 |
| A_23_P55099 | NM_002737 | PRKCA | 4.9796 | 0.015322314 |
| A_23_P115805 | NM_201564 | SYCE1 | 4.9822 | 0.032725034 |
| A_24_P186079 | AK096324 | FLJ39005 | 4.9859 | 0.042085978 |
| A_23_P349321 | NM_022166 | XYLT1 | 5.0191 | 0.034389212 |
| A_24_P833067 | THC2680363 | THC2680363 | 5.0528 | 0.028365784 |
| A_23_P155556 | NM_001040199 | CLDND1 | 5.0934 | 9.26E−07 |
| A_23_P140562 | NM_001040655 | TTC23 | 5.0981 | 0.048935535 |
| A_24_P940738 | A_24_P940738 | A_24_P940738 | 5.0999 | 0.045385099 |
| A_24_P237896 | XR_018580 | LOC392787 | 5.1478 | 0.026832768 |
| A_23_P369237 | NM_004797 | ADIPOQ | 5.1706 | 0.01354588 |
| A_23_P257445 | AF119871 | AF119871 | 5.1814 | 0.001033493 |
| A_24_P306644 | XR_018532 | XR_018532 | 5.1933 | 0.02205048 |
| A_32_P148745 | AK027618 | FLJ14712 | 5.2056 | 0.000295385 |
| A_32_P4595 | AF010236 | AF010236 | 5.2232 | 0.009370349 |
| A_24_P919974 | NM_000537 | REN | 5.243 | 0.005734509 |
| A_23_P398943 | NM_001047605 | KIR3DX1 | 5.2532 | 0.019497045 |
| A_32_P93841 | BG682198 | BG682198 | 5.2572 | 0.02722624 |
| A_24_P124624 | NM_002543 | OLR1 | 5.2873 | 0.03129368 |
| A_23_P20927 | NM_003747 | TNKS | 5.3 | 0.047895269 |
| A_32_P15288 | BX114329 | BX114329 | 5.3538 | 0.026930392 |
| A_23_P55099 | NM_002737 | PRKCA | 5.374 | 0.024933605 |
| A_32_P119869 | BG185346 | BG185346 | 5.3912 | 0.002634288 |
| A_24_P221745 | BC008048 | LOC440934 | 5.4046 | 0.043621919 |
| A_23_P81094 | THC2603089 | THC2603089 | 5.4056 | 0.007503631 |
| A_23_P56433 | NM_032411 | C2orf40 | 5.4288 | 0.046026574 |
| A_24_P229884 | BC022385 | LOC440335 | 5.4654 | 0.022670903 |
| A_24_P202637 | BC017357 | ZNF765 | 5.5083 | 0.041645594 |
| A_23_P301328 | ENST00000400399 | ENST00000400399 | 5.5214 | 0.000332181 |
| A_24_P929729 | X65665 | SOX3 | 5.5617 | 0.03442692 |
| A_32_P136776 | AK000872 | LOC644192 | 5.5867 | 0.02606886 |
| A_24_P538839 | BC020376 | BC020376 | 5.6275 | 0.000352583 |
| A_24_P932785 | AF318320 | GATA4 | 5.7113 | 0.011869846 |
| A_24_P941217 | AK096323 | SGPP2 | 5.7271 | 0.043490073 |
| A_32_P233379 | CD671494 | CD671494 | 5.7454 | 0.047220947 |
| A_24_P924932 | NM_001010922 | BCL2L15 | 5.7718 | 0.04786343 |
| A_32_P96124 | A_32_P96124 | A_32_P96124 | 5.7774 | 0.000576367 |
| A_32_P232808 | AF289566 | AF289566 | 5.8376 | 0.049132812 |
| A_23_P257387 | NM_006529 | GLRA3 | 5.8615 | 0.041729187 |
| AT_ssH_GD_5 | AT_ssH_GD_5 | AT_ssH_GD_5 | 5.9508 | 0.010862368 |
| A_24_P111106 | NM_000800 | FGF1 | 5.9646 | 0.012021333 |
| A_23_P253168 | A_23_P253168 | A_23_P253168 | 5.9827 | 0.045922235 |
| A_24_P926529 | BC029395 | MPHOSPH6 | 6.0337 | 0.012376702 |
| A_32_P209702 | THC2681437 | THC2681437 | 6.0922 | 0.038496372 |
| A_23_P4850 | NM_145233 | ZNF625 | 6.0931 | 0.029574305 |
| A_23_P311410 | AL831885 | FLCN | 6.1072 | 0.00030197 |
| A_23_P123015 | AK026020 | WBSCR23 | 6.1277 | 0.037125572 |
| A_24_P216456 | NM_001004343 | MAP1LC3C | 6.199 | 0.03003372 |
| A_23_P331400 | NM_199340 | LRRC37A3 | 6.3067 | 0.014048684 |
| A_23_P331770 | NM_018561 | USP49 | 6.3114 | 0.005427464 |
| A_24_P307653 | NM_182767 | SLC6A15 | 6.317 | 0.028701288 |
| A_24_P144773 | NM_144726 | RNF145 | 6.3437 | 0.001566012 |
| A_23_P128396 | NM_194271 | RNF34 | 6.4331 | 8.07E−05 |
| A_24_P943411 | A_24_P943411 | A_24_P943411 | 6.4876 | 0.02652615 |
| A_24_P915338 | AF209507 | AF209507 | 6.5009 | 0.043775664 |
| A_32_P66684 | THC2689970 | THC2689970 | 6.5351 | 0.018317179 |
| A_23_P381505 | AK075525 | FLJ14712 | 6.5402 | 0.031687892 |
| A_23_P56659 | NM_133637 | DQX1 | 6.5695 | 0.008129882 |
| A_32_P195647 | NM_030624 | KLHL15 | 6.5915 | 0.032694275 |
| A_23_P153128 | AF295728 | C18orf2 | 6.6248 | 0.030545772 |
| A_23_P303833 | NM_174934 | SCN4B | 6.663 | 0.00107702 |
| A_32_P108066 | THC2616558 | THC2616558 | 6.6786 | 0.035010939 |
| A_32_P16018 | THC2729895 | THC2729895 | 6.6991 | 0.023660055 |
| A_24_P325992 | NM_002310 | LIFR | 6.7289 | 0.032581726 |
| A_24_P634768 | AK026416 | FLJ22763 | 6.7572 | 0.019201222 |
| (−)3xSLv1 | NegativeControl | NegativeControl | 6.7736 | 0.04353629 |
| A_23_P51311 | NM_001010857 | LELP1 | 6.8492 | 0.048014036 |
| ETG10_236652 | ETG10_236652 | ETG10_236652 | 7.1255 | 0.003634884 |
| A_23_P358709 | NM_020731 | AHRR | 7.1962 | 3.10E−05 |
| A_24_P418216 | XR_018299 | XR_018299 | 7.4522 | 0.00582353 |
| A_32_P42705 | BC017721 | LOC100144602 | 7.4749 | 0.019642723 |
| A_24_P37300 | AF052115 | AF052115 | 7.5274 | 0.002673278 |
| A_23_P256425 | NM_014479 | ADAMDEC1 | 7.5286 | 0.003348314 |
| A_24_P945298 | AK128700 | ZNF788 | 7.5641 | 0.032313631 |
| A_24_P262321 | NM_173622 | CDRT4 | 7.5806 | 0.026951642 |
| A_24_P260122 | NM_145753 | PHLDB2 | 7.6035 | 0.034145543 |
| A_24_P523368 | BG461173 | BG461173 | 7.7762 | 0.000182967 |
| A_24_P285522 | NM_003618 | MAP4K3 | 7.7816 | 0.029593204 |
| A_24_P324538 | A_24_P324538 | A_24_P324538 | 7.8027 | 0.024455017 |
| A_32_P142195 | A_32_P142195 | A_32_P142195 | 7.8043 | 0.025841592 |
| A_24_P717824 | A_24_P717824 | A_24_P717824 | 7.8274 | 0.007752257 |
| A_23_P128613 | NM_024089 | KDELC1 | 7.8275 | 0.02951137 |
| A_23_P128613 | NM_024089 | KDELC1 | 7.8369 | 0.01358842 |
| A_23_P63096 | NM_001004319 | VHLL | 7.8435 | 0.04973222 |
| A_32_P47157 | AK054925 | LOC92973 | 7.8672 | 0.021134189 |
| AT_L_5 | AT_L_5 | AT_L_5 | 7.9384 | 0.049242309 |
| A_32_P117957 | CV323908 | CV323908 | 7.9891 | 0.030787292 |
| A_23_P204654 | NM_000899 | KITLG | 8.1119 | 0.030095847 |
| A_32_P426675 | BC009366 | BC009366 | 8.3702 | 0.023375082 |
| AT_T_M | AT_T_M | AT_T_M | 8.3793 | 0.037522768 |
| A_23_P95244 | AX721299 | AX721299 | 8.4844 | 0.034916838 |
| A_24_P865672 | CR595483 | CR595483 | 8.5004 | 0.014032334 |
| A_24_P162226 | NM_015347 | RIMBP2 | 8.6012 | 0.010439127 |
| A_24_P59236 | DQ227570 | RESP18 | 8.6409 | 0.004747219 |
| A_32_P81676 | AL832535 | LOC157627 | 8.6839 | 0.000128664 |
| A_24_P125469 | NM_006033 | LIPG | 8.697 | 0.038306921 |
| A_24_P418699 | ENST00000332666 | ENST00000332666 | 8.7335 | 0.007407263 |
| A_23_P128613 | NM_024089 | KDELC1 | 8.7999 | 0.037566289 |
| A_32_P195501 | THC2659392 | THC2659392 | 8.8731 | 0.005052351 |
| A_24_P917729 | BC009775 | AGK | 8.8772 | 0.046667055 |
| A_24_P268323 | NM_173532 | FNDC7 | 8.8895 | 0.02342991 |
| A_23_P257881 | A_23_P257881 | A_23_P257881 | 8.9742 | 0.023992281 |
| A_24_P350832 | AJ437018 | AGTPBP1 | 9.1007 | 0.000401876 |
| A_24_P767725 | XR_017120 | LOC646090 | 9.1298 | 0.024370161 |
| A_32_P185530 | A_32_P185530 | A_32_P185530 | 9.2436 | 0.04364456 |
| A_24_P911872 | AK094726 | AK094726 | 9.2653 | 0.02403276 |
| A_24_P944113 | AI401434 | AI401434 | 9.3034 | 0.0203907 |
| A_23_P150619 | NM_006458 | TRIM3 | 9.3069 | 0.01214165 |
| A_32_P190303 | NM_198461 | LONRF2 | 9.3104 | 0.017638836 |
| A_24_P747625 | BQ003493 | BQ003493 | 9.4853 | 0.019022612 |
| A_32_P185393 | THC2747684 | THC2747684 | 9.6241 | 0.023675952 |
| A_23_P214803 | NM_014841 | SNAP91 | 9.6916 | 0.020286878 |
| A_23_P32474 | NM_022842 | CDCP1 | 9.7593 | 0.047745465 |
| A_24_P771821 | BF478043 | BF478043 | 9.955 | 0.031090389 |
| A_23_P144005 | NM_015931 | C3orf32 | 10.013 | 0.001012253 |
| A_23_P13493 | NM_152430 | OR51E1 | 10.013 | 0.042796762 |
| A_23_P5968 | NM_003008 | SEMG2 | 10.082 | 0.042530448 |
| A_24_P248031 | BC017945 | BC017945 | 10.144 | 0.040656693 |
| A_24_P900555 | XR_019510 | XR_019510 | 10.163 | 0.042101611 |
| A_32_P47410 | BX648527 | FLJ43080 | 10.233 | 0.023701913 |
| A_24_P925342 | AB209275 | MAN1C1 | 10.242 | 0.047222716 |
| A_24_P222165 | AK074381 | GAB1 | 10.329 | 0.036805584 |
| A_23_P11081 | NM_003886 | AKAP4 | 10.405 | 0.00821241 |
| A_23_P259270 | NM_003948 | CDKL2 | 10.617 | 0.047767592 |
| A_23_P148255 | NM_153488 | MAGEA2B | 10.628 | 0.006440463 |
| A_23_P62099 | NM_016249 | MAGEC2 | 10.644 | 0.027787934 |
| A_24_P407439 | AA020958 | AA020958 | 10.799 | 0.037369204 |
| A_24_P915030 | A_24_P915030 | A_24_P915030 | 10.829 | 0.03084932 |
| A_24_P177932 | NM_001080479 | RGNEF | 10.853 | 0.049836697 |
| A_23_P71213 | NM_130768 | ASZ1 | 10.858 | 0.018812431 |
| A_23_P155807 | NM_024936 | ZCCHC4 | 10.863 | 0.007168019 |
| A_32_P495952 | BC020909 | BC020909 | 10.969 | 0.013356828 |
| A_23_P16630 | NM_017506 | OR7A5 | 11.545 | 0.042296746 |
| A_32_P25647 | BX088949 | BX088949 | 11.66 | 0.02728166 |
| A_23_P140797 | NM_001796 | CDH8 | 11.671 | 0.010634362 |
| A_24_P919265 | A_24_P919265 | A_24_P919265 | 11.691 | 0.025711428 |
| A_23_P255027 | BC007304 | MGC15705 | 11.747 | 0.006566641 |
| A_24_P923128 | THC2562425 | THC2562425 | 11.759 | 0.025679346 |
| A_24_P114142 | NM_005076 | CNTN2 | 11.811 | 0.022911652 |
| A_23_P77212 | NM_022784 | IQCH | 11.823 | 0.026591738 |
| A_23_P251974 | NM_007323 | ZFYVE9 | 11.911 | 0.026546736 |
| A_32_P233877 | NM_207411 | XKR5 | 12.075 | 0.027730306 |
| A_32_P32537 | BF805854 | BF805854 | 12.106 | 0.008290121 |
| A_23_P252062 | NM_138711 | PPARG | 12.13 | 0.001440753 |
| A_32_P77762 | BX091687 | BX091687 | 12.155 | 0.02338744 |
| A_23_P49145 | NM_152338 | ZG16 | 12.163 | 0.004973854 |
| A_23_P145159 | NM_001371 | DNAH8 | 12.186 | 0.019902805 |
| A_24_P944336 | BC036071 | ELAVL4 | 12.191 | 0.035295821 |
| A_24_P608931 | AW043836 | AW043836 | 12.196 | 0.023695309 |
| A_24_P510081 | AK026477 | AK026477 | 12.233 | 0.024783528 |
| A_24_P44992 | ENST00000400371 | ENST00000400371 | 12.271 | 0.035130999 |
| A_24_P917530 | A_24_P917530 | A_24_P917530 | 12.326 | 0.029455701 |
| A_24_P915883 | A_24_P915883 | A_24_P915883 | 12.329 | 0.006755719 |
| A_23_P217468 | NM_002025 | AFF2 | 12.346 | 0.040646677 |
| A_23_P45068 | AF006822 | MYT2 | 12.411 | 0.032305981 |
| A_32_P199462 | NM_001099334 | LOC389073 | 12.423 | 0.030256049 |
| A_23_P55099 | NM_002737 | PRKCA | 12.464 | 0.001768482 |
| A_24_P242440 | NM_003780 | B4GALT2 | 12.539 | 0.03817505 |
| A_24_P206328 | NM_005020 | PDE1C | 12.567 | 0.001689837 |
| A_24_P857669 | AB002438 | AB002438 | 12.646 | 0.032553653 |
| A_24_P926272 | W95720 | W95720 | 12.704 | 0.004265021 |
| A_23_P130027 | NM_017957 | EPN3 | 12.803 | 0.02269279 |
| A_23_P48022 | NM_023918 | TAS2R8 | 12.804 | 0.014602615 |
| A_24_P118052 | NM_199254 | TPTE2 | 12.935 | 0.018290142 |
| A_32_P28151 | THC2670307 | THC2670307 | 12.988 | 0.023000867 |
| A_24_P270747 | NM_153688 | ZFP1 | 13.073 | 0.04454237 |
| A_24_P937931 | A_24_P937931 | A_24_P937931 | 13.116 | 0.021567781 |
| A_24_P922647 | A_24_P922647 | A_24_P922647 | 13.143 | 0.026723974 |
| A_32_P234132 | THC2752010 | THC2752010 | 13.197 | 0.025817894 |
| A_24_P924979 | CB052856 | CB052856 | 13.206 | 0.045383911 |
| A_24_P247640 | THC2489251 | THC2489251 | 13.27 | 0.049723153 |
| A_24_P123833 | NM_012431 | SEMA3E | 13.307 | 0.032641545 |
| A_23_P122052 | NM_001008397 | LOC493869 | 13.361 | 0.031957198 |
| A_24_P912186 | A_24_P912186 | A_24_P912186 | 13.362 | 0.034536746 |
| A_24_P910718 | A_24_P910718 | A_24_P910718 | 13.374 | 0.037034431 |
| A_23_P122052 | NM_001008397 | LOC493869 | 13.423 | 0.03941416 |
| A_23_P158851 | NM_032961 | PCDH10 | 13.442 | 0.029937011 |
| A_23_P158555 | NM_006514 | SCN10A | 13.524 | 0.015490486 |
| A_32_P346172 | NM_173479 | WDR88 | 13.582 | 0.042226989 |
| A_24_P246710 | NM_017954 | CADPS2 | 13.716 | 0.025268489 |
| A_32_P113845 | A_32_P113845 | A_32_P113845 | 13.731 | 0.033812196 |
| A_24_P818378 | A_24_P818378 | A_24_P818378 | 13.903 | 0.004679401 |
| A_23_P203698 | AK128620 | MOGAT2 | 14.083 | 0.035206836 |
| A_32_P88262 | NM_020949 | SLC7A14 | 14.123 | 0.009863368 |
| A_32_P46571 | NM_017821 | RHBDL2 | 14.25 | 0.027627439 |
| A_24_P937806 | A_24_P937806 | A_24_P937806 | 14.263 | 0.029383836 |
| A_23_P56913 | NM_001029881 | CIB4 | 14.273 | 0.008925835 |
| A_24_P918364 | A_24_P918364 | A_24_P918364 | 14.28 | 0.015028553 |
| A_24_P922758 | A_24_P922758 | A_24_P922758 | 14.31 | 0.036240958 |
| A_24_P216453 | AY163814 | SCAMPER | 14.315 | 0.042988297 |
| A_24_P941759 | NM_017769 | KIAA1333 | 14.354 | 0.021191812 |
| A_32_P158875 | AK091766 | LOC255480 | 14.378 | 0.043162381 |
| A_32_P40890 | THC2659529 | THC2659529 | 14.38 | 0.014697843 |
| AT_P_M | AT_P_M | AT_P_M | 14.391 | 0.026492769 |
| A_32_P54128 | THC2657493 | THC2657493 | 14.415 | 0.031767637 |
| A_23_P15619 | NM_032391 | C17orf92 | 14.435 | 0.032116835 |
| (−)3xSLv1 | NegativeControl | NegativeControl | 14.499 | 0.030648254 |
| A_24_P921862 | A_24_P921862 | A_24_P921862 | 14.544 | 0.03292804 |
| A_32_P43769 | BX095557 | BX095557 | 14.594 | 0.032655308 |
| A_24_P263878 | NM_007314 | ABL2 | 14.67 | 0.037152303 |
| A_24_P940365 | BC041481 | FLJ35848 | 14.738 | 0.034717847 |
| A_23_P29697 | NM_001248 | ENTPD3 | 14.738 | 0.030568975 |
| A_24_P228978 | AK094895 | C1QTNF3 | 14.855 | 0.004445566 |
| A_32_P141849 | A_32_P141849 | A_32_P141849 | 14.977 | 0.007437944 |
| A_24_P935575 | AY358233 | UNQ6484 | 15.005 | 0.033765817 |
| A_24_P937478 | U49441 | U49441 | 15.039 | 0.044296991 |
| RC8 | RC8 | RC8 | 15.126 | 0.019869998 |
| A_32_P218671 | THC2672257 | THC2672257 | 15.154 | 0.039029755 |
| A_32_P17917 | THC2777277 | THC2777277 | 15.208 | 0.009157247 |
| A_23_P141076 | NM_033151 | ABCC11 | 15.434 | 0.00861449 |
| A_23_P34897 | ENST00000325769 | ENST00000325769 | 15.518 | 0.013554061 |
| A_24_P709536 | BC002644 | MGC4859 | 15.677 | 0.029448337 |
| A_24_P744259 | BF692226 | BF692226 | 15.688 | 0.027320733 |
| A_24_P10444 | NM_173623 | TTLL6 | 15.797 | 0.037882588 |
| A_24_P239219 | AF420438 | AF420438 | 16.08 | 0.028539548 |
| A_32_P839537 | AF086464 | AF086464 | 16.28 | 0.049075953 |
| A_23_P68167 | NM_001298 | CNGA3 | 16.374 | 0.042397321 |
| A_23_P93122 | NM_005588 | MEP1A | 16.383 | 0.049306704 |
| A_32_P5568 | XM_001128764 | LOC728904 | 16.404 | 0.023230713 |
| A_23_P79591 | NM_000384 | APOB | 16.481 | 0.041451643 |
| A_23_P10995 | NM_014483 | RBMS3 | 16.548 | 0.045150891 |
| A_24_P153898 | NM_130803 | MEN1 | 16.565 | 0.033902626 |
| A_32_P65045 | AF074982 | AF074982 | 16.62 | 0.015929476 |
| A_32_P140521 | NM_173812 | DPY19L2 | 16.758 | 0.033937536 |
| A_32_P221774 | BX099483 | BX099483 | 16.81 | 0.011838221 |
| A_32_P424932 | AK023104 | AK023104 | 16.959 | 0.046581286 |
| A_23_P109785 | NM_003889 | NR1I2 | 17.011 | 0.033938283 |
| A_32_P121350 | BM681332 | BM681332 | 17.059 | 0.02199862 |
| A_32_P148616 | THC2660032 | THC2660032 | 17.067 | 0.028331528 |
| AT_P_3 | AT_P_3 | AT_P_3 | 17.244 | 0.040901272 |
| A_23_P134162 | NM_016356 | DCDC2 | 17.298 | 0.044335542 |
| A_24_P845631 | A_24_P845631 | A_24_P845631 | 17.321 | 0.015832631 |
| A_24_P391991 | NM_001105282 | LOC340286 | 17.657 | 0.007741406 |
| A_32_P6868 | NM_015472 | WWTR1 | 17.701 | 0.049343106 |
| A_32_P118847 | NR_021491 | FLJ42709 | 18.076 | 0.016162178 |
| A_23_P166027 | NM_012444 | SPO11 | 18.125 | 0.012236313 |
| A_23_P403488 | NM_176821 | NLRP10 | 18.128 | 0.035256772 |
| A_24_P365955 | AF258576 | LOC200261 | 18.133 | 0.017411973 |
| A_24_P796321 | CR746510 | LEUTX | 18.675 | 0.017933293 |
| A_24_P400842 | NM_207517 | ADAMTSL3 | 19.561 | 0.02456866 |
| RC11 | RC11 | RC11 | 19.628 | 0.042299165 |
| A_24_P919567 | BG743700 | BG743700 | 19.762 | 0.048948147 |
| A_32_P156237 | BC010054 | RPSA | 19.865 | 0.049733047 |
| AT_L_5 | AT_L_5 | AT_L_5 | 19.895 | 0.032088432 |
| A_32_P53433 | BF956667 | BF956667 | 20.229 | 0.032431547 |
| A_32_P79648 | BC005300 | DTNA | 20.246 | 0.028463944 |
| A_23_P25150 | NM_006897 | HOXC9 | 20.635 | 0.005096861 |
| A_23_P7250 | NM_001263 | CDS1 | 20.866 | 0.046281966 |
| A_24_P492458 | BX101405 | BX101405 | 21.236 | 0.026462822 |
| A_24_P585747 | CR604826 | CR604826 | 21.288 | 0.023444355 |
| A_23_P39881 | AL136789 | C2orf14 | 21.523 | 0.025752097 |
| A_23_P307161 | AF118078 | PR01848 | 21.841 | 0.017533066 |
| A_24_P673119 | A_24_P673119 | A_24_P673119 | 22.141 | 0.021134414 |
| A_23_P167683 | NM_000514 | GDNF | 22.746 | 0.002878573 |
| A_24_P76981 | AF119914 | AF119914 | 23.479 | 0.046103716 |
| A_23_P1173 | NM_004132 | HABP2 | 23.814 | 0.021305901 |
| A_24_P918552 | CK818049 | CK818049 | 24.314 | 0.045675502 |
| A_23_P382240 | NM_178505 | TMEM26 | 24.347 | 0.030470298 |
| A_23_P325100 | NM_021240 | DMRT3 | 24.791 | 0.041238043 |
| A_23_P118967 | NM_005559 | LAMA1 | 24.863 | 0.014320943 |
| A_23_P7835 | NM_004440 | EPHA7 | 25.198 | 0.01860067 |
| A_32_P91005 | BM697215 | BM697215 | 25.634 | 0.033293503 |
| A_23_P119464 | NM_033103 | RHPN2 | 25.754 | 0.022732977 |
| A_24_P365506 | NM_017671 | FERMT1 | 25.831 | 0.003032872 |
| A_32_P114475 | BQ045216 | BQ045216 | 26.623 | 0.021202676 |
| A_32_P81674 | AK091593 | LOC157627 | 27.235 | 0.001749581 |
| A_23_P82868 | NM_000930 | PLAT | 28.714 | 0.012124958 |
| A_24_P315954 | XR_018525 | XR_018525 | 32.87 | 0.049666197 |
| A_23_P215790 | NM_005228 | EGFR | 33.961 | 0.033551345 |
| A_23_P142849 | NM_005168 | RND3 | 35.687 | 0.023545167 |
| A_23_P412515 | NM_012129 | CLDN12 | 38.12 | 0.032399248 |
| A_24_P163168 | NM_016205 | PDGFC | 38.937 | 0.036067665 |
| A_23_P408376 | NM_025015 | HSPA12A | 39.843 | 0.000536102 |
| A_24_P824642 | A_24_P824642 | A_24_P824642 | 46.823 | 0.040604053 |
| A_24_P931030 | BC035145 | LOC283624 | 51.405 | 0.041379762 |
| A_32_P128952 | A_32_P128952 | A_32_P128952 | 53.374 | 0.032921336 |
| A_32_P379105 | NM_182699 | DDX53 | 56.085 | 0.032513652 |
| A_23_P351270 | NM_002581 | PAPPA | 58.207 | 0.022804938 |
| A_23_P90980 | NM_005383 | NEU2 | 59.58 | 0.001804918 |
| A_23_P380309 | NM_173629 | C18orf26 | 81.657 | 0.025260445 |
| A_24_P911051 | THC2538873 | THC2538873 | 94.682 | 0.017511095 |

A small number of genes (36) had more than one associated probe, of which one or more probes displayed up-regulation and one or more displayed down-regulation.

In a subsequent microarray analysis on RNA isolated from 150 female catheter lab patients enrolled in the PREDICT trial, six genes were selected for further evaluation via RT-qPCR due to their association with self-reported smoking status: SASH1; P2RY6; MUC1; LRRN3; MGAT3; AND CLDND1.

Among microarray probes that were biologically annotated and had non-zero expression, these were selected because they exhibited the strongest absolute correlation with smoking status (r>0.425). All have higher expression with smoking status. Five of these six RT-qPCR probe designs were successful; the design for MGAT3 was not, and this gene was not included. These 5 genes are designated as Set 1. Corresponding Agilent Whole Genome Array probes and Gene Name for the Set 1 markers (and MGAT3) are provided in Table 2.

TABLE 2

| Probe | Gene Name |
|---|---|
| A_23_P93442 | SASH1 |
| A_23_P64611 | P2RY6 |
| A_23_P137856 | MUC1 |
| A_23_P31376 | LRRN3 |
| A_24_P245838 | MGAT3 (not used) |
| A_23_P155556 | CLDND1 |

Phase II—RT-qPCR Analysis.

In an RT-qPCR analysis of RNA isolated from 1039 PREDICT patients, 261 genes, including the Set 1 genes, were assessed for association with smoking status (Table 3). The additional 255 genes were selected for association with coronary disease, associated traits (e.g., lipid levels) or as cell markers. Expression values for the 261 genes were normalized to the mean of ACLY and TFCP2; expression values for genes were truncated if values exceeded the 0.01 and 0.99 quantile. Of the genes, 135 showed a significant association with smoking status in an age- and sex-adjusted logistic regression model; 59 of the 80 significant array genes remained significant by qRT-PCR. Of this set, all but 3 (HIST1H2AC, NONO, PAPD4) agreed with the array data in directionality of gene expression. LRRN3 remained the gene most significantly associated with smoking status, followed by CLDND1, SASH1, and P2RY6 (p<0.001). Note that in Table 3, below, for the genes GNAS and FTH1, an “x” suffix in the gene symbol denotes that the assay for the given gene was designed against an exonic sequence; an “n” suffix in the gene symbol denotes that the assay for the given gene was designed against an intronic sequence.

TABLE 3
261 Gene Set

| | | | | | | |
|---|---|---|---|---|---|---|
| ABCA1 | ABCG1 | ABLIM1 | ACLY | ACOX2 | ADORA3 | AF086547 |
| AF161365 | AF289562 | AGER | AGPAT3 | AK2 | AKAP8L | AKNA |
| ALS2CR2 | AMN | ANAPC5 | ANKRD55 | ANP32C | APEX1 | APOE |
| APOL4 | AQP9 | AR | ARG1 | ARHGEF10L | ASGR1 | ATP5G3 |
| B3GAT1 | BPNT1 | C5AR1 | C5orf4 | C6ORF25 | CALML4 | CAPN10 |
| CASP5 | CAT | CCL2 | CCL5 | CCNB1 | CCND2 | CCR3 |
| CCR7 | CD11B | CD161 | CD163 | CD177 | CD19 | CD1D |
| CD248 | CD33 | CD34 | CD3D | CD44 | CD45RA | CD62P |
| CD63 | CD79B | CD8 | CD83 | CD84 | CD86 | CDCA7 |
| CENTB5 | CLDND1 | CLEC4E | CMTM5 | COL13A1 | COX4I1 | COX6C |
| CPA3 | CR2 | CR596746 | CREB1 | CRIP1 | CX3CR1 | CXCR5 |
| CXXC1 | CYP4F2 | DGKA | DGKH | DIAPH1 | DIAPH2 | DPYSL4 |
| DTD1 | DUSP6 | E01979 | ELF1 | EMR1 | ENPP3 | EPHB1 |
| ESR1 | EXOC3L2 | F11R | FAM20B | FANK1 | FOXP3 | FTH1n |
| FTH1x | GALNT3 | GAPDH | GATA2 | GFOD1 | GNASn | GNASx |
| GOPC | GPER | GPR56 | GPX4 | GTFCP2 | GUSB | GZMB |
| HDAC10 | HDC | HDLBP | HIST1H2AC | HMGN3 | HMOX1 | HNRNPF |
| HNRNPH2 | HNRPF | IF127 | IFIT3 | IFNAR1 | IL13RA1 | IL17RC |
| IL18RAP | IL3RA | IL4 | IL6 | IL7R | IL8RB | ILK |
| IQCE | IRF7 | ITGA2B | ITPK1 | KCNE1 | KCNE3 | KLRC4 |
| LDLR | LEF1 | LEMD2 | LIN9 | LITAF | LOC440461 | LPCAT1 |
| LRP1 | LRRC25 | LRRN3 | LTK | LY75 | MBOAT7 | MCM3 |
| MFSD9 | MIER1 | MPEG1 | MRPL11 | MRPS12 | MS4A7 | MSN |
| MTCH1 | MUC1 | MYLIP | NBPF11 | NCF4 | NCOR2 | ND3 |
| NDUFA1 | NDUFS7 | NEFH | NELL2 | NF1 | NISCH | NME1 |
| NMI | NONO | NRCAM | NUDT16 | OLIG1 | OPRL1 | OSBPL6 |
| P2RY6 | PACSIN2 | PAPD4 | PDGFD | PDGFRB | PDK1 | PECAM1 |
| PECR | PFKFB2 | PLA2G6 | PLCD1 | POLD3 | PPP2R2B | PTX3 |
| RAB32 | RABGAP1L | RAD54B | RBM17 | RHCE | RNPS1 | RORC |
| RP9 | RPL28 | RPP14 | RPS4Y1 | S100A12 | S100A8 | SAMD3 |
| SASH1 | SCML4 | SECISBP2 | SEPX1 | SERGEF | SERPINB2 | SETD2 |
| SF3B5 | SFRS10 | SILV | SIRT2 | SIRT4 | SLAMF7 | SNRPA |
| SOAT2 | SORCS3 | SORT1 | SPECC1 | SPHAR | SPIB | SPOCK2 |
| SREBF1 | SRP9 | SSRP1 | STX10 | SYNE1 | TARDBP | TFCP2 |
| TFIP11 | TLR2 | TLR4 | TMC8 | TMED9 | TMEFF2 | TMTC3 |
| TNFAIP6 | TNFRSF10C | TNFRSF17 | TNXB | TOP2A | TP53BP1 | TPOR |
| TRAF2 | TREML4 | TXNDC5 | UCK2 | VCAN | VEZF1 | WDR18 |
| ZNF16 | ZNF3 | | | | | |

Determining Discriminating Threshold Values for Individual Markers.

In certain embodiments, smoking status is determined by analyzing the expression level of a single marker or a group of markers to determine whether the expression level is significantly different from a threshold level established by analysis of the expression level of the same marker or markers in a non-smoking subject or population of non-smoking subjects. A significant difference between a subject's value for the marker or markers and the threshold value indicates smoking status. Conversely, methods can be developed to set thresholds using a smoking subject or population of non-smoking subjects. In this embodiment a significant difference indicates non-smoking status of the subject providing the test sample.

### Example 1

### Predictive Modeling

A predictive model for smoking status was built using stepwise forward logistic regression (9), with smoking status as the dependent variable and age, sex, and gene expression as the independent variables. A patient was defined as a smoker if their self-reported smoking status was a current smoker or one who had recently quit (within the previous two months). Five genes were selected by the model, designated as Set 2. Three of the Set 1 genes were selected by the model (CLDND1, LRRN3, MUC1). The remaining 2 genes (GOPC, LEFT) were included in the 259 gene analysis due to CAD association and as a CD8+naïve cell marker, respectively. The probe and primer sequences used to assess the expression levels of the Set 2 genes are given in Table 4.

TABLE 4

| | Gene | Forward Primer | Reverse Primer | Reporter Sequence |
|---|---|---|---|---|
| RefSeq | Name | Sequence 5′ → 3′ | Sequence 5′ → 3′ | 5′ → 3′ |
| NM_019895 | CLDND1 | GCCACGGGC | CATAACAACTTAC | CACAGACCT |
| | | ATTCTCCAT | TGAGCCCAGTGT | GCAAGGAG |
| | | (SEQ ID NO: 1) | (SEQ ID NO: 2) | (SEQ ID NO: 3) |
| NM_018334 | LRRN3 | CTGCCTCTCTC | CTCACCTAATGCA | ATAGCTGTGT |
| | | CAGAAATGAACT | AAGGTTGGTTTC | CCACCATCAC |
| | | (SEQ ID NO: 4) | (SEQ ID NO: 5) | (SEQ ID NO: 6) |
| NM_016269 | LEF1 | CAAGAATGACAG | GCTGCCTT | TCGTTTTCCA |
| | | CTGCCTACATCT | GGCTTTGCA | CCATGTTTC |
| | | (SEQ ID NO: 7) | (SEQ ID NO: 8) | (SEQ ID NO: 9) |
| NM_002456 | MUC1 | CCCCTAGCAGTA | GCTGCTGCCA | CCCCTATGAG |
| | | CCGATCGTA | CCATTACCT | AAGGTTTC |
| | | (SEQ ID NO: 10) | (SEQ ID NO: 11) | (SEQ ID NO: 12) |
| NM_020399 | GOPC | GTGCTAGTTGCA | ATGTGTGTCAGTT | CTTGTAATACTT |
| | | AAGACACAAGTG | ACTGCCTTCTT | TGATTTCCC |
| | | (SEQ ID NO: 13) | (SEQ ID NO: 14) | (SEQ ID NO: 15) |

The resulting model formula is:

log(Pr(Smoker)/(1−Pr(Smoker))=15.7306+0.3876*SEX−3.3368*CLDND1−3.4034*LRRN3−1.4847*MUC1+5.9209*GOPC+2.7166*LEF1,

where SEX=1 if male, 0 if female; Pr=probability.

For predicting self-reported status, the model has a mean AUC of 0.932 in ten-fold cross-validation. At a cutoff probability of 0.5, the fitted sensitivity of the model was 0.784, with a specificity of 0.953. More details of model performance are provided in the Table 5. Model performance was validated using 180 independent PREDICT subjects, with an AUC of 0.82 (95% CI 0.65-0.94), a sensitivity of 0.63 and a specificity of 0.94.

TABLE 5

| Self-Reported | Number | Predicted Status | | Percent |
|---|---|---|---|---|
| Status | of Patients | Smoker | Non-Smoker | Agreement |
| Current Smoker | 195 | 157 | 38 | 81% |
| Quit Recently | 27 | 17 | 10 | 62% |
| Former | 341 | 29 | 312 | 91% |
| Never | 476 | 9 | 467 | 98% |
| Total | 1039 | | | 92% |

### Example 2

### Cross-Validated Sub-Model Development

Ten-fold cross-validated sub-models were developed for all subsets of the Set 2 genes using the approach described in Example 1 (i.e., stepwise forward logistic regression (9), with smoking status as the dependent variable and age, sex, and gene expression as the independent variables). The performance of these sub-models is set out in Table 6.

TABLE 6

| CLDND1 | LRRN3 | MUC1 | GOPC | LEF1 | AUC |
|---|---|---|---|---|---|
| 1 | | | | | 0.833 |
| | 1 | | | | 0.859 |
| 1 | 1 | | | | 0.884 |
| | | 1 | | | 0.734 |
| 1 | | 1 | | | 0.844 |
| | 1 | 1 | | | 0.878 |
| 1 | 1 | 1 | | | 0.888 |
| | | | 1 | | 0.54 |
| 1 | | | 1 | | 0.857 |
| | 1 | | 1 | | 0.879 |
| 1 | 1 | | 1 | | 0.915 |
| | | 1 | 1 | | 0.73 |
| 1 | | 1 | 1 | | 0.865 |
| | 1 | 1 | 1 | | 0.897 |
| 1 | 1 | 1 | 1 | | 0.917 |
| | | | | 1 | 0.634 |
| 1 | | | | 1 | 0.831 |
| | 1 | | | 1 | 0.912 |
| 1 | 1 | | | 1 | 0.918 |
| | | 1 | | 1 | 0.747 |
| 1 | | 1 | | 1 | 0.841 |
| | 1 | 1 | | 1 | 0.923 |
| 1 | 1 | 1 | | 1 | 0.924 |
| | | | 1 | 1 | 0.642 |
| 1 | | | 1 | 1 | 0.863 |
| | 1 | | 1 | 1 | 0.916 |
| 1 | 1 | | 1 | 1 | 0.928 |
| | | 1 | 1 | 1 | 0.75 |
| 1 | | 1 | 1 | 1 | 0.869 |
| | 1 | 1 | 1 | 1 | 0.926 |

The equations corresponding to these sub-models are set out below in Table 7.

They are applied in a similar manner as the equation set out in Example 1 and are used to solve for log (Pr(Smoker)/(1−Pr(Smoker)). For example, the formula associated with submodel 1 is: log (Pr(Smoker)/(1−Pr(Smoker))=3.411334+0.5660*SEX−6.4940*CLDND1, where SEX=1 if male, 0 if female; Pr=probability.

TABLE 7

| Submodel | Intercept | Sex | CLDND1 | LRRN3 | MUC1 | GOPC | LEF1 |
|---|---|---|---|---|---|---|---|
| 1 | 3.411334 | 0.565969 | −6.49396 | NA | NA | NA | NA |
| 2 | 2.61478 | 0.780253 | NA | −2.10677 | NA | NA | NA |
| 3 | 4.59527 | 0.77818 | −4.1712 | −1.57259 | NA | NA | NA |
| 4 | 12.42875 | 0.737512 | NA | NA | −2.17005 | NA | NA |
| 5 | 10.20879 | 0.720202 | −5.61562 | NA | −1.18637 | NA | NA |
| 6 | 14.47653 | 1.083409 | NA | −2.00953 | −1.93001 | NA | NA |
| 7 | 12.45022 | 0.974022 | −3.09362 | −1.62647 | −1.36313 | NA | NA |
| 8 | −0.38902 | 0.418774 | NA | NA | NA | −1.10845 | NA |
| 9 | 1.325027 | 0.380818 | −7.88323 | NA | NA | 4.903013 | NA |
| 10 | 0.111031 | 0.596036 | NA | −2.50111 | NA | 5.231238 | NA |
| 11 | 1.244433 | 0.489895 | −5.84709 | −2.00238 | NA | 8.606524 | NA |
| 12 | 12.67808 | 0.759998 | NA | NA | −2.15908 | −0.51445 | NA |
| 13 | 7.773203 | 0.526467 | −7.04575 | NA | −1.11259 | 4.773315 | NA |
| 14 | 12.25793 | 0.896207 | NA | −2.44872 | −2.01558 | 5.763504 | NA |
| 15 | 8.447132 | 0.668232 | −4.84284 | −2.04127 | −1.2398 | 8.471973 | NA |
| 16 | −2.17829 | 0.638749 | NA | NA | NA | NA | −1.08098 |
| 17 | 2.884638 | 0.642944 | −6.26935 | NA | NA | NA | −0.3679 |
| 18 | 9.671707 | 0.281206 | NA | −4.00991 | NA | NA | 3.595369 |
| 19 | 10.37175 | 0.348651 | −3.40865 | −3.29712 | NA | NA | 3.179526 |
| 20 | 11.03694 | 0.960992 | NA | NA | −2.10223 | NA | −0.94278 |
| 21 | 9.67367 | 0.793541 | −5.41932 | NA | −1.17746 | NA | −0.33745 |
| 22 | 21.94679 | 0.513516 | NA | −3.91827 | −1.96486 | NA | 3.721327 |
| 23 | 20.054 | 0.495412 | −2.06062 | −3.48288 | −1.59925 | NA | 3.436186 |
| 24 | −3.00575 | 0.623444 | NA | NA | NA | 1.096089 | −1.22835 |
| 25 | −1.1565 | 0.541509 | −7.78844 | NA | NA | 6.953846 | −1.13715 |
| 26 | 7.568068 | 0.186808 | NA | −4.12955 | NA | 3.364823 | 3.332477 |
| 27 | 6.490723 | 0.234579 | −4.69859 | −3.21159 | NA | 6.318137 | 2.42355 |
| 28 | 10.076 | 0.94251 | NA | NA | −2.11189 | 1.354226 | −1.12336 |
| 29 | 5.007036 | 0.6789 | −7.00759 | NA | −1.03156 | 6.641832 | −1.06194 |
| 30 | 19.86698 | 0.431392 | NA | −4.06979 | −2.02711 | 3.946928 | 3.407628 |

### Example 3

### Cross-Validated Models Using Highly-Correlated Substitute Markers

Models were developed based on the model described in Example 1, in which one of the markers was substituted with a highly correlated marker; the criterion for selecting the highly correlated marker was maximum (Pearson) correlation R to the original gene. Table 8 shows the mean AUC in ten-fold cross-validated models where each gene in the smoking model was replaced by a gene selected from 253 study genes that was not already included in the smoking model. Correlations (expressed as R values) are also included in Table 8.

TABLE 8

| Original | Substitute | AUC | R² |
|---|---|---|---|
| CLDND1 | IL7R | 0.928 | 0.5127363 |
| LRRN3 | CCR7 | 0.868 | 0.703982 |
| MUC1 | FOXP3 | 0.927 | 0.4166257 |
| GOPC | MCM3 | 0.925 | 0.7775874 |
| LEF1 | CCR7 | 0.928 | 0.944785 |

### Example 4

### Threshold Determinations for Informative Markers

The reported mean Cts and standard deviations of the informative set of 259 markers in the smoking and non-smoking population are as shown in Table 9. These values have been normalized and truncated as described above in the “Phase II—RT-qPCR Analysis” section.

TABLE 9

| | Non- | Non- | | |
|---|---|---|---|---|
| | Smoking | Smoking | Smoking | Smoking |
| Gene | Mean | St Dev | Mean | St Dev |
| TFCP2 | 1.125596 | 0.146217 | 1.105504054 | 0.148529952 |
| RPS4Y1 | −0.58487 | 3.237528 | −0.976826087 | 2.669099207 |
| GTFCP2 | 8.710473 | 0.735477 | 8.733363964 | 0.757677366 |
| TNFRSF10C | −4.27986 | 0.431794 | −4.388873423 | 0.4331237 |
| ARHGEF10L | 2.367465 | 0.336897 | 2.373927477 | 0.312019963 |
| B3GAT1 | 2.755056 | 0.910727 | 3.151548198 | 0.919998385 |
| NCF4 | −2.16508 | 0.393844 | −2.307054955 | 0.393445844 |
| HNRPF | −1.5215 | 0.144993 | −1.556747072 | 0.139517389 |
| ARG1 | 1.896007 | 0.874045 | 1.784203829 | 0.93971967 |
| ADORA3 | 3.659335 | 0.778664 | 3.622131757 | 0.814026083 |
| NDUFS7 | 0.084403 | 0.135686 | 0.089475676 | 0.120693414 |
| APOL4 | −0.28188 | 0.268937 | −0.255613739 | 0.254215689 |
| IL4 | 7.562976 | 0.786824 | 7.549558559 | 0.852439085 |
| LTK | 3.114488 | 0.753085 | 3.043172072 | 0.67187502 |
| CD79B | 0.250163 | 0.569544 | 0.208981757 | 0.527849968 |
| IL8RB | −4.71562 | 0.442697 | −4.816301577 | 0.432829152 |
| AGPAT3 | 0.390438 | 0.263195 | 0.484493243 | 0.254724623 |
| SLAMF7 | −0.26956 | 0.414154 | −0.046911036 | 0.423108735 |
| CASP5 | 2.893515 | 0.7591 | 2.706239189 | 0.77556682 |
| AQP9 | −3.43946 | 0.462339 | −3.556288288 | 0.475855099 |
| NEFH | 6.085764 | 0.514344 | 6.031367117 | 0.443362656 |
| CLDND1 | 0.829002 | 0.181864 | 0.561819144 | 0.214623193 |
| APOE | 8.127503 | 0.709796 | 8.113110135 | 0.673617206 |
| CD86 | 1.959998 | 0.302797 | 2.046366441 | 0.298524984 |
| SF3B5 | 0.095047 | 0.108356 | 0.100627928 | 0.096233737 |
| OSBPL6 | 7.668752 | 0.804047 | 7.788405631 | 0.857836177 |
| DIAPH1 | −1.49705 | 0.149158 | −1.473285586 | 0.155703854 |
| KCNE1 | 6.368764 | 0.603992 | 6.46412973 | 0.571747072 |
| ITGA2B | 0.1201 | 0.773454 | 0.441109009 | 0.831043434 |
| SPECC1 | 1.06176 | 0.413616 | 1.255337838 | 0.38707037 |
| RAD54B | 6.403795 | 0.489584 | 6.311536486 | 0.518046685 |
| KCNE3 | 0.166354 | 0.393329 | 0.140327027 | 0.357144726 |
| ATP5G3 | −1.42123 | 0.145889 | −1.418379279 | 0.143869165 |
| DGKH | 3.37517 | 0.307179 | 3.30683964 | 0.291443203 |
| CD8 | −0.69421 | 0.74527 | −0.759033108 | 0.65653581 |
| TLR4 | −1.67978 | 0.429446 | −1.725411261 | 0.422643199 |
| GFOD1 | 2.834806 | 0.359844 | 3.040817342 | 0.36979134 |
| TXNDC5 | 0.753651 | 0.533727 | 0.572440315 | 0.570311476 |
| CD163 | 3.743367 | 0.430262 | 3.726241441 | 0.436296063 |
| SYNE1 | 2.892392 | 0.46739 | 2.990459459 | 0.468410152 |
| LRRN3 | 2.299103 | 0.628185 | 1.285129955 | 0.73670413 |
| NCOR2 | 1.384834 | 0.140541 | 1.404293243 | 0.133145991 |
| CD161 | −0.08199 | 0.475383 | 0.14259009 | 0.425363955 |
| MUC1 | 6.462191 | 0.40658 | 6.144355856 | 0.39716938 |
| NUDT16 | 1.923331 | 0.291711 | 1.976746622 | 0.280701617 |
| MSN | −4.0388 | 0.193462 | −4.027445946 | 0.201631799 |
| CRIP1 | −2.36444 | 0.378105 | −2.382880405 | 0.35234144 |
| PFKFB2 | 3.664807 | 0.394426 | 3.727288063 | 0.37890164 |
| KLRC4 | 0.377383 | 0.672051 | 0.540327928 | 0.661954652 |
| RAB32 | 1.562078 | 0.267471 | 1.571133333 | 0.252166164 |
| S100A12 | −2.76593 | 0.677066 | −3.010933784 | 0.69557978 |
| LPCAT1 | −1.48829 | 0.231926 | −1.418833784 | 0.211848873 |
| DUSP6 | −1.29352 | 0.297617 | −1.239263964 | 0.308498732 |
| ESR1 | 7.119984 | 0.558057 | 7.205291892 | 0.612637061 |
| CR2 | 4.48686 | 0.754381 | 4.386742342 | 0.68565516 |
| IL6 | 8.632731 | 0.937011 | 8.512101802 | 0.785070037 |
| ACLY | −1.02098 | 0.068758 | −1.014087838 | 0.060962216 |
| GPX4 | −0.88231 | 0.32922 | −0.823274324 | 0.30745841 |
| CLEC4E | −1.13914 | 0.597314 | −1.032295946 | 0.610510227 |
| CD248 | 6.698602 | 0.849312 | 6.458838964 | 0.789709524 |
| CAT | −2.26215 | 0.405814 | −2.243329505 | 0.430694321 |
| PDGFRB | 3.577047 | 0.772309 | 3.776035135 | 0.847678191 |
| IL18RAP | −1.22419 | 0.768513 | −1.265853829 | 0.777313528 |
| CD45RA | −4.42276 | 0.214768 | −4.436934234 | 0.216347649 |
| TNFRSF17 | 5.601019 | 0.87457 | 5.292971171 | 0.843997637 |
| CD33 | 1.214859 | 0.413968 | 1.242921622 | 0.409814846 |
| CXCR5 | 2.855219 | 0.674005 | 2.66262455 | 0.606519631 |
| IL7R | −2.14918 | 0.416869 | −2.345188739 | 0.346727152 |
| LITAF | −3.41176 | 0.374577 | −3.508939865 | 0.364043265 |
| RPP14 | 1.878987 | 0.122101 | 1.869304955 | 0.111592671 |
| HMOX1 | 2.681434 | 0.375634 | 2.814602928 | 0.396654231 |
| AF289562 | 5.437364 | 0.508596 | 5.043204955 | 0.499503353 |
| CD3D | −0.10736 | 0.391777 | −0.217402027 | 0.336372984 |
| TNFAIP6 | 0.680172 | 0.76847 | 0.54751036 | 0.76836694 |
| RPL28 | −5.753 | 0.196765 | −5.816542793 | 0.192424138 |
| AF161365 | 4.458622 | 1.613475 | 4.409285135 | 1.560077908 |
| ACOX2 | 7.980393 | 0.812675 | 8.10778536 | 0.857615003 |
| GPER | 8.049317 | 0.95915 | 8.113020495 | 0.842408025 |
| ASGR1 | 4.751193 | 0.421625 | 4.700075676 | 0.390829125 |
| P2RY6 | 6.372312 | 0.544723 | 5.775368919 | 0.647766148 |
| NRCAM | 8.080202 | 1.387639 | 7.662772297 | 1.24874828 |
| S100A8 | −6.87861 | 0.444134 | −7.01310518 | 0.455958669 |
| SASH1 | 4.045093 | 0.606764 | 3.364506982 | 0.608530939 |
| LRRC25 | −1.12352 | 0.307479 | −1.11460518 | 0.305827143 |
| SPIB | 6.591512 | 0.707007 | 6.469470721 | 0.616648066 |
| GUSB | 1.391091 | 0.157754 | 1.403323649 | 0.15751305 |
| SIRT4 | 6.620658 | 0.33789 | 6.515320721 | 0.312679991 |
| PECAM1 | −1.04633 | 0.374087 | −1.060084234 | 0.370758328 |
| TMC8 | −0.40109 | 0.251376 | −0.43226509 | 0.226331544 |
| CD11B | −0.53127 | 0.344585 | −0.569431081 | 0.352877908 |
| SERPINB2 | 4.522124 | 0.59019 | 4.325362387 | 0.618927823 |
| LEMD2 | 2.978249 | 0.154894 | 2.935083559 | 0.149951186 |
| EPHB1 | 3.085858 | 0.447108 | 3.047672523 | 0.448540067 |
| HDLBP | −0.51087 | 0.254057 | −0.466999775 | 0.238503356 |
| SPHAR | 4.240357 | 0.256746 | 4.254421847 | 0.258094618 |
| IQCE | 2.379599 | 0.301142 | 2.411414189 | 0.312543713 |
| WDR18 | 2.22436 | 0.255728 | 2.187703604 | 0.238715854 |
| AF086547 | 6.523929 | 0.669136 | 6.286678604 | 0.605289131 |
| CD34 | 10.44413 | 0.902898 | 10.63648287 | 0.956125811 |
| OPRL1 | 1.441805 | 0.302414 | 1.415645045 | 0.304292652 |
| IFI27 | 0.508477 | 1.519958 | 0.632646396 | 1.842287337 |
| MIER1 | 4.027133 | 0.306648 | 4.035628378 | 0.293707719 |
| RHCE | 7.279536 | 0.838124 | 7.589659459 | 0.965013379 |
| LY75 | 0.691582 | 0.299268 | 0.697472748 | 0.304303778 |
| ND3 | −3.54007 | 0.48996 | −3.409531952 | 0.504735744 |
| CCL5 | −3.36958 | 0.413112 | −3.218958559 | 0.423076316 |
| SIRT2 | −0.04306 | 0.138889 | 0.010926351 | 0.136992705 |
| SETD2 | 0.476999 | 0.155674 | 0.475031081 | 0.157287376 |
| CD44 | −4.02499 | 0.173881 | −4.064556532 | 0.192543812 |
| MRPS12 | 2.080735 | 0.223968 | 2.064821847 | 0.20866223 |
| SCML4 | 4.63569 | 0.316513 | 4.528416441 | 0.306553769 |
| MBOAT7 | −3.98951 | 0.370947 | −4.066620721 | 0.381900653 |
| MS4A7 | 0.626127 | 0.468409 | 0.940858108 | 0.484871439 |
| MCM3 | 1.07203 | 0.201976 | 1.043799324 | 0.184269323 |
| STX10 | −1.55852 | 0.282992 | −1.606639414 | 0.289111205 |
| EXOC3L2 | 6.625546 | 0.700683 | 6.828664865 | 0.741545034 |
| HIST1H2AC | −2.27576 | 0.373767 | −2.171192117 | 0.366012602 |
| RNPS1 | 2.634163 | 0.199824 | 2.602028604 | 0.17485955 |
| PDK1 | 0.090928 | 0.273011 | 0.055498649 | 0.285614552 |
| DTD1 | 3.321172 | 0.236266 | 3.331502027 | 0.226797101 |
| FAM20B | 1.00578 | 0.414103 | 1.169663739 | 0.421967935 |
| CD19 | 2.053676 | 0.750295 | 1.980651577 | 0.666407872 |
| PACSIN2 | −0.30731 | 0.326557 | −0.35651036 | 0.341105287 |
| RP9 | 3.565285 | 0.24521 | 3.548375676 | 0.22211064 |
| TARDBP | −1.02328 | 0.119876 | −1.053574099 | 0.116215742 |
| ALS2CR2 | −4.54852 | 0.561263 | −4.236203153 | 0.627862018 |
| CXXC1 | 0.110369 | 0.132436 | 0.067280631 | 0.118963183 |
| COX6C | 3.640311 | 0.269763 | 3.644170946 | 0.252033897 |
| OLIG1 | 0.988353 | 0.534205 | 1.05028964 | 0.535547788 |
| TPOR | 4.40245 | 0.74843 | 4.697403604 | 0.773724715 |
| SFRS10 | 0.168181 | 0.152397 | 0.16718964 | 0.149542633 |
| MTCH1 | −1.09532 | 0.169724 | −1.063543018 | 0.175957267 |
| CD177 | 5.014015 | 1.818601 | 4.498923198 | 1.891294359 |
| GOPC | 0.646138 | 0.12817 | 0.643237838 | 0.124815442 |
| LOC440461 | 5.207979 | 0.474061 | 5.134113288 | 0.454574163 |
| HNRNPH2 | −1.30821 | 0.266402 | −1.330696622 | 0.269339044 |
| SAMD3 | 1.319707 | 0.434309 | 1.488155856 | 0.409620231 |
| CALML4 | 1.773353 | 0.261768 | 1.845455856 | 0.252942821 |
| GAPDH | −2.61194 | 0.243281 | −2.699326577 | 0.259566984 |
| AMN | 7.202804 | 0.536499 | 7.077812613 | 0.490848497 |
| PAPD4 | −0.31633 | 0.183545 | −0.273289865 | 0.183674711 |
| AKNA | −2.55266 | 0.166677 | −2.570526351 | 0.160783134 |
| LIN9 | 4.832509 | 0.237713 | 4.834842342 | 0.234018534 |
| CD83 | 3.311019 | 0.373875 | 3.394490766 | 0.372174066 |
| SRP9 | 2.858857 | 0.28931 | 2.884091441 | 0.290261374 |
| RBM17 | 0.871725 | 0.141758 | 0.841051126 | 0.145092009 |
| IFNAR1 | −0.35189 | 0.32553 | −0.430553829 | 0.337870386 |
| SECISBP2 | 0.580786 | 0.189824 | 0.622390541 | 0.185196636 |
| SSRP1 | 0.479218 | 0.179224 | 0.439537162 | 0.168708193 |
| EMR1 | 0.418363 | 0.520399 | 0.523793468 | 0.527656388 |
| C60RF25 | −1.23839 | 0.652059 | −0.972559459 | 0.69920626 |
| NISCH | 0.77996 | 0.13702 | 0.729527027 | 0.137049257 |
| F11R | −1.02868 | 0.289412 | −0.997102928 | 0.281548075 |
| CMTM5 | 1.223707 | 0.623909 | 1.53599482 | 0.696096375 |
| IL3RA | 2.855232 | 0.721332 | 2.939437838 | 0.696488388 |
| RABGAP1L | −1.53732 | 0.432924 | −1.383878378 | 0.393545877 |
| PPP2R2B | 3.781557 | 0.552527 | 3.995704955 | 0.538368454 |
| GALNT3 | 1.388498 | 0.308642 | 1.407643018 | 0.304585505 |
| ILK | −0.74977 | 0.246929 | −0.65746509 | 0.253023787 |
| CCR3 | 7.841666 | 0.889357 | 7.86578018 | 0.91647511 |
| ELF1 | −2.53641 | 0.182335 | −2.54412973 | 0.190380181 |
| FOXP3 | 2.035403 | 0.463382 | 1.697540541 | 0.494839978 |
| NMI | −0.56271 | 0.360461 | −0.613682432 | 0.376996873 |
| SNRPA | 0.105145 | 0.146042 | 0.055614865 | 0.138100561 |
| AKAP8L | −0.60328 | 0.163987 | −0.587923423 | 0.163914758 |
| C5AR1 | −2.61855 | 0.399434 | −2.662788514 | 0.403387089 |
| NDUFA1 | −0.67182 | 0.162867 | −0.660734685 | 0.148177014 |
| E01979 | −2.67616 | 0.259085 | −2.665088063 | 0.249054895 |
| SEPX1 | −0.41993 | 0.406557 | −0.493091892 | 0.38564448 |
| CD62P | 3.721248 | 0.649737 | 4.118711486 | 0.729052333 |
| IFIT3 | 0.23842 | 1.18242 | 0.283440991 | 1.339833454 |
| COX4I1 | −2.39436 | 0.174301 | −2.446354054 | 0.163642322 |
| TREML4 | 4.697655 | 2.126498 | 4.64068964 | 2.084084981 |
| RORC | 3.63029 | 0.696619 | 3.4194 | 0.685704776 |
| MRPL11 | 2.532977 | 0.187339 | 2.484686261 | 0.180963152 |
| CD63 | −2.35276 | 0.250825 | −2.370822072 | 0.250794382 |
| SOAT2 | 6.686092 | 0.806516 | 6.242241216 | 0.798995643 |
| DIAPH2 | 3.474493 | 0.212862 | 3.472038288 | 0.19935658 |
| MFSD9 | 2.573817 | 0.52283 | 2.564026351 | 0.542160873 |
| CAPN10 | 2.830346 | 0.166177 | 2.791031982 | 0.153554063 |
| CD1D | 1.050567 | 0.316671 | 1.040843018 | 0.330610282 |
| TMED9 | −0.21229 | 0.130832 | −0.211245721 | 0.118317659 |
| ZNF16 | 4.413172 | 0.221988 | 4.373307883 | 0.204724333 |
| TFIP11 | 2.28308 | 0.22865 | 2.249861937 | 0.204377495 |
| IL17RC | 6.244819 | 0.563749 | 6.349556757 | 0.53490333 |
| DPYSL4 | 6.707837 | 1.504658 | 6.688766892 | 1.432249514 |
| SREBF1 | 6.569555 | 0.38363 | 6.448862613 | 0.411325066 |
| LRP1 | 0.36794 | 0.312214 | 0.33545473 | 0.304482084 |
| LEF1 | −0.79958 | 0.504772 | −0.997103604 | 0.448617038 |
| GNASx | −4.06309 | 0.36153 | −3.939269369 | 0.368916188 |
| TRAF2 | 3.207773 | 0.202676 | 3.146210811 | 0.190548656 |
| TNXB | 3.631028 | 0.641216 | 3.895052703 | 0.656759548 |
| FTH1n | 0.086012 | 0.326464 | 0.049940541 | 0.345079008 |
| ANKRD55 | 4.234666 | 0.453042 | 4.296562387 | 0.469057567 |
| NME1 | 2.191169 | 0.300938 | 2.113855631 | 0.28579282 |
| ABCG1 | 0.795011 | 0.574699 | 0.618190541 | 0.498685501 |
| CREB1 | −1.0601 | 0.186553 | −1.016782432 | 0.179594748 |
| FANK1 | 8.816375 | 0.668986 | 8.401322072 | 0.652855298 |
| UCK2 | 3.130967 | 0.227908 | 3.164131306 | 0.232735251 |
| TLR2 | −1.11483 | 0.451642 | −1.125605631 | 0.473311795 |
| CYP4F2 | −2.15939 | 0.576827 | −2.237032658 | 0.546386676 |
| NELL2 | 1.129627 | 0.570065 | 0.88444009 | 0.521224197 |
| PLCD1 | 2.158703 | 0.214572 | 2.099186486 | 0.194407111 |
| SPOCK2 | −1.29809 | 0.306079 | −1.402984685 | 0.256893671 |
| GATA2 | 1.273369 | 0.55071 | 1.295314865 | 0.596428823 |
| CCR7 | 0.156229 | 0.56791 | 0.02839009 | 0.498479516 |
| IL13RA1 | −0.96071 | 0.410738 | −0.980468468 | 0.43047763 |
| DGKA | −1.64074 | 0.300994 | −1.751491892 | 0.27504889 |
| GPR56 | −0.92342 | 0.64933 | −0.527438063 | 0.648358113 |
| ITPK1 | 7.333648 | 0.612266 | 7.396810586 | 0.585007453 |
| ENPP3 | 5.341159 | 0.668836 | 5.311447072 | 0.666695156 |
| POLD3 | 2.040477 | 0.194494 | 2.071801802 | 0.18729534 |
| CD84 | 0.047531 | 0.239851 | 0.043278604 | 0.215592269 |
| VCAN | −0.86714 | 0.384889 | −0.938474775 | 0.388425453 |
| APEX1 | 1.429239 | 0.203455 | 1.390574324 | 0.190065635 |
| SERGEF | 3.37937 | 0.170243 | 3.339512162 | 0.161181778 |
| CENTB5 | 3.529176 | 0.187672 | 3.458763964 | 0.187462574 |
| PLA2G6 | 2.834793 | 0.214971 | 2.806927252 | 0.19310565 |
| C5orf4 | 0.738108 | 0.682775 | 1.144945495 | 0.711108489 |
| SORCS3 | 7.373485 | 1.000594 | 7.37939009 | 0.939232025 |
| PTX3 | 5.298692 | 0.390884 | 5.291436937 | 0.412715615 |
| TOP2A | 5.19909 | 0.392742 | 5.302955405 | 0.42955525 |
| BPNT1 | 2.155689 | 0.167464 | 2.12165518 | 0.153651361 |
| CDCA7 | 4.126752 | 0.397615 | 4.039365991 | 0.387128426 |
| TP53BP1 | 2.634165 | 0.173978 | 2.584755405 | 0.150807134 |
| CCL2 | 6.833726 | 1.067369 | 6.733059955 | 1.16394245 |
| PDGFD | 4.513274 | 0.524896 | 4.875308559 | 0.520526123 |
| FTH1x | −6.87578 | 0.354032 | −6.81383018 | 0.353371039 |
| COL13A1 | 7.693673 | 1.1374 | 8.254777477 | 1.340729243 |
| ANAPC5 | 0.032274 | 0.150999 | 0.051391216 | 0.133450456 |
| ABLIM1 | −0.17644 | 0.367444 | −0.253376802 | 0.329312755 |
| ZNF3 | 2.035715 | 0.151311 | 2.054795721 | 0.144119692 |
| GZMB | −1.05518 | 0.624452 | −0.731598423 | 0.624933093 |
| NONO | −1.62749 | 0.100734 | −1.655666216 | 0.095555294 |
| ANP32C | −1.5067 | 0.331052 | −1.562725676 | 0.329978103 |
| IRF7 | −1.30634 | 0.443322 | −1.362412838 | 0.501576497 |
| SORT1 | 0.920913 | 0.328125 | 1.019922973 | 0.317628966 |
| MYLIP | 1.088378 | 0.348307 | 1.012116441 | 0.318602651 |
| PECR | 3.488009 | 0.19168 | 3.399232658 | 0.185450566 |
| MPEG1 | −2.09217 | 0.301773 | −2.074497297 | 0.277890276 |
| VEZF1 | −0.05276 | 0.132662 | −0.073274324 | 0.131847334 |
| HDC | 1.560602 | 0.690624 | 1.625079054 | 0.738798879 |
| AK2 | 0.544261 | 0.13758 | 0.539173423 | 0.134652631 |
| CX3CR1 | −1.63698 | 0.387206 | −1.362444144 | 0.435806138 |
| NBPF11 | −3.13942 | 0.239579 | −3.21753491 | 0.225299542 |
| TMTC3 | 4.02985 | 0.206131 | 4.011082658 | 0.196227961 |
| CCNB1 | 5.382786 | 0.330228 | 5.398343694 | 0.337211144 |
| NF1 | 1.12147 | 0.211252 | 1.063884685 | 0.195504155 |
| GNASn | 3.615647 | 0.186252 | 3.554693243 | 0.161860477 |
| LDLR | 2.695296 | 0.392241 | 2.661415766 | 0.40418233 |
| HMGN3 | 1.341151 | 0.226634 | 1.373718468 | 0.221852704 |
| AGER | 0.292687 | 0.267322 | 0.208092568 | 0.249055818 |
| CPA3 | 3.98113 | 0.674483 | 3.986708559 | 0.706708609 |
| AR | 6.420052 | 0.782809 | 7.024873874 | 0.833609715 |
| CCND2 | −0.30664 | 0.25641 | −0.425374324 | 0.231373464 |
| ABCA1 | 2.431074 | 0.659182 | 2.240588739 | 0.61039595 |
| HDAC10 | 0.429343 | 0.166129 | 0.38398964 | 0.149463325 |
| CR596746 | 1.470934 | 0.223389 | 1.43511464 | 0.217549784 |
| HNRNPF | −1.16791 | 0.145766 | −1.202212162 | 0.138139227 |

### Example 5

### Biochemical Validation of Predictive Models

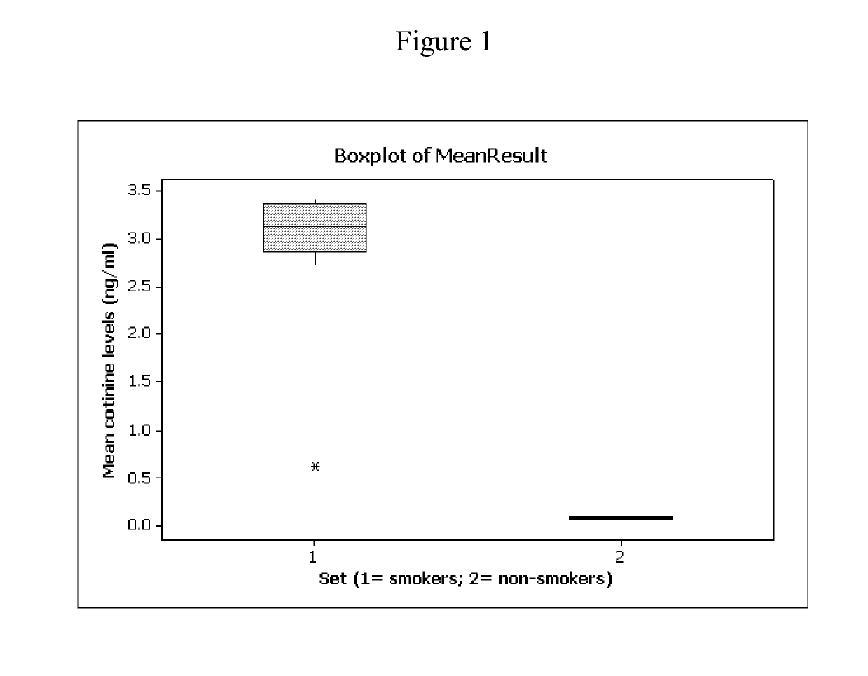

To check on the accuracy of self-reported smoking status, a biochemical method was used. Levels of cotinine, a relatively stable metabolite of nicotine, have been show to correlate with self-reported smoking status and can be easily measured by enzyme-linked immunoassays (ELISA)(10). Twenty samples were assayed in total: Ten samples from self-reported non-smokers with less than 0.3% fitted probability of smoking by gene expression, and ten samples from self-reported smokers with a greater than 99% fitted probability of smoking by gene expression. Strong concordance was seen between self-reported status and cotinine levels with the exception of 1 self-reported smoker with lower cotinine levels (FIG. 1).

Using a threshold of 10 ng/ml, cotinine levels provided an AUC of 0.89 (95% CI 0.81-0.97), a sensitivity of 0.81 and a specificity of 0.97. Moderate concordance was observed between the gene expression model and cotinine (91% agreement, 95% CI 85.97-94.83, kappa=0.53); where both methods reported positive smoking status, 85% (11) subjects were self-reported smokers, 1 had recently quit, and 1 was a former smoker.

### Incorporation by Reference Statement

All references cited in this specification, including scientific publications, published patent applications, and issued patents, are hereby incorporated in their entirety for all purposes.

### REFERENCES

1. Mathers C D, Loncar D. PLoS Med 2006, 3(11):e442
2. Centers for Disease Control and Prevention. Smoking-Attributable Mortality, Years of Potential Life Lost, and Productivity Losses—United States, 2000-2004. Morbidity and Mortality Weekly Report 2008, 57(45):1226-8
3. Sherman C B. Med Clin North Am 1992, 76(2):355-375.
4. Newcomb P A, Carbone P P. Med Clin North Am 1992, 76(2):305-331.
5. McBride P E. Med Clin North Am 1992, 76(2):333-353.
6. Charlesworth et al. BMC Medical Genomics 2010, 3:29
7. The MAQC Consortium, Nat. Biotechnol. 2006, 4(9):1151-1161.
8. Huber, P. Robust Statistics, Wiley Series in Probability and Statistics, 1981.
9. Draper, N R., Smith H. Applied Regression Analysis, Wiley Series in Probability and Statistics, 1981.
10. Vartiainen, E., J Epidemiol Community Health 2002, 56:167-170.
11. Forey, B. A., Thornton, A. J., Lee, P. N., BMC Pulm Med 2011, 11:36-96.
12. Brody, J. S., Steiling K., Annu Rev Physiol 2011, 73:437-456.

1. A computer-implemented method for scoring a sample obtained from a subject, wherein said score indicates said subject's smoking status, comprising:
obtaining a dataset associated with said sample, wherein said dataset comprises data representing quantitative expression level values for two or more of marker 1, marker 2, marker 3, marker 4, and marker 5, wherein marker 1 is CLDND1 or IL7R, wherein marker 2 is LRRN3 or CCR7, wherein marker 3 is MUC1 or FOXP3, wherein marker 4 is GOPC or MCM3, and wherein marker 5 is LEF1 or CCR7; and
determining, by a computer processor, a score via mathematical combination of the data from said dataset, wherein said score is indicative of said subject's smoking status.

2. The method of claim 1, wherein said dataset comprises data representing quantitative expression level values for marker 1, marker 2, marker 3, marker 4 and marker 5, wherein marker 1 is CLDND1, wherein marker 2 is LRRN3, wherein marker 3 is MUC1, wherein marker 4 is GOPC, and wherein marker 5 is LEF1.

3-6. (canceled)

7. The method of claim 1, further comprising, determining, the subject's risk for developing a smoking-related disease.

8. The method of claim 7, wherein said smoking-related disease is at least one of chronic obstructive pulmonary disease, cardiovascular disease, atherosclerosis, chronic bronchitis, emphysema, lung cancer, and asthma.

9. (canceled)

10. The method of claim 1, wherein the dataset further comprises data representing a clinical factor.

11. The method of claim 10, wherein the clinical factor is selected from the group consisting of: gender and hypertension.

12. (canceled)

13. The method of claim 1, wherein said mathematical combination is an algorithm based on a predictive model.

14. The method of claim 13, wherein said predictive model is selected from the group consisting of a partial least squares model, a logistic regression model, a linear regression model, a linear discriminant analysis model, a ridge regression model, and a tree-based recursive partitioning model.

15. The method of claim 1, wherein said mathematical combination is an algorithm that has the formula log(pr(Smoker)/(1−Pr(Smoker))=15.78306+0.3876*SEX−3.3368*CLDND1−3.4034*LRRN3−1.4847*MUC1+5.9209*GOPC+2.27166*LEF1, wherein SEX=1 if male, and 0 if female and Pr is probability.

16-43. (canceled)

44. A system for scoring a sample obtained from a subject, wherein said score indicates said subject's smoking status, comprising:
a storage memory for storing a dataset associated with said sample, wherein said dataset comprises data representing quantitative expression level values for two or more of marker 1, marker 2, marker 3, marker 4, and marker 5, wherein marker 1 is CLDND1 or IL7R, wherein marker 2 is LRRN3 or CCR7, wherein marker 3 is MUC1 or FOXP3, wherein marker 4 is GOPC or MCM3, and wherein marker 5 is LEF1 or CCR7; and
a processor communicatively coupled to the storage memory for determining a score via mathematical combination of the data from said dataset from said dataset, wherein said score is indicative of said subject's smoking status.

45. The system of claim 44, wherein said dataset comprises data representing quantitative expression level values for marker 1, marker 2, marker 3, marker 4 and marker 5, wherein marker 1 is CLDND1, wherein marker 2 is LRRN3, wherein marker 3 is MUC1, wherein marker 4 is GOPC, and wherein marker 5 is LEF1.

46-49. (canceled)

50. The system of claim 44, further comprising, determining the subject's risk for developing a smoking-related disease.

51. The system of claim 50, wherein said smoking-related disease is at least one of chronic obstructive pulmonary disease, cardiovascular disease, atherosclerosis, chronic bronchitis, emphysema, lung cancer, and/or asthma.

52. (canceled)

53. The system of claim 44, wherein the dataset further comprises data representing a clinical factor.

54. The system of claim 53, wherein the clinical factor is selected from the group consisting of: gender and hypertension.

55. (canceled)

56. The system of claim 44, wherein said mathematical combination is an algorithm based on a predictive model.

57. The system of claim 56, wherein said predictive model is selected from the group consisting of a partial least squares model, a logistic regression model, a linear regression model, a linear discriminant analysis model, a ridge regression model, and a tree-based recursive partitioning model.

58. The system of claim 44, wherein said mathematical combination is an algorithm that has the formula log(pr(Smoker)/(1−Pr(Smoker))=15.78306+0.3876*SEX−3.3368*CLDND1−3.4034*LRRN3−1.4847*MUC1+5.9209*GOPC+2.27166*LEF1, wherein SEX=1 if male, and 0 if female and Pr is probability.

59-65. (canceled)

66. A computer-readable storage medium storing computer-executable program code, the program code comprising:
program code for storing a dataset associated with a sample from a subject, wherein said dataset comprises data representing quantitative expression level values for two or more of marker 1, marker 2, marker 3, marker 4, and marker 5, wherein marker 1 is CLDND1 or IL7R, wherein marker 2 is LRRN3 or CCR7, wherein marker 3 is MUC1 or FOXP3, wherein marker 4 is GOPC or MCM3, and wherein marker 5 is LEF1 or CCR7; and
program code for determining a score via mathematical combination of the data from said dataset, wherein said score is indicative of said subject's smoking status.

67. The medium of claim 66, wherein said dataset comprises data representing quantitative expression level values for marker 1, marker 2, marker 3, marker 4 and marker 5, wherein marker 1 is CLDND1, wherein marker 2 is LRRN3, wherein marker 3 is MUC1, wherein marker 4 is GOPC, and wherein marker 5 is LEF1.

68-225. (canceled)


SEQUENCE LISTING (15 sequences)

SEQ ID NO: 1 - DNA, 18 bp, Artificial Sequence
  Description of Artificial Sequence Synthetic primer
gccacgggcattctccat

SEQ ID NO: 2 - DNA, 25 bp, Artificial Sequence
  Description of Artificial Sequence Synthetic primer
cataacaacttactgagcccagtgt

SEQ ID NO: 3 - DNA, 17 bp, Artificial Sequence
  Description of Artificial Sequence Synthetic reporter oligonucleotide
cacagacctgcaaggag

SEQ ID NO: 4 - DNA, 23 bp, Artificial Sequence
  Description of Artificial Sequence Synthetic primer
ctgcctctctccagaaatgaact

SEQ ID NO: 5 - DNA, 25 bp, Artificial Sequence
  Description of Artificial Sequence Synthetic primer
ctcacctaatgcaaaggttggtttc

SEQ ID NO: 6 - DNA, 20 bp, Artificial Sequence
  Description of Artificial Sequence Synthetic reporter oligonucleotide
atagctgtgtccaccatcac

SEQ ID NO: 7 - DNA, 24 bp, Artificial Sequence
  Description of Artificial Sequence Synthetic primer
caagaatgacagctgcctacatct

SEQ ID NO: 8 - DNA, 17 bp, Artificial Sequence
  Description of Artificial Sequence Synthetic primer
gctgccttggctttgca

SEQ ID NO: 9 - DNA, 19 bp, Artificial Sequence
  Description of Artificial Sequence Synthetic reporter oligonucleotide
tcgttttccaccatgtttc

SEQ ID NO: 10 - DNA, 21 bp, Artificial Sequence
  Description of Artificial Sequence Synthetic primer
cccctagcagtaccgatcgta

SEQ ID NO: 11 - DNA, 19 bp, Artificial Sequence
  Description of Artificial Sequence Synthetic primer
gctgctgccaccattacct

SEQ ID NO: 12 - DNA, 18 bp, Artificial Sequence
  Description of Artificial Sequence Synthetic reporter oligonucleotide
cccctatgagaaggtttc

SEQ ID NO: 13 - DNA, 24 bp, Artificial Sequence
  Description of Artificial Sequence Synthetic primer
gtgctagttgcaaagacacaagtg

SEQ ID NO: 14 - DNA, 24 bp, Artificial Sequence
  Description of Artificial Sequence Synthetic primer
atgtgtgtcagttactgccttctt

SEQ ID NO: 15 - DNA, 21 bp, Artificial Sequence
  Description of Artificial Sequence Synthetic reporter oligonucleotide
cttgtaatactttgatttccc